SEQUENCE LISTING (2633 sequences)

SEQ ID NO: 1 - DNA, 2643 bp, Homo sapiens
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 2 - DNA, 1332 bp, Homo sapiens
atggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctga

SEQ ID NO: 3 - DNA, 33654 bp, Homo sapiens
  misc_feature (26)..(26): “n” = any nucleotide
gggaatgagtctccatttcttctgcnatntgctgaggttgcctcctcggccagagaacataaaagtgttattcagtaactctctccttttcccttggatgtgggaaggaacgggcgcaccacctttcctccctcttccatttccatgctgcctttttgaagttgcaagagatgctaaaagaaggtccaaccccaagtgagagcctggcctttgcctggaggggtaggggcaggtggcctatatcgccagggtcctggaaaggaacctcttacccactcactcccaattcctatccaactcaagtcactccagtaggctgcttttgagatgcaaatacaaaatgctttcttctccccctctactttttcattgaaatgctttgcatctcagaaaagaggaaaaaatgatgtgtttaagacaggagtgtctcacccgccatcaagcctgataaggaattgttaaattttaattagacaatttgattccttcaagtgccgaactttcagggaaggcagcaccaagacttaagctgagacggaagcataggaaacacgttgctggggtttatgctgcatccagggcttctgtcccccacctttctagctctgtgccccagatgaaatagaaatgaggtggtggggatagagggagggagagagagagagagagagagagagagagagagagagagagagaaagagaggatcagagaaaatcctctctgttctcccaggacctgccagctttcttcctggatcccaggctgtcctaacagattgacccttttcaccccactccatcagcctggggactaagtgttgctacaccttccaagtttctcctgagagaaaatgtcaggtttgggtgtcagcctcaaactgctgagccttaaaggagcttttttcccttccaatacactttataggttggggcgnctgccagttttggggcgtgactgggaaagcagncccttccatacctcatctataccccagaaaggactgcccactccctgtcccctcctctactcctggggggcagtgcagtgggtggcctgagcagggtggcctgcctggggcagcctgggtgggctagcttagccccgggtatctgggctcttccagtacttctcaacttgagagctagctaatttccaaattagggcacgcacagaaaatagtagcatttgtaaggcatatagggtgaatagcagaagctgctgaggttggaagtgcctgcctctacatcccaattgacctaatatttctatgcaccttgatgcattctagacctctgattggggaagttccactctgaagcttctgaagagagccactattatagaaggctggagttaggctagatgttaggaagaacttgacatgcaggggtcctgagcactagaatggatgactctgaaagatatttctgtagatctttgcccttctgagctagggcagagcaaagagtcctggagacagatgaggagatctttggctctttgatgaccagtagcttgcaggtcaccacacaaatgcagtcttctctttctcctggcttccaccagggcagctgagaaagaaaagcctaggaggcctcagaggacccatggaaccttgtctgcaagcatctgcatggtgagggtggaatgaggagcagcaattacccccactttcctgcacagaaaggtgccttaggggagaggcagccacccctggagtgaggcctggcagctacaggattagattgttcatcctccacccaggaaacactgtcaagtagctcaacctgtaacagctggaaaataacagtgtccatgcatgtatttggtgctcactaagttcttgttgactggggattgtgatttggagttttgtaagcttttatgcatgcccaggggccacctgggagaaagatggagatgacatttggagggtggtgagcaagttgatcatattttcacaatcaaaaatcaaaatgcttgttttgataacaatagatttttaaatatactgtgccactttatttgtctcaaaaaaatttctcatgtaagaataatgcaccttgggttatatatttagccantctccaytttaaaaaaagaacaagaaattgggaccatcatggcaaatacaaggtgccatgtgttatgtggctttgcattttccctcaacaagctccagagtggacaaagaactcaccaggcttctttggagccagaaccagcccagcgtaactggagcagcactgagaacctttcctcactccgctaccatgaacacaagctccagccaggatggagaagctactcacttgtgtattcttttgggatgccccttggagcccaacaatctgtgcagcccggagacagaaggacaaaagggagggatgggctggagctgcagcagggaagcctggggaccagcaggttttctcaggcctcttgagaaaataccagcttagaaatgctgggaaggccctgtattgctatccactctacaatgcacttcccmctgcccccaccattttaatgtctctgaaatcagaatgcatttkacaatggatgacatcttggagctctactggggttggatggcattwttttctttcctagtgatatatraaatagtggtgcatcgtcattcgattttgtcttagagtctatgaaatgtggtgattagtatgacagtcatcgrctatagcagctgggctgggagagctgtatcttccaggccagctgctacctcatctcaagggtcacttgagtttgtcctacagccagggcccagggacaggctttgtcttttccttccwttacttcctcccaaggagttggtggctgcatcttgaaatccatctcccttcatctagccacctatttttcaggatgggccataggaggcagtgggcccaggaggacctcagaccctagtgagctgcatttgaacagcaacagtcacttcatcttgctgagccgttggcttgtggtctgattgcttgtttcatttcattcttttgcttggtttgatttttaaagttgacattctcaagggcttacctgtgcctggcacatggccatgagtcatcttacttaatactcacaattgttgttatctctattttgtgtatgggaaaactgggctcaggaaagccaaataatctggctgaggccccaaagttagtaaatggtagagwtgggacttgaacccaggttttttagtcctcaactcgaatgttatactagcacctctcaggctagagtaccttctctgtggcagtggatttgctgatgtctgaattcctgctcagcttgatgtttacatggtgagagccagctaggctgcccctgcttgtgagaggtgaaagccccaagtagataaggtgctgaagacacgatagaaaccaggactgacaccaggtgttaaaagatagccctgtactggccgggtgcggtggctcacgcctgtaatcccagcactctgggaggccgaggcaggcagataatgaggtcaggagatcgagaccatcctgactaacacggtgaaaccccgtctctactaaaaatacaaaaaaattagctggacatggtggcgggcacctgtagtcctggctactcgggaggctgaggcaggagaatggcgtgaatccaggaggcggagcttgcagtgagccgagatagccccactgcactccagcccgggtgacagagcaagactccatctcaataacaaaaaaacccccaaacaagcaaacagacaaaaagatagccctgtaccaaggtacttataggggttacaggtatttaggcatttgccatgttttcccttttttttcctatggtgtaaatatactacgtttgtattagcaacaaaaagcaacgagaacaaccaataataaataatagattagaaaatcaaatgtgtgaggcctttctcagaacttgggtacagggactctcccttaccctgcagaagtaaccagcactccagagaatgaatgctcaccctcaaccccacctgacctgcagtgctcacatttgccatctacattgtactcataattttaaatgctagactagtgcctgtaatcccagcactttgggaggccaaggcaggagtatctcttgaggccaggagtgtgagaccagcctgggcaacatagtgagacccccatctctacaaaacatttttaaaaattagccaggcatcgtggcacatgcctgtatttccagctactcaggaggctgaggtggtaggatcacttgaggccaggagttgaggctgcagtgagctatgattgtgccactgcactccagcctgggtggcaaagtgggattctgtctctattaaaaaaaaattaaatcccagtttacatcctccctcctccaggaagttctccacaaacgccccagccacacacatgtgcatatgctcctgaacatcaactgtagtggacacggtggagtgccacccagatccccctgcaggaccacagccctcatccccaactactgggagtgttggcaactgacagtttttggcaattgcccactgctgaggagagatgcctctttcaaggcctcgctgctttccctgggacagcagaagcttgtgacatctatgagaggggattaaaggccctaccctgttgccctcatttaggcaactggcagggtgccctgtggggtcacctgatgcctttcttgtgacttctccctctggcctgtcctgcttcctcacttctttgcacatcttgatcctgagagcattcccctgtacatttcctgcaggctgatttcctcttcagagtctacttctagggacctgtattagtctgctctggctgctatgacaaaattccacagattgggtggattgaacaacagaaatctgtttcctcatagttctggaggctgggacgtcccagatcaaggtgtcaacagggctgattttgcttaaggcctctctccatggctagcagatggccgccttcttgctgcattcccacttggtctctcctcttgtgcgcgcatccctgatgtctctttttatgtccaaagctcctcttataaggactccagtcagattgggttagggcccaccctaagcgtctcattttaacataatcaactctttaaaggccctgtatcctaatacagtcacattctgaggtactgggggttaggacttcaacatatacattttgtgggttgggggaagagcatagtgcagactgtaacaggacccaacatgcagcacctatggcacccttggggtgccttactttttcatcacttgttaatctggtagcctatgttagcaggtgcctttcaagtgggaaggggttttcttcccagttgcactaacagaacctttgattcagttcagcaaacatctgttgaatacgtactgcccacaaaccagagtcaagagttgggaaagaacaagatagtatggtttttatttgttcattccaagaacaagaggaggaatgagcaagacatgttgtaactcttgtgatgtctacaccccagtgaaaaaaagagactcctaaacaggtgtctcctgcagcactggggtgtgtttcagtgctgcaggaccctgaggagggtgggtgatggctcaggcaggaggatatgggtggattctaagacaggaggattctaggcaagactcactttgaaggatgcatagggactggaaaagcagagggaagggaagtgaacagaagagttttccaaatccctttcttattaaagtagggataataattagacaagatcaagcatgttccaggtagtatggccatagacagggataataattaaaagcctccgagggttgttttgaggataaatgagacagtatatccaaaaaacttagcaaactatcaaaccctttacaagtatatgatgctatcatcagcagcagggagtggggggagaagaagagagcgatggtcaggatgggggcagtaagtgtgcctcacacgcctcaggcccctgacactgctttgcacagtgctttatgcccagcaggttcaataaatgccgattgagtaaatgaatgtcagaagcagccccaggcacctgatcaatacatacatgcaggcctcccgctgtgggctccaaatgggaagagggctcagttggttttgcagaatacagcatcaatatgacatatgcttcattaccttagaacaggaagatttgctgcgtggtggcattccatgtcaccccacatggaattaggcactttgcttttccccgaggctccagagtgatggtggttggtaggacatgatccatattcttctactaaagctggaagagtgggtgtgtcagacagggatgagagtgtagagattgggcagaccctcttccaccatcctctctgacccattgctggaggtgtgccctgttcattgattggacatggcaccacattcatcccaaatggcatcaattggtggttttcaatttgcagtagcaaagtacctggaagtcatgtgctttgtatgaaacgccttggaatgctgataagtttaattctattctgtaaaagaggaagacttttgttagctgaaaagccaactatattatccatgcattatgcttcagtcacaaccaaaactccggctgtcaagttcatcaactcctgatgcctcccaagtctgctcctgagcccctctggtcccttttctctgtgaatatgcgacaccagcctcccagagccccacgcagaaatcctagcattaccctcaactccttatctccctgcttgtcccatacccacttttgccccacacctatccaaggaccaaggaccatgaaattgacttgcagtttagctcctaattctgaacaggtctctccatcctcgtttccactatcttcgttcagatgaccacaatttctaacctaaattacagccaaaacatcactctcctctgagctctcttctacaccatacactttctagaacaccagtccaatagggtcatgtctctgggcccaacctctcagggactctccagctgacaagacacaatctggcctgtaaatgtccctgcagtttaattatcctcaacattactgccataccctacatttttggccaaacagaattgtttgctgtttgatgatttgataccatgtcatattgtctgtctggaacatccccccacccctctcatttagcaggctaagtcctccttctacttcatcccagatgatacccacttcaggaagtctttcccttctgtgggatgagatgcccattctgcagggctttcactccctgcatcctgccaaacctcatcatctcttacctggattcctgctacagcctcccagttggtgtcccgcttccactctgggcccctcctctccgttctccacagtgctgtcagaatcacctattcaaaaggcgaatccgatcatgtggttcctgctgcccttaggatcatgtataaactcctagcatgacttttaaggccctctatgatcttgcctattgcaacctccccagactcaacccttgccaggtccctctgcatcagctatccagaatctctttgaggccctccacctgctgtctacctctctacctctgtgctttgcatatactgcttccaatgtctagaccttctgctgactcctagttctttacctggctaattcctacacatccttcagttgtctgtctgttgaacatcacttcctctagaaagccttccctgaatacctgaactaggttatgtatctctctccctgttccatttcctactccctatcacccttagatgtaattgcttgattaattggctgttgtttctcctagaatgtgagctacagaaccatgtatattttgttcctgcctatatttctagaactatatagaacaatgcttaataaatatttatagaatcaagaatgaatgaatacccatttctgtttcgatgactagaaggtagcaagcctggttaactgaagtgtgtggtgcatgggtcgtgcttaagaagtgtttgttgaatgaataaataaatgatggatcagctctgttcaagctcatcttattatgcatttttaaaaaattgtgtttgctcatttgtcctaccagctgtaaaaacttgctagcaaaagagtggcagaaggcccaacatttttagaaaattcttagccgtaaaccttaaaatcccgagttggaaaattctcattattaaatgccaggagttggtcttcttcctggagacctctgcaagaggccctctcactgacaccttgtgtccatttttcctggccagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtaggtgggccccctgcttgctccagcactttctccagcagggccctgcactggacactggggactctagctccccactggcttttaccaatgagtttcctggtgcttccccaggtggtttttccttcactctgggcttactttttctccttacgaaatgggtagattgtttccttaataatcccaactaccatttgtaaagtgcttactaagtgctgggcctcacgtagggactttaaatatagcattttcctatataaccctcacagcagcttagaggctggcattattgccaccattttgcagttgataaaccaggtggtcagagatgttaagtaactgctgcagcatcacacggctggcaagtccaagctggaattctggcctcagagttagttccttaggtcatattggaaataggagtaaccggcaaggatccccaaggaggggcatgtttatgctcccaggcccctgaaaccttcactcccatctccccacttcagaaatgggtctgctgctttatgctccccatatcccctctccttcccacagcttatcctggggccctgtccaggacctgcaggtagaggctgccatggactgtgctgtagcccttttgttaggaagaattgtgtagtcactcatttcttggcccagctctgcacctcaaaatggggagtaggcagcacagtctagacactgtgcctggtgcattgctgacacagacccagtgaatactcttggtaaccctgggagtccatgctactcttctcactttacaaaataggaaacaggccgagcacggtggctcacgcctgtaatcctagcactttgggaggccgaggcaggcagatcacgaggccaggagttcaagaccagcctggccaacatggtgaaaccccatctctattaaaaatgcaacaattagccaggtgtggtggcatatgcctgtaatcccagatacttgggaggctgaggcaggagaattgcttgaatctgggaggcagaggttgcagtgagctgagatcataccattgcactccagcctgggcgacagagcaagactctgtctcaaaaaaaaaataaaattaaattaaaacaaaaacaaaataggaaatagaagctttggagaagttgcctttcttcacccaggtcacagggagaaatgctgatggagaaatcccaaagcagatattattctccaggaagacaccttcagagccagcgagcagatgtgggagcatttagggatcatcggtgggcacaagctggatcctgcccctttctctgcccagctttctaggatggcatgtccaggactcaagcaatctgggagtctaggtgcatgggaaggcatgggatcaggcatttaggaaagtgccggtgctctgcccactgaaccgttaccttccttccctgagcaggagaggaattgaccagtgcctctgaggccatccctgcctgagagggaaggggttgttgaaagaaaatgagaaagctttgtaggtttaaacaggggagaaatctagatgaggacgctcaggtgaggaggcgagactggtggaaagtggcacacctccctgtccctgggcccccaaggggctcgtgcgcttactgttctcaccaccaccccgggcccaagggagctctgatccatcaccccttggcttcctttactacagccaatgcaggctgcaacttccaaaatgacctgactggatattaaggaagaaccaaaaggaaacacaaatacaaaaccacataaaaatattcagttgacacaggccaggagtccaattccccctggatccatgtttatgagggsccctagactctgtccatgccttcttaattcttacgcttgatcccctccttcttccctcttccaaaggagggtcctaggtctcagaccatagggraaaatggtagagcaaacaaactctctgtgcctcagtttcctcatctggaaaatgcagataataatagtatcatacatcatagcattgttgaggatcgaattagttgatgtatgaaaagtgattagaatgatacatggctcacagtgagcactgtgtaaatgtcagccatggcgatgatgataaagatgaagatgacaatagacatccagcactgctccacatagggaggactctgtctttcctattcaccgcccccttgggataaagaggaaggagagagtactccctatgttcgccccaggcaagtaggtaccctcttcccagaggaacgtgtgttctacccagggctctgggcatggcttctgttagcttccagaccccctgtccctacccaggcacttgggtagggacaaatccatacatgggctatagggggttcccaggaatctgtaagtctttcgaacagatcctttaaacatgcttgttcaccttattttaaaggtgaagctgagtgtgtcaggggaggagggtgcagaagccattctcagagggcagggstcagacttccccacagcatctcatcaaactagagctgggcactgagccttgtttaatggacattatctcagcgatttgttgtccctagagaacgtcccaggtgacgtctcaccctgccctgatgtcctatcagcgcaccctcacaaccacgcactgtcccctctctaatacatgctcttgtcatccagattttttcctcctgttccatttccagcaatgccatgccaacatctgtctggcttaggactaagtgtgcgttatttatcttgctaagtgtatgaaaagcacgctggttttggagtgagaggaccaggttcaattcccagctccattcctttccatctagatgctattggaagagttcctgatgtccacatgtgccattccatgtcagcctcacaaccctttaaggagcggcttcctgtgtgcatttattttctagatgaggagattgagaagatgaacaaatatctcagagttattcattcactcagtcagcactgactgagcactccgaggtcttaccccaatccttgtgcaatgggctaaggcactgacctgctggggatgcagaggttgacagggcccctacagtgcagtctgctgccggtgacagtggagcagtccccataggaggtgtggggcataaggcccagcatgagggtgtcagggaagactttcagggagaaatgatgctttcggaaaaattatgcaggagagggagggagacacttgcattctcaagaaagccacccagcagggagagggagtagctgbctggaggtatggaggagaggtggttatgtcatttgcctctgaggcttactgtctgcattctaagatataagcatcaagtgtttggaacagtgcctgacacatggtaagtccttagtattattacagttattaggacttagctgagccagctcagggcctgtactgcaggtctcagctttatgtgagcaagagcattaaggaatgatgcctggatgcctgggggtgtgaagaaaagagccttgggttcgactagggaacctggggccactccttcctctgctactaaatcaccaagtgatcttgttctgttttcttctctgaccctccctagttttgtccacccttgaaataatcatctttccttttcacatttcatgcttaccaagtacttgtcacctaattatctcctctcttgataagctagatggtyccttccagggcagcttagtagagagcatgggatgtgatgtttcagattccagctctgctgcacacctgccaggtgaacttggccacgttacatggcctctctgggcttcagttccctcacctatgagtgggataagcaagcccttcttgtaaaagttttaagaacaatacatgagataaagtgcaatgcccacagtagatgtcttatggacagtggccaccaatgcattttcttgagtctttaagttagcagccctaactactctccaaggcagcttcccctgggaccacagaggaaatctctttgttattctgggctccagataggccagtgcaaggagagatttaagatgtccatgaaggcaggactgtgtctgaattgctcacaactgtgtccaggagcctaggatagaggctggaacacagtaatttgctcaataagtgtttgtaggaaagaagggatggcagaaaggagggaaggagaggaggaagacagggaggaaggccctgatatctcaacctagatccccagtggctcccaagtactggtcccaggaggggcttcaaagtgggatgtaaatcaggtagtgctggttttctgcccagtggtaccctaaaggcacactctgtttatgccaggaaagccctttacgtaccctcctgcctccacccgcaaaatgggcatccagctgttagctcctgccaacctggtcagcgcagcagcacagggagctgggagagaggccctggtacggggcccccatgtgagctcccaggaagacagtgccgcatgagaggcttgctgtgttctggggcaagctgctgtttctgccacacaggatgacagcagctgcattgcccatcattgagcttaaatgccagcatttggttggatgcctaggagcagaagagagggcagtgatcagaatgagcaaggttataattagccctgctgccaagctacacggtgacacgaagtctgccctactccgggttgcgggagggggctttcacgctcctctctgctcctattggtagaagccaaaggtgcatctccctgtcctgactccatcctcagggaactggggctgctccaagcttccagagcgctagggacagctgccgtgagtgtgtgcctgcgtaatctgccatgagtgtgtgcctgtgtgtgaatgggtgttacgtggggaggggggagggcggtgcgttcatgtgtgtgtgtgtgtgtgtgtgtgtgtgtgtgcctgtgtgcatgtgtgtatgagaaacgtgctcctgccaggagctagactcttcatttcccatcctaccaaggacacagctcatcacagcaacccctccgcctaccccagcccactcgggcgttcctcgagcactagcccagatctgccttctttcgttctctgggtataagaaggagcagaaatttcttctctgtttcatggcgagtcttcatcttcaccattattcttaacagatcattttggggccccgactctgccatgtactgggccaaccactgaagggcttgcctggctcagccagagcctgggccagagcaggactaggaactgagcccacaggccttggggaaatcactgcactttttggaggcttggtttcctcattggttctcagtttcctcacgggcattctaaaattttacctggaatctaatttacagaggtatggctgagtgataacacagggaagccagtgccattgggaagagtttaacgtgatacacaatgtccctagaaaccagatgctcagcccgccatgtggggtgcagggggaagcagcaggcttgggtcaagagtcagaatcaggggtgagggaaagcccaggctacagccctgttggggctcccacaggaagtgcaaggcagggcagattaagcaacctagggttggctagtctcaacatcctggcaggctttgggctacaggggtgatctctagttgtctctctggtacctgagataatttgggacaggggaaatattgtcttggtgtgtgtgtgaattagagaaacggggtggctggcttgcatttgagaggcatccttccaagtcagtcattttctgtcttcaggaattaactagctctgggacggtaagtctctctctgggtctgtgaggctcccaagtgccagaatatcaggatcagagaagatagaaaaatatagtcaatataggtgtctctgtgatgaatgggtgccaaatacacaaatacagaatctaagaaaacacatggggttaacaaaggttgcagagattaaggtttaaagttgaggccctccgtataagttggctgtctttcttctagcacagtaattggcaataagtggtcttatgtatctgggagaagataagtgagggscaagggcctcagacacccatgggcatcactccccacaccccagctggatgcccacaagacttgcagctgcctcctgagtctgtggcctcatcggctccaggagtaccaggctacaggacctaagctaatctcccactcctgctgtccatccattatgttgctttgtccccaggtggtaggtgagtggaaattcagcaggattcactgtgacatcttcgtcactctggacgtcatgatgtgcacggcgagcatcctgaacttgtgtgccatcagcatcgacaggtgagcccagccagggtggaagaggttgctctggccactcaccattctggctgccccagcttttctcaaagccaggctgtgtctcgtgtctgtgatgctgtgcagctggctgtgtgcatgggggtccatgtgtttgtgtgtatgggtgtgcatgtgtgtatgtttggctaggagagcacacactccctaaagagaagccatctatggacagtgatgctgccaagcatactcagacagtgactggtacaaaaggggaaagattgtgattctgacggaagccctagctctagcacatcttactcatgtgaccttgggcagatgatggacgttttcagagctctcagtttcattttctagaaacctacattgatgacacctgtggcctggagctgcagtatctggttatgcattttatgtccttggcacatggccaagggtcccaggccatgcacaccttgctaagctgcatccttggagccttccactaatttgcacagatatgaccctacctccctcacaccattcttggaagatgaaataagttaatgratgagaagggctgggtaaaaaaaaaaatgtgatgaaggattgtagacaattgccattgttatttcctgctaactaaggcttaaatcttgctctgaggggcctggctaacaatttatcaacaaacatttccaccttatgggctcaacacaagtgcagtgggtacagtttcaaaagtgcaaagatgcaggccatggttcttcgaaaagcctgaaatccaggtgggsccggttagaataatagatatgagctaagagtaaacaatagctcaccagtgattaagtgtttgggctttgccaactgtcctcgccatctgagtttggggacaggagggagggttgagttagcatctcaggcaagcttcatagaggtgtgtcaactgtttagccccaaggagtgagggtgtctctggtgtgtgcatattgtggagtgaggggtccctgggcctgcaccccagattcagggtcccccgcccttgcaggtacacagctgtggccatgcccatgctgtacaatacgcgctacagctccaagcgccgggtcaccgtcatgatctccatcgtctgggtcctgtccttcaccatctcctgcccactcctcttcggactcaataacgcaggtacattctgcttttgtttgcctgaggctcagctggccctgggcccctgctccctcgaagggccctgagggagcagagtccctggcacagatatgggtggaggcccaatggaggcctatcactaccctggactgagtccaggctacgtgtcctgggccaagccccactcaagagttgttctaggttggcagagaggaaacagtggcccaggacacaacggagttggatgaggagtggggataatggttcaggggtatgtctgggaattttcaactttggttattaagttgaagaggcaagacgtgcttcacaaactgcatgtgtgtatatgtgcacatgaacatgggatggcggaggctactgggcttccatccactcctgacaatagtatttttgtaatgaagctgcagccctcagggcctgctaaacccaggtcaggccttagccacctggagcaggaaagctagaagtgaataggaggtaagctgaccacatgcccatttctgtgggaggcagagtcaggagtgtctcagacagaggcaacacaaagcccccgccagcgtggaacttcctctcatcagggaggcaagagcgatggcaggtgtgcacctctggagatcgtaacaacaaaagatacagtgaagccttgaaaatgtgtggttcggtatctattataagcatcacaggaattctaagtgggaataacgctaaacatacttggattaatgcacaaggccctgagatagaggacaggcgtctggtagacctagaagggcacgcaaacatatgaaacacataggaacacaagtgagttcaacagacagagccaagttatcttgctgcaaacattaaaaggtggccaacctctcccaatacacaggtcagactaaaaagatggtttactcttttaaaagttttcttgtgtcattctttctggatacatcggcttcacttgttatgcccagacatggcaaaactaatgaccaagtaatgagggaatagtaatggaaagacttgggagcagtccatcatcacagcttaactttttgctcacaaccgtgtttttaatactctggtatctgctgtgcgtttgtgtatatctaagatgaccaggcagccttaaacatctagttgcgttcatattctctgtaaaatcgctccttgttcctggaaggacatgaatgggctcttgtggaattatggccggtgggcccgctgactccctgcctgcccgggctctccctcccccagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggtctcaagacaccccccaaccaactccaagggtccccacctaaccattaccaagagggctcatcttatgctcaggtgggggcttgggaaacctcagcaagggttaggtctaggctaaaggaattcccaggagccgggcaagggcagattttgaaggcatggacctcagtggagcaatctgagtctccaggagagggaggcagggtccatgacactaaataacaaggggaagtctttgcttggagatctttgtgactgaaggcggcaactcttgcttggtacccccgtgggctcctctcttcctcttttctggtttctctgtctcactttagctccctctgactcctccatcctctttcctttaccttccgtactctctgtcctcccctccaaacacacatcacttttcctgacttcctctccactatgtctctcctgtgcttctctttcatttcccccctgatgtcttgtgaattctccccttcactcagtccttcacaaggaagacagtgtgtgggcacagaaggaacaagagctcttgggctagacgcatcaggttcagatcctgtcactgacactttttttgctgagtgaccttaggcaagtttcttaccttctatgagcctgtttcctcatctgttaaatgggaatcaaaataccagcctcacagggtggtcttgaggattccacgtgagaagggacgtgagtgtgcctagcacagtgccaggcccttagcaggtgctccataaaaaaccaggtccttgtgttcctcgtcatacccaccactttttgtctcattccagccttcccccttgccccaactgcctcctctggcccccacccttctgatctctgagcccttctgcccagtctgagttccatggactgctgcactttgggtttctgtcccccctccatccccaccactttgtgtgacccatgtgctggctcactccacagggcaactgtactcaccccgaggacatgaaactctgcaccgttatcatgaagtctaatgggagtttcccagtgaacaggcggagagtggtaagtgctcaggccaggacccagagccaggtctttctgcccctagggaagcccactggccatggttctgagacctcagaagctggccaatgggagaagcaccccagaaacccccaccttgcctcagctgaaggcagactcaccgtgcacacctccaagcaggcatgaagtgagacacctcggttctgcaaggcatggatgtgtacgagaaaatggttggccataccaacgtaataaaaatgataataatggctattcacatttctcaaacatctaccatatccctattatctcatcaaatcctcaccacgaccccgggaggtaagtctctttgatcgaaccgatcttcagttgacagaagaggaaacaggctcagaaagattaggcaactcacccgtctcaagagttggtgacactaagcccagacctgtgtgactctgaaatccacacctgtgttctttccactgacatgagctgccttatggatgggcaggttctggggtaggacgagcagagcagctgcggggactggtggcggassagtttgtgtacatagagccctcaggtgcggaagcmmagcagaccccagcctctgccaggtggtagctgtaccaacatgcaagcagcaggcattccatcctccagagggatggagaacagggccagagaacccacagagggccgcatacaaaatccaggtctggtgtcctgccttcacctgcactgcaagggcaggactctaagaagctgtttatgaggcaggtgccaaaacagagcctcagagtcagggccaaggcagcagcccagtcatgccacctaggcacatagtgaggctgcactttagaagttcagactaacacctccaaggcctcaaacaagagaacctatggaagaacccaggaggccatgagtggatccatgccagggctctctaggtcaccccagcagggaagatgtggggccccaggggtcagccttttggcacctagattagtctatccaggagaatatggagcccacgtgtgtacgcaggtgcaggtacccatgaagtgggagcactgggccccttgtttgacagggagaacaggggtgggcaccctctttgcagtcggaacatgagtttctggaggcagggccaaacatcatcagctcttgaccccagcagccactgtggcacctggcactttgctaacagtaagtgttgctcaggccatgagagacaagtccctggtattcagccctggcagaacagaagtggggtattgaggctgcatgaggattgccatgggaaaaaggacaggggcaatcctgcaggggctgccatgggtcctgggtcccatgcctcagtgacatccttgcctccctggcagggtgaccctgtggtgtttgcaggagtcttcagagggtgaaagggaggggccagtgagatgggtggctgatgcctgggaacttgtccggctttacccagagccctctgcctctggtgcaggaggctgcccggcgagcccaggagctggagatggagatgctctccagcaccagcccacccgagaggacccggtacagccccatcccacccagccaccaccagctgactctccccgacccgtcccaccayggtctccacagcactccygacagccccgccaaaccagagaagaatgggcatgccaaagaccaccccaagattgccaagatctttgagatccagaccatgcccaatggcaaarcccggacctccctcaagaccatgagccgtaggaagctctcccagcagaaggagaagaaagccactcagatgctcgccattgttctcggtgagtcggccctggctgctggccacagcccggtctgtgaaaggtcccattccctgccatgtccttcctaaccatggctgcaggatcatggggntagcatttcctcaggcacaggccaagcccattgacatacagacagccctattaggtagattctgtaattgtgctcactttacagatgggcaaacagagttttagagtggttataaaacctgaccaagaggacccatggtttgaactcaggcagtctgactccagagtctggaaatttgactagtctattatgctggcaccagaacctcatgggcctatgtgcccaggaaagtcactttctctctctaggctcctcatctgtagagtaggttacttctcagggctgtcatggcaatcagttggtgaaagcattttctaaactacaaagcactatccatatgtaagtgtcactattatggtttttattactatggctctttttgaggaattgggaaattcagttcacttgcactcaaacaagactccatgggaccagagctgtgaaaaataaatgagatgttggggagtggcccccaatctaaatgaaaagcatccctgtccataaagcaggatttgtcaatagaagtctagaaaaccacatagtaaaggtcaaaacccaaaaggtggactgatatgggtggctgggaaaaggagggtcccctgtgactctgtcttccaggaagcacctgaatcccctttgactgccggatgatcctggggcgtgagtggggcagctgttacctcccttcttttatgaatgagagcacttgatcccatgggactactcacgatcatgggactgggatatggtaggctaggattggagccaggccctctgctgcagtccataggggctcagctctgtggccctactcttcccagacaccattcagtctccagggatgcctcctgtgcctgtagggcattccatgtacacattgcttccaggctaaatatcacacctgtgcaggaggtcctggaaaaagtgcacctgagttaggtgagcagagcaggtaggtggctgccctgggtgagagggcatggttcaaggcagggactagcaagggtgtacccaccaagactggggaaattgggggaaggctagagcgagatcatctggggacctggatagaccggagttcagctctcagccttgctgctcaagagtgaatgaccttgggtatattgcagagctgttgtgagtgtttttcttatgtaccttatgaggatgctataagaactaagtggcatgggtctatagccctcaaagatgacctggaagtagagtagatatttccatttctactggctaacctgggtcagaatggtaaccttttggatttattgttaaaccatgtacccttttctaggaggtgggaaagggacaaatgagggaagtgaggctgaaggagctgaagggatacttcactgcaaatgggtgtcagaggcaggtctaggatccaagaccgggactcctcctacagtgcttctgtggctacagcccaccgtcttggcatacgagccagggcgcactgggtgtgggtgttcccagccgtgcctccccggctctggggaccagcctgaccatgccctctcccccaggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccrgccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccagscaacttcagtcctgggagacccatgtaaataccagactgcaggttggaccccagagattcccaagscaaaaaccttagctccctcccrcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtagaaggcagcagtgccacttctgtgcttggcattcaagtataggaagacccctgtgtctgcagggtctaacccaagggaagccaggttgcccccactgntccacctcccctgttgcagctcctgcttcctctgaaggactcatcctttgccctcttacccaccagggcagagaaggctctgtggaaaaggtggccttggatgcactggcattgcctgtgtctgcctatgtcccttgncctgtcttctgtcccatgtcaggatccccttcctctagggcaggctgggagaagcagggaaggccctgaccactgcggcctggacagttctccctcctctcagcttccagggcggtcccaagctccaagccttccgggggaaaaacttggtactgccccaacaacagaaacttggctttctacaaatgaagcgtaaatcancccagtgagggaggaatattcttaccaccttgagaataaccgcagtgatgacaaacaaggtgccagcacccacgggctcaggcgctggggagctgtcagggcgtaatttgcatgctaaatacaatatttttagcaccaaagtttggagcacttaacttgccctgaacagttaattatggactttgatcttctccttaaacctaaaggtagcactaagccctgggagaggctcctgtccccaggagcaccctgattctggaaagtgagcaaaacaggcccctagtctaactcggactgggtcataacaccaaggacccagtgaccatctcctctggaaagcatcaggtccccaaggggtctagaagccccagggacccaacccatccccattgnacacataccatgctcaatgtctgtgaaagatcttggcctggatggacgcttaaaagtatatcccacaattaggaatcttatgagggtatacaggcttatcagatgtgangattggaagagatgacaaagagaagcagaggaaagaagaaggaagggagggagggagagagggagggacggaagggagacccagagcagagtgagaagagcattgacagggagcagaggggaagagggcagngcaggggcggnaggcggtgcaggggaaagttgcccacagttgtcacgaggcttcatgtctttctgccagacagcagattgacagctagagtgggcaggggagggctgggctccaccctctccccccctcagcacttcaggggcaaagaaatgggaggagtaggacccgacacgacacgggaacacaaggtggaagggggtggcccaggctctgactctctccagagaggtcctgacgatggtctcttgctcttgacgacaggatggaaaggaagcctccagttttcactcctctttgcctttccctaggtttctgtctgtgnctgtgccgctctgtagagtgccttttaatcaaaggatcattcatttgcctttacagcaggtagagtctgcacccttgtcccctgccctgccctttcctagagcacagcccaggatcaccacctaagggccaggcacagtggggcactttgcatatgtcgtcttagatgggaatggaaatcacacagtcacaaaggagcagacccagaggtaactgcaagtcttcaaggcttcaggccctcgttcttcctcctgggtcctgaaggactctcaggtggccctgggctggggaaagttcctgggaattagaagacgagttgtctagcagactaagaagttgcattgcttcgtccacccatctgctgtcttccttagggaatatttattgagcaattcttgtgctccaggccctgggtgaggtgctgggattaaaacagtcaacaatggtcaccggccctgccttcgtgagtccagtctagtgggatcacaacaaagtgagactctgcttcccccaaggcagagcccagggacggcagacacagcacaggcacatgctgcgcacttccaaggcacctttctcctagatgacgtgtgacacagcagccctggtgtgccttggccttgaacaattaggtagggcaccagtaggggcaggaagactgacagcatggaccactacttcctgcgtggctggggcaggaggccctgaaagaagcagctacagatcctgctttccaggtggtctcaccacaggcacagccagttgcctgcaactaagaacactgaagcccggcctgctttgggggcatttccagcatcccctcctatctggaatctcttcccaaaacatccctgtccccagggactgcatagctcctttcacctggtgggccagccctctctgtgtgccactggctccctgccntcctgggtcccaggacctgggctgagtgtgcaactttagcttccatctgcacagggccctctctgcgggctttgccctccctgatggcatgttcttctcctaacccactggagtgaggggcattcttatcgtctccacttacagaagagaagcctctcatacagaaagggtagtttccctttcaggctaaatcttctgattttacctcttatagtgtccattccagccctggcttctgaccagcgtgagtgcagctgaaagaggctaggatggtcagaagattttttttaaaagagaaagaatacttgttttggaccagacctgtagggtaacagcctggggtgcatgtaaacagaacttgggcctctccagggagcaagtgggtaggtggtgggggcagagcagttattagtggggcttcaggagtgagagggccagtctgggcatcggtgtgccagagggtcctctcctgccaaccaggtggacctggctctgctacaytagtttytgggggtgcaggggaagcaggaagctggtggggggagagtggaccatctttggagtagggagacctatcctggctttgtggggttagaggggacgtgggaagggggtacctggtaggaacaccttcctgcttcctctctcatcacagttctgctacttgscaaataaggcctcattggagcagaggcccagagaggcgcaggagagggaatggcaaggagggatgtggagagacctttcactcacagctggagccagagacaaccccaagttcataggaagtgctgtgtgaccgctccacttgcatgtggctttctcccactttcacatctggcttctgacacccagagctgcgtgtgaggccagggagagggggccacctccctggcaccccaggnccaggggtgtcagagctgcacaggaagtgaaggaatcctatcaggaaggggtgaggtggtcccattccaggaatgggagggcaggcctgggccaccgactgtgcagagacaaagctggccatccataccttctggattttcctgatcaggcccaactccaaacactctgtcctgttncccccaggagtgtgtaagttgggagggtcatttgggggccaggctctgtnctgcgtccagggcaggggaaagcatgggaggggcaagagaggcaggacactcccctggagaagacaagtcccttgttggttttctcccagggctgacagcacagacctgacccaggctctgggtagaggaggcatgactggctagtggggctagcctttgccctataactgacctgatccgtaggtgtttcccaggggncctcggtgtctcagcctacaccaaggcactgaaggagagtctccctttcctttcctgaatgtatcggcaaagaaaaatgggcagaggcttctaagagcatccttgcaaaaggctcccaagggatgggctgtctatgtcttattgcaagagaaacattcctctgatttaggaggaactgcaagaaatgaatgtgttgagtgcttattaactgccttgccctctgactggcagggcatcaggtttaattcttacaatgacccagagataaacattattgctgctagatgataccatcaagctaagatagttttagcaacctgagatctncaactanaaatggctcagcgaagacatgcacccaggtctgcctgcccgagtccactagacccagccacccgcagtttccctcnccgacctcagcccagcctggttcctgccctctgtccctaacattaccttacaccaaaaacccctgctgcctgtaatccgattcagcaagcagggaggaagcaccctctgagctccagacaccagggcactaagagaggactggatggaacacagaaacaagtagggcgtggtgatgtggcatgcaatgagcaaggaaggtggacccaagagaagctctgtcaacctcactgcactgggcatcgggacagaatgcccctgaggaaggagtgagcatctccagggggcaagcagagacgtcttcgtgggcaggaacaggcaagttggatctggaagggcagatcggatcccggcaggcaccggtggaagaagaggacacagcattagcaggaagaagggcctggggttggtgggtgctgcagcaatgctggctgggtttcagagtgggctnggagccctaactcactggaggccctcacttattggtgagatgtgttctataggtaagcacagggggnatcaggaaaggaatgttcctgaacacaagatcaccttggagctgycatctgtgtgttcaggaagtcctggggntccctccttcctctcctwcttgccacaggcctggagccctgtttcctccccttctcagacctgcccattatcattctcttcctggggctcagtgncctcaggttggctttggagagggacacggtctccccagtctcctcatttcccgtaggccttttcagccgngaggagttttctgctgatccctgcagtcctgcctcccttcaagacagtcgcggcagnctgtttaggcctctgccctacactttgntgtggagacagnttgmcaagnctccctgytcctttaggattcccagcctcataactggagggttccaccaacagcttccatctcccagcacaagaagggatccaggtggaacaatctcagctatctgcatctctggggatggctaggctaggggatgccatctcccaggacccaggaatgtcctccttgaggtgggctggattggatcaagcacagtttaggatgggaaaggaggctgtcatatgagtaaagggtaatgaaggggttacctgagtaacactggtgagaaaggacaggaacagaaagctggaagaggagagaagtgatggtgagtaccaatgtgtgctggagataggcgtgatctaattgcatccccaagataacaaatctgttataaatacagatgccgaaatctggaaagtctgggtaaagtccacagctagcaaaaggcagaattgggatgccaagccaggtgtgccttgctctgaagccccatagggcagggtccccatctcccaccactgtcccccatttcctcatcctgcacacagatgaagaggtctttaggatgcatggngtgttctgattgnatcctccttctgctgggaaactgacaggaggtcccaactgtccctcatcctgcatgggagntcccaaatcagaatgaacattaaggtctctccattgaatacgcacctcccacacctgcacctcaacagnactgtccctccttcagntgggcactctctgctccccatagcagntgtgtntntttcacattccccagccaatcctctccccttntatccagtccaggctttgctcctcttntgctctgtccttgtcctcattcaacatatgcatcttccaggaaaatctttcctgatcgaccccacgcaacatggcaactgcctggcggaggcaagacaggtatagactgtgctgtccactgtccccatgtggctagatacccacatttaatttattgaaataaaaaaacttgattccttggttacactagccacatttcaagtgctcaggagcatctgtggctggcggctatcctactgagcagtgtggatgtagaaggcttccatcctcccagtgctgatccagaatggctcctaagttaggagtttggataccttcaactaaaatagggtataaagggtgaagtcaggttctccctccaagtggagatgcccagtaagaaattggggagctctttccccccgatccccaatctcccctaaatagggcttgaacttctactggagaccctctccttgggaatttcaagggcctcattcaaacccaattttaaacatccctagggaagctctagaaggcagctgtgttgaaatgggactcaaggtggataccgtgtgtgtttgtgtgtgtgtctgtgtctggctaggagtggcttgatagggcagaagaagacactggtaggcaatgaagggccatgttgtccagggctggcctgaagtcacctctaagagttatctcttagaggaaggaagtggaggaagcaggggtgtgagaatctctgcctagactcaggatcaccagagctgaaatgattcccagggacagtcctgtccacatctccacagcatccctgccattcattggacactaggtgcaaaaggttcatctactttttctaactcttttcttcaaggccttttctcagttctcagctctttccaataggatcagccagccactcttctccctctttggttccctcctgatcactcccactgatggatctgagcacccttggctggaagtatgtagccccagagccttctctggctctcatagagtcctagatttggagttagatctggaagggcagataggatcctggcagacactggtgggagacttcagagtgcaaagagaagcaaacagccctgtgcctcctccacttccctcctcttgctcccgcatgttctgggaaagcttagaactcctaggggaatctttaactacaagttttgcctaaggcttagtctcagttgagactaaagagatttgagcctgggcagctagcaggrggagtagggaagatggaacagtcaggtggagtgtcccctggaaacccagttcgtcagggagtccaaatgggaagaggaaaaaggacttctggacaacccaagctgctgggccaactgatgtctgcaaggttatggctaaggaagggcacagagagaaaccatgggatgctccagtgcttctcagccctgggtgctcttcagaatcacttgggaattgttaatgatgctcacaccaaggtcctaccctggaggttctggtttaattggttgagggtagggccaggcgttggtatattttaaaagccccttgcccaggagattttaacgtgcaggctcggtttataaccatggctctaggacacactccagcttcctgggctcacatctgactttccatggtggccataagctatggcaaggtggtggtaaagggcccagggcccaacaggctggagctgtggtccagaggccaaaggatggaggacacaaagctgctagctctactgaaagggacttgttaattcaacaggattgcacgtgggcaacacattttcntctgccccacctctctcaccccatgctgatgccaggctgctagctgctgtccccatgcccagactggagtgcccagcccaggagaatgacacgcagaggtgggcctgccaatcagcttggctgagagctggccaatagttgggacagatgattccttttctctgacaattgcaaaacatccttgagtgggaggagctgcttctactatgctgaccagcagtgtagggagacagaaggagcaggataggaccccaggaaactcatcctgtccctactccctccaaattgttggctgctctcctggaagctgaactagggccctggtcacataaagttagtgtgaacagctccaagccacaaccagcaggacaggacaggacagctgatgggtggcctgggtgtggtcacaaaagtgagccctgcaagctccatactgagtccagacaacccacccctcaccatcatcaccccctccaactccctctgctgcctctgcctcctgtctttatgtgaactcacacacacatatgaacacacatataccctacattagacaaatcgcatacacacacaagcacaccatactaacgctgctggcctctatatgcaacttggttccacaggccaatccattctctaaacaggagcctcaggagcctcagctttcttgacttcaggagtttgaaatctggctgtaaaactggacacgatgtaggatgaagggtggtgctggggaccagggacctcacatctcacatagggtgacttatccacagttggagtgcacccttcctatatcctcaaaatgccatgagggagacccaggcattgggaccaggggcactggattaggtgtggtcagtgtggcaacagatggctggctgggactttagtgagcagaaacctcctttgaacttttagcttctgccccttgagcaaagcacatgtgtctggctctccccgccccagcctgaggtaaggagccaagagcccaagacgtgagatgggaggatcctgcttccctcaccgctgtgcctccctccatctccagagggggtctgctaggatgcaagggcccctgtgaactcagaagcgagtggcaaaaggtggtaggtgtcttggattggagagggtattcatttggggagcttgcatccccataaaggaggaggaggaggcctggggtctgttggaagtgactggaggatgtgttgaaggcagctctccaagccagagccccttcctcagctacagccacagtgatacctctccccatgcccccatcacccaggttcctgtctcctctcaaggtggaaggggcaggtgaagactgcagacagggaagatgccctgccagaagcccagctgcactttcaccaattcntactccatccaggcggagaggccccaagtagtctaaatttctttctttctttctttttnatatggagtctcgctctgttgcccaggctggagtgcagtggtgcgatctcggctcactgcaacctctgcctcctgggttcaaggaattctcctgcctcagcctccctggtagttgggattacaggcacgtgccaccatacccagctaaattttgtatttttagcagagacagggttttgccatgttggccaggctggcctcaaactcttgatctcaggtgatctgcctgcctcagcctcccaaagtgctgggattacagacgtgagccaccacggctggccaagttgtctaaatttccatctcggctcctggcttagaaccacccagagtggccactgacggctccttgccctctaggaaggacatgatgccctgctttcggctgcggagggccagttgcaggggtgtgcagctcactccatcctggacgtccagctgggcgcctgcctcgaccagcactttgaggatggctgtgttgcccttgagggcggccaggtgggcgggtgtccagcccaccttgttgcgggcgtggacatttgcgtgatgttctakgaggttgatgacactcaggaaggtgctcctctggaccgccaggtggaggggtgtccagcctgactgctctgcagcattggggtcagccccacactgcagcagtgctgacaccaccgcctcctccccgtggcgtgcagctaggtgcaggggagtccagttcacagctccaagagcacccatgtttgcgtggctctctgccagcagatggatgatctccargtggcccttgtangctgctagatgcaggggtgtccaaccctggtgggtgggcagctcaagctggctccgtacctgagcacatcttgcagatcaggtatttgcccctggcagctgcagtgtgcagtgggccatagccgctctggtcaagggcatcagggaccgctccactcttcagcaggtgttggatggccctcactttgccccgctctactgccaggtgcagtggtgttctcaggtttctctgctgancatccaactcagccccctggctggtcagcatcttgaccaggctaacatggccaaagtaggcggccacatggaggggggtcttgccctcagcctcacgcaggttggggtcagcctgacgggagaccagaagccgtgccacattctcaaagttattctgtgcagccagntgaagangggtccacccttcacgttcctgggcatccacacaggccccgtggtccaagaacangcgcgcantgcggtcatccccattctgggctgcaaantgcantggggcc

SEQ ID NO: 4 - DNA, 20 bp, Homo sapiens
atggtctccacagcactctc

SEQ ID NO: 5 - DNA, 20 bp, Homo sapiens
atggtctccacagcactctt

SEQ ID NO: 6 - DNA, 20 bp, Homo sapiens
cattgggcatggtctggatc

SEQ ID NO: 7 - protein, 443 aa, Homo sapiens
  MISC_FEATURE (351)..(351): “n” = any nucleotide
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKXRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 8 - DNA, 20 bp, Homo sapiens
ggcagccgtccggggccgcc

SEQ ID NO: 9 - DNA, 20 bp, Homo sapiens
gcagccgtccggggccgcca

SEQ ID NO: 10 - DNA, 20 bp, Homo sapiens
cagccgtccggggccgccac

SEQ ID NO: 11 - DNA, 20 bp, Homo sapiens
agccgtccggggccgccact

SEQ ID NO: 12 - DNA, 20 bp, Homo sapiens
gccgtccggggccgccactc

SEQ ID NO: 13 - DNA, 20 bp, Homo sapiens
ccgtccggggccgccactct

SEQ ID NO: 14 - DNA, 20 bp, Homo sapiens
cgtccggggccgccactctc

SEQ ID NO: 15 - DNA, 20 bp, Homo sapiens
gtccggggccgccactctcc

SEQ ID NO: 16 - DNA, 20 bp, Homo sapiens
tccggggccgccactctcct

SEQ ID NO: 17 - DNA, 20 bp, Homo sapiens
ccggggccgccactctcctc

SEQ ID NO: 18 - DNA, 20 bp, Homo sapiens
cggggccgccactctcctcg

SEQ ID NO: 19 - DNA, 20 bp, Homo sapiens
ggggccgccactctcctcgg

SEQ ID NO: 20 - DNA, 20 bp, Homo sapiens
gggccgccactctcctcggc

SEQ ID NO: 21 - DNA, 20 bp, Homo sapiens
ggccgccactctcctcggcc

SEQ ID NO: 22 - DNA, 20 bp, Homo sapiens
gccgccactctcctcggccg

SEQ ID NO: 23 - DNA, 20 bp, Homo sapiens
ccgccactctcctcggccgg

SEQ ID NO: 24 - DNA, 20 bp, Homo sapiens
gccactctcctcggccggtc

SEQ ID NO: 25 - DNA, 20 bp, Homo sapiens
ccactctcctcggccggtcc

SEQ ID NO: 26 - DNA, 20 bp, Homo sapiens
cactctcctcggccggtccc

SEQ ID NO: 27 - DNA, 20 bp, Homo sapiens
actctcctcggccggtccct

SEQ ID NO: 28 - DNA, 20 bp, Homo sapiens
ctctcctcggccggtccctg

SEQ ID NO: 29 - DNA, 20 bp, Homo sapiens
tctcctcggccggtccctgg

SEQ ID NO: 30 - DNA, 20 bp, Homo sapiens
ctcctcggccggtccctggc

SEQ ID NO: 31 - DNA, 20 bp, Homo sapiens
tcctcggccggtccctggct

SEQ ID NO: 32 - DNA, 20 bp, Homo sapiens
cctcggccggtccctggctc

SEQ ID NO: 33 - DNA, 20 bp, Homo sapiens
ctcggccggtccctggctcc

SEQ ID NO: 34 - DNA, 20 bp, Homo sapiens
tcggccggtccctggctccc

SEQ ID NO: 35 - DNA, 20 bp, Homo sapiens
cggccggtccctggctcccg

SEQ ID NO: 36 - DNA, 20 bp, Homo sapiens
ggccggtccctggctcccgg

SEQ ID NO: 37 - DNA, 20 bp, Homo sapiens
gccggtccctggctcccgga

SEQ ID NO: 38 - DNA, 20 bp, Homo sapiens
ccggtccctggctcccggag

SEQ ID NO: 39 - DNA, 20 bp, Homo sapiens
cggtccctggctcccggagg

SEQ ID NO: 40 - DNA, 20 bp, Homo sapiens
ggtccctggctcccggaggc

SEQ ID NO: 41 - DNA, 20 bp, Homo sapiens
gtccctggctcccggaggcg

SEQ ID NO: 42 - DNA, 20 bp, Homo sapiens
tccctggctcccggaggcgg

SEQ ID NO: 43 - DNA, 20 bp, Homo sapiens
ccctggctcccggaggcggc

SEQ ID NO: 44 - DNA, 20 bp, Homo sapiens
cctggctcccggaggcggcc

SEQ ID NO: 45 - DNA, 20 bp, Homo sapiens
ctggctcccggaggcggccg

SEQ ID NO: 46 - DNA, 20 bp, Homo sapiens
tggctcccggaggcggccgc

SEQ ID NO: 47 - DNA, 20 bp, Homo sapiens
ggctcccggaggcggccgcg

SEQ ID NO: 48 - DNA, 20 bp, Homo sapiens
gctcccggaggcggccgcgc

SEQ ID NO: 49 - DNA, 20 bp, Homo sapiens
ctcccggaggcggccgcgcg

SEQ ID NO: 50 - DNA, 20 bp, Homo sapiens
tcccggaggcggccgcgcgt

SEQ ID NO: 51 - DNA, 20 bp, Homo sapiens
cccggaggcggccgcgcgtg

SEQ ID NO: 52 - DNA, 20 bp, Homo sapiens
ccggaggcggccgcgcgtgg

SEQ ID NO: 53 - DNA, 20 bp, Homo sapiens
cggaggcggccgcgcgtgga

SEQ ID NO: 54 - DNA, 20 bp, Homo sapiens
ggaggcggccgcgcgtggat

SEQ ID NO: 55 - DNA, 20 bp, Homo sapiens
gaggcggccgcgcgtggatg

SEQ ID NO: 56 - DNA, 20 bp, Homo sapiens
aggcggccgcgcgtggatgc

SEQ ID NO: 57 - DNA, 20 bp, Homo sapiens
ggcggccgcgcgtggatgcg

SEQ ID NO: 58 - DNA, 20 bp, Homo sapiens
gcggccgcgcgtggatgcgg

SEQ ID NO: 59 - DNA, 20 bp, Homo sapiens
cggccgcgcgtggatgcggc

SEQ ID NO: 60 - DNA, 20 bp, Homo sapiens
ggccgcgcgtggatgcggcg

SEQ ID NO: 61 - DNA, 20 bp, Homo sapiens
gccgcgcgtggatgcggcgg

SEQ ID NO: 62 - DNA, 20 bp, Homo sapiens
ccgcgcgtggatgcggcggg

SEQ ID NO: 63 - DNA, 20 bp, Homo sapiens
cgcgcgtggatgcggcggga

SEQ ID NO: 64 - DNA, 20 bp, Homo sapiens
gcgcgtggatgcggcgggag

SEQ ID NO: 65 - DNA, 20 bp, Homo sapiens
cgcgtggatgcggcgggagc

SEQ ID NO: 66 - DNA, 20 bp, Homo sapiens
gcgtggatgcggcgggagct

SEQ ID NO: 67 - DNA, 20 bp, Homo sapiens
cgtggatgcggcgggagctg

SEQ ID NO: 68 - DNA, 20 bp, Homo sapiens
gtggatgcggcgggagctgg

SEQ ID NO: 69 - DNA, 20 bp, Homo sapiens
tggatgcggcgggagctgga

SEQ ID NO: 70 - DNA, 20 bp, Homo sapiens
ggatgcggcgggagctggaa

SEQ ID NO: 71 - DNA, 20 bp, Homo sapiens
gatgcggcgggagctggaag

SEQ ID NO: 72 - DNA, 20 bp, Homo sapiens
atgcggcgggagctggaagc

SEQ ID NO: 73 - DNA, 20 bp, Homo sapiens
tgcggcgggagctggaagcc

SEQ ID NO: 74 - DNA, 20 bp, Homo sapiens
gcggcgggagctggaagcct

SEQ ID NO: 75 - DNA, 20 bp, Homo sapiens
cggcgggagctggaagcctc

SEQ ID NO: 76 - DNA, 20 bp, Homo sapiens
ggcgggagctggaagcctca

SEQ ID NO: 77 - DNA, 20 bp, Homo sapiens
gcgggagctggaagcctcaa

SEQ ID NO: 78 - DNA, 20 bp, Homo sapiens
cgggagctggaagcctcaag

SEQ ID NO: 79 - DNA, 20 bp, Homo sapiens
gggagctggaagcctcaagc

SEQ ID NO: 80 - DNA, 20 bp, Homo sapiens
ggagctggaagcctcaagca

SEQ ID NO: 81 - DNA, 20 bp, Homo sapiens
gagctggaagcctcaagcag

SEQ ID NO: 82 - DNA, 20 bp, Homo sapiens
agctggaagcctcaagcagc

SEQ ID NO: 83 - DNA, 20 bp, Homo sapiens
gctggaagcctcaagcagcc

SEQ ID NO: 84 - DNA, 20 bp, Homo sapiens
ctggaagcctcaagcagccg

SEQ ID NO: 85 - DNA, 20 bp, Homo sapiens
tggaagcctcaagcagccgg

SEQ ID NO: 86 - DNA, 20 bp, Homo sapiens
ggaagcctcaagcagccggc

SEQ ID NO: 87 - DNA, 20 bp, Homo sapiens
gaagcctcaagcagccggcg

SEQ ID NO: 88 - DNA, 20 bp, Homo sapiens
aagcctcaagcagccggcgc

SEQ ID NO: 89 - DNA, 20 bp, Homo sapiens
agcctcaagcagccggcgcc

SEQ ID NO: 90 - DNA, 20 bp, Homo sapiens
gcctcaagcagccggcgccg

SEQ ID NO: 91 - DNA, 20 bp, Homo sapiens
cctcaagcagccggcgccgt

SEQ ID NO: 92 - DNA, 20 bp, Homo sapiens
ctcaagcagccggcgccgtc

SEQ ID NO: 93 - DNA, 20 bp, Homo sapiens
tcaagcagccggcgccgtct

SEQ ID NO: 94 - DNA, 20 bp, Homo sapiens
caagcagccggcgccgtctc

SEQ ID NO: 95 - DNA, 20 bp, Homo sapiens
aagcagccggcgccgtctct

SEQ ID NO: 96 - DNA, 20 bp, Homo sapiens
agcagccggcgccgtctctg

SEQ ID NO: 97 - DNA, 20 bp, Homo sapiens
gcagccggcgccgtctctgc

SEQ ID NO: 98 - DNA, 20 bp, Homo sapiens
cagccggcgccgtctctgcc

SEQ ID NO: 99 - DNA, 20 bp, Homo sapiens
agccggcgccgtctctgccc

SEQ ID NO: 100 - DNA, 20 bp, Homo sapiens
gccggcgccgtctctgcccc

SEQ ID NO: 101 - DNA, 20 bp, Homo sapiens
ccggcgccgtctctgccccg

SEQ ID NO: 102 - DNA, 20 bp, Homo sapiens
cggcgccgtctctgccccgg

SEQ ID NO: 103 - DNA, 20 bp, Homo sapiens
ggcgccgtctctgccccggg

SEQ ID NO: 104 - DNA, 20 bp, Homo sapiens
gcgccgtctctgccccgggg

SEQ ID NO: 105 - DNA, 20 bp, Homo sapiens
cgccgtctctgccccggggc

SEQ ID NO: 106 - DNA, 20 bp, Homo sapiens
gccgtctctgccccggggcg

SEQ ID NO: 107 - DNA, 20 bp, Homo sapiens
ccgtctctgccccggggcgc

SEQ ID NO: 108 - DNA, 20 bp, Homo sapiens
cgtctctgccccggggcgcc

SEQ ID NO: 109 - DNA, 20 bp, Homo sapiens
gtctctgccccggggcgccc

SEQ ID NO: 110 - DNA, 20 bp, Homo sapiens
tctctgccccggggcgccct

SEQ ID NO: 111 - DNA, 20 bp, Homo sapiens
ctctgccccggggcgcccta

SEQ ID NO: 112 - DNA, 20 bp, Homo sapiens
tctgccccggggcgccctat

SEQ ID NO: 113 - DNA, 20 bp, Homo sapiens
ctgccccggggcgccctatg

SEQ ID NO: 114 - DNA, 20 bp, Homo sapiens
tgccccggggcgccctatgg

SEQ ID NO: 115 - DNA, 20 bp, Homo sapiens
gccccggggcgccctatggc

SEQ ID NO: 116 - DNA, 20 bp, Homo sapiens
ccccggggcgccctatggct

SEQ ID NO: 117 - DNA, 20 bp, Homo sapiens
cccggggcgccctatggctt

SEQ ID NO: 118 - DNA, 20 bp, Homo sapiens
ccggggcgccctatggcttg

SEQ ID NO: 119 - DNA, 20 bp, Homo sapiens
cggggcgccctatggcttga

SEQ ID NO: 120 - DNA, 20 bp, Homo sapiens
ggggcgccctatggcttgaa

SEQ ID NO: 121 - DNA, 20 bp, Homo sapiens
gggcgccctatggcttgaag

SEQ ID NO: 122 - DNA, 20 bp, Homo sapiens
ggcgccctatggcttgaaga

SEQ ID NO: 123 - DNA, 20 bp, Homo sapiens
gcgccctatggcttgaagag

SEQ ID NO: 124 - DNA, 20 bp, Homo sapiens
cgccctatggcttgaagagc

SEQ ID NO: 125 - DNA, 20 bp, Homo sapiens
gccctatggcttgaagagcc

SEQ ID NO: 126 - DNA, 20 bp, Homo sapiens
ccctatggcttgaagagcct

SEQ ID NO: 127 - DNA, 20 bp, Homo sapiens
cctatggcttgaagagcctg

SEQ ID NO: 128 - DNA, 20 bp, Homo sapiens
ctatggcttgaagagcctgg

SEQ ID NO: 129 - DNA, 20 bp, Homo sapiens
tatggcttgaagagcctggc

SEQ ID NO: 130 - DNA, 20 bp, Homo sapiens
atggcttgaagagcctggcc

SEQ ID NO: 131 - DNA, 20 bp, Homo sapiens
tggcttgaagagcctggcca

SEQ ID NO: 132 - DNA, 20 bp, Homo sapiens
ggcttgaagagcctggccac

SEQ ID NO: 133 - DNA, 20 bp, Homo sapiens
gcttgaagagcctggccacc

SEQ ID NO: 134 - DNA, 20 bp, Homo sapiens
cttgaagagcctggccaccc

SEQ ID NO: 135 - DNA, 20 bp, Homo sapiens
ttgaagagcctggccaccca

SEQ ID NO: 136 - DNA, 20 bp, Homo sapiens
tgaagagcctggccacccag

SEQ ID NO: 137 - DNA, 20 bp, Homo sapiens
gaagagcctggccacccagt

SEQ ID NO: 138 - DNA, 20 bp, Homo sapiens
aagagcctggccacccagtg

SEQ ID NO: 139 - DNA, 20 bp, Homo sapiens
agagcctggccacccagtgg

SEQ ID NO: 140 - DNA, 20 bp, Homo sapiens
gagcctggccacccagtggc

SEQ ID NO: 141 - DNA, 19 bp, Homo sapiens
gcctggccacccagtggct

SEQ ID NO: 142 - DNA, 19 bp, Homo sapiens
cctggccacccagtggctc

SEQ ID NO: 143 - DNA, 19 bp, Homo sapiens
ctggccacccagtggctcc

SEQ ID NO: 144 - DNA, 20 bp, Homo sapiens
tggccacccagtggctccac

SEQ ID NO: 145 - DNA, 20 bp, Homo sapiens
ggccacccagtggctccacc

SEQ ID NO: 146 - DNA, 20 bp, Homo sapiens
gccacccagtggctccaccg

SEQ ID NO: 147 - DNA, 20 bp, Homo sapiens
ccacccagtggctccaccgc

SEQ ID NO: 148 - DNA, 20 bp, Homo sapiens
cacccagtggctccaccgcc

SEQ ID NO: 149 - DNA, 20 bp, Homo sapiens
acccagtggctccaccgccc

SEQ ID NO: 150 - DNA, 20 bp, Homo sapiens
cccagtggctccaccgccct

SEQ ID NO: 151 - DNA, 20 bp, Homo sapiens
ccagtggctccaccgccctg

SEQ ID NO: 152 - DNA, 20 bp, Homo sapiens
cagtggctccaccgccctga

SEQ ID NO: 153 - DNA, 20 bp, Homo sapiens
agtggctccaccgccctgat

SEQ ID NO: 154 - DNA, 20 bp, Homo sapiens
gtggctccaccgccctgatg

SEQ ID NO: 155 - DNA, 20 bp, Homo sapiens
tggctccaccgccctgatgg

SEQ ID NO: 156 - DNA, 20 bp, Homo sapiens
ggctccaccgccctgatgga

SEQ ID NO: 157 - DNA, 20 bp, Homo sapiens
gctccaccgccctgatggat

SEQ ID NO: 158 - DNA, 20 bp, Homo sapiens
ctccaccgccctgatggatc

SEQ ID NO: 159 - DNA, 20 bp, Homo sapiens
tccaccgccctgatggatcc

SEQ ID NO: 160 - DNA, 20 bp, Homo sapiens
ccaccgccctgatggatcca

SEQ ID NO: 161 - DNA, 20 bp, Homo sapiens
caccgccctgatggatccac

SEQ ID NO: 162 - DNA, 20 bp, Homo sapiens
accgccctgatggatccact

SEQ ID NO: 163 - DNA, 20 bp, Homo sapiens
ccgccctgatggatccactg

SEQ ID NO: 164 - DNA, 20 bp, Homo sapiens
cgccctgatggatccactga

SEQ ID NO: 165 - DNA, 20 bp, Homo sapiens
gccctgatggatccactgaa

SEQ ID NO: 166 - DNA, 20 bp, Homo sapiens
ccctgatggatccactgaat

SEQ ID NO: 167 - DNA, 20 bp, Homo sapiens
cctgatggatccactgaatc

SEQ ID NO: 168 - DNA, 20 bp, Homo sapiens
ctgatggatccactgaatct

SEQ ID NO: 169 - DNA, 20 bp, Homo sapiens
tgatggatccactgaatctg

SEQ ID NO: 170 - DNA, 20 bp, Homo sapiens
gatggatccactgaatctgt

SEQ ID NO: 171 - DNA, 20 bp, Homo sapiens
atggatccactgaatctgtc

SEQ ID NO: 172 - DNA, 20 bp, Homo sapiens
tggatccactgaatctgtcc

SEQ ID NO: 173 - DNA, 20 bp, Homo sapiens
ggatccactgaatctgtcct

SEQ ID NO: 174 - DNA, 20 bp, Homo sapiens
gatccactgaatctgtcctg

SEQ ID NO: 175 - DNA, 20 bp, Homo sapiens
atccactgaatctgtcctgg

SEQ ID NO: 176 - DNA, 20 bp, Homo sapiens
tccactgaatctgtcctggt

SEQ ID NO: 177 - DNA, 20 bp, Homo sapiens
ccactgaatctgtcctggta

SEQ ID NO: 178 - DNA, 20 bp, Homo sapiens
cactgaatctgtcctggtat

SEQ ID NO: 179 - DNA, 20 bp, Homo sapiens
actgaatctgtcctggtatg

SEQ ID NO: 180 - DNA, 20 bp, Homo sapiens
ctgaatctgtcctggtatga

SEQ ID NO: 181 - DNA, 20 bp, Homo sapiens
tgaatctgtcctggtatgat

SEQ ID NO: 182 - DNA, 20 bp, Homo sapiens
gaatctgtcctggtatgatg

SEQ ID NO: 183 - DNA, 20 bp, Homo sapiens
aatctgtcctggtatgatga

SEQ ID NO: 184 - DNA, 20 bp, Homo sapiens
atctgtcctggtatgatgat

SEQ ID NO: 185 - DNA, 20 bp, Homo sapiens
tctgtcctggtatgatgatg

SEQ ID NO: 186 - DNA, 20 bp, Homo sapiens
ctgtcctggtatgatgatga

SEQ ID NO: 187 - DNA, 20 bp, Homo sapiens
tgtcctggtatgatgatgat

SEQ ID NO: 188 - DNA, 20 bp, Homo sapiens
gtcctggtatgatgatgatc

SEQ ID NO: 189 - DNA, 20 bp, Homo sapiens
tcctggtatgatgatgatct

SEQ ID NO: 190 - DNA, 20 bp, Homo sapiens
cctggtatgatgatgatctg

SEQ ID NO: 191 - DNA, 20 bp, Homo sapiens
ctggtatgatgatgatctgg

SEQ ID NO: 192 - DNA, 20 bp, Homo sapiens
tggtatgatgatgatctgga

SEQ ID NO: 193 - DNA, 20 bp, Homo sapiens
ggtatgatgatgatctggag

SEQ ID NO: 194 - DNA, 20 bp, Homo sapiens
gtatgatgatgatctggaga

SEQ ID NO: 195 - DNA, 20 bp, Homo sapiens
tatgatgatgatctggagag

SEQ ID NO: 196 - DNA, 20 bp, Homo sapiens
atgatgatgatctggagagg

SEQ ID NO: 197 - DNA, 20 bp, Homo sapiens
tgatgatgatctggagaggc

SEQ ID NO: 198 - DNA, 20 bp, Homo sapiens
gatgatgatctggagaggca

SEQ ID NO: 199 - DNA, 20 bp, Homo sapiens
atgatgatctggagaggcag

SEQ ID NO: 200 - DNA, 20 bp, Homo sapiens
tgatgatctggagaggcaga

SEQ ID NO: 201 - DNA, 20 bp, Homo sapiens
gatgatctggagaggcagaa

SEQ ID NO: 202 - DNA, 20 bp, Homo sapiens
atgatctggagaggcagaac

SEQ ID NO: 203 - DNA, 20 bp, Homo sapiens
tgatctggagaggcagaact

SEQ ID NO: 204 - DNA, 20 bp, Homo sapiens
gatctggagaggcagaactg

SEQ ID NO: 205 - DNA, 20 bp, Homo sapiens
atctggagaggcagaactgg

SEQ ID NO: 206 - DNA, 20 bp, Homo sapiens
tctggagaggcagaactgga

SEQ ID NO: 207 - DNA, 20 bp, Homo sapiens
ctggagaggcagaactggag

SEQ ID NO: 208 - DNA, 20 bp, Homo sapiens
tggagaggcagaactggagc

SEQ ID NO: 209 - DNA, 20 bp, Homo sapiens
ggagaggcagaactggagcc

SEQ ID NO: 210 - DNA, 20 bp, Homo sapiens
gagaggcagaactggagccg

SEQ ID NO: 211 - DNA, 20 bp, Homo sapiens
agaggcagaactggagccgg

SEQ ID NO: 212 - DNA, 20 bp, Homo sapiens
gaggcagaactggagccggc

SEQ ID NO: 213 - DNA, 20 bp, Homo sapiens
aggcagaactggagccggcc

SEQ ID NO: 214 - DNA, 20 bp, Homo sapiens
ggcagaactggagccggccc

SEQ ID NO: 215 - DNA, 20 bp, Homo sapiens
gcagaactggagccggccct

SEQ ID NO: 216 - DNA, 20 bp, Homo sapiens
cagaactggagccggccctt

SEQ ID NO: 217 - DNA, 20 bp, Homo sapiens
agaactggagccggcccttc

SEQ ID NO: 218 - DNA, 20 bp, Homo sapiens
gaactggagccggcccttca

SEQ ID NO: 219 - DNA, 20 bp, Homo sapiens
aactggagccggcccttcaa

SEQ ID NO: 220 - DNA, 20 bp, Homo sapiens
actggagccggcccttcaac

SEQ ID NO: 221 - DNA, 20 bp, Homo sapiens
ctggagccggcccttcaacg

SEQ ID NO: 222 - DNA, 20 bp, Homo sapiens
tggagccggcccttcaacgg

SEQ ID NO: 223 - DNA, 20 bp, Homo sapiens
ggagccggcccttcaacggg

SEQ ID NO: 224 - DNA, 20 bp, Homo sapiens
gagccggcccttcaacgggt

SEQ ID NO: 225 - DNA, 20 bp, Homo sapiens
agccggcccttcaacgggtc

SEQ ID NO: 226 - DNA, 20 bp, Homo sapiens
gccggcccttcaacgggtca

SEQ ID NO: 227 - DNA, 20 bp, Homo sapiens
ccggcccttcaacgggtcag

SEQ ID NO: 228 - DNA, 20 bp, Homo sapiens
cggcccttcaacgggtcaga

SEQ ID NO: 229 - DNA, 20 bp, Homo sapiens
ggcccttcaacgggtcagac

SEQ ID NO: 230 - DNA, 20 bp, Homo sapiens
gcccttcaacgggtcagacg

SEQ ID NO: 231 - DNA, 20 bp, Homo sapiens
cccttcaacgggtcagacgg

SEQ ID NO: 232 - DNA, 20 bp, Homo sapiens
ccttcaacgggtcagacggg

SEQ ID NO: 233 - DNA, 20 bp, Homo sapiens
cttcaacgggtcagacggga

SEQ ID NO: 234 - DNA, 20 bp, Homo sapiens
ttcaacgggtcagacgggaa

SEQ ID NO: 235 - DNA, 20 bp, Homo sapiens
tcaacgggtcagacgggaag

SEQ ID NO: 236 - DNA, 20 bp, Homo sapiens
caacgggtcagacgggaagg

SEQ ID NO: 237 - DNA, 20 bp, Homo sapiens
aacgggtcagacgggaaggc

SEQ ID NO: 238 - DNA, 20 bp, Homo sapiens
acgggtcagacgggaaggcg

SEQ ID NO: 239 - DNA, 20 bp, Homo sapiens
cgggtcagacgggaaggcgg

SEQ ID NO: 240 - DNA, 20 bp, Homo sapiens
gggtcagacgggaaggcgga

SEQ ID NO: 241 - DNA, 20 bp, Homo sapiens
ggtcagacgggaaggcggac

SEQ ID NO: 242 - DNA, 20 bp, Homo sapiens
gtcagacgggaaggcggaca

SEQ ID NO: 243 - DNA, 20 bp, Homo sapiens
tcagacgggaaggcggacag

SEQ ID NO: 244 - DNA, 20 bp, Homo sapiens
cagacgggaaggcggacaga

SEQ ID NO: 245 - DNA, 20 bp, Homo sapiens
agacgggaaggcggacagac

SEQ ID NO: 246 - DNA, 20 bp, Homo sapiens
gacgggaaggcggacagacc

SEQ ID NO: 247 - DNA, 20 bp, Homo sapiens
acgggaaggcggacagaccc

SEQ ID NO: 248 - DNA, 20 bp, Homo sapiens
cgggaaggcggacagacccc

SEQ ID NO: 249 - DNA, 20 bp, Homo sapiens
gggaaggcggacagacccca

SEQ ID NO: 250 - DNA, 20 bp, Homo sapiens
ggaaggcggacagaccccac

SEQ ID NO: 251 - DNA, 20 bp, Homo sapiens
gaaggcggacagaccccact

SEQ ID NO: 252 - DNA, 20 bp, Homo sapiens
aaggcggacagaccccacta

SEQ ID NO: 253 - DNA, 20 bp, Homo sapiens
aggcggacagaccccactac

SEQ ID NO: 254 - DNA, 20 bp, Homo sapiens
ggcggacagaccccactaca

SEQ ID NO: 255 - DNA, 20 bp, Homo sapiens
gcggacagaccccactacaa

SEQ ID NO: 256 - DNA, 20 bp, Homo sapiens
cggacagaccccactacaac

SEQ ID NO: 257 - DNA, 20 bp, Homo sapiens
ggacagaccccactacaact

SEQ ID NO: 258 - DNA, 20 bp, Homo sapiens
gacagaccccactacaacta

SEQ ID NO: 259 - DNA, 20 bp, Homo sapiens
acagaccccactacaactac

SEQ ID NO: 260 - DNA, 20 bp, Homo sapiens
cagaccccactacaactact

SEQ ID NO: 261 - DNA, 20 bp, Homo sapiens
agaccccactacaactacta

SEQ ID NO: 262 - DNA, 20 bp, Homo sapiens
gaccccactacaactactat

SEQ ID NO: 263 - DNA, 20 bp, Homo sapiens
accccactacaactactatg

SEQ ID NO: 264 - DNA, 20 bp, Homo sapiens
ccccactacaactactatgc

SEQ ID NO: 265 - DNA, 20 bp, Homo sapiens
cccactacaactactatgcc

SEQ ID NO: 266 - DNA, 20 bp, Homo sapiens
ccactacaactactatgcca

SEQ ID NO: 267 - DNA, 20 bp, Homo sapiens
cactacaactactatgccac

SEQ ID NO: 268 - DNA, 20 bp, Homo sapiens
actacaactactatgccaca

SEQ ID NO: 269 - DNA, 20 bp, Homo sapiens
ctacaactactatgccacac

SEQ ID NO: 270 - DNA, 20 bp, Homo sapiens
tacaactactatgccacact

SEQ ID NO: 271 - DNA, 20 bp, Homo sapiens
acaactactatgccacactg

SEQ ID NO: 272 - DNA, 20 bp, Homo sapiens
caactactatgccacactgc

SEQ ID NO: 273 - DNA, 20 bp, Homo sapiens
aactactatgccacactgct

SEQ ID NO: 274 - DNA, 20 bp, Homo sapiens
actactatgccacactgctc

SEQ ID NO: 275 - DNA, 20 bp, Homo sapiens
ctactatgccacactgctca

SEQ ID NO: 276 - DNA, 20 bp, Homo sapiens
tactatgccacactgctcac

SEQ ID NO: 277 - DNA, 20 bp, Homo sapiens
actatgccacactgctcacc

SEQ ID NO: 278 - DNA, 20 bp, Homo sapiens
ctatgccacactgctcaccc

SEQ ID NO: 279 - DNA, 20 bp, Homo sapiens
tatgccacactgctcaccct

SEQ ID NO: 280 - DNA, 20 bp, Homo sapiens
atgccacactgctcaccctg

SEQ ID NO: 281 - DNA, 20 bp, Homo sapiens
tgccacactgctcaccctgc

SEQ ID NO: 282 - DNA, 20 bp, Homo sapiens
gccacactgctcaccctgct

SEQ ID NO: 283 - DNA, 20 bp, Homo sapiens
ccacactgctcaccctgctc

SEQ ID NO: 284 - DNA, 20 bp, Homo sapiens
cacactgctcaccctgctca

SEQ ID NO: 285 - DNA, 21 bp, Homo sapiens
acactgctcaccctgctcatc

SEQ ID NO: 286 - DNA, 20 bp, Homo sapiens
cactgctcaccctgctcatc

SEQ ID NO: 287 - DNA, 20 bp, Homo sapiens
actgctcaccctgctcatcg

SEQ ID NO: 288 - DNA, 20 bp, Homo sapiens
ctgctcaccctgctcatcgc

SEQ ID NO: 289 - DNA, 20 bp, Homo sapiens
tgctcaccctgctcatcgct

SEQ ID NO: 290 - DNA, 20 bp, Homo sapiens
gctcaccctgctcatcgctg

SEQ ID NO: 291 - DNA, 20 bp, Homo sapiens
ctcaccctgctcatcgctgt

SEQ ID NO: 292 - DNA, 20 bp, Homo sapiens
tcaccctgctcatcgctgtc

SEQ ID NO: 293 - DNA, 20 bp, Homo sapiens
caccctgctcatcgctgtca

SEQ ID NO: 294 - DNA, 20 bp, Homo sapiens
accctgctcatcgctgtcat

SEQ ID NO: 295 - DNA, 20 bp, Homo sapiens
ccctgctcatcgctgtcatc

SEQ ID NO: 296 - DNA, 20 bp, Homo sapiens
cctgctcatcgctgtcatcg

SEQ ID NO: 297 - DNA, 20 bp, Homo sapiens
ctgctcatcgctgtcatcgt

SEQ ID NO: 298 - DNA, 20 bp, Homo sapiens
tgctcatcgctgtcatcgtc

SEQ ID NO: 299 - DNA, 20 bp, Homo sapiens
gctcatcgctgtcatcgtct

SEQ ID NO: 300 - DNA, 20 bp, Homo sapiens
ctcatcgctgtcatcgtctt

SEQ ID NO: 301 - DNA, 20 bp, Homo sapiens
tcatcgctgtcatcgtcttc

SEQ ID NO: 302 - DNA, 20 bp, Homo sapiens
catcgctgtcatcgtcttcg

SEQ ID NO: 303 - DNA, 20 bp, Homo sapiens
atcgctgtcatcgtcttcgg

SEQ ID NO: 304 - DNA, 20 bp, Homo sapiens
tcgctgtcatcgtcttcggc

SEQ ID NO: 305 - DNA, 20 bp, Homo sapiens
cgctgtcatcgtcttcggca

SEQ ID NO: 306 - DNA, 20 bp, Homo sapiens
gctgtcatcgtcttcggcaa

SEQ ID NO: 307 - DNA, 20 bp, Homo sapiens
ctgtcatcgtcttcggcaac

SEQ ID NO: 308 - DNA, 20 bp, Homo sapiens
tgtcatcgtcttcggcaacg

SEQ ID NO: 309 - DNA, 20 bp, Homo sapiens
gtcatcgtcttcggcaacgt

SEQ ID NO: 310 - DNA, 20 bp, Homo sapiens
tcatcgtcttcggcaacgtg

SEQ ID NO: 311 - DNA, 20 bp, Homo sapiens
catcgtcttcggcaacgtgc

SEQ ID NO: 312 - DNA, 20 bp, Homo sapiens
atcgtcttcggcaacgtgct

SEQ ID NO: 313 - DNA, 20 bp, Homo sapiens
tcgtcttcggcaacgtgctg

SEQ ID NO: 314 - DNA, 20 bp, Homo sapiens
cgtcttcggcaacgtgctgg

SEQ ID NO: 315 - DNA, 20 bp, Homo sapiens
gtcttcggcaacgtgctggt

SEQ ID NO: 316 - DNA, 20 bp, Homo sapiens
tcttcggcaacgtgctggtg

SEQ ID NO: 317 - DNA, 20 bp, Homo sapiens
cttcggcaacgtgctggtgt

SEQ ID NO: 318 - DNA, 20 bp, Homo sapiens
ttcggcaacgtgctggtgtg

SEQ ID NO: 319 - DNA, 20 bp, Homo sapiens
tcggcaacgtgctggtgtgc

SEQ ID NO: 320 - DNA, 20 bp, Homo sapiens
cggcaacgtgctggtgtgca

SEQ ID NO: 321 - DNA, 20 bp, Homo sapiens
ggcaacgtgctggtgtgcat

SEQ ID NO: 322 - DNA, 20 bp, Homo sapiens
gcaacgtgctggtgtgcatg

SEQ ID NO: 323 - DNA, 20 bp, Homo sapiens
caacgtgctggtgtgcatgg

SEQ ID NO: 324 - DNA, 20 bp, Homo sapiens
aacgtgctggtgtgcatggc

SEQ ID NO: 325 - DNA, 20 bp, Homo sapiens
acgtgctggtgtgcatggct

SEQ ID NO: 326 - DNA, 20 bp, Homo sapiens
cgtgctggtgtgcatggctg

SEQ ID NO: 327 - DNA, 19 bp, Homo sapiens
tgctggtgtgcatggctgt

SEQ ID NO: 328 - DNA, 20 bp, Homo sapiens
tgctggtgtgcatggctgtg

SEQ ID NO: 329 - DNA, 20 bp, Homo sapiens
gctggtgtgcatggctgtgt

SEQ ID NO: 330 - DNA, 20 bp, Homo sapiens
ctggtgtgcatggctgtgtc

SEQ ID NO: 331 - DNA, 20 bp, Homo sapiens
tggtgtgcatggctgtgtcc

SEQ ID NO: 332 - DNA, 20 bp, Homo sapiens
ggtgtgcatggctgtgtccc

SEQ ID NO: 333 - DNA, 20 bp, Homo sapiens
gtgtgcatggctgtgtcccg

SEQ ID NO: 334 - DNA, 20 bp, Homo sapiens
tgtgcatggctgtgtcccgc

SEQ ID NO: 335 - DNA, 20 bp, Homo sapiens
gtgcatggctgtgtcccgcg

SEQ ID NO: 336 - DNA, 20 bp, Homo sapiens
tgcatggctgtgtcccgcga

SEQ ID NO: 337 - DNA, 20 bp, Homo sapiens
gcatggctgtgtcccgcgag

SEQ ID NO: 338 - DNA, 20 bp, Homo sapiens
catggctgtgtcccgcgaga

SEQ ID NO: 339 - DNA, 20 bp, Homo sapiens
atggctgtgtcccgcgagaa

SEQ ID NO: 340 - DNA, 20 bp, Homo sapiens
tggctgtgtcccgcgagaag

SEQ ID NO: 341 - DNA, 20 bp, Homo sapiens
ggctgtgtcccgcgagaagg

SEQ ID NO: 342 - DNA, 20 bp, Homo sapiens
gctgtgtcccgcgagaaggc

SEQ ID NO: 343 - DNA, 20 bp, Homo sapiens
ctgtgtcccgcgagaaggcg

SEQ ID NO: 344 - DNA, 20 bp, Homo sapiens
tgtgtcccgcgagaaggcgc

SEQ ID NO: 345 - DNA, 20 bp, Homo sapiens
gtgtcccgcgagaaggcgct

SEQ ID NO: 346 - DNA, 20 bp, Homo sapiens
tgtcccgcgagaaggcgctg

SEQ ID NO: 347 - DNA, 20 bp, Homo sapiens
gtcccgcgagaaggcgctgc

SEQ ID NO: 348 - DNA, 20 bp, Homo sapiens
tcccgcgagaaggcgctgca

SEQ ID NO: 349 - DNA, 20 bp, Homo sapiens
cccgcgagaaggcgctgcag

SEQ ID NO: 350 - DNA, 20 bp, Homo sapiens
ccgcgagaaggcgctgcaga

SEQ ID NO: 351 - DNA, 20 bp, Homo sapiens
cgcgagaaggcgctgcagac

SEQ ID NO: 352 - DNA, 20 bp, Homo sapiens
gcgagaaggcgctgcagacc

SEQ ID NO: 353 - DNA, 20 bp, Homo sapiens
cgagaaggcgctgcagacca

SEQ ID NO: 354 - DNA, 20 bp, Homo sapiens
gagaaggcgctgcagaccac

SEQ ID NO: 355 - DNA, 20 bp, Homo sapiens
agaaggcgctgcagaccacc

SEQ ID NO: 356 - DNA, 20 bp, Homo sapiens
gaaggcgctgcagaccacca

SEQ ID NO: 357 - DNA, 20 bp, Homo sapiens
aaggcgctgcagaccaccac

SEQ ID NO: 358 - DNA, 20 bp, Homo sapiens
aggcgctgcagaccaccacc

SEQ ID NO: 359 - DNA, 20 bp, Homo sapiens
ggcgctgcagaccaccacca

SEQ ID NO: 360 - DNA, 20 bp, Homo sapiens
gcgctgcagaccaccaccaa

SEQ ID NO: 361 - DNA, 20 bp, Homo sapiens
cgctgcagaccaccaccaac

SEQ ID NO: 362 - DNA, 20 bp, Homo sapiens
gctgcagaccaccaccaact

SEQ ID NO: 363 - DNA, 20 bp, Homo sapiens
ctgcagaccaccaccaacta

SEQ ID NO: 364 - DNA, 20 bp, Homo sapiens
tgcagaccaccaccaactac

SEQ ID NO: 365 - DNA, 20 bp, Homo sapiens
gcagaccaccaccaactacc

SEQ ID NO: 366 - DNA, 20 bp, Homo sapiens
cagaccaccaccaactacct

SEQ ID NO: 367 - DNA, 20 bp, Homo sapiens
agaccaccaccaactacctg

SEQ ID NO: 368 - DNA, 20 bp, Homo sapiens
gaccaccaccaactacctga

SEQ ID NO: 369 - DNA, 20 bp, Homo sapiens
accaccaccaactacctgat

SEQ ID NO: 370 - DNA, 20 bp, Homo sapiens
ccaccaccaactacctgatc

SEQ ID NO: 371 - DNA, 20 bp, Homo sapiens
caccaccaactacctgatcg

SEQ ID NO: 372 - DNA, 20 bp, Homo sapiens
accaccaactacctgatcgt

SEQ ID NO: 373 - DNA, 20 bp, Homo sapiens
ccaccaactacctgatcgtc

SEQ ID NO: 374 - DNA, 20 bp, Homo sapiens
caccaactacctgatcgtca

SEQ ID NO: 375 - DNA, 20 bp, Homo sapiens
accaactacctgatcgtcag

SEQ ID NO: 376 - DNA, 20 bp, Homo sapiens
ccaactacctgatcgtcagc

SEQ ID NO: 377 - DNA, 20 bp, Homo sapiens
caactacctgatcgtcagcc

SEQ ID NO: 378 - DNA, 20 bp, Homo sapiens
aactacctgatcgtcagcct

SEQ ID NO: 379 - DNA, 20 bp, Homo sapiens
actacctgatcgtcagcctc

SEQ ID NO: 380 - DNA, 20 bp, Homo sapiens
ctacctgatcgtcagcctcg

SEQ ID NO: 381 - DNA, 20 bp, Homo sapiens
tacctgatcgtcagcctcgc

SEQ ID NO: 382 - DNA, 20 bp, Homo sapiens
acctgatcgtcagcctcgca

SEQ ID NO: 383 - DNA, 20 bp, Homo sapiens
cctgatcgtcagcctcgcag

SEQ ID NO: 384 - DNA, 20 bp, Homo sapiens
ctgatcgtcagcctcgcagt

SEQ ID NO: 385 - DNA, 20 bp, Homo sapiens
tgatcgtcagcctcgcagtg

SEQ ID NO: 386 - DNA, 20 bp, Homo sapiens
gatcgtcagcctcgcagtgg

SEQ ID NO: 387 - DNA, 20 bp, Homo sapiens
atcgtcagcctcgcagtggc

SEQ ID NO: 388 - DNA, 20 bp, Homo sapiens
tcgtcagcctcgcagtggcc

SEQ ID NO: 389 - DNA, 20 bp, Homo sapiens
cgtcagcctcgcagtggccg

SEQ ID NO: 390 - DNA, 20 bp, Homo sapiens
gtcagcctcgcagtggccga

SEQ ID NO: 391 - DNA, 20 bp, Homo sapiens
tcagcctcgcagtggccgac

SEQ ID NO: 392 - DNA, 20 bp, Homo sapiens
cagcctcgcagtggccgacc

SEQ ID NO: 393 - DNA, 20 bp, Homo sapiens
agcctcgcagtggccgacct

SEQ ID NO: 394 - DNA, 20 bp, Homo sapiens
gcctcgcagtggccgacctc

SEQ ID NO: 395 - DNA, 20 bp, Homo sapiens
cctcgcagtggccgacctcc

SEQ ID NO: 396 - DNA, 20 bp, Homo sapiens
ctcgcagtggccgacctcct

SEQ ID NO: 397 - DNA, 20 bp, Homo sapiens
tcgcagtggccgacctcctc

SEQ ID NO: 398 - DNA, 20 bp, Homo sapiens
cgcagtggccgacctcctcg

SEQ ID NO: 399 - DNA, 20 bp, Homo sapiens
gcagtggccgacctcctcgt

SEQ ID NO: 400 - DNA, 20 bp, Homo sapiens
cagtggccgacctcctcgtc

SEQ ID NO: 401 - DNA, 20 bp, Homo sapiens
agtggccgacctcctcgtcg

SEQ ID NO: 402 - DNA, 20 bp, Homo sapiens
gtggccgacctcctcgtcgc

SEQ ID NO: 403 - DNA, 20 bp, Homo sapiens
tggccgacctcctcgtcgcc

SEQ ID NO: 404 - DNA, 20 bp, Homo sapiens
ggccgacctcctcgtcgcca

SEQ ID NO: 405 - DNA, 20 bp, Homo sapiens
gccgacctcctcgtcgccac

SEQ ID NO: 406 - DNA, 20 bp, Homo sapiens
ccgacctcctcgtcgccaca

SEQ ID NO: 407 - DNA, 20 bp, Homo sapiens
cgacctcctcgtcgccacac

SEQ ID NO: 408 - DNA, 20 bp, Homo sapiens
gacctcctcgtcgccacact

SEQ ID NO: 409 - DNA, 20 bp, Homo sapiens
acctcctcgtcgccacactg

SEQ ID NO: 410 - DNA, 20 bp, Homo sapiens
cctcctcgtcgccacactgg

SEQ ID NO: 411 - DNA, 20 bp, Homo sapiens
ctcctcgtcgccacactggt

SEQ ID NO: 412 - DNA, 20 bp, Homo sapiens
tcctcgtcgccacactggtc

SEQ ID NO: 413 - DNA, 20 bp, Homo sapiens
cctcgtcgccacactggtca

SEQ ID NO: 414 - DNA, 20 bp, Homo sapiens
ctcgtcgccacactggtcat

SEQ ID NO: 415 - DNA, 20 bp, Homo sapiens
tcgtcgccacactggtcatg

SEQ ID NO: 416 - DNA, 20 bp, Homo sapiens
cgtcgccacactggtcatgc

SEQ ID NO: 417 - DNA, 20 bp, Homo sapiens
gtcgccacactggtcatgcc

SEQ ID NO: 418 - DNA, 20 bp, Homo sapiens
tcgccacactggtcatgccc

SEQ ID NO: 419 - DNA, 20 bp, Homo sapiens
cgccacactggtcatgccct

SEQ ID NO: 420 - DNA, 20 bp, Homo sapiens
gccacactggtcatgccctg

SEQ ID NO: 421 - DNA, 20 bp, Homo sapiens
ccacactggtcatgccctgg

SEQ ID NO: 422 - DNA, 20 bp, Homo sapiens
cacactggtcatgccctggg

SEQ ID NO: 423 - DNA, 20 bp, Homo sapiens
acactggtcatgccctgggt

SEQ ID NO: 424 - DNA, 20 bp, Homo sapiens
cactggtcatgccctgggtt

SEQ ID NO: 425 - DNA, 20 bp, Homo sapiens
actggtcatgccctgggttg

SEQ ID NO: 426 - DNA, 20 bp, Homo sapiens
ctggtcatgccctgggttgt

SEQ ID NO: 427 - DNA, 20 bp, Homo sapiens
tggtcatgccctgggttgtc

SEQ ID NO: 428 - DNA, 20 bp, Homo sapiens
ggtcatgccctgggttgtct

SEQ ID NO: 429 - DNA, 20 bp, Homo sapiens
gtcatgccctgggttgtcta

SEQ ID NO: 430 - DNA, 20 bp, Homo sapiens
tcatgccctgggttgtctac

SEQ ID NO: 431 - DNA, 19 bp, Homo sapiens
catgccctgggttgtctac

SEQ ID NO: 432 - DNA, 19 bp, Homo sapiens
atgccctgggttgtctacc

SEQ ID NO: 433 - DNA, 19 bp, Homo sapiens
tgccctgggttgtctacct

SEQ ID NO: 434 - DNA, 19 bp, Homo sapiens
gccctgggttgtctacctg

SEQ ID NO: 435 - DNA, 19 bp, Homo sapiens
ccctgggttgtctacctgg

SEQ ID NO: 436 - DNA, 20 bp, Homo sapiens
cctgggttgtctacctggag

SEQ ID NO: 437 - DNA, 20 bp, Homo sapiens
ctgggttgtctacctggagg

SEQ ID NO: 438 - DNA, 20 bp, Homo sapiens
tgggttgtctacctggaggt

SEQ ID NO: 439 - DNA, 20 bp, Homo sapiens
gggttgtctacctggaggtg

SEQ ID NO: 440 - DNA, 20 bp, Homo sapiens
ggttgtctacctggaggtgg

SEQ ID NO: 441 - DNA, 20 bp, Homo sapiens
gttgtctacctggaggtggt

SEQ ID NO: 442 - DNA, 20 bp, Homo sapiens
ttgtctacctggaggtggta

SEQ ID NO: 443 - DNA, 20 bp, Homo sapiens
tgtctacctggaggtggtag

SEQ ID NO: 444 - DNA, 20 bp, Homo sapiens
gtctacctggaggtggtagg

SEQ ID NO: 445 - DNA, 20 bp, Homo sapiens
tctacctggaggtggtaggt

SEQ ID NO: 446 - DNA, 20 bp, Homo sapiens
ctacctggaggtggtaggtg

SEQ ID NO: 447 - DNA, 20 bp, Homo sapiens
tacctggaggtggtaggtga

SEQ ID NO: 448 - DNA, 20 bp, Homo sapiens
acctggaggtggtaggtgag

SEQ ID NO: 449 - DNA, 20 bp, Homo sapiens
cctggaggtggtaggtgagt

SEQ ID NO: 450 - DNA, 20 bp, Homo sapiens
ctggaggtggtaggtgagtg

SEQ ID NO: 451 - DNA, 20 bp, Homo sapiens
tggaggtggtaggtgagtgg

SEQ ID NO: 452 - DNA, 20 bp, Homo sapiens
ggaggtggtaggtgagtgga

SEQ ID NO: 453 - DNA, 20 bp, Homo sapiens
gaggtggtaggtgagtggaa

SEQ ID NO: 454 - DNA, 20 bp, Homo sapiens
aggtggtaggtgagtggaaa

SEQ ID NO: 455 - DNA, 20 bp, Homo sapiens
ggtggtaggtgagtggaaat

SEQ ID NO: 456 - DNA, 20 bp, Homo sapiens
gtggtaggtgagtggaaatt

SEQ ID NO: 457 - DNA, 20 bp, Homo sapiens
tggtaggtgagtggaaattc

SEQ ID NO: 458 - DNA, 20 bp, Homo sapiens
ggtaggtgagtggaaattca

SEQ ID NO: 459 - DNA, 20 bp, Homo sapiens
gtaggtgagtggaaattcag

SEQ ID NO: 460 - DNA, 20 bp, Homo sapiens
taggtgagtggaaattcagc

SEQ ID NO: 461 - DNA, 20 bp, Homo sapiens
aggtgagtggaaattcagca

SEQ ID NO: 462 - DNA, 20 bp, Homo sapiens
ggtgagtggaaattcagcag

SEQ ID NO: 463 - DNA, 20 bp, Homo sapiens
gtgagtggaaattcagcagg

SEQ ID NO: 464 - DNA, 20 bp, Homo sapiens
tgagtggaaattcagcagga

SEQ ID NO: 465 - DNA, 20 bp, Homo sapiens
gagtggaaattcagcaggat

SEQ ID NO: 466 - DNA, 20 bp, Homo sapiens
agtggaaattcagcaggatt

SEQ ID NO: 467 - DNA, 20 bp, Homo sapiens
gtggaaattcagcaggattc

SEQ ID NO: 468 - DNA, 20 bp, Homo sapiens
tggaaattcagcaggattca

SEQ ID NO: 469 - DNA, 20 bp, Homo sapiens
ggaaattcagcaggattcac

SEQ ID NO: 470 - DNA, 20 bp, Homo sapiens
gaaattcagcaggattcact

SEQ ID NO: 471 - DNA, 20 bp, Homo sapiens
aaattcagcaggattcactg

SEQ ID NO: 472 - DNA, 20 bp, Homo sapiens
aattcagcaggattcactgt

SEQ ID NO: 473 - DNA, 20 bp, Homo sapiens
attcagcaggattcactgtg

SEQ ID NO: 474 - DNA, 20 bp, Homo sapiens
ttcagcaggattcactgtga

SEQ ID NO: 475 - DNA, 20 bp, Homo sapiens
tcagcaggattcactgtgac

SEQ ID NO: 476 - DNA, 20 bp, Homo sapiens
cagcaggattcactgtgaca

SEQ ID NO: 477 - DNA, 20 bp, Homo sapiens
agcaggattcactgtgacat

SEQ ID NO: 478 - DNA, 20 bp, Homo sapiens
gcaggattcactgtgacatc

SEQ ID NO: 479 - DNA, 20 bp, Homo sapiens
caggattcactgtgacatct

SEQ ID NO: 480 - DNA, 20 bp, Homo sapiens
aggattcactgtgacatctt

SEQ ID NO: 481 - DNA, 20 bp, Homo sapiens
ggattcactgtgacatcttc

SEQ ID NO: 482 - DNA, 20 bp, Homo sapiens
gattcactgtgacatcttcg

SEQ ID NO: 483 - DNA, 20 bp, Homo sapiens
attcactgtgacatcttcgt

SEQ ID NO: 484 - DNA, 20 bp, Homo sapiens
ttcactgtgacatcttcgtc

SEQ ID NO: 485 - DNA, 20 bp, Homo sapiens
tcactgtgacatcttcgtca

SEQ ID NO: 486 - DNA, 20 bp, Homo sapiens
cactgtgacatcttcgtcac

SEQ ID NO: 487 - DNA, 20 bp, Homo sapiens
actgtgacatcttcgtcact

SEQ ID NO: 488 - DNA, 20 bp, Homo sapiens
ctgtgacatcttcgtcactc

SEQ ID NO: 489 - DNA, 20 bp, Homo sapiens
tgtgacatcttcgtcactct

SEQ ID NO: 490 - DNA, 20 bp, Homo sapiens
gtgacatcttcgtcactctg

SEQ ID NO: 491 - DNA, 20 bp, Homo sapiens
tgacatcttcgtcactctgg

SEQ ID NO: 492 - DNA, 20 bp, Homo sapiens
gacatcttcgtcactctgga

SEQ ID NO: 493 - DNA, 20 bp, Homo sapiens
acatcttcgtcactctggac

SEQ ID NO: 494 - DNA, 20 bp, Homo sapiens
catcttcgtcactctggacg

SEQ ID NO: 495 - DNA, 20 bp, Homo sapiens
atcttcgtcactctggacgt

SEQ ID NO: 496 - DNA, 20 bp, Homo sapiens
tcttcgtcactctggacgtc

SEQ ID NO: 497 - DNA, 20 bp, Homo sapiens
cttcgtcactctggacgtca

SEQ ID NO: 498 - DNA, 20 bp, Homo sapiens
ttcgtcactctggacgtcat

SEQ ID NO: 499 - DNA, 20 bp, Homo sapiens
tcgtcactctggacgtcatg

SEQ ID NO: 500 - DNA, 20 bp, Homo sapiens
cgtcactctggacgtcatga

SEQ ID NO: 501 - DNA, 20 bp, Homo sapiens
gtcactctggacgtcatgat

SEQ ID NO: 502 - DNA, 20 bp, Homo sapiens
tcactctggacgtcatgatg

SEQ ID NO: 503 - DNA, 20 bp, Homo sapiens
cactctggacgtcatgatgt

SEQ ID NO: 504 - DNA, 20 bp, Homo sapiens
actctggacgtcatgatgtg

SEQ ID NO: 505 - DNA, 20 bp, Homo sapiens
ctctggacgtcatgatgtgc

SEQ ID NO: 506 - DNA, 20 bp, Homo sapiens
tctggacgtcatgatgtgca

SEQ ID NO: 507 - DNA, 20 bp, Homo sapiens
ctggacgtcatgatgtgcac

SEQ ID NO: 508 - DNA, 20 bp, Homo sapiens
tggacgtcatgatgtgcacg

SEQ ID NO: 509 - DNA, 20 bp, Homo sapiens
ggacgtcatgatgtgcacgg

SEQ ID NO: 510 - DNA, 20 bp, Homo sapiens
gacgtcatgatgtgcacggc

SEQ ID NO: 511 - DNA, 20 bp, Homo sapiens
acgtcatgatgtgcacggcg

SEQ ID NO: 512 - DNA, 20 bp, Homo sapiens
cgtcatgatgtgcacggcga

SEQ ID NO: 513 - DNA, 20 bp, Homo sapiens
gtcatgatgtgcacggcgag

SEQ ID NO: 514 - DNA, 20 bp, Homo sapiens
tcatgatgtgcacggcgagc

SEQ ID NO: 515 - DNA, 20 bp, Homo sapiens
catgatgtgcacggcgagca

SEQ ID NO: 516 - DNA, 20 bp, Homo sapiens
atgatgtgcacggcgagcat

SEQ ID NO: 517 - DNA, 20 bp, Homo sapiens
tgatgtgcacggcgagcatc

SEQ ID NO: 518 - DNA, 20 bp, Homo sapiens
gatgtgcacggcgagcatcc

SEQ ID NO: 519 - DNA, 20 bp, Homo sapiens
atgtgcacggcgagcatcct

SEQ ID NO: 520 - DNA, 20 bp, Homo sapiens
tgtgcacggcgagcatcctg

SEQ ID NO: 521 - DNA, 20 bp, Homo sapiens
gtgcacggcgagcatcctga

SEQ ID NO: 522 - DNA, 20 bp, Homo sapiens
tgcacggcgagcatcctgaa

SEQ ID NO: 523 - DNA, 20 bp, Homo sapiens
gcacggcgagcatcctgaac

SEQ ID NO: 524 - DNA, 20 bp, Homo sapiens
cacggcgagcatcctgaact

SEQ ID NO: 525 - DNA, 20 bp, Homo sapiens
acggcgagcatcctgaactt

SEQ ID NO: 526 - DNA, 20 bp, Homo sapiens
cggcgagcatcctgaacttg

SEQ ID NO: 527 - DNA, 20 bp, Homo sapiens
ggcgagcatcctgaacttgt

SEQ ID NO: 528 - DNA, 20 bp, Homo sapiens
gcgagcatcctgaacttgtg

SEQ ID NO: 529 - DNA, 20 bp, Homo sapiens
cgagcatcctgaacttgtgt

SEQ ID NO: 530 - DNA, 20 bp, Homo sapiens
gagcatcctgaacttgtgtg

SEQ ID NO: 531 - DNA, 20 bp, Homo sapiens
agcatcctgaacttgtgtgc

SEQ ID NO: 532 - DNA, 20 bp, Homo sapiens
gcatcctgaacttgtgtgcc

SEQ ID NO: 533 - DNA, 20 bp, Homo sapiens
catcctgaacttgtgtgcca

SEQ ID NO: 534 - DNA, 20 bp, Homo sapiens
atcctgaacttgtgtgccat

SEQ ID NO: 535 - DNA, 20 bp, Homo sapiens
tcctgaacttgtgtgccatc

SEQ ID NO: 536 - DNA, 20 bp, Homo sapiens
cctgaacttgtgtgccatca

SEQ ID NO: 537 - DNA, 20 bp, Homo sapiens
ctgaacttgtgtgccatcag

SEQ ID NO: 538 - DNA, 20 bp, Homo sapiens
tgaacttgtgtgccatcagc

SEQ ID NO: 539 - DNA, 20 bp, Homo sapiens
gaacttgtgtgccatcagca

SEQ ID NO: 540 - DNA, 20 bp, Homo sapiens
aacttgtgtgccatcagcat

SEQ ID NO: 541 - DNA, 20 bp, Homo sapiens
acttgtgtgccatcagcatc

SEQ ID NO: 542 - DNA, 20 bp, Homo sapiens
cttgtgtgccatcagcatcg

SEQ ID NO: 543 - DNA, 20 bp, Homo sapiens
ttgtgtgccatcagcatcga

SEQ ID NO: 544 - DNA, 20 bp, Homo sapiens
tgtgtgccatcagcatcgac

SEQ ID NO: 545 - DNA, 20 bp, Homo sapiens
gtgtgccatcagcatcgaca

SEQ ID NO: 546 - DNA, 20 bp, Homo sapiens
tgtgccatcagcatcgacag

SEQ ID NO: 547 - DNA, 20 bp, Homo sapiens
gtgccatcagcatcgacagg

SEQ ID NO: 548 - DNA, 20 bp, Homo sapiens
tgccatcagcatcgacaggt

SEQ ID NO: 549 - DNA, 20 bp, Homo sapiens
gccatcagcatcgacaggta

SEQ ID NO: 550 - DNA, 20 bp, Homo sapiens
ccatcagcatcgacaggtac

SEQ ID NO: 551 - DNA, 20 bp, Homo sapiens
catcagcatcgacaggtaca

SEQ ID NO: 552 - DNA, 20 bp, Homo sapiens
atcagcatcgacaggtacac

SEQ ID NO: 553 - DNA, 20 bp, Homo sapiens
tcagcatcgacaggtacaca

SEQ ID NO: 554 - DNA, 20 bp, Homo sapiens
cagcatcgacaggtacacag

SEQ ID NO: 555 - DNA, 20 bp, Homo sapiens
agcatcgacaggtacacagc

SEQ ID NO: 556 - DNA, 20 bp, Homo sapiens
gcatcgacaggtacacagct

SEQ ID NO: 557 - DNA, 20 bp, Homo sapiens
catcgacaggtacacagctg

SEQ ID NO: 558 - DNA, 20 bp, Homo sapiens
atcgacaggtacacagctgt

SEQ ID NO: 559 - DNA, 20 bp, Homo sapiens
tcgacaggtacacagctgtg

SEQ ID NO: 560 - DNA, 20 bp, Homo sapiens
cgacaggtacacagctgtgg

SEQ ID NO: 561 - DNA, 20 bp, Homo sapiens
gacaggtacacagctgtggc

SEQ ID NO: 562 - DNA, 20 bp, Homo sapiens
acaggtacacagctgtggcc

SEQ ID NO: 563 - DNA, 20 bp, Homo sapiens
caggtacacagctgtggcca

SEQ ID NO: 564 - DNA, 20 bp, Homo sapiens
aggtacacagctgtggccat

SEQ ID NO: 565 - DNA, 20 bp, Homo sapiens
ggtacacagctgtggccatg

SEQ ID NO: 566 - DNA, 20 bp, Homo sapiens
ggtacacagctgtggccatg

SEQ ID NO: 567 - DNA, 20 bp, Homo sapiens
tacacagctgtggccatgcc

SEQ ID NO: 568 - DNA, 20 bp, Homo sapiens
acacagctgtggccatgccc

SEQ ID NO: 569 - DNA, 20 bp, Homo sapiens
cacagctgtggccatgccca

SEQ ID NO: 570 - DNA, 20 bp, Homo sapiens
acagctgtggccatgcccat

SEQ ID NO: 571 - DNA, 20 bp, Homo sapiens
cagctgtggccatgcccatg

SEQ ID NO: 572 - DNA, 20 bp, Homo sapiens
agctgtggccatgcccatgc

SEQ ID NO: 573 - DNA, 20 bp, Homo sapiens
gctgtggccatgcccatgct

SEQ ID NO: 574 - DNA, 20 bp, Homo sapiens
ctgtggccatgcccatgctg

SEQ ID NO: 575 - DNA, 20 bp, Homo sapiens
tgtggccatgcccatgctgt

SEQ ID NO: 576 - DNA, 20 bp, Homo sapiens
gtggccatgcccatgctgta

SEQ ID NO: 577 - DNA, 20 bp, Homo sapiens
tggccatgcccatgctgtac

SEQ ID NO: 578 - DNA, 20 bp, Homo sapiens
ggccatgcccatgctgtaca

SEQ ID NO: 579 - DNA, 20 bp, Homo sapiens
gccatgcccatgctgtacaa

SEQ ID NO: 580 - DNA, 20 bp, Homo sapiens
ccatgcccatgctgtacaat

SEQ ID NO: 581 - DNA, 20 bp, Homo sapiens
catgcccatgctgtacaata

SEQ ID NO: 582 - DNA, 20 bp, Homo sapiens
atgcccatgctgtacaatac

SEQ ID NO: 583 - DNA, 20 bp, Homo sapiens
tgcccatgctgtacaatacg

SEQ ID NO: 584 - DNA, 20 bp, Homo sapiens
gcccatgctgtacaatacgc

SEQ ID NO: 585 - DNA, 20 bp, Homo sapiens
cccatgctgtacaatacgcg

SEQ ID NO: 586 - DNA, 20 bp, Homo sapiens
ccatgctgtacaatacgcgc

SEQ ID NO: 587 - DNA, 20 bp, Homo sapiens
catgctgtacaatacgcgct

SEQ ID NO: 588 - DNA, 20 bp, Homo sapiens
atgctgtacaatacgcgcta

SEQ ID NO: 589 - DNA, 20 bp, Homo sapiens
tgctgtacaatacgcgctac

SEQ ID NO: 590 - DNA, 20 bp, Homo sapiens
gctgtacaatacgcgctaca

SEQ ID NO: 591 - DNA, 20 bp, Homo sapiens
ctgtacaatacgcgctacag

SEQ ID NO: 592 - DNA, 20 bp, Homo sapiens
tgtacaatacgcgctacagc

SEQ ID NO: 593 - DNA, 20 bp, Homo sapiens
gtacaatacgcgctacagct

SEQ ID NO: 594 - DNA, 20 bp, Homo sapiens
tacaatacgcgctacagctc

SEQ ID NO: 595 - DNA, 20 bp, Homo sapiens
acaatacgcgctacagctcc

SEQ ID NO: 596 - DNA, 20 bp, Homo sapiens
caatacgcgctacagctcca

SEQ ID NO: 597 - DNA, 20 bp, Homo sapiens
aatacgcgctacagctccaa

SEQ ID NO: 598 - DNA, 20 bp, Homo sapiens
atacgcgctacagctccaag

SEQ ID NO: 599 - DNA, 20 bp, Homo sapiens
tacgcgctacagctccaagc

SEQ ID NO: 600 - DNA, 20 bp, Homo sapiens
acgcgctacagctccaagcg

SEQ ID NO: 601 - DNA, 20 bp, Homo sapiens
cgcgctacagctccaagcgc

SEQ ID NO: 602 - DNA, 20 bp, Homo sapiens
gcgctacagctccaagcgcc

SEQ ID NO: 603 - DNA, 20 bp, Homo sapiens
cgctacagctccaagcgccg

SEQ ID NO: 604 - DNA, 20 bp, Homo sapiens
gctacagctccaagcgccgg

SEQ ID NO: 605 - DNA, 20 bp, Homo sapiens
ctacagctccaagcgccggg

SEQ ID NO: 606 - DNA, 20 bp, Homo sapiens
tacagctccaagcgccgggt

SEQ ID NO: 607 - DNA, 20 bp, Homo sapiens
acagctccaagcgccgggtc

SEQ ID NO: 608 - DNA, 20 bp, Homo sapiens
cagctccaagcgccgggtca

SEQ ID NO: 609 - DNA, 20 bp, Homo sapiens
agctccaagcgccgggtcac

SEQ ID NO: 610 - DNA, 20 bp, Homo sapiens
gctccaagcgccgggtcacc

SEQ ID NO: 611 - DNA, 20 bp, Homo sapiens
ctccaagcgccgggtcaccg

SEQ ID NO: 612 - DNA, 20 bp, Homo sapiens
tccaagcgccgggtcaccgt

SEQ ID NO: 613 - DNA, 20 bp, Homo sapiens
ccaagcgccgggtcaccgtc

SEQ ID NO: 614 - DNA, 20 bp, Homo sapiens
caagcgccgggtcaccgtca

SEQ ID NO: 615 - DNA, 20 bp, Homo sapiens
aagcgccgggtcaccgtcat

SEQ ID NO: 616 - DNA, 20 bp, Homo sapiens
agcgccgggtcaccgtcatg

SEQ ID NO: 617 - DNA, 20 bp, Homo sapiens
gcgccgggtcaccgtcatga

SEQ ID NO: 618 - DNA, 20 bp, Homo sapiens
cgccgggtcaccgtcatgat

SEQ ID NO: 619 - DNA, 20 bp, Homo sapiens
gccgggtcaccgtcatgatc

SEQ ID NO: 620 - DNA, 20 bp, Homo sapiens
ccgggtcaccgtcatgatct

SEQ ID NO: 621 - DNA, 20 bp, Homo sapiens
cgggtcaccgtcatgatctc

SEQ ID NO: 622 - DNA, 20 bp, Homo sapiens
gggtcaccgtcatgatctcc

SEQ ID NO: 623 - DNA, 20 bp, Homo sapiens
ggtcaccgtcatgatctcca

SEQ ID NO: 624 - DNA, 20 bp, Homo sapiens
gtcaccgtcatgatctccat

SEQ ID NO: 625 - DNA, 20 bp, Homo sapiens
tcaccgtcatgatctccatc

SEQ ID NO: 626 - DNA, 20 bp, Homo sapiens
caccgtcatgatctccatcg

SEQ ID NO: 627 - DNA, 20 bp, Homo sapiens
accgtcatgatctccatcgt

SEQ ID NO: 628 - DNA, 20 bp, Homo sapiens
ccgtcatgatctccatcgtc

SEQ ID NO: 629 - DNA, 20 bp, Homo sapiens
cgtcatgatctccatcgtct

SEQ ID NO: 630 - DNA, 20 bp, Homo sapiens
gtcatgatctccatcgtctg

SEQ ID NO: 631 - DNA, 20 bp, Homo sapiens
tcatgatctccatcgtctgg

SEQ ID NO: 632 - DNA, 20 bp, Homo sapiens
catgatctccatcgtctggg

SEQ ID NO: 633 - DNA, 20 bp, Homo sapiens
atgatctccatcgtctgggt

SEQ ID NO: 634 - DNA, 20 bp, Homo sapiens
tgatctccatcgtctgggtc

SEQ ID NO: 635 - DNA, 20 bp, Homo sapiens
gatctccatcgtctgggtcc

SEQ ID NO: 636 - DNA, 20 bp, Homo sapiens
atctccatcgtctgggtcct

SEQ ID NO: 637 - DNA, 20 bp, Homo sapiens
tctccatcgtctgggtcctg

SEQ ID NO: 638 - DNA, 20 bp, Homo sapiens
ctccatcgtctgggtcctgt

SEQ ID NO: 639 - DNA, 20 bp, Homo sapiens
tccatcgtctgggtcctgtc

SEQ ID NO: 640 - DNA, 20 bp, Homo sapiens
ccatcgtctgggtcctgtcc

SEQ ID NO: 641 - DNA, 20 bp, Homo sapiens
catcgtctgggtcctgtcct

SEQ ID NO: 642 - DNA, 20 bp, Homo sapiens
atcgtctgggtcctgtcctt

SEQ ID NO: 643 - DNA, 20 bp, Homo sapiens
tcgtctgggtcctgtccttc

SEQ ID NO: 644 - DNA, 20 bp, Homo sapiens
cgtctgggtcctgtccttca

SEQ ID NO: 645 - DNA, 20 bp, Homo sapiens
gtctgggtcctgtccttcac

SEQ ID NO: 646 - DNA, 20 bp, Homo sapiens
tctgggtcctgtccttcacc

SEQ ID NO: 647 - DNA, 20 bp, Homo sapiens
ctgggtcctgtccttcacca

SEQ ID NO: 648 - DNA, 20 bp, Homo sapiens
tgggtcctgtccttcaccat

SEQ ID NO: 649 - DNA, 20 bp, Homo sapiens
gggtcctgtccttcaccatc

SEQ ID NO: 650 - DNA, 20 bp, Homo sapiens
ggtcctgtccttcaccatct

SEQ ID NO: 651 - DNA, 20 bp, Homo sapiens
gtcctgtccttcaccatctc

SEQ ID NO: 652 - DNA, 20 bp, Homo sapiens
tcctgtccttcaccatctcc

SEQ ID NO: 653 - DNA, 20 bp, Homo sapiens
cctgtccttcaccatctcct

SEQ ID NO: 654 - DNA, 20 bp, Homo sapiens
ctgtccttcaccatctcctg

SEQ ID NO: 655 - DNA, 20 bp, Homo sapiens
tgtccttcaccatctcctgc

SEQ ID NO: 656 - DNA, 20 bp, Homo sapiens
gtccttcaccatctcctgcc

SEQ ID NO: 657 - DNA, 20 bp, Homo sapiens
tccttcaccatctcctgccc

SEQ ID NO: 658 - DNA, 20 bp, Homo sapiens
ccttcaccatctcctgccca

SEQ ID NO: 659 - DNA, 20 bp, Homo sapiens
cttcaccatctcctgcccac

SEQ ID NO: 660 - DNA, 20 bp, Homo sapiens
ttcaccatctcctgcccact

SEQ ID NO: 661 - DNA, 20 bp, Homo sapiens
tcaccatctcctgcccactc

SEQ ID NO: 662 - DNA, 20 bp, Homo sapiens
caccatctcctgcccactcc

SEQ ID NO: 663 - DNA, 20 bp, Homo sapiens
accatctcctgcccactcct

SEQ ID NO: 664 - DNA, 20 bp, Homo sapiens
ccatctcctgcccactcctc

SEQ ID NO: 665 - DNA, 20 bp, Homo sapiens
catctcctgcccactcctct

SEQ ID NO: 666 - DNA, 20 bp, Homo sapiens
atctcctgcccactcctctt

SEQ ID NO: 667 - DNA, 20 bp, Homo sapiens
tctcctgcccactcctcttc

SEQ ID NO: 668 - DNA, 20 bp, Homo sapiens
ctcctgcccactcctcttcg

SEQ ID NO: 669 - DNA, 20 bp, Homo sapiens
tcctgcccactcctcttcgg

SEQ ID NO: 670 - DNA, 20 bp, Homo sapiens
cctgcccactcctcttcgga

SEQ ID NO: 671 - DNA, 20 bp, Homo sapiens
ctgcccactcctcttcggac

SEQ ID NO: 672 - DNA, 20 bp, Homo sapiens
tgcccactcctcttcggact

SEQ ID NO: 673 - DNA, 20 bp, Homo sapiens
gcccactcctcttcggactc

SEQ ID NO: 674 - DNA, 20 bp, Homo sapiens
cccactcctcttcggactca

SEQ ID NO: 675 - DNA, 20 bp, Homo sapiens
ccactcctcttcggactcaa

SEQ ID NO: 676 - DNA, 20 bp, Homo sapiens
cactcctcttcggactcaat

SEQ ID NO: 677 - DNA, 20 bp, Homo sapiens
actcctcttcggactcaata

SEQ ID NO: 678 - DNA, 20 bp, Homo sapiens
ctcctcttcggactcaataa

SEQ ID NO: 679 - DNA, 20 bp, Homo sapiens
tcctcttcggactcaataac

SEQ ID NO: 680 - DNA, 20 bp, Homo sapiens
cctcttcggactcaataacg

SEQ ID NO: 681 - DNA, 20 bp, Homo sapiens
ctcttcggactcaataacgc

SEQ ID NO: 682 - DNA, 20 bp, Homo sapiens
tcttcggactcaataacgca

SEQ ID NO: 683 - DNA, 20 bp, Homo sapiens
cttcggactcaataacgcag

SEQ ID NO: 684 - DNA, 20 bp, Homo sapiens
ttcggactcaataacgcaga

SEQ ID NO: 685 - DNA, 20 bp, Homo sapiens
tcggactcaataacgcagac

SEQ ID NO: 686 - DNA, 20 bp, Homo sapiens
cggactcaataacgcagacc

SEQ ID NO: 687 - DNA, 20 bp, Homo sapiens
ggactcaataacgcagacca

SEQ ID NO: 688 - DNA, 20 bp, Homo sapiens
gactcaataacgcagaccag

SEQ ID NO: 689 - DNA, 20 bp, Homo sapiens
actcaataacgcagaccaga

SEQ ID NO: 690 - DNA, 20 bp, Homo sapiens
ctcaataacgcagaccagaa

SEQ ID NO: 691 - DNA, 20 bp, Homo sapiens
tcaataacgcagaccagaac

SEQ ID NO: 692 - DNA, 20 bp, Homo sapiens
caataacgcagaccagaacg

SEQ ID NO: 693 - DNA, 20 bp, Homo sapiens
aataacgcagaccagaacga

SEQ ID NO: 694 - DNA, 20 bp, Homo sapiens
ataacgcagaccagaacgag

SEQ ID NO: 695 - DNA, 20 bp, Homo sapiens
taacgcagaccagaacgagt

SEQ ID NO: 696 - DNA, 20 bp, Homo sapiens
aacgcagaccagaacgagtg

SEQ ID NO: 697 - DNA, 20 bp, Homo sapiens
acgcagaccagaacgagtgc

SEQ ID NO: 698 - DNA, 20 bp, Homo sapiens
cgcagaccagaacgagtgca

SEQ ID NO: 699 - DNA, 20 bp, Homo sapiens
gcagaccagaacgagtgcat

SEQ ID NO: 700 - DNA, 20 bp, Homo sapiens
cagaccagaacgagtgcatc

SEQ ID NO: 701 - DNA, 20 bp, Homo sapiens
agaccagaacgagtgcatca

SEQ ID NO: 702 - DNA, 20 bp, Homo sapiens
gaccagaacgagtgcatcat

SEQ ID NO: 703 - DNA, 20 bp, Homo sapiens
accagaacgagtgcatcatt

SEQ ID NO: 704 - DNA, 20 bp, Homo sapiens
ccagaacgagtgcatcattg

SEQ ID NO: 705 - DNA, 20 bp, Homo sapiens
cagaacgagtgcatcattgc

SEQ ID NO: 706 - DNA, 20 bp, Homo sapiens
agaacgagtgcatcattgcc

SEQ ID NO: 707 - DNA, 20 bp, Homo sapiens
gaacgagtgcatcattgcca

SEQ ID NO: 708 - DNA, 20 bp, Homo sapiens
aacgagtgcatcattgccaa

SEQ ID NO: 709 - DNA, 20 bp, Homo sapiens
acgagtgcatcattgccaac

SEQ ID NO: 710 - DNA, 20 bp, Homo sapiens
cgagtgcatcattgccaacc

SEQ ID NO: 711 - DNA, 20 bp, Homo sapiens
gagtgcatcattgccaaccc

SEQ ID NO: 712 - DNA, 20 bp, Homo sapiens
agtgcatcattgccaacccg

SEQ ID NO: 713 - DNA, 20 bp, Homo sapiens
gtgcatcattgccaacccgg

SEQ ID NO: 714 - DNA, 20 bp, Homo sapiens
tgcatcattgccaacccggc

SEQ ID NO: 715 - DNA, 20 bp, Homo sapiens
gcatcattgccaacccggcc

SEQ ID NO: 716 - DNA, 20 bp, Homo sapiens
catcattgccaacccggcct

SEQ ID NO: 717 - DNA, 20 bp, Homo sapiens
atcattgccaacccggcctt

SEQ ID NO: 718 - DNA, 20 bp, Homo sapiens
tcattgccaacccggccttc

SEQ ID NO: 719 - DNA, 20 bp, Homo sapiens
cattgccaacccggccttcg

SEQ ID NO: 720 - DNA, 20 bp, Homo sapiens
attgccaacccggccttcgt

SEQ ID NO: 721 - DNA, 20 bp, Homo sapiens
ttgccaacccggccttcgtg

SEQ ID NO: 722 - DNA, 20 bp, Homo sapiens
tgccaacccggccttcgtgg

SEQ ID NO: 723 - DNA, 20 bp, Homo sapiens
gccaacccggccttcgtggt

SEQ ID NO: 724 - DNA, 20 bp, Homo sapiens
ccaacccggccttcgtggtc

SEQ ID NO: 725 - DNA, 20 bp, Homo sapiens
caacccggccttcgtggtct

SEQ ID NO: 726 - DNA, 20 bp, Homo sapiens
aacccggccttcgtggtcta

SEQ ID NO: 727 - DNA, 20 bp, Homo sapiens
acccggccttcgtggtctac

SEQ ID NO: 728 - DNA, 20 bp, Homo sapiens
cccggccttcgtggtctact

SEQ ID NO: 729 - DNA, 20 bp, Homo sapiens
ccggccttcgtggtctactc

SEQ ID NO: 730 - DNA, 20 bp, Homo sapiens
cggccttcgtggtctactcc

SEQ ID NO: 731 - DNA, 20 bp, Homo sapiens
ggccttcgtggtctactcct

SEQ ID NO: 732 - DNA, 20 bp, Homo sapiens
gccttcgtggtctactcctc

SEQ ID NO: 733 - DNA, 20 bp, Homo sapiens
ccttcgtggtctactcctcc

SEQ ID NO: 734 - DNA, 20 bp, Homo sapiens
cttcgtggtctactcctcca

SEQ ID NO: 735 - DNA, 20 bp, Homo sapiens
ttcgtggtctactcctccat

SEQ ID NO: 736 - DNA, 20 bp, Homo sapiens
tcgtggtctactcctccatc

SEQ ID NO: 737 - DNA, 20 bp, Homo sapiens
cgtggtctactcctccatcg

SEQ ID NO: 738 - DNA, 20 bp, Homo sapiens
gtggtctactcctccatcgt

SEQ ID NO: 739 - DNA, 20 bp, Homo sapiens
tggtctactcctccatcgtc

SEQ ID NO: 740 - DNA, 20 bp, Homo sapiens
ggtctactcctccatcgtct

SEQ ID NO: 741 - DNA, 20 bp, Homo sapiens
gtctactcctccatcgtctc

SEQ ID NO: 742 - DNA, 20 bp, Homo sapiens
tctactcctccatcgtctcc

SEQ ID NO: 743 - DNA, 20 bp, Homo sapiens
ctactcctccatcgtctcct

SEQ ID NO: 744 - DNA, 20 bp, Homo sapiens
tactcctccatcgtctcctt

SEQ ID NO: 745 - DNA, 20 bp, Homo sapiens
actcctccatcgtctccttc

SEQ ID NO: 746 - DNA, 20 bp, Homo sapiens
ctcctccatcgtctccttct

SEQ ID NO: 747 - DNA, 20 bp, Homo sapiens
tcctccatcgtctccttcta

SEQ ID NO: 748 - DNA, 20 bp, Homo sapiens
cctccatcgtctccttctac

SEQ ID NO: 749 - DNA, 20 bp, Homo sapiens
ctccatcgtctccttctacg

SEQ ID NO: 750 - DNA, 20 bp, Homo sapiens
tccatcgtctccttctacgt

SEQ ID NO: 751 - DNA, 20 bp, Homo sapiens
ccatcgtctccttctacgtg

SEQ ID NO: 752 - DNA, 20 bp, Homo sapiens
catcgtctccttctacgtgc

SEQ ID NO: 753 - DNA, 20 bp, Homo sapiens
atcgtctccttctacgtgcc

SEQ ID NO: 754 - DNA, 20 bp, Homo sapiens
tcgtctccttctacgtgccc

SEQ ID NO: 755 - DNA, 20 bp, Homo sapiens
cgtctccttctacgtgccct

SEQ ID NO: 756 - DNA, 20 bp, Homo sapiens
gtctccttctacgtgccctt

SEQ ID NO: 757 - DNA, 20 bp, Homo sapiens
tctccttctacgtgcccttc

SEQ ID NO: 758 - DNA, 20 bp, Homo sapiens
ctccttctacgtgcccttca

SEQ ID NO: 759 - DNA, 20 bp, Homo sapiens
tccttctacgtgcccttcat

SEQ ID NO: 760 - DNA, 20 bp, Homo sapiens
ccttctacgtgcccttcatt

SEQ ID NO: 761 - DNA, 20 bp, Homo sapiens
cttctacgtgcccttcattg

SEQ ID NO: 762 - DNA, 20 bp, Homo sapiens
ttctacgtgcccttcattgt

SEQ ID NO: 763 - DNA, 20 bp, Homo sapiens
tctacgtgcccttcattgtc

SEQ ID NO: 764 - DNA, 20 bp, Homo sapiens
ctacgtgcccttcattgtca

SEQ ID NO: 765 - DNA, 20 bp, Homo sapiens
tacgtgcccttcattgtcac

SEQ ID NO: 766 - DNA, 20 bp, Homo sapiens
acgtgcccttcattgtcacc

SEQ ID NO: 767 - DNA, 20 bp, Homo sapiens
cgtgcccttcattgtcaccc

SEQ ID NO: 768 - DNA, 20 bp, Homo sapiens
gtgcccttcattgtcaccct

SEQ ID NO: 769 - DNA, 20 bp, Homo sapiens
tgcccttcattgtcaccctg

SEQ ID NO: 770 - DNA, 20 bp, Homo sapiens
gcccttcattgtcaccctgc

SEQ ID NO: 771 - DNA, 20 bp, Homo sapiens
cccttcattgtcaccctgct

SEQ ID NO: 772 - DNA, 20 bp, Homo sapiens
ccttcattgtcaccctgctg

SEQ ID NO: 773 - DNA, 20 bp, Homo sapiens
cttcattgtcaccctgctgg

SEQ ID NO: 774 - DNA, 20 bp, Homo sapiens
ttcattgtcaccctgctggt

SEQ ID NO: 775 - DNA, 20 bp, Homo sapiens
tcattgtcaccctgctggtc

SEQ ID NO: 776 - DNA, 20 bp, Homo sapiens
cattgtcaccctgctggtct

SEQ ID NO: 777 - DNA, 20 bp, Homo sapiens
attgtcaccctgctggtcta

SEQ ID NO: 778 - DNA, 20 bp, Homo sapiens
ttgtcaccctgctggtctac

SEQ ID NO: 779 - DNA, 20 bp, Homo sapiens
tgtcaccctgctggtctaca

SEQ ID NO: 780 - DNA, 20 bp, Homo sapiens
gtcaccctgctggtctacat

SEQ ID NO: 781 - DNA, 20 bp, Homo sapiens
tcaccctgctggtctacatc

SEQ ID NO: 782 - DNA, 20 bp, Homo sapiens
caccctgctggtctacatca

SEQ ID NO: 783 - DNA, 20 bp, Homo sapiens
accctgctggtctacatcaa

SEQ ID NO: 784 - DNA, 20 bp, Homo sapiens
ccctgctggtctacatcaag

SEQ ID NO: 785 - DNA, 20 bp, Homo sapiens
cctgctggtctacatcaaga

SEQ ID NO: 786 - DNA, 20 bp, Homo sapiens
ctgctggtctacatcaagat

SEQ ID NO: 787 - DNA, 20 bp, Homo sapiens
tgctggtctacatcaagatc

SEQ ID NO: 788 - DNA, 20 bp, Homo sapiens
gctggtctacatcaagatct

SEQ ID NO: 789 - DNA, 20 bp, Homo sapiens
ctggtctacatcaagatcta

SEQ ID NO: 790 - DNA, 20 bp, Homo sapiens
tggtctacatcaagatctac

SEQ ID NO: 791 - DNA, 20 bp, Homo sapiens
ggtctacatcaagatctaca

SEQ ID NO: 792 - DNA, 20 bp, Homo sapiens
gtctacatcaagatctacat

SEQ ID NO: 793 - DNA, 20 bp, Homo sapiens
tctacatcaagatctacatt

SEQ ID NO: 794 - DNA, 20 bp, Homo sapiens
ctacatcaagatctacattg

SEQ ID NO: 795 - DNA, 20 bp, Homo sapiens
tacatcaagatctacattgt

SEQ ID NO: 796 - DNA, 20 bp, Homo sapiens
acatcaagatctacattgtc

SEQ ID NO: 797 - DNA, 20 bp, Homo sapiens
catcaagatctacattgtcc

SEQ ID NO: 798 - DNA, 20 bp, Homo sapiens
atcaagatctacattgtcct

SEQ ID NO: 799 - DNA, 20 bp, Homo sapiens
tcaagatctacattgtcctc

SEQ ID NO: 800 - DNA, 20 bp, Homo sapiens
caagatctacattgtcctcc

SEQ ID NO: 801 - DNA, 20 bp, Homo sapiens
aagatctacattgtcctccg

SEQ ID NO: 802 - DNA, 20 bp, Homo sapiens
agatctacattgtcctccgc

SEQ ID NO: 803 - DNA, 20 bp, Homo sapiens
gatctacattgtcctccgca

SEQ ID NO: 804 - DNA, 20 bp, Homo sapiens
atctacattgtcctccgcag

SEQ ID NO: 805 - DNA, 20 bp, Homo sapiens
tctacattgtcctccgcaga

SEQ ID NO: 806 - DNA, 20 bp, Homo sapiens
ctacattgtcctccgcagac

SEQ ID NO: 807 - DNA, 20 bp, Homo sapiens
tacattgtcctccgcagacg

SEQ ID NO: 808 - DNA, 20 bp, Homo sapiens
acattgtcctccgcagacgc

SEQ ID NO: 809 - DNA, 20 bp, Homo sapiens
cattgtcctccgcagacgcc

SEQ ID NO: 810 - DNA, 20 bp, Homo sapiens
attgtcctccgcagacgccg

SEQ ID NO: 811 - DNA, 20 bp, Homo sapiens
ttgtcctccgcagacgccgc

SEQ ID NO: 812 - DNA, 20 bp, Homo sapiens
tgtcctccgcagacgccgca

SEQ ID NO: 813 - DNA, 20 bp, Homo sapiens
gtcctccgcagacgccgcaa

SEQ ID NO: 814 - DNA, 21 bp, Homo sapiens
gtcctccgcagacgccgcaag

SEQ ID NO: 815 - DNA, 20 bp, Homo sapiens
tcctccgcagacgccgcaag

SEQ ID NO: 816 - DNA, 20 bp, Homo sapiens
cctccgcagacgccgcaagc

SEQ ID NO: 817 - DNA, 20 bp, Homo sapiens
ctccgcagacgccgcaagcg

SEQ ID NO: 818 - DNA, 20 bp, Homo sapiens
tccgcagacgccgcaagcga

SEQ ID NO: 819 - DNA, 20 bp, Homo sapiens
ccgcagacgccgcaagcgag

SEQ ID NO: 820 - DNA, 20 bp, Homo sapiens
cgcagacgccgcaagcgagt

SEQ ID NO: 821 - DNA, 20 bp, Homo sapiens
gcagacgccgcaagcgagtc

SEQ ID NO: 822 - DNA, 20 bp, Homo sapiens
cagacgccgcaagcgagtca

SEQ ID NO: 823 - DNA, 20 bp, Homo sapiens
agacgccgcaagcgagtcaa

SEQ ID NO: 824 - DNA, 20 bp, Homo sapiens
gacgccgcaagcgagtcaac

SEQ ID NO: 825 - DNA, 20 bp, Homo sapiens
acgccgcaagcgagtcaaca

SEQ ID NO: 826 - DNA, 20 bp, Homo sapiens
cgccgcaagcgagtcaacac

SEQ ID NO: 827 - DNA, 20 bp, Homo sapiens
gccgcaagcgagtcaacacc

SEQ ID NO: 828 - DNA, 20 bp, Homo sapiens
ccgcaagcgagtcaacacca

SEQ ID NO: 829 - DNA, 20 bp, Homo sapiens
cgcaagcgagtcaacaccaa

SEQ ID NO: 830 - DNA, 20 bp, Homo sapiens
gcaagcgagtcaacaccaaa

SEQ ID NO: 831 - DNA, 20 bp, Homo sapiens
caagcgagtcaacaccaaac

SEQ ID NO: 832 - DNA, 20 bp, Homo sapiens
aagcgagtcaacaccaaacg

SEQ ID NO: 833 - DNA, 20 bp, Homo sapiens
agcgagtcaacaccaaacgc

SEQ ID NO: 834 - DNA, 20 bp, Homo sapiens
gcgagtcaacaccaaacgca

SEQ ID NO: 835 - DNA, 20 bp, Homo sapiens
cgagtcaacaccaaacgcag

SEQ ID NO: 836 - DNA, 20 bp, Homo sapiens
gagtcaacaccaaacgcagc

SEQ ID NO: 837 - DNA, 20 bp, Homo sapiens
agtcaacaccaaacgcagca

SEQ ID NO: 838 - DNA, 20 bp, Homo sapiens
gtcaacaccaaacgcagcag

SEQ ID NO: 839 - DNA, 20 bp, Homo sapiens
tcaacaccaaacgcagcagc

SEQ ID NO: 840 - DNA, 20 bp, Homo sapiens
caacaccaaacgcagcagcc

SEQ ID NO: 841 - DNA, 20 bp, Homo sapiens
aacaccaaacgcagcagccg

SEQ ID NO: 842 - DNA, 20 bp, Homo sapiens
acaccaaacgcagcagccga

SEQ ID NO: 843 - DNA, 20 bp, Homo sapiens
caccaaacgcagcagccgag

SEQ ID NO: 844 - DNA, 20 bp, Homo sapiens
accaaacgcagcagccgagc

SEQ ID NO: 845 - DNA, 20 bp, Homo sapiens
ccaaacgcagcagccgagct

SEQ ID NO: 846 - DNA, 20 bp, Homo sapiens
caaacgcagcagccgagctt

SEQ ID NO: 847 - DNA, 20 bp, Homo sapiens
aaacgcagcagccgagcttt

SEQ ID NO: 848 - DNA, 20 bp, Homo sapiens
aacgcagcagccgagctttc

SEQ ID NO: 849 - DNA, 20 bp, Homo sapiens
acgcagcagccgagctttca

SEQ ID NO: 850 - DNA, 20 bp, Homo sapiens
cgcagcagccgagctttcag

SEQ ID NO: 851 - DNA, 20 bp, Homo sapiens
gcagcagccgagctttcagg

SEQ ID NO: 852 - DNA, 20 bp, Homo sapiens
cagcagccgagctttcaggg

SEQ ID NO: 853 - DNA, 20 bp, Homo sapiens
agcagccgagctttcagggc

SEQ ID NO: 854 - DNA, 20 bp, Homo sapiens
gcagccgagctttcagggcc

SEQ ID NO: 855 - DNA, 20 bp, Homo sapiens
cagccgagctttcagggccc

SEQ ID NO: 856 - DNA, 20 bp, Homo sapiens
agccgagctttcagggccca

SEQ ID NO: 857 - DNA, 20 bp, Homo sapiens
gccgagctttcagggcccac

SEQ ID NO: 858 - DNA, 20 bp, Homo sapiens
ccgagctttcagggcccacc

SEQ ID NO: 859 - DNA, 20 bp, Homo sapiens
cgagctttcagggcccacct

SEQ ID NO: 860 - DNA, 20 bp, Homo sapiens
gagctttcagggcccacctg

SEQ ID NO: 861 - DNA, 20 bp, Homo sapiens
agctttcagggcccacctga

SEQ ID NO: 862 - DNA, 20 bp, Homo sapiens
gctttcagggcccacctgag

SEQ ID NO: 863 - DNA, 20 bp, Homo sapiens
ctttcagggcccacctgagg

SEQ ID NO: 864 - DNA, 20 bp, Homo sapiens
tttcagggcccacctgaggg

SEQ ID NO: 865 - DNA, 20 bp, Homo sapiens
ttcagggcccacctgagggc

SEQ ID NO: 866 - DNA, 20 bp, Homo sapiens
tcagggcccacctgagggct

SEQ ID NO: 867 - DNA, 20 bp, Homo sapiens
cagggcccacctgagggctc

SEQ ID NO: 868 - DNA, 20 bp, Homo sapiens
agggcccacctgagggctcc

SEQ ID NO: 869 - DNA, 20 bp, Homo sapiens
gggcccacctgagggctcca

SEQ ID NO: 870 - DNA, 20 bp, Homo sapiens
ggcccacctgagggctccac

SEQ ID NO: 871 - DNA, 20 bp, Homo sapiens
gcccacctgagggctccact

SEQ ID NO: 872 - DNA, 20 bp, Homo sapiens
cccacctgagggctccacta

SEQ ID NO: 873 - DNA, 20 bp, Homo sapiens
ccacctgagggctccactaa

SEQ ID NO: 874 - DNA, 20 bp, Homo sapiens
cacctgagggctccactaaa

SEQ ID NO: 875 - DNA, 20 bp, Homo sapiens
acctgagggctccactaaag

SEQ ID NO: 876 - DNA, 20 bp, Homo sapiens
cctgagggctccactaaagg

SEQ ID NO: 877 - DNA, 20 bp, Homo sapiens
ctgagggctccactaaaggg

SEQ ID NO: 878 - DNA, 20 bp, Homo sapiens
tgagggctccactaaagggc

SEQ ID NO: 879 - DNA, 20 bp, Homo sapiens
gagggctccactaaagggca

SEQ ID NO: 880 - DNA, 20 bp, Homo sapiens
agggctccactaaagggcaa

SEQ ID NO: 881 - DNA, 20 bp, Homo sapiens
gggctccactaaagggcaac

SEQ ID NO: 882 - DNA, 20 bp, Homo sapiens
ggctccactaaagggcaact

SEQ ID NO: 883 - DNA, 20 bp, Homo sapiens
gctccactaaagggcaactg

SEQ ID NO: 884 - DNA, 20 bp, Homo sapiens
ctccactaaagggcaactgt

SEQ ID NO: 885 - DNA, 20 bp, Homo sapiens
tccactaaagggcaactgta

SEQ ID NO: 886 - DNA, 20 bp, Homo sapiens
ccactaaagggcaactgtac

SEQ ID NO: 887 - DNA, 20 bp, Homo sapiens
cactaaagggcaactgtact

SEQ ID NO: 888 - DNA, 20 bp, Homo sapiens
actaaagggcaactgtactc

SEQ ID NO: 889 - DNA, 20 bp, Homo sapiens
ctaaagggcaactgtactca

SEQ ID NO: 890 - DNA, 20 bp, Homo sapiens
taaagggcaactgtactcac

SEQ ID NO: 891 - DNA, 20 bp, Homo sapiens
aaagggcaactgtactcacc

SEQ ID NO: 892 - DNA, 20 bp, Homo sapiens
aagggcaactgtactcaccc

SEQ ID NO: 893 - DNA, 20 bp, Homo sapiens
agggcaactgtactcacccc

SEQ ID NO: 894 - DNA, 20 bp, Homo sapiens
gggcaactgtactcaccccg

SEQ ID NO: 895 - DNA, 20 bp, Homo sapiens
ggcaactgtactcaccccga

SEQ ID NO: 896 - DNA, 20 bp, Homo sapiens
gcaactgtactcaccccgag

SEQ ID NO: 897 - DNA, 20 bp, Homo sapiens
caactgtactcaccccgagg

SEQ ID NO: 898 - DNA, 20 bp, Homo sapiens
aactgtactcaccccgagga

SEQ ID NO: 899 - DNA, 20 bp, Homo sapiens
actgtactcaccccgaggac

SEQ ID NO: 900 - DNA, 20 bp, Homo sapiens
ctgtactcaccccgaggaca

SEQ ID NO: 901 - DNA, 20 bp, Homo sapiens
tgtactcaccccgaggacat

SEQ ID NO: 902 - DNA, 20 bp, Homo sapiens
gtactcaccccgaggacatg

SEQ ID NO: 903 - DNA, 20 bp, Homo sapiens
tactcaccccgaggacatga

SEQ ID NO: 904 - DNA, 20 bp, Homo sapiens
actcaccccgaggacatgaa

SEQ ID NO: 905 - DNA, 20 bp, Homo sapiens
ctcaccccgaggacatgaaa

SEQ ID NO: 906 - DNA, 20 bp, Homo sapiens
tcaccccgaggacatgaaac

SEQ ID NO: 907 - DNA, 20 bp, Homo sapiens
caccccgaggacatgaaact

SEQ ID NO: 908 - DNA, 20 bp, Homo sapiens
accccgaggacatgaaactc

SEQ ID NO: 909 - DNA, 20 bp, Homo sapiens
ccccgaggacatgaaactct

SEQ ID NO: 910 - DNA, 20 bp, Homo sapiens
cccgaggacatgaaactctg

SEQ ID NO: 911 - DNA, 20 bp, Homo sapiens
ccgaggacatgaaactctgc

SEQ ID NO: 912 - DNA, 20 bp, Homo sapiens
cgaggacatgaaactctgca

SEQ ID NO: 913 - DNA, 20 bp, Homo sapiens
gaggacatgaaactctgcac

SEQ ID NO: 914 - DNA, 20 bp, Homo sapiens
aggacatgaaactctgcacc

SEQ ID NO: 915 - DNA, 20 bp, Homo sapiens
ggacatgaaactctgcaccg

SEQ ID NO: 916 - DNA, 20 bp, Homo sapiens
gacatgaaactctgcaccgt

SEQ ID NO: 917 - DNA, 20 bp, Homo sapiens
acatgaaactctgcaccgtt

SEQ ID NO: 918 - DNA, 20 bp, Homo sapiens
catgaaactctgcaccgtta

SEQ ID NO: 919 - DNA, 20 bp, Homo sapiens
atgaaactctgcaccgttat

SEQ ID NO: 920 - DNA, 20 bp, Homo sapiens
tgaaactctgcaccgttatc

SEQ ID NO: 921 - DNA, 20 bp, Homo sapiens
tgaaactctgcaccgttatc

SEQ ID NO: 922 - DNA, 20 bp, Homo sapiens
gaaactctgcaccgttatca

SEQ ID NO: 923 - DNA, 20 bp, Homo sapiens
aaactctgcaccgttatcat

SEQ ID NO: 924 - DNA, 20 bp, Homo sapiens
aactctgcaccgttatcatg

SEQ ID NO: 925 - DNA, 20 bp, Homo sapiens
actctgcaccgttatcatga

SEQ ID NO: 926 - DNA, 20 bp, Homo sapiens
actctgcaccgttatcatga

SEQ ID NO: 927 - DNA, 20 bp, Homo sapiens
tctgcaccgttatcatgaag

SEQ ID NO: 928 - DNA, 20 bp, Homo sapiens
ctgcaccgttatcatgaagt

SEQ ID NO: 929 - DNA, 20 bp, Homo sapiens
tgcaccgttatcatgaagtc

SEQ ID NO: 930 - DNA, 20 bp, Homo sapiens
gcaccgttatcatgaagtct

SEQ ID NO: 931 - DNA, 20 bp, Homo sapiens
caccgttatcatgaagtcta

SEQ ID NO: 932 - DNA, 20 bp, Homo sapiens
accgttatcatgaagtctaa

SEQ ID NO: 933 - DNA, 20 bp, Homo sapiens
ccgttatcatgaagtctaat

SEQ ID NO: 934 - DNA, 20 bp, Homo sapiens
cgttatcatgaagtctaatg

SEQ ID NO: 935 - DNA, 20 bp, Homo sapiens
gttatcatgaagtctaatgg

SEQ ID NO: 936 - DNA, 20 bp, Homo sapiens
ttatcatgaagtctaatggg

SEQ ID NO: 937 - DNA, 20 bp, Homo sapiens
tatcatgaagtctaatggga

SEQ ID NO: 938 - DNA, 20 bp, Homo sapiens
atcatgaagtctaatgggag

SEQ ID NO: 939 - DNA, 20 bp, Homo sapiens
tcatgaagtctaatgggagt

SEQ ID NO: 940 - DNA, 20 bp, Homo sapiens
catgaagtctaatgggagtt

SEQ ID NO: 941 - DNA, 20 bp, Homo sapiens
atgaagtctaatgggagttt

SEQ ID NO: 942 - DNA, 20 bp, Homo sapiens
tgaagtctaatgggagtttc

SEQ ID NO: 943 - DNA, 20 bp, Homo sapiens
gaagtctaatgggagtttcc

SEQ ID NO: 944 - DNA, 20 bp, Homo sapiens
aagtctaatgggagtttccc

SEQ ID NO: 945 - DNA, 20 bp, Homo sapiens
agtctaatgggagtttccca

SEQ ID NO: 946 - DNA, 20 bp, Homo sapiens
gtctaatgggagtttcccag

SEQ ID NO: 947 - DNA, 20 bp, Homo sapiens
tctaatgggagtttcccagt

SEQ ID NO: 948 - DNA, 20 bp, Homo sapiens
ctaatgggagtttcccagtg

SEQ ID NO: 949 - DNA, 20 bp, Homo sapiens
taatgggagtttcccagtga

SEQ ID NO: 950 - DNA, 20 bp, Homo sapiens
aatgggagtttcccagtgaa

SEQ ID NO: 951 - DNA, 20 bp, Homo sapiens
atgggagtttcccagtgaac

SEQ ID NO: 952 - DNA, 20 bp, Homo sapiens
tgggagtttcccagtgaaca

SEQ ID NO: 953 - DNA, 20 bp, Homo sapiens
gggagtttcccagtgaacag

SEQ ID NO: 954 - DNA, 20 bp, Homo sapiens
ggagtttcccagtgaacagg

SEQ ID NO: 955 - DNA, 20 bp, Homo sapiens
gagtttcccagtgaacaggc

SEQ ID NO: 956 - DNA, 20 bp, Homo sapiens
agtttcccagtgaacaggcg

SEQ ID NO: 957 - DNA, 20 bp, Homo sapiens
gtttcccagtgaacaggcgg

SEQ ID NO: 958 - DNA, 20 bp, Homo sapiens
tttcccagtgaacaggcgga

SEQ ID NO: 959 - DNA, 20 bp, Homo sapiens
ttcccagtgaacaggcggag

SEQ ID NO: 960 - DNA, 20 bp, Homo sapiens
tcccagtgaacaggcggaga

SEQ ID NO: 961 - DNA, 20 bp, Homo sapiens
cccagtgaacaggcggagag

SEQ ID NO: 962 - DNA, 20 bp, Homo sapiens
ccagtgaacaggcggagagt

SEQ ID NO: 963 - DNA, 20 bp, Homo sapiens
cagtgaacaggcggagagtg

SEQ ID NO: 964 - DNA, 20 bp, Homo sapiens
agtgaacaggcggagagtgg

SEQ ID NO: 965 - DNA, 20 bp, Homo sapiens
gtgaacaggcggagagtgga

SEQ ID NO: 966 - DNA, 20 bp, Homo sapiens
tgaacaggcggagagtggag

SEQ ID NO: 967 - DNA, 20 bp, Homo sapiens
gaacaggcggagagtggagg

SEQ ID NO: 968 - DNA, 20 bp, Homo sapiens
aacaggcggagagtggaggc

SEQ ID NO: 969 - DNA, 20 bp, Homo sapiens
acaggcggagagtggaggct

SEQ ID NO: 970 - DNA, 20 bp, Homo sapiens
caggcggagagtggaggctg

SEQ ID NO: 971 - DNA, 20 bp, Homo sapiens
aggcggagagtggaggctgc

SEQ ID NO: 972 - DNA, 20 bp, Homo sapiens
ggcggagagtggaggctgcc

SEQ ID NO: 973 - DNA, 20 bp, Homo sapiens
gcggagagtggaggctgccc

SEQ ID NO: 974 - DNA, 20 bp, Homo sapiens
cggagagtggaggctgcccg

SEQ ID NO: 975 - DNA, 20 bp, Homo sapiens
ggagagtggaggctgcccgg

SEQ ID NO: 976 - DNA, 20 bp, Homo sapiens
gagagtggaggctgcccggc

SEQ ID NO: 977 - DNA, 20 bp, Homo sapiens
agagtggaggctgcccggcg

SEQ ID NO: 978 - DNA, 20 bp, Homo sapiens
gagtggaggctgcccggcga

SEQ ID NO: 979 - DNA, 20 bp, Homo sapiens
agtggaggctgcccggcgag

SEQ ID NO: 980 - DNA, 20 bp, Homo sapiens
gtggaggctgcccggcgagc

SEQ ID NO: 981 - DNA, 20 bp, Homo sapiens
tggaggctgcccggcgagcc

SEQ ID NO: 982 - DNA, 20 bp, Homo sapiens
ggaggctgcccggcgagccc

SEQ ID NO: 983 - DNA, 20 bp, Homo sapiens
gaggctgcccggcgagccca

SEQ ID NO: 984 - DNA, 20 bp, Homo sapiens
aggctgcccggcgagcccag

SEQ ID NO: 985 - DNA, 20 bp, Homo sapiens
ggctgcccggcgagcccagg

SEQ ID NO: 986 - DNA, 20 bp, Homo sapiens
gctgcccggcgagcccagga

SEQ ID NO: 987 - DNA, 20 bp, Homo sapiens
ctgcccggcgagcccaggag

SEQ ID NO: 988 - DNA, 20 bp, Homo sapiens
tgcccggcgagcccaggagc

SEQ ID NO: 989 - DNA, 20 bp, Homo sapiens
gcccggcgagcccaggagct

SEQ ID NO: 990 - DNA, 20 bp, Homo sapiens
cccggcgagcccaggagctg

SEQ ID NO: 991 - DNA, 20 bp, Homo sapiens
ccggcgagcccaggagctgg

SEQ ID NO: 992 - DNA, 20 bp, Homo sapiens
cggcgagcccaggagctgga

SEQ ID NO: 993 - DNA, 20 bp, Homo sapiens
ggcgagcccaggagctggag

SEQ ID NO: 994 - DNA, 20 bp, Homo sapiens
gcgagcccaggagctggaga

SEQ ID NO: 995 - DNA, 20 bp, Homo sapiens
cgagcccaggagctggagat

SEQ ID NO: 996 - DNA, 20 bp, Homo sapiens
gagcccaggagctggagatg

SEQ ID NO: 997 - DNA, 20 bp, Homo sapiens
agcccaggagctggagatgg

SEQ ID NO: 998 - DNA, 20 bp, Homo sapiens
gcccaggagctggagatgga

SEQ ID NO: 999 - DNA, 20 bp, Homo sapiens
cccaggagctggagatggag

SEQ ID NO: 1000 - DNA, 20 bp, Homo sapiens
ccaggagctggagatggaga

SEQ ID NO: 1001 - DNA, 20 bp, Homo sapiens
caggagctggagatggagat

SEQ ID NO: 1002 - DNA, 20 bp, Homo sapiens
aggagctggagatggagatg

SEQ ID NO: 1003 - DNA, 20 bp, Homo sapiens
ggagctggagatggagatgc

SEQ ID NO: 1004 - DNA, 20 bp, Homo sapiens
gagctggagatggagatgct

SEQ ID NO: 1005 - DNA, 20 bp, Homo sapiens
agctggagatggagatgctc

SEQ ID NO: 1006 - DNA, 20 bp, Homo sapiens
gctggagatggagatgctct

SEQ ID NO: 1007 - DNA, 20 bp, Homo sapiens
ctggagatggagatgctctc

SEQ ID NO: 1008 - DNA, 20 bp, Homo sapiens
tggagatggagatgctctcc

SEQ ID NO: 1009 - DNA, 20 bp, Homo sapiens
ggagatggagatgctctcca

SEQ ID NO: 1010 - DNA, 20 bp, Homo sapiens
gagatggagatgctctccag

SEQ ID NO: 1011 - DNA, 20 bp, Homo sapiens
agatggagatgctctccagc

SEQ ID NO: 1012 - DNA, 20 bp, Homo sapiens
gatggagatgctctccagca

SEQ ID NO: 1013 - DNA, 20 bp, Homo sapiens
atggagatgctctccagcac

SEQ ID NO: 1014 - DNA, 20 bp, Homo sapiens
tggagatgctctccagcacc

SEQ ID NO: 1015 - DNA, 20 bp, Homo sapiens
ggagatgctctccagcacca

SEQ ID NO: 1016 - DNA, 20 bp, Homo sapiens
gagatgctctccagcaccag

SEQ ID NO: 1017 - DNA, 20 bp, Homo sapiens
agatgctctccagcaccagc

SEQ ID NO: 1018 - DNA, 20 bp, Homo sapiens
gatgctctccagcaccagcc

SEQ ID NO: 1019 - DNA, 20 bp, Homo sapiens
atgctctccagcaccagccc

SEQ ID NO: 1020 - DNA, 20 bp, Homo sapiens
tgctctccagcaccagccca

SEQ ID NO: 1021 - DNA, 20 bp, Homo sapiens
gctctccagcaccagcccac

SEQ ID NO: 1022 - DNA, 20 bp, Homo sapiens
ctctccagcaccagcccacc

SEQ ID NO: 1023 - DNA, 20 bp, Homo sapiens
tctccagcaccagcccaccc

SEQ ID NO: 1024 - DNA, 20 bp, Homo sapiens
ctccagcaccagcccacccg

SEQ ID NO: 1025 - DNA, 20 bp, Homo sapiens
tccagcaccagcccacccga

SEQ ID NO: 1026 - DNA, 20 bp, Homo sapiens
ccagcaccagcccacccgag

SEQ ID NO: 1027 - DNA, 20 bp, Homo sapiens
cagcaccagcccacccgaga

SEQ ID NO: 1028 - DNA, 20 bp, Homo sapiens
agcaccagcccacccgagag

SEQ ID NO: 1029 - DNA, 20 bp, Homo sapiens
gcaccagcccacccgagagg

SEQ ID NO: 1030 - DNA, 20 bp, Homo sapiens
caccagcccacccgagagga

SEQ ID NO: 1031 - DNA, 20 bp, Homo sapiens
accagcccacccgagaggac

SEQ ID NO: 1032 - DNA, 20 bp, Homo sapiens
ccagcccacccgagaggacc

SEQ ID NO: 1033 - DNA, 20 bp, Homo sapiens
cagcccacccgagaggaccc

SEQ ID NO: 1034 - DNA, 20 bp, Homo sapiens
agcccacccgagaggacccg

SEQ ID NO: 1035 - DNA, 20 bp, Homo sapiens
gcccacccgagaggacccgg

SEQ ID NO: 1036 - DNA, 20 bp, Homo sapiens
cccacccgagaggacccggt

SEQ ID NO: 1037 - DNA, 20 bp, Homo sapiens
ccacccgagaggacccggta

SEQ ID NO: 1038 - DNA, 20 bp, Homo sapiens
cacccgagaggacccggtac

SEQ ID NO: 1039 - DNA, 20 bp, Homo sapiens
acccgagaggacccggtaca

SEQ ID NO: 1040 - DNA, 20 bp, Homo sapiens
cccgagaggacccggtacag

SEQ ID NO: 1041 - DNA, 20 bp, Homo sapiens
ccgagaggacccggtacagc

SEQ ID NO: 1042 - DNA, 20 bp, Homo sapiens
cgagaggacccggtacagcc

SEQ ID NO: 1043 - DNA, 20 bp, Homo sapiens
gagaggacccggtacagccc

SEQ ID NO: 1044 - DNA, 20 bp, Homo sapiens
agaggacccggtacagcccc

SEQ ID NO: 1045 - DNA, 20 bp, Homo sapiens
gaggacccggtacagcccca

SEQ ID NO: 1046 - DNA, 20 bp, Homo sapiens
aggacccggtacagccccat

SEQ ID NO: 1047 - DNA, 20 bp, Homo sapiens
ggacccggtacagccccatc

SEQ ID NO: 1048 - DNA, 20 bp, Homo sapiens
gacccggtacagccccatcc

SEQ ID NO: 1049 - DNA, 20 bp, Homo sapiens
acccggtacagccccatccc

SEQ ID NO: 1050 - DNA, 20 bp, Homo sapiens
cccggtacagccccatccca

SEQ ID NO: 1051 - DNA, 20 bp, Homo sapiens
ccggtacagccccatcccac

SEQ ID NO: 1052 - DNA, 20 bp, Homo sapiens
cggtacagccccatcccacc

SEQ ID NO: 1053 - DNA, 20 bp, Homo sapiens
ggtacagccccatcccaccc

SEQ ID NO: 1054 - DNA, 20 bp, Homo sapiens
gtacagccccatcccaccca

SEQ ID NO: 1055 - DNA, 20 bp, Homo sapiens
tacagccccatcccacccag

SEQ ID NO: 1056 - DNA, 20 bp, Homo sapiens
acagccccatcccacccagc

SEQ ID NO: 1057 - DNA, 20 bp, Homo sapiens
cagccccatcccacccagcc

SEQ ID NO: 1058 - DNA, 20 bp, Homo sapiens
agccccatcccacccagcca

SEQ ID NO: 1059 - DNA, 20 bp, Homo sapiens
gccccatcccacccagccac

SEQ ID NO: 1060 - DNA, 20 bp, Homo sapiens
ccccatcccacccagccacc

SEQ ID NO: 1061 - DNA, 20 bp, Homo sapiens
cccatcccacccagccacca

SEQ ID NO: 1062 - DNA, 20 bp, Homo sapiens
ccatcccacccagccaccac

SEQ ID NO: 1063 - DNA, 20 bp, Homo sapiens
catcccacccagccaccacc

SEQ ID NO: 1064 - DNA, 20 bp, Homo sapiens
atcccacccagccaccacca

SEQ ID NO: 1065 - DNA, 20 bp, Homo sapiens
tcccacccagccaccaccag

SEQ ID NO: 1066 - DNA, 20 bp, Homo sapiens
cccacccagccaccaccagc

SEQ ID NO: 1067 - DNA, 20 bp, Homo sapiens
ccacccagccaccaccagct

SEQ ID NO: 1068 - DNA, 20 bp, Homo sapiens
cacccagccaccaccagctg

SEQ ID NO: 1069 - DNA, 20 bp, Homo sapiens
acccagccaccaccagctga

SEQ ID NO: 1070 - DNA, 20 bp, Homo sapiens
cccagccaccaccagctgac

SEQ ID NO: 1071 - DNA, 20 bp, Homo sapiens
ccagccaccaccagctgact

SEQ ID NO: 1072 - DNA, 20 bp, Homo sapiens
cagccaccaccagctgactc

SEQ ID NO: 1073 - DNA, 20 bp, Homo sapiens
agccaccaccagctgactct

SEQ ID NO: 1074 - DNA, 20 bp, Homo sapiens
gccaccaccagctgactctc

SEQ ID NO: 1075 - DNA, 20 bp, Homo sapiens
ccaccaccagctgactctcc

SEQ ID NO: 1076 - DNA, 20 bp, Homo sapiens
caccaccagctgactctccc

SEQ ID NO: 1077 - DNA, 20 bp, Homo sapiens
accaccagctgactctcccc

SEQ ID NO: 1078 - DNA, 20 bp, Homo sapiens
ccaccagctgactctccccg

SEQ ID NO: 1079 - DNA, 20 bp, Homo sapiens
caccagctgactctccccga

SEQ ID NO: 1080 - DNA, 20 bp, Homo sapiens
accagctgactctccccgac

SEQ ID NO: 1081 - DNA, 20 bp, Homo sapiens
ccagctgactctccccgacc

SEQ ID NO: 1082 - DNA, 20 bp, Homo sapiens
cagctgactctccccgaccc

SEQ ID NO: 1083 - DNA, 20 bp, Homo sapiens
agctgactctccccgacccg

SEQ ID NO: 1084 - DNA, 20 bp, Homo sapiens
gctgactctccccgacccgt

SEQ ID NO: 1085 - DNA, 20 bp, Homo sapiens
ctgactctccccgacccgtc

SEQ ID NO: 1086 - DNA, 20 bp, Homo sapiens
tgactctccccgacccgtcc

SEQ ID NO: 1087 - DNA, 20 bp, Homo sapiens
gactctccccgacccgtccc

SEQ ID NO: 1088 - DNA, 20 bp, Homo sapiens
actctccccgacccgtccca

SEQ ID NO: 1089 - DNA, 20 bp, Homo sapiens
ctctccccgacccgtcccac

SEQ ID NO: 1090 - DNA, 20 bp, Homo sapiens
tctccccgacccgtcccacc

SEQ ID NO: 1091 - DNA, 20 bp, Homo sapiens
ctccccgacccgtcccacca

SEQ ID NO: 1092 - DNA, 20 bp, Homo sapiens
tccccgacccgtcccaccat

SEQ ID NO: 1093 - DNA, 20 bp, Homo sapiens
ccccgacccgtcccaccatg

SEQ ID NO: 1094 - DNA, 20 bp, Homo sapiens
cccgacccgtcccaccatgg

SEQ ID NO: 1095 - DNA, 20 bp, Homo sapiens
ccgacccgtcccaccatggt

SEQ ID NO: 1096 - DNA, 20 bp, Homo sapiens
cgacccgtcccaccatggtc

SEQ ID NO: 1097 - DNA, 20 bp, Homo sapiens
gacccgtcccaccatggtct

SEQ ID NO: 1098 - DNA, 20 bp, Homo sapiens
acccgtcccaccatggtctc

SEQ ID NO: 1099 - DNA, 20 bp, Homo sapiens
cccgtcccaccatggtctcc

SEQ ID NO: 1100 - DNA, 20 bp, Homo sapiens
ccgtcccaccatggtctcca

SEQ ID NO: 1101 - DNA, 20 bp, Homo sapiens
cgtcccaccatggtctccac

SEQ ID NO: 1102 - DNA, 20 bp, Homo sapiens
gtcccaccatggtctccaca

SEQ ID NO: 1103 - DNA, 20 bp, Homo sapiens
tcccaccatggtctccacag

SEQ ID NO: 1104 - DNA, 20 bp, Homo sapiens
cccaccatggtctccacagc

SEQ ID NO: 1105 - DNA, 20 bp, Homo sapiens
ccaccatggtctccacagca

SEQ ID NO: 1106 - DNA, 20 bp, Homo sapiens
caccatggtctccacagcac

SEQ ID NO: 1107 - DNA, 20 bp, Homo sapiens
accatggtctccacagcact

SEQ ID NO: 1108 - DNA, 20 bp, Homo sapiens
ccatggtctccacagcactc

SEQ ID NO: 1109 - DNA, 20 bp, Homo sapiens
catggtctccacagcactcc

SEQ ID NO: 1110 - DNA, 20 bp, Homo sapiens
atggtctccacagcactccc

SEQ ID NO: 1111 - DNA, 20 bp, Homo sapiens
tggtctccacagcactcccg

SEQ ID NO: 1112 - DNA, 20 bp, Homo sapiens
ggtctccacagcactcccga

SEQ ID NO: 1113 - DNA, 20 bp, Homo sapiens
gtctccacagcactcccgac

SEQ ID NO: 1114 - DNA, 20 bp, Homo sapiens
tctccacagcactcccgaca

SEQ ID NO: 1115 - DNA, 20 bp, Homo sapiens
ctccacagcactcccgacag

SEQ ID NO: 1116 - DNA, 20 bp, Homo sapiens
tccacagcactcccgacagc

SEQ ID NO: 1117 - DNA, 20 bp, Homo sapiens
ccacagcactcccgacagcc

SEQ ID NO: 1118 - DNA, 20 bp, Homo sapiens
cacagcactcccgacagccc

SEQ ID NO: 1119 - DNA, 20 bp, Homo sapiens
acagcactcccgacagcccc

SEQ ID NO: 1120 - DNA, 20 bp, Homo sapiens
cagcactcccgacagccccg

SEQ ID NO: 1121 - DNA, 20 bp, Homo sapiens
agcactcccgacagccccgc

SEQ ID NO: 1122 - DNA, 20 bp, Homo sapiens
gcactcccgacagccccgcc

SEQ ID NO: 1123 - DNA, 20 bp, Homo sapiens
cactcccgacagccccgcca

SEQ ID NO: 1124 - DNA, 20 bp, Homo sapiens
actcccgacagccccgccaa

SEQ ID NO: 1125 - DNA, 20 bp, Homo sapiens
ctcccgacagccccgccaaa

SEQ ID NO: 1126 - DNA, 20 bp, Homo sapiens
tcccgacagccccgccaaac

SEQ ID NO: 1127 - DNA, 20 bp, Homo sapiens
cccgacagccccgccaaacc

SEQ ID NO: 1128 - DNA, 20 bp, Homo sapiens
ccgacagccccgccaaacca

SEQ ID NO: 1129 - DNA, 20 bp, Homo sapiens
cgacagccccgccaaaccag

SEQ ID NO: 1130 - DNA, 20 bp, Homo sapiens
gacagccccgccaaaccaga

SEQ ID NO: 1131 - DNA, 20 bp, Homo sapiens
acagccccgccaaaccagag

SEQ ID NO: 1132 - DNA, 20 bp, Homo sapiens
cagccccgccaaaccagaga

SEQ ID NO: 1133 - DNA, 20 bp, Homo sapiens
agccccgccaaaccagagaa

SEQ ID NO: 1134 - DNA, 20 bp, Homo sapiens
gccccgccaaaccagagaag

SEQ ID NO: 1135 - DNA, 20 bp, Homo sapiens
ccccgccaaaccagagaaga

SEQ ID NO: 1136 - DNA, 20 bp, Homo sapiens
cccgccaaaccagagaagaa

SEQ ID NO: 1137 - DNA, 20 bp, Homo sapiens
ccgccaaaccagagaagaat

SEQ ID NO: 1138 - DNA, 20 bp, Homo sapiens
cgccaaaccagagaagaatg

SEQ ID NO: 1139 - DNA, 20 bp, Homo sapiens
gccaaaccagagaagaatgg

SEQ ID NO: 1140 - DNA, 20 bp, Homo sapiens
ccaaaccagagaagaatggg

SEQ ID NO: 1141 - DNA, 20 bp, Homo sapiens
caaaccagagaagaatgggc

SEQ ID NO: 1142 - DNA, 20 bp, Homo sapiens
aaaccagagaagaatgggca

SEQ ID NO: 1143 - DNA, 20 bp, Homo sapiens
aaccagagaagaatgggcat

SEQ ID NO: 1144 - DNA, 20 bp, Homo sapiens
accagagaagaatgggcatg

SEQ ID NO: 1145 - DNA, 20 bp, Homo sapiens
ccagagaagaatgggcatgc

SEQ ID NO: 1146 - DNA, 20 bp, Homo sapiens
cagagaagaatgggcatgcc

SEQ ID NO: 1147 - DNA, 20 bp, Homo sapiens
agagaagaatgggcatgcca

SEQ ID NO: 1148 - DNA, 20 bp, Homo sapiens
gagaagaatgggcatgccaa

SEQ ID NO: 1149 - DNA, 20 bp, Homo sapiens
agaagaatgggcatgccaaa

SEQ ID NO: 1150 - DNA, 20 bp, Homo sapiens
gaagaatgggcatgccaaag

SEQ ID NO: 1151 - DNA, 20 bp, Homo sapiens
aagaatgggcatgccaaaga

SEQ ID NO: 1152 - DNA, 20 bp, Homo sapiens
agaatgggcatgccaaagac

SEQ ID NO: 1153 - DNA, 20 bp, Homo sapiens
gaatgggcatgccaaagacc

SEQ ID NO: 1154 - DNA, 20 bp, Homo sapiens
aatgggcatgccaaagacca

SEQ ID NO: 1155 - DNA, 20 bp, Homo sapiens
atgggcatgccaaagaccac

SEQ ID NO: 1156 - DNA, 20 bp, Homo sapiens
tgggcatgccaaagaccacc

SEQ ID NO: 1157 - DNA, 20 bp, Homo sapiens
gggcatgccaaagaccaccc

SEQ ID NO: 1158 - DNA, 20 bp, Homo sapiens
ggcatgccaaagaccacccc

SEQ ID NO: 1159 - DNA, 20 bp, Homo sapiens
gcatgccaaagaccacccca

SEQ ID NO: 1160 - DNA, 20 bp, Homo sapiens
catgccaaagaccaccccaa

SEQ ID NO: 1161 - DNA, 20 bp, Homo sapiens
atgccaaagaccaccccaag

SEQ ID NO: 1162 - DNA, 20 bp, Homo sapiens
tgccaaagaccaccccaaga

SEQ ID NO: 1163 - DNA, 20 bp, Homo sapiens
gccaaagaccaccccaagat

SEQ ID NO: 1164 - DNA, 20 bp, Homo sapiens
ccaaagaccaccccaagatt

SEQ ID NO: 1165 - DNA, 20 bp, Homo sapiens
caaagaccaccccaagattg

SEQ ID NO: 1166 - DNA, 20 bp, Homo sapiens
aaagaccaccccaagattgc

SEQ ID NO: 1167 - DNA, 20 bp, Homo sapiens
aagaccaccccaagattgcc

SEQ ID NO: 1168 - DNA, 20 bp, Homo sapiens
agaccaccccaagattgcca

SEQ ID NO: 1169 - DNA, 20 bp, Homo sapiens
gaccaccccaagattgccaa

SEQ ID NO: 1170 - DNA, 20 bp, Homo sapiens
accaccccaagattgccaag

SEQ ID NO: 1171 - DNA, 20 bp, Homo sapiens
ccaccccaagattgccaaga

SEQ ID NO: 1172 - DNA, 20 bp, Homo sapiens
caccccaagattgccaagat

SEQ ID NO: 1173 - DNA, 20 bp, Homo sapiens
accccaagattgccaagatc

SEQ ID NO: 1174 - DNA, 20 bp, Homo sapiens
ccccaagattgccaagatct

SEQ ID NO: 1175 - DNA, 20 bp, Homo sapiens
cccaagattgccaagatctt

SEQ ID NO: 1176 - DNA, 20 bp, Homo sapiens
ccaagattgccaagatcttt

SEQ ID NO: 1177 - DNA, 20 bp, Homo sapiens
caagattgccaagatctttg

SEQ ID NO: 1178 - DNA, 20 bp, Homo sapiens
aagattgccaagatctttga

SEQ ID NO: 1179 - DNA, 20 bp, Homo sapiens
agattgccaagatctttgag

SEQ ID NO: 1180 - DNA, 20 bp, Homo sapiens
gattgccaagatctttgaga

SEQ ID NO: 1181 - DNA, 20 bp, Homo sapiens
attgccaagatctttgagat

SEQ ID NO: 1182 - DNA, 20 bp, Homo sapiens
ttgccaagatctttgagatc

SEQ ID NO: 1183 - DNA, 20 bp, Homo sapiens
tgccaagatctttgagatcc

SEQ ID NO: 1184 - DNA, 20 bp, Homo sapiens
gccaagatctttgagatcca

SEQ ID NO: 1185 - DNA, 20 bp, Homo sapiens
ccaagatctttgagatccag

SEQ ID NO: 1186 - DNA, 20 bp, Homo sapiens
caagatctttgagatccaga

SEQ ID NO: 1187 - DNA, 20 bp, Homo sapiens
caagatctttgagatccaga

SEQ ID NO: 1188 - DNA, 20 bp, Homo sapiens
agatctttgagatccagacc

SEQ ID NO: 1189 - DNA, 20 bp, Homo sapiens
gatctttgagatccagacca

SEQ ID NO: 1190 - DNA, 20 bp, Homo sapiens
atctttgagatccagaccat

SEQ ID NO: 1191 - DNA, 20 bp, Homo sapiens
tctttgagatccagaccatg

SEQ ID NO: 1192 - DNA, 20 bp, Homo sapiens
ctttgagatccagaccatgc

SEQ ID NO: 1193 - DNA, 20 bp, Homo sapiens
tttgagatccagaccatgcc

SEQ ID NO: 1194 - DNA, 20 bp, Homo sapiens
ttgagatccagaccatgccc

SEQ ID NO: 1195 - DNA, 20 bp, Homo sapiens
tgagatccagaccatgccca

SEQ ID NO: 1196 - DNA, 20 bp, Homo sapiens
gagatccagaccatgcccaa

SEQ ID NO: 1197 - DNA, 20 bp, Homo sapiens
agatccagaccatgcccaat

SEQ ID NO: 1198 - DNA, 20 bp, Homo sapiens
gatccagaccatgcccaatg

SEQ ID NO: 1199 - DNA, 20 bp, Homo sapiens
atccagaccatgcccaatgg

SEQ ID NO: 1200 - DNA, 20 bp, Homo sapiens
tccagaccatgcccaatggc

SEQ ID NO: 1201 - DNA, 20 bp, Homo sapiens
ccagaccatgcccaatggca

SEQ ID NO: 1202 - DNA, 20 bp, Homo sapiens
cagaccatgcccaatggcaa

SEQ ID NO: 1203 - DNA, 20 bp, Homo sapiens
agaccatgcccaatggcaaa

SEQ ID NO: 1204 - DNA, 20 bp, Homo sapiens
gaccatgcccaatggcaaaa

SEQ ID NO: 1205 - DNA, 20 bp, Homo sapiens
accatgcccaatggcaaaac

SEQ ID NO: 1206 - DNA, 20 bp, Homo sapiens
ccatgcccaatggcaaaacc

SEQ ID NO: 1207 - DNA, 20 bp, Homo sapiens
catgcccaatggcaaaaccc

SEQ ID NO: 1208 - DNA, 20 bp, Homo sapiens
atgcccaatggcaaaacccg

SEQ ID NO: 1209 - DNA, 20 bp, Homo sapiens
tgcccaatggcaaaacccgg

SEQ ID NO: 1210 - DNA, 20 bp, Homo sapiens
gcccaatggcaaaacccgga

SEQ ID NO: 1211 - DNA, 20 bp, Homo sapiens
cccaatggcaaaacccggac

SEQ ID NO: 1212 - DNA, 20 bp, Homo sapiens
ccaatggcaaaacccggacc

SEQ ID NO: 1213 - DNA, 20 bp, Homo sapiens
caatggcaaaacccggacct

SEQ ID NO: 1214 - DNA, 20 bp, Homo sapiens
aatggcaaaacccggacctc

SEQ ID NO: 1215 - DNA, 20 bp, Homo sapiens
atggcaaaacccggacctcc

SEQ ID NO: 1216 - DNA, 20 bp, Homo sapiens
tggcaaaacccggacctccc

SEQ ID NO: 1217 - DNA, 20 bp, Homo sapiens
ggcaaaacccggacctccct

SEQ ID NO: 1218 - DNA, 20 bp, Homo sapiens
gcaaaacccggacctccctc

SEQ ID NO: 1219 - DNA, 20 bp, Homo sapiens
caaaacccggacctccctca

SEQ ID NO: 1220 - DNA, 20 bp, Homo sapiens
aaaacccggacctccctcaa

SEQ ID NO: 1221 - DNA, 20 bp, Homo sapiens
aaacccggacctccctcaag

SEQ ID NO: 1222 - DNA, 20 bp, Homo sapiens
aacccggacctccctcaaga

SEQ ID NO: 1223 - DNA, 20 bp, Homo sapiens
acccggacctccctcaagac

SEQ ID NO: 1224 - DNA, 20 bp, Homo sapiens
cccggacctccctcaagacc

SEQ ID NO: 1225 - DNA, 20 bp, Homo sapiens
ccggacctccctcaagacca

SEQ ID NO: 1226 - DNA, 20 bp, Homo sapiens
cggacctccctcaagaccat

SEQ ID NO: 1227 - DNA, 20 bp, Homo sapiens
ggacctccctcaagaccatg

SEQ ID NO: 1228 - DNA, 20 bp, Homo sapiens
gacctccctcaagaccatga

SEQ ID NO: 1229 - DNA, 20 bp, Homo sapiens
acctccctcaagaccatgag

SEQ ID NO: 1230 - DNA, 20 bp, Homo sapiens
cctccctcaagaccatgagc

SEQ ID NO: 1231 - DNA, 20 bp, Homo sapiens
ctccctcaagaccatgagcc

SEQ ID NO: 1232 - DNA, 20 bp, Homo sapiens
tccctcaagaccatgagccg

SEQ ID NO: 1233 - DNA, 20 bp, Homo sapiens
ccctcaagaccatgagccgt

SEQ ID NO: 1234 - DNA, 20 bp, Homo sapiens
cctcaagaccatgagccgta

SEQ ID NO: 1235 - DNA, 20 bp, Homo sapiens
ctcaagaccatgagccgtag

SEQ ID NO: 1236 - DNA, 20 bp, Homo sapiens
tcaagaccatgagccgtagg

SEQ ID NO: 1237 - DNA, 20 bp, Homo sapiens
caagaccatgagccgtagga

SEQ ID NO: 1238 - DNA, 20 bp, Homo sapiens
aagaccatgagccgtaggaa

SEQ ID NO: 1239 - DNA, 20 bp, Homo sapiens
agaccatgagccgtaggaag

SEQ ID NO: 1240 - DNA, 20 bp, Homo sapiens
gaccatgagccgtaggaagc

SEQ ID NO: 1241 - DNA, 20 bp, Homo sapiens
accatgagccgtaggaagct

SEQ ID NO: 1242 - DNA, 20 bp, Homo sapiens
ccatgagccgtaggaagctc

SEQ ID NO: 1243 - DNA, 20 bp, Homo sapiens
catgagccgtaggaagctct

SEQ ID NO: 1244 - DNA, 20 bp, Homo sapiens
atgagccgtaggaagctctc

SEQ ID NO: 1245 - DNA, 20 bp, Homo sapiens
tgagccgtaggaagctctcc

SEQ ID NO: 1246 - DNA, 20 bp, Homo sapiens
gagccgtaggaagctctccc

SEQ ID NO: 1247 - DNA, 20 bp, Homo sapiens
agccgtaggaagctctccca

SEQ ID NO: 1248 - DNA, 20 bp, Homo sapiens
gccgtaggaagctctcccag

SEQ ID NO: 1249 - DNA, 20 bp, Homo sapiens
ccgtaggaagctctcccagc

SEQ ID NO: 1250 - DNA, 20 bp, Homo sapiens
cgtaggaagctctcccagca

SEQ ID NO: 1251 - DNA, 20 bp, Homo sapiens
gtaggaagctctcccagcag

SEQ ID NO: 1252 - DNA, 20 bp, Homo sapiens
taggaagctctcccagcaga

SEQ ID NO: 1253 - DNA, 20 bp, Homo sapiens
aggaagctctcccagcagaa

SEQ ID NO: 1254 - DNA, 20 bp, Homo sapiens
ggaagctctcccagcagaag

SEQ ID NO: 1255 - DNA, 20 bp, Homo sapiens
gaagctctcccagcagaagg

SEQ ID NO: 1256 - DNA, 20 bp, Homo sapiens
aagctctcccagcagaagga

SEQ ID NO: 1257 - DNA, 20 bp, Homo sapiens
agctctcccagcagaaggag

SEQ ID NO: 1258 - DNA, 20 bp, Homo sapiens
gctctcccagcagaaggaga

SEQ ID NO: 1259 - DNA, 20 bp, Homo sapiens
ctctcccagcagaaggagaa

SEQ ID NO: 1260 - DNA, 20 bp, Homo sapiens
tctcccagcagaaggagaag

SEQ ID NO: 1261 - DNA, 20 bp, Homo sapiens
ctcccagcagaaggagaaga

SEQ ID NO: 1262 - DNA, 20 bp, Homo sapiens
tcccagcagaaggagaagaa

SEQ ID NO: 1263 - DNA, 20 bp, Homo sapiens
cccagcagaaggagaagaaa

SEQ ID NO: 1264 - DNA, 20 bp, Homo sapiens
ccagcagaaggagaagaaag

SEQ ID NO: 1265 - DNA, 20 bp, Homo sapiens
cagcagaaggagaagaaagc

SEQ ID NO: 1266 - DNA, 20 bp, Homo sapiens
agcagaaggagaagaaagcc

SEQ ID NO: 1267 - DNA, 20 bp, Homo sapiens
gcagaaggagaagaaagcca

SEQ ID NO: 1268 - DNA, 20 bp, Homo sapiens
cagaaggagaagaaagccac

SEQ ID NO: 1269 - DNA, 20 bp, Homo sapiens
agaaggagaagaaagccact

SEQ ID NO: 1270 - DNA, 20 bp, Homo sapiens
gaaggagaagaaagccactc

SEQ ID NO: 1271 - DNA, 20 bp, Homo sapiens
aaggagaagaaagccactca

SEQ ID NO: 1272 - DNA, 20 bp, Homo sapiens
aggagaagaaagccactcag

SEQ ID NO: 1273 - DNA, 20 bp, Homo sapiens
ggagaagaaagccactcaga

SEQ ID NO: 1274 - DNA, 20 bp, Homo sapiens
gagaagaaagccactcagat

SEQ ID NO: 1275 - DNA, 20 bp, Homo sapiens
agaagaaagccactcagatg

SEQ ID NO: 1276 - DNA, 20 bp, Homo sapiens
gaagaaagccactcagatgc

SEQ ID NO: 1277 - DNA, 20 bp, Homo sapiens
aagaaagccactcagatgct

SEQ ID NO: 1278 - DNA, 20 bp, Homo sapiens
agaaagccactcagatgctc

SEQ ID NO: 1279 - DNA, 20 bp, Homo sapiens
gaaagccactcagatgctcg

SEQ ID NO: 1280 - DNA, 20 bp, Homo sapiens
aaagccactcagatgctcgc

SEQ ID NO: 1281 - DNA, 20 bp, Homo sapiens
aagccactcagatgctcgcc

SEQ ID NO: 1282 - DNA, 20 bp, Homo sapiens
agccactcagatgctcgcca

SEQ ID NO: 1283 - DNA, 20 bp, Homo sapiens
gccactcagatgctcgccat

SEQ ID NO: 1284 - DNA, 20 bp, Homo sapiens
ccactcagatgctcgccatt

SEQ ID NO: 1285 - DNA, 20 bp, Homo sapiens
cactcagatgctcgccattg

SEQ ID NO: 1286 - DNA, 20 bp, Homo sapiens
actcagatgctcgccattgt

SEQ ID NO: 1287 - DNA, 20 bp, Homo sapiens
ctcagatgctcgccattgtt

SEQ ID NO: 1288 - DNA, 20 bp, Homo sapiens
tcagatgctcgccattgttc

SEQ ID NO: 1289 - DNA, 20 bp, Homo sapiens
cagatgctcgccattgttct

SEQ ID NO: 1290 - DNA, 20 bp, Homo sapiens
agatgctcgccattgttctc

SEQ ID NO: 1291 - DNA, 20 bp, Homo sapiens
gatgctcgccattgttctcg

SEQ ID NO: 1292 - DNA, 20 bp, Homo sapiens
atgctcgccattgttctcgg

SEQ ID NO: 1293 - DNA, 20 bp, Homo sapiens
tgctcgccattgttctcggc

SEQ ID NO: 1294 - DNA, 20 bp, Homo sapiens
gctcgccattgttctcggcg

SEQ ID NO: 1295 - DNA, 20 bp, Homo sapiens
ctcgccattgttctcggcgt

SEQ ID NO: 1296 - DNA, 20 bp, Homo sapiens
tcgccattgttctcggcgtg

SEQ ID NO: 1297 - DNA, 20 bp, Homo sapiens
cgccattgttctcggcgtgt

SEQ ID NO: 1298 - DNA, 20 bp, Homo sapiens
gccattgttctcggcgtgtt

SEQ ID NO: 1299 - DNA, 20 bp, Homo sapiens
ccattgttctcggcgtgttc

SEQ ID NO: 1300 - DNA, 20 bp, Homo sapiens
cattgttctcggcgtgttca

SEQ ID NO: 1301 - DNA, 20 bp, Homo sapiens
attgttctcggcgtgttcat

SEQ ID NO: 1302 - DNA, 20 bp, Homo sapiens
ttgttctcggcgtgttcatc

SEQ ID NO: 1303 - DNA, 20 bp, Homo sapiens
tgttctcggcgtgttcatca

SEQ ID NO: 1304 - DNA, 20 bp, Homo sapiens
gttctcggcgtgttcatcat

SEQ ID NO: 1305 - DNA, 20 bp, Homo sapiens
ttctcggcgtgttcatcatc

SEQ ID NO: 1306 - DNA, 20 bp, Homo sapiens
tctcggcgtgttcatcatct

SEQ ID NO: 1307 - DNA, 20 bp, Homo sapiens
ctcggcgtgttcatcatctg

SEQ ID NO: 1308 - DNA, 20 bp, Homo sapiens
tcggcgtgttcatcatctgc

SEQ ID NO: 1309 - DNA, 21 bp, Homo sapiens
tcggcgtgttcatcatctgct

SEQ ID NO: 1310 - DNA, 20 bp, Homo sapiens
ggcgtgttcatcatctgctg

SEQ ID NO: 1311 - DNA, 20 bp, Homo sapiens
gcgtgttcatcatctgctgg

SEQ ID NO: 1312 - DNA, 20 bp, Homo sapiens
cgtgttcatcatctgctggc

SEQ ID NO: 1313 - DNA, 20 bp, Homo sapiens
gtgttcatcatctgctggct

SEQ ID NO: 1314 - DNA, 20 bp, Homo sapiens
tgttcatcatctgctggctg

SEQ ID NO: 1315 - DNA, 20 bp, Homo sapiens
gttcatcatctgctggctgc

SEQ ID NO: 1316 - DNA, 20 bp, Homo sapiens
ttcatcatctgctggctgcc

SEQ ID NO: 1317 - DNA, 20 bp, Homo sapiens
tcatcatctgctggctgccc

SEQ ID NO: 1318 - DNA, 20 bp, Homo sapiens
catcatctgctggctgccct

SEQ ID NO: 1319 - DNA, 20 bp, Homo sapiens
atcatctgctggctgccctt

SEQ ID NO: 1320 - DNA, 20 bp, Homo sapiens
tcatctgctggctgcccttc

SEQ ID NO: 1321 - DNA, 20 bp, Homo sapiens
catctgctggctgcccttct

SEQ ID NO: 1322 - DNA, 20 bp, Homo sapiens
atctgctggctgcccttctt

SEQ ID NO: 1323 - DNA, 20 bp, Homo sapiens
tctgctggctgcccttcttc

SEQ ID NO: 1324 - DNA, 20 bp, Homo sapiens
ctgctggctgcccttcttca

SEQ ID NO: 1325 - DNA, 20 bp, Homo sapiens
tgctggctgcccttcttcat

SEQ ID NO: 1326 - DNA, 20 bp, Homo sapiens
gctggctgcccttcttcatc

SEQ ID NO: 1327 - DNA, 20 bp, Homo sapiens
ctggctgcccttcttcatca

SEQ ID NO: 1328 - DNA, 20 bp, Homo sapiens
tggctgcccttcttcatcac

SEQ ID NO: 1329 - DNA, 20 bp, Homo sapiens
ggctgcccttcttcatcaca

SEQ ID NO: 1330 - DNA, 20 bp, Homo sapiens
gctgcccttcttcatcacac

SEQ ID NO: 1331 - DNA, 20 bp, Homo sapiens
ctgcccttcttcatcacaca

SEQ ID NO: 1332 - DNA, 20 bp, Homo sapiens
tgcccttcttcatcacacac

SEQ ID NO: 1333 - DNA, 20 bp, Homo sapiens
gcccttcttcatcacacaca

SEQ ID NO: 1334 - DNA, 20 bp, Homo sapiens
cccttcttcatcacacacat

SEQ ID NO: 1335 - DNA, 20 bp, Homo sapiens
ccttcttcatcacacacatc

SEQ ID NO: 1336 - DNA, 20 bp, Homo sapiens
cttcttcatcacacacatcc

SEQ ID NO: 1337 - DNA, 20 bp, Homo sapiens
ttcttcatcacacacatcct

SEQ ID NO: 1338 - DNA, 20 bp, Homo sapiens
tcttcatcacacacatcctg

SEQ ID NO: 1339 - DNA, 20 bp, Homo sapiens
cttcatcacacacatcctga

SEQ ID NO: 1340 - DNA, 20 bp, Homo sapiens
ttcatcacacacatcctgaa

SEQ ID NO: 1341 - DNA, 20 bp, Homo sapiens
tcatcacacacatcctgaac

SEQ ID NO: 1342 - DNA, 20 bp, Homo sapiens
catcacacacatcctgaaca

SEQ ID NO: 1343 - DNA, 20 bp, Homo sapiens
atcacacacatcctgaacat

SEQ ID NO: 1344 - DNA, 20 bp, Homo sapiens
tcacacacatcctgaacata

SEQ ID NO: 1345 - DNA, 20 bp, Homo sapiens
cacacacatcctgaacatac

SEQ ID NO: 1346 - DNA, 20 bp, Homo sapiens
acacacatcctgaacataca

SEQ ID NO: 1347 - DNA, 20 bp, Homo sapiens
cacacatcctgaacatacac

SEQ ID NO: 1348 - DNA, 20 bp, Homo sapiens
acacatcctgaacatacact

SEQ ID NO: 1349 - DNA, 20 bp, Homo sapiens
cacatcctgaacatacactg

SEQ ID NO: 1350 - DNA, 20 bp, Homo sapiens
acatcctgaacatacactgt

SEQ ID NO: 1351 - DNA, 20 bp, Homo sapiens
catcctgaacatacactgtg

SEQ ID NO: 1352 - DNA, 20 bp, Homo sapiens
atcctgaacatacactgtga

SEQ ID NO: 1353 - DNA, 20 bp, Homo sapiens
tcctgaacatacactgtgac

SEQ ID NO: 1354 - DNA, 20 bp, Homo sapiens
cctgaacatacactgtgact

SEQ ID NO: 1355 - DNA, 20 bp, Homo sapiens
ctgaacatacactgtgactg

SEQ ID NO: 1356 - DNA, 20 bp, Homo sapiens
tgaacatacactgtgactgc

SEQ ID NO: 1357 - DNA, 20 bp, Homo sapiens
gaacatacactgtgactgca

SEQ ID NO: 1358 - DNA, 20 bp, Homo sapiens
aacatacactgtgactgcaa

SEQ ID NO: 1359 - DNA, 20 bp, Homo sapiens
acatacactgtgactgcaac

SEQ ID NO: 1360 - DNA, 20 bp, Homo sapiens
catacactgtgactgcaaca

SEQ ID NO: 1361 - DNA, 20 bp, Homo sapiens
atacactgtgactgcaacat

SEQ ID NO: 1362 - DNA, 20 bp, Homo sapiens
tacactgtgactgcaacatc

SEQ ID NO: 1363 - DNA, 20 bp, Homo sapiens
acactgtgactgcaacatcc

SEQ ID NO: 1364 - DNA, 20 bp, Homo sapiens
cactgtgactgcaacatccc

SEQ ID NO: 1365 - DNA, 20 bp, Homo sapiens
actgtgactgcaacatcccg

SEQ ID NO: 1366 - DNA, 20 bp, Homo sapiens
ctgtgactgcaacatcccgc

SEQ ID NO: 1367 - DNA, 20 bp, Homo sapiens
tgtgactgcaacatcccgcc

SEQ ID NO: 1368 - DNA, 20 bp, Homo sapiens
gtgactgcaacatcccgcct

SEQ ID NO: 1369 - DNA, 20 bp, Homo sapiens
tgactgcaacatcccgcctg

SEQ ID NO: 1370 - DNA, 20 bp, Homo sapiens
gactgcaacatcccgcctgt

SEQ ID NO: 1371 - DNA, 20 bp, Homo sapiens
actgcaacatcccgcctgtc

SEQ ID NO: 1372 - DNA, 20 bp, Homo sapiens
ctgcaacatcccgcctgtcc

SEQ ID NO: 1373 - DNA, 20 bp, Homo sapiens
tgcaacatcccgcctgtcct

SEQ ID NO: 1374 - DNA, 20 bp, Homo sapiens
gcaacatcccgcctgtcctg

SEQ ID NO: 1375 - DNA, 20 bp, Homo sapiens
caacatcccgcctgtcctgt

SEQ ID NO: 1376 - DNA, 20 bp, Homo sapiens
aacatcccgcctgtcctgta

SEQ ID NO: 1377 - DNA, 20 bp, Homo sapiens
acatcccgcctgtcctgtac

SEQ ID NO: 1378 - DNA, 20 bp, Homo sapiens
catcccgcctgtcctgtaca

SEQ ID NO: 1379 - DNA, 20 bp, Homo sapiens
atcccgcctgtcctgtacag

SEQ ID NO: 1380 - DNA, 20 bp, Homo sapiens
tcccgcctgtcctgtacagc

SEQ ID NO: 1381 - DNA, 20 bp, Homo sapiens
cccgcctgtcctgtacagcg

SEQ ID NO: 1382 - DNA, 20 bp, Homo sapiens
ccgcctgtcctgtacagcgc

SEQ ID NO: 1383 - DNA, 20 bp, Homo sapiens
cgcctgtcctgtacagcgcc

SEQ ID NO: 1384 - DNA, 20 bp, Homo sapiens
gcctgtcctgtacagcgcct

SEQ ID NO: 1385 - DNA, 20 bp, Homo sapiens
cctgtcctgtacagcgcctt

SEQ ID NO: 1386 - DNA, 20 bp, Homo sapiens
ctgtcctgtacagcgccttc

SEQ ID NO: 1387 - DNA, 20 bp, Homo sapiens
tgtcctgtacagcgccttca

SEQ ID NO: 1388 - DNA, 20 bp, Homo sapiens
gtcctgtacagcgccttcac

SEQ ID NO: 1389 - DNA, 20 bp, Homo sapiens
tcctgtacagcgccttcacg

SEQ ID NO: 1390 - DNA, 20 bp, Homo sapiens
cctgtacagcgccttcacgt

SEQ ID NO: 1391 - DNA, 20 bp, Homo sapiens
ctgtacagcgccttcacgtg

SEQ ID NO: 1392 - DNA, 20 bp, Homo sapiens
tgtacagcgccttcacgtgg

SEQ ID NO: 1393 - DNA, 20 bp, Homo sapiens
gtacagcgccttcacgtggc

SEQ ID NO: 1394 - DNA, 20 bp, Homo sapiens
tacagcgccttcacgtggct

SEQ ID NO: 1395 - DNA, 20 bp, Homo sapiens
acagcgccttcacgtggctg

SEQ ID NO: 1396 - DNA, 20 bp, Homo sapiens
cagcgccttcacgtggctgg

SEQ ID NO: 1397 - DNA, 20 bp, Homo sapiens
agcgccttcacgtggctggg

SEQ ID NO: 1398 - DNA, 20 bp, Homo sapiens
gcgccttcacgtggctgggc

SEQ ID NO: 1399 - DNA, 20 bp, Homo sapiens
cgccttcacgtggctgggct

SEQ ID NO: 1400 - DNA, 20 bp, Homo sapiens
gccttcacgtggctgggcta

SEQ ID NO: 1401 - DNA, 20 bp, Homo sapiens
ccttcacgtggctgggctat

SEQ ID NO: 1402 - DNA, 20 bp, Homo sapiens
cttcacgtggctgggctatg

SEQ ID NO: 1403 - DNA, 20 bp, Homo sapiens
ttcacgtggctgggctatgt

SEQ ID NO: 1404 - DNA, 20 bp, Homo sapiens
tcacgtggctgggctatgtc

SEQ ID NO: 1405 - DNA, 20 bp, Homo sapiens
cacgtggctgggctatgtca

SEQ ID NO: 1406 - DNA, 20 bp, Homo sapiens
acgtggctgggctatgtcaa

SEQ ID NO: 1407 - DNA, 20 bp, Homo sapiens
cgtggctgggctatgtcaac

SEQ ID NO: 1408 - DNA, 20 bp, Homo sapiens
gtggctgggctatgtcaaca

SEQ ID NO: 1409 - DNA, 20 bp, Homo sapiens
tggctgggctatgtcaacag

SEQ ID NO: 1410 - DNA, 20 bp, Homo sapiens
ggctgggctatgtcaacagc

SEQ ID NO: 1411 - DNA, 20 bp, Homo sapiens
gctgggctatgtcaacagcg

SEQ ID NO: 1412 - DNA, 20 bp, Homo sapiens
ctgggctatgtcaacagcgc

SEQ ID NO: 1413 - DNA, 20 bp, Homo sapiens
tgggctatgtcaacagcgcc

SEQ ID NO: 1414 - DNA, 20 bp, Homo sapiens
gggctatgtcaacagcgccg

SEQ ID NO: 1415 - DNA, 20 bp, Homo sapiens
ggctatgtcaacagcgccgt

SEQ ID NO: 1416 - DNA, 20 bp, Homo sapiens
gctatgtcaacagcgccgtg

SEQ ID NO: 1417 - DNA, 20 bp, Homo sapiens
ctatgtcaacagcgccgtga

SEQ ID NO: 1418 - DNA, 20 bp, Homo sapiens
tatgtcaacagcgccgtgaa

SEQ ID NO: 1419 - DNA, 20 bp, Homo sapiens
atgtcaacagcgccgtgaac

SEQ ID NO: 1420 - DNA, 20 bp, Homo sapiens
tgtcaacagcgccgtgaacc

SEQ ID NO: 1421 - DNA, 20 bp, Homo sapiens
gtcaacagcgccgtgaaccc

SEQ ID NO: 1422 - DNA, 20 bp, Homo sapiens
tcaacagcgccgtgaacccc

SEQ ID NO: 1423 - DNA, 20 bp, Homo sapiens
caacagcgccgtgaacccca

SEQ ID NO: 1424 - DNA, 20 bp, Homo sapiens
aacagcgccgtgaaccccat

SEQ ID NO: 1425 - DNA, 20 bp, Homo sapiens
acagcgccgtgaaccccatc

SEQ ID NO: 1426 - DNA, 20 bp, Homo sapiens
cagcgccgtgaaccccatca

SEQ ID NO: 1427 - DNA, 20 bp, Homo sapiens
agcgccgtgaaccccatcat

SEQ ID NO: 1428 - DNA, 20 bp, Homo sapiens
gcgccgtgaaccccatcatc

SEQ ID NO: 1429 - DNA, 20 bp, Homo sapiens
cgccgtgaaccccatcatct

SEQ ID NO: 1430 - DNA, 20 bp, Homo sapiens
gccgtgaaccccatcatcta

SEQ ID NO: 1431 - DNA, 20 bp, Homo sapiens
ccgtgaaccccatcatctac

SEQ ID NO: 1432 - DNA, 20 bp, Homo sapiens
cgtgaaccccatcatctaca

SEQ ID NO: 1433 - DNA, 20 bp, Homo sapiens
gtgaaccccatcatctacac

SEQ ID NO: 1434 - DNA, 20 bp, Homo sapiens
tgaaccccatcatctacacc

SEQ ID NO: 1435 - DNA, 20 bp, Homo sapiens
gaaccccatcatctacacca

SEQ ID NO: 1436 - DNA, 20 bp, Homo sapiens
aaccccatcatctacaccac

SEQ ID NO: 1437 - DNA, 20 bp, Homo sapiens
accccatcatctacaccacc

SEQ ID NO: 1438 - DNA, 20 bp, Homo sapiens
ccccatcatctacaccacct

SEQ ID NO: 1439 - DNA, 20 bp, Homo sapiens
cccatcatctacaccacctt

SEQ ID NO: 1440 - DNA, 20 bp, Homo sapiens
ccatcatctacaccaccttc

SEQ ID NO: 1441 - DNA, 20 bp, Homo sapiens
catcatctacaccaccttca

SEQ ID NO: 1442 - DNA, 20 bp, Homo sapiens
atcatctacaccaccttcaa

SEQ ID NO: 1443 - DNA, 20 bp, Homo sapiens
tcatctacaccaccttcaac

SEQ ID NO: 1444 - DNA, 20 bp, Homo sapiens
catctacaccaccttcaaca

SEQ ID NO: 1445 - DNA, 20 bp, Homo sapiens
atctacaccaccttcaacat

SEQ ID NO: 1446 - DNA, 20 bp, Homo sapiens
tctacaccaccttcaacatt

SEQ ID NO: 1447 - DNA, 20 bp, Homo sapiens
ctacaccaccttcaacattg

SEQ ID NO: 1448 - DNA, 20 bp, Homo sapiens
tacaccaccttcaacattga

SEQ ID NO: 1449 - DNA, 20 bp, Homo sapiens
acaccaccttcaacattgag

SEQ ID NO: 1450 - DNA, 20 bp, Homo sapiens
caccaccttcaacattgagt

SEQ ID NO: 1451 - DNA, 20 bp, Homo sapiens
accaccttcaacattgagtt

SEQ ID NO: 1452 - DNA, 20 bp, Homo sapiens
ccaccttcaacattgagttc

SEQ ID NO: 1453 - DNA, 20 bp, Homo sapiens
caccttcaacattgagttcc

SEQ ID NO: 1454 - DNA, 20 bp, Homo sapiens
accttcaacattgagttccg

SEQ ID NO: 1455 - DNA, 20 bp, Homo sapiens
ccttcaacattgagttccgc

SEQ ID NO: 1456 - DNA, 20 bp, Homo sapiens
cttcaacattgagttccgca

SEQ ID NO: 1457 - DNA, 20 bp, Homo sapiens
ttcaacattgagttccgcaa

SEQ ID NO: 1458 - DNA, 20 bp, Homo sapiens
tcaacattgagttccgcaag

SEQ ID NO: 1459 - DNA, 20 bp, Homo sapiens
caacattgagttccgcaagg

SEQ ID NO: 1460 - DNA, 20 bp, Homo sapiens
aacattgagttccgcaaggc

SEQ ID NO: 1461 - DNA, 20 bp, Homo sapiens
acattgagttccgcaaggcc

SEQ ID NO: 1462 - DNA, 20 bp, Homo sapiens
cattgagttccgcaaggcct

SEQ ID NO: 1463 - DNA, 20 bp, Homo sapiens
attgagttccgcaaggcctt

SEQ ID NO: 1464 - DNA, 20 bp, Homo sapiens
ttgagttccgcaaggccttc

SEQ ID NO: 1465 - DNA, 20 bp, Homo sapiens
tgagttccgcaaggccttcc

SEQ ID NO: 1466 - DNA, 20 bp, Homo sapiens
gagttccgcaaggccttcct

SEQ ID NO: 1467 - DNA, 20 bp, Homo sapiens
agttccgcaaggccttcctg

SEQ ID NO: 1468 - DNA, 20 bp, Homo sapiens
gttccgcaaggccttcctga

SEQ ID NO: 1469 - DNA, 20 bp, Homo sapiens
ttccgcaaggccttcctgaa

SEQ ID NO: 1470 - DNA, 20 bp, Homo sapiens
tccgcaaggccttcctgaag

SEQ ID NO: 1471 - DNA, 20 bp, Homo sapiens
ccgcaaggccttcctgaaga

SEQ ID NO: 1472 - DNA, 20 bp, Homo sapiens
cgcaaggccttcctgaagat

SEQ ID NO: 1473 - DNA, 20 bp, Homo sapiens
gcaaggccttcctgaagatc

SEQ ID NO: 1474 - DNA, 20 bp, Homo sapiens
caaggccttcctgaagatcc

SEQ ID NO: 1475 - DNA, 20 bp, Homo sapiens
aaggccttcctgaagatcct

SEQ ID NO: 1476 - DNA, 20 bp, Homo sapiens
aggccttcctgaagatcctc

SEQ ID NO: 1477 - DNA, 20 bp, Homo sapiens
ggccttcctgaagatcctcc

SEQ ID NO: 1478 - DNA, 20 bp, Homo sapiens
gccttcctgaagatcctcca

SEQ ID NO: 1479 - DNA, 20 bp, Homo sapiens
ccttcctgaagatcctccac

SEQ ID NO: 1480 - DNA, 20 bp, Homo sapiens
cttcctgaagatcctccact

SEQ ID NO: 1481 - DNA, 20 bp, Homo sapiens
ttcctgaagatcctccactg

SEQ ID NO: 1482 - DNA, 20 bp, Homo sapiens
tcctgaagatcctccactgc

SEQ ID NO: 1483 - DNA, 20 bp, Homo sapiens
cctgaagatcctccactgct

SEQ ID NO: 1484 - DNA, 20 bp, Homo sapiens
ctgaagatcctccactgctg

SEQ ID NO: 1485 - DNA, 20 bp, Homo sapiens
tgaagatcctccactgctga

SEQ ID NO: 1486 - DNA, 20 bp, Homo sapiens
gaagatcctccactgctgac

SEQ ID NO: 1487 - DNA, 20 bp, Homo sapiens
aagatcctccactgctgact

SEQ ID NO: 1488 - DNA, 20 bp, Homo sapiens
agatcctccactgctgactc

SEQ ID NO: 1489 - DNA, 20 bp, Homo sapiens
gatcctccactgctgactct

SEQ ID NO: 1490 - DNA, 20 bp, Homo sapiens
atcctccactgctgactctg

SEQ ID NO: 1491 - DNA, 20 bp, Homo sapiens
tcctccactgctgactctgc

SEQ ID NO: 1492 - DNA, 20 bp, Homo sapiens
cctccactgctgactctgct

SEQ ID NO: 1493 - DNA, 20 bp, Homo sapiens
ctccactgctgactctgctg

SEQ ID NO: 1494 - DNA, 20 bp, Homo sapiens
tccactgctgactctgctgc

SEQ ID NO: 1495 - DNA, 20 bp, Homo sapiens
ccactgctgactctgctgcc

SEQ ID NO: 1496 - DNA, 20 bp, Homo sapiens
cactgctgactctgctgcct

SEQ ID NO: 1497 - DNA, 20 bp, Homo sapiens
actgctgactctgctgcctg

SEQ ID NO: 1498 - DNA, 20 bp, Homo sapiens
ctgctgactctgctgcctgc

SEQ ID NO: 1499 - DNA, 20 bp, Homo sapiens
tgctgactctgctgcctgcc

SEQ ID NO: 1500 - DNA, 20 bp, Homo sapiens
gctgactctgctgcctgccc

SEQ ID NO: 1501 - DNA, 20 bp, Homo sapiens
ctgactctgctgcctgcccg

SEQ ID NO: 1502 - DNA, 20 bp, Homo sapiens
tgactctgctgcctgcccgc

SEQ ID NO: 1503 - DNA, 20 bp, Homo sapiens
gactctgctgcctgcccgca

SEQ ID NO: 1504 - DNA, 20 bp, Homo sapiens
actctgctgcctgcccgcac

SEQ ID NO: 1505 - DNA, 20 bp, Homo sapiens
ctctgctgcctgcccgcaca

SEQ ID NO: 1506 - DNA, 20 bp, Homo sapiens
tctgctgcctgcccgcacag

SEQ ID NO: 1507 - DNA, 20 bp, Homo sapiens
ctgctgcctgcccgcacagc

SEQ ID NO: 1508 - DNA, 20 bp, Homo sapiens
tgctgcctgcccgcacagca

SEQ ID NO: 1509 - DNA, 20 bp, Homo sapiens
gctgcctgcccgcacagcag

SEQ ID NO: 1510 - DNA, 20 bp, Homo sapiens
ctgcctgcccgcacagcagc

SEQ ID NO: 1511 - DNA, 20 bp, Homo sapiens
tgcctgcccgcacagcagcc

SEQ ID NO: 1512 - DNA, 20 bp, Homo sapiens
gcctgcccgcacagcagcct

SEQ ID NO: 1513 - DNA, 20 bp, Homo sapiens
cctgcccgcacagcagcctg

SEQ ID NO: 1514 - DNA, 20 bp, Homo sapiens
ctgcccgcacagcagcctgc

SEQ ID NO: 1515 - DNA, 20 bp, Homo sapiens
tgcccgcacagcagcctgct

SEQ ID NO: 1516 - DNA, 20 bp, Homo sapiens
gcccgcacagcagcctgctt

SEQ ID NO: 1517 - DNA, 20 bp, Homo sapiens
cccgcacagcagcctgcttc

SEQ ID NO: 1518 - DNA, 20 bp, Homo sapiens
ccgcacagcagcctgcttcc

SEQ ID NO: 1519 - DNA, 20 bp, Homo sapiens
cgcacagcagcctgcttccc

SEQ ID NO: 1520 - DNA, 20 bp, Homo sapiens
gcacagcagcctgcttccca

SEQ ID NO: 1521 - DNA, 20 bp, Homo sapiens
cacagcagcctgcttcccac

SEQ ID NO: 1522 - DNA, 20 bp, Homo sapiens
acagcagcctgcttcccacc

SEQ ID NO: 1523 - DNA, 20 bp, Homo sapiens
cagcagcctgcttcccacct

SEQ ID NO: 1524 - DNA, 20 bp, Homo sapiens
agcagcctgcttcccacctc

SEQ ID NO: 1525 - DNA, 20 bp, Homo sapiens
gcagcctgcttcccacctcc

SEQ ID NO: 1526 - DNA, 20 bp, Homo sapiens
cagcctgcttcccacctccc

SEQ ID NO: 1527 - DNA, 20 bp, Homo sapiens
agcctgcttcccacctccct

SEQ ID NO: 1528 - DNA, 20 bp, Homo sapiens
gcctgcttcccacctccctg

SEQ ID NO: 1529 - DNA, 20 bp, Homo sapiens
cctgcttcccacctccctgc

SEQ ID NO: 1530 - DNA, 20 bp, Homo sapiens
ctgcttcccacctccctgcc

SEQ ID NO: 1531 - DNA, 20 bp, Homo sapiens
tgcttcccacctccctgccc

SEQ ID NO: 1532 - DNA, 20 bp, Homo sapiens
gcttcccacctccctgccca

SEQ ID NO: 1533 - DNA, 20 bp, Homo sapiens
cttcccacctccctgcccag

SEQ ID NO: 1534 - DNA, 20 bp, Homo sapiens
ttcccacctccctgcccagg

SEQ ID NO: 1535 - DNA, 20 bp, Homo sapiens
tcccacctccctgcccaggc

SEQ ID NO: 1536 - DNA, 20 bp, Homo sapiens
cccacctccctgcccaggcc

SEQ ID NO: 1537 - DNA, 20 bp, Homo sapiens
ccacctccctgcccaggccg

SEQ ID NO: 1538 - DNA, 20 bp, Homo sapiens
cacctccctgcccaggccgg

SEQ ID NO: 1539 - DNA, 20 bp, Homo sapiens
acctccctgcccaggccggc

SEQ ID NO: 1540 - DNA, 20 bp, Homo sapiens
cctccctgcccaggccggcc

SEQ ID NO: 1541 - DNA, 20 bp, Homo sapiens
ctccctgcccaggccggcca

SEQ ID NO: 1542 - DNA, 20 bp, Homo sapiens
tccctgcccaggccggccag

SEQ ID NO: 1543 - DNA, 20 bp, Homo sapiens
ccctgcccaggccggccagc

SEQ ID NO: 1544 - DNA, 20 bp, Homo sapiens
cctgcccaggccggccagcc

SEQ ID NO: 1545 - DNA, 20 bp, Homo sapiens
ctgcccaggccggccagcct

SEQ ID NO: 1546 - DNA, 20 bp, Homo sapiens
tgcccaggccggccagcctc

SEQ ID NO: 1547 - DNA, 20 bp, Homo sapiens
gcccaggccggccagcctca

SEQ ID NO: 1548 - DNA, 20 bp, Homo sapiens
cccaggccggccagcctcac

SEQ ID NO: 1549 - DNA, 20 bp, Homo sapiens
ccaggccggccagcctcacc

SEQ ID NO: 1550 - DNA, 20 bp, Homo sapiens
caggccggccagcctcaccc

SEQ ID NO: 1551 - DNA, 20 bp, Homo sapiens
aggccggccagcctcaccct

SEQ ID NO: 1552 - DNA, 20 bp, Homo sapiens
ggccggccagcctcaccctt

SEQ ID NO: 1553 - DNA, 20 bp, Homo sapiens
gccggccagcctcacccttg

SEQ ID NO: 1554 - DNA, 20 bp, Homo sapiens
ccggccagcctcacccttgc

SEQ ID NO: 1555 - DNA, 20 bp, Homo sapiens
cggccagcctcacccttgcg

SEQ ID NO: 1556 - DNA, 20 bp, Homo sapiens
ggccagcctcacccttgcga

SEQ ID NO: 1557 - DNA, 20 bp, Homo sapiens
gccagcctcacccttgcgaa

SEQ ID NO: 1558 - DNA, 20 bp, Homo sapiens
ccagcctcacccttgcgaac

SEQ ID NO: 1559 - DNA, 20 bp, Homo sapiens
cagcctcacccttgcgaacc

SEQ ID NO: 1560 - DNA, 20 bp, Homo sapiens
agcctcacccttgcgaaccg

SEQ ID NO: 1561 - DNA, 20 bp, Homo sapiens
gcctcacccttgcgaaccgt

SEQ ID NO: 1562 - DNA, 20 bp, Homo sapiens
cctcacccttgcgaaccgtg

SEQ ID NO: 1563 - DNA, 20 bp, Homo sapiens
ctcacccttgcgaaccgtga

SEQ ID NO: 1564 - DNA, 20 bp, Homo sapiens
tcacccttgcgaaccgtgag

SEQ ID NO: 1565 - DNA, 20 bp, Homo sapiens
cacccttgcgaaccgtgagc

SEQ ID NO: 1566 - DNA, 20 bp, Homo sapiens
acccttgcgaaccgtgagca

SEQ ID NO: 1567 - DNA, 20 bp, Homo sapiens
cccttgcgaaccgtgagcag

SEQ ID NO: 1568 - DNA, 20 bp, Homo sapiens
ccttgcgaaccgtgagcagg

SEQ ID NO: 1569 - DNA, 20 bp, Homo sapiens
cttgcgaaccgtgagcagga

SEQ ID NO: 1570 - DNA, 20 bp, Homo sapiens
ttgcgaaccgtgagcaggaa

SEQ ID NO: 1571 - DNA, 20 bp, Homo sapiens
tgcgaaccgtgagcaggaag

SEQ ID NO: 1572 - DNA, 20 bp, Homo sapiens
gcgaaccgtgagcaggaagg

SEQ ID NO: 1573 - DNA, 20 bp, Homo sapiens
cgaaccgtgagcaggaaggc

SEQ ID NO: 1574 - DNA, 20 bp, Homo sapiens
gaaccgtgagcaggaaggcc

SEQ ID NO: 1575 - DNA, 20 bp, Homo sapiens
aaccgtgagcaggaaggcct

SEQ ID NO: 1576 - DNA, 20 bp, Homo sapiens
accgtgagcaggaaggcctg

SEQ ID NO: 1577 - DNA, 20 bp, Homo sapiens
ccgtgagcaggaaggcctgg

SEQ ID NO: 1578 - DNA, 20 bp, Homo sapiens
cgtgagcaggaaggcctggg

SEQ ID NO: 1579 - DNA, 20 bp, Homo sapiens
gtgagcaggaaggcctgggt

SEQ ID NO: 1580 - DNA, 20 bp, Homo sapiens
tgagcaggaaggcctgggtg

SEQ ID NO: 1581 - DNA, 20 bp, Homo sapiens
gagcaggaaggcctgggtgg

SEQ ID NO: 1582 - DNA, 20 bp, Homo sapiens
agcaggaaggcctgggtgga

SEQ ID NO: 1583 - DNA, 20 bp, Homo sapiens
gcaggaaggcctgggtggat

SEQ ID NO: 1584 - DNA, 20 bp, Homo sapiens
caggaaggcctgggtggatc

SEQ ID NO: 1585 - DNA, 20 bp, Homo sapiens
aggaaggcctgggtggatcg

SEQ ID NO: 1586 - DNA, 20 bp, Homo sapiens
ggaaggcctgggtggatcgg

SEQ ID NO: 1587 - DNA, 20 bp, Homo sapiens
gaaggcctgggtggatcggc

SEQ ID NO: 1588 - DNA, 20 bp, Homo sapiens
aaggcctgggtggatcggcc

SEQ ID NO: 1589 - DNA, 20 bp, Homo sapiens
aggcctgggtggatcggcct

SEQ ID NO: 1590 - DNA, 20 bp, Homo sapiens
ggcctgggtggatcggcctc

SEQ ID NO: 1591 - DNA, 20 bp, Homo sapiens
gcctgggtggatcggcctcc

SEQ ID NO: 1592 - DNA, 20 bp, Homo sapiens
cctgggtggatcggcctcct

SEQ ID NO: 1593 - DNA, 20 bp, Homo sapiens
ctgggtggatcggcctcctc

SEQ ID NO: 1594 - DNA, 20 bp, Homo sapiens
tgggtggatcggcctcctct

SEQ ID NO: 1595 - DNA, 20 bp, Homo sapiens
gggtggatcggcctcctctt

SEQ ID NO: 1596 - DNA, 20 bp, Homo sapiens
ggtggatcggcctcctcttc

SEQ ID NO: 1597 - DNA, 20 bp, Homo sapiens
gtggatcggcctcctcttca

SEQ ID NO: 1598 - DNA, 20 bp, Homo sapiens
tggatcggcctcctcttcac

SEQ ID NO: 1599 - DNA, 20 bp, Homo sapiens
ggatcggcctcctcttcacc

SEQ ID NO: 1600 - DNA, 20 bp, Homo sapiens
gatcggcctcctcttcaccc

SEQ ID NO: 1601 - DNA, 20 bp, Homo sapiens
atcggcctcctcttcacccc

SEQ ID NO: 1602 - DNA, 20 bp, Homo sapiens
tcggcctcctcttcaccccg

SEQ ID NO: 1603 - DNA, 20 bp, Homo sapiens
cggcctcctcttcaccccgg

SEQ ID NO: 1604 - DNA, 20 bp, Homo sapiens
ggcctcctcttcaccccggc

SEQ ID NO: 1605 - DNA, 20 bp, Homo sapiens
gcctcctcttcaccccggca

SEQ ID NO: 1606 - DNA, 20 bp, Homo sapiens
cctcctcttcaccccggcag

SEQ ID NO: 1607 - DNA, 20 bp, Homo sapiens
ctcctcttcaccccggcagg

SEQ ID NO: 1608 - DNA, 20 bp, Homo sapiens
tcctcttcaccccggcaggc

SEQ ID NO: 1609 - DNA, 20 bp, Homo sapiens
cctcttcaccccggcaggcc

SEQ ID NO: 1610 - DNA, 20 bp, Homo sapiens
ctcttcaccccggcaggccc

SEQ ID NO: 1611 - DNA, 20 bp, Homo sapiens
tcttcaccccggcaggccct

SEQ ID NO: 1612 - DNA, 20 bp, Homo sapiens
cttcaccccggcaggccctg

SEQ ID NO: 1613 - DNA, 20 bp, Homo sapiens
ttcaccccggcaggccctgc

SEQ ID NO: 1614 - DNA, 20 bp, Homo sapiens
tcaccccggcaggccctgca

SEQ ID NO: 1615 - DNA, 20 bp, Homo sapiens
caccccggcaggccctgcag

SEQ ID NO: 1616 - DNA, 20 bp, Homo sapiens
accccggcaggccctgcagt

SEQ ID NO: 1617 - DNA, 20 bp, Homo sapiens
ccccggcaggccctgcagtg

SEQ ID NO: 1618 - DNA, 20 bp, Homo sapiens
cccggcaggccctgcagtgt

SEQ ID NO: 1619 - DNA, 20 bp, Homo sapiens
ccggcaggccctgcagtgtt

SEQ ID NO: 1620 - DNA, 20 bp, Homo sapiens
cggcaggccctgcagtgttc

SEQ ID NO: 1621 - DNA, 20 bp, Homo sapiens
ggcaggccctgcagtgttcg

SEQ ID NO: 1622 - DNA, 20 bp, Homo sapiens
gcaggccctgcagtgttcgc

SEQ ID NO: 1623 - DNA, 20 bp, Homo sapiens
caggccctgcagtgttcgct

SEQ ID NO: 1624 - DNA, 20 bp, Homo sapiens
aggccctgcagtgttcgctt

SEQ ID NO: 1625 - DNA, 20 bp, Homo sapiens
ggccctgcagtgttcgcttg

SEQ ID NO: 1626 - DNA, 20 bp, Homo sapiens
gccctgcagtgttcgcttgg

SEQ ID NO: 1627 - DNA, 20 bp, Homo sapiens
ccctgcagtgttcgcttggc

SEQ ID NO: 1628 - DNA, 20 bp, Homo sapiens
cctgcagtgttcgcttggct

SEQ ID NO: 1629 - DNA, 20 bp, Homo sapiens
ctgcagtgttcgcttggctc

SEQ ID NO: 1630 - DNA, 20 bp, Homo sapiens
tgcagtgttcgcttggctcc

SEQ ID NO: 1631 - DNA, 20 bp, Homo sapiens
gcagtgttcgcttggctcca

SEQ ID NO: 1632 - DNA, 20 bp, Homo sapiens
cagtgttcgcttggctccat

SEQ ID NO: 1633 - DNA, 20 bp, Homo sapiens
agtgttcgcttggctccatg

SEQ ID NO: 1634 - DNA, 20 bp, Homo sapiens
gtgttcgcttggctccatgc

SEQ ID NO: 1635 - DNA, 20 bp, Homo sapiens
tgttcgcttggctccatgct

SEQ ID NO: 1636 - DNA, 20 bp, Homo sapiens
gttcgcttggctccatgctc

SEQ ID NO: 1637 - DNA, 20 bp, Homo sapiens
ttcgcttggctccatgctcc

SEQ ID NO: 1638 - DNA, 20 bp, Homo sapiens
tcgcttggctccatgctcct

SEQ ID NO: 1639 - DNA, 20 bp, Homo sapiens
cgcttggctccatgctcctc

SEQ ID NO: 1640 - DNA, 20 bp, Homo sapiens
gcttggctccatgctcctca

SEQ ID NO: 1641 - DNA, 20 bp, Homo sapiens
cttggctccatgctcctcac

SEQ ID NO: 1642 - DNA, 20 bp, Homo sapiens
ttggctccatgctcctcact

SEQ ID NO: 1643 - DNA, 20 bp, Homo sapiens
tggctccatgctcctcactg

SEQ ID NO: 1644 - DNA, 20 bp, Homo sapiens
ggctccatgctcctcactgc

SEQ ID NO: 1645 - DNA, 20 bp, Homo sapiens
gctccatgctcctcactgcc

SEQ ID NO: 1646 - DNA, 20 bp, Homo sapiens
ctccatgctcctcactgccc

SEQ ID NO: 1647 - DNA, 20 bp, Homo sapiens
tccatgctcctcactgcccg

SEQ ID NO: 1648 - DNA, 20 bp, Homo sapiens
ccatgctcctcactgcccgc

SEQ ID NO: 1649 - DNA, 20 bp, Homo sapiens
catgctcctcactgcccgca

SEQ ID NO: 1650 - DNA, 20 bp, Homo sapiens
atgctcctcactgcccgcac

SEQ ID NO: 1651 - DNA, 20 bp, Homo sapiens
tgctcctcactgcccgcaca

SEQ ID NO: 1652 - DNA, 20 bp, Homo sapiens
gctcctcactgcccgcacac

SEQ ID NO: 1653 - DNA, 20 bp, Homo sapiens
ctcctcactgcccgcacacc

SEQ ID NO: 1654 - DNA, 20 bp, Homo sapiens
tcctcactgcccgcacaccc

SEQ ID NO: 1655 - DNA, 20 bp, Homo sapiens
cctcactgcccgcacaccct

SEQ ID NO: 1656 - DNA, 20 bp, Homo sapiens
ctcactgcccgcacaccctc

SEQ ID NO: 1657 - DNA, 20 bp, Homo sapiens
tcactgcccgcacaccctca

SEQ ID NO: 1658 - DNA, 20 bp, Homo sapiens
cactgcccgcacaccctcac

SEQ ID NO: 1659 - DNA, 20 bp, Homo sapiens
actgcccgcacaccctcact

SEQ ID NO: 1660 - DNA, 20 bp, Homo sapiens
ctgcccgcacaccctcactc

SEQ ID NO: 1661 - DNA, 20 bp, Homo sapiens
tgcccgcacaccctcactct

SEQ ID NO: 1662 - DNA, 20 bp, Homo sapiens
gcccgcacaccctcactctg

SEQ ID NO: 1663 - DNA, 20 bp, Homo sapiens
cccgcacaccctcactctgc

SEQ ID NO: 1664 - DNA, 20 bp, Homo sapiens
ccgcacaccctcactctgcc

SEQ ID NO: 1665 - DNA, 20 bp, Homo sapiens
cgcacaccctcactctgcca

SEQ ID NO: 1666 - DNA, 20 bp, Homo sapiens
gcacaccctcactctgccag

SEQ ID NO: 1667 - DNA, 20 bp, Homo sapiens
cacaccctcactctgccagg

SEQ ID NO: 1668 - DNA, 20 bp, Homo sapiens
acaccctcactctgccaggg

SEQ ID NO: 1669 - DNA, 20 bp, Homo sapiens
caccctcactctgccagggc

SEQ ID NO: 1670 - DNA, 20 bp, Homo sapiens
accctcactctgccagggca

SEQ ID NO: 1671 - DNA, 20 bp, Homo sapiens
ccctcactctgccagggcag

SEQ ID NO: 1672 - DNA, 20 bp, Homo sapiens
cctcactctgccagggcagt

SEQ ID NO: 1673 - DNA, 20 bp, Homo sapiens
ctcactctgccagggcagtg

SEQ ID NO: 1674 - DNA, 20 bp, Homo sapiens
tcactctgccagggcagtgc

SEQ ID NO: 1675 - DNA, 20 bp, Homo sapiens
cactctgccagggcagtgct

SEQ ID NO: 1676 - DNA, 20 bp, Homo sapiens
actctgccagggcagtgcta

SEQ ID NO: 1677 - DNA, 20 bp, Homo sapiens
ctctgccagggcagtgctag

SEQ ID NO: 1678 - DNA, 20 bp, Homo sapiens
tctgccagggcagtgctagt

SEQ ID NO: 1679 - DNA, 20 bp, Homo sapiens
ctgccagggcagtgctagtg

SEQ ID NO: 1680 - DNA, 20 bp, Homo sapiens
tgccagggcagtgctagtga

SEQ ID NO: 1681 - DNA, 20 bp, Homo sapiens
gccagggcagtgctagtgag

SEQ ID NO: 1682 - DNA, 20 bp, Homo sapiens
ccagggcagtgctagtgagc

SEQ ID NO: 1683 - DNA, 20 bp, Homo sapiens
cagggcagtgctagtgagct

SEQ ID NO: 1684 - DNA, 20 bp, Homo sapiens
agggcagtgctagtgagctg

SEQ ID NO: 1685 - DNA, 20 bp, Homo sapiens
gggcagtgctagtgagctgg

SEQ ID NO: 1686 - DNA, 20 bp, Homo sapiens
ggcagtgctagtgagctggg

SEQ ID NO: 1687 - DNA, 20 bp, Homo sapiens
gcagtgctagtgagctgggc

SEQ ID NO: 1688 - DNA, 20 bp, Homo sapiens
cagtgctagtgagctgggca

SEQ ID NO: 1689 - DNA, 20 bp, Homo sapiens
agtgctagtgagctgggcat

SEQ ID NO: 1690 - DNA, 20 bp, Homo sapiens
gtgctagtgagctgggcatg

SEQ ID NO: 1691 - DNA, 20 bp, Homo sapiens
tgctagtgagctgggcatgg

SEQ ID NO: 1692 - DNA, 20 bp, Homo sapiens
gctagtgagctgggcatggt

SEQ ID NO: 1693 - DNA, 20 bp, Homo sapiens
ctagtgagctgggcatggta

SEQ ID NO: 1694 - DNA, 20 bp, Homo sapiens
tagtgagctgggcatggtac

SEQ ID NO: 1695 - DNA, 20 bp, Homo sapiens
agtgagctgggcatggtacc

SEQ ID NO: 1696 - DNA, 20 bp, Homo sapiens
gtgagctgggcatggtacca

SEQ ID NO: 1697 - DNA, 20 bp, Homo sapiens
tgagctgggcatggtaccag

SEQ ID NO: 1698 - DNA, 20 bp, Homo sapiens
gagctgggcatggtaccagc

SEQ ID NO: 1699 - DNA, 20 bp, Homo sapiens
agctgggcatggtaccagcc

SEQ ID NO: 1700 - DNA, 20 bp, Homo sapiens
gctgggcatggtaccagccc

SEQ ID NO: 1701 - DNA, 20 bp, Homo sapiens
ctgggcatggtaccagccct

SEQ ID NO: 1702 - DNA, 20 bp, Homo sapiens
tgggcatggtaccagccctg

SEQ ID NO: 1703 - DNA, 20 bp, Homo sapiens
gggcatggtaccagccctgg

SEQ ID NO: 1704 - DNA, 20 bp, Homo sapiens
ggcatggtaccagccctggg

SEQ ID NO: 1705 - DNA, 20 bp, Homo sapiens
gcatggtaccagccctgggg

SEQ ID NO: 1706 - DNA, 20 bp, Homo sapiens
catggtaccagccctggggc

SEQ ID NO: 1707 - DNA, 20 bp, Homo sapiens
atggtaccagccctggggct

SEQ ID NO: 1708 - DNA, 20 bp, Homo sapiens
tggtaccagccctggggctg

SEQ ID NO: 1709 - DNA, 20 bp, Homo sapiens
ggtaccagccctggggctgg

SEQ ID NO: 1710 - DNA, 20 bp, Homo sapiens
gtaccagccctggggctggg

SEQ ID NO: 1711 - DNA, 20 bp, Homo sapiens
taccagccctggggctgggc

SEQ ID NO: 1712 - DNA, 20 bp, Homo sapiens
accagccctggggctgggcc

SEQ ID NO: 1713 - DNA, 20 bp, Homo sapiens
ccagccctggggctgggccc

SEQ ID NO: 1714 - DNA, 20 bp, Homo sapiens
cagccctggggctgggcccc

SEQ ID NO: 1715 - DNA, 20 bp, Homo sapiens
agccctggggctgggccccc

SEQ ID NO: 1716 - DNA, 20 bp, Homo sapiens
gccctggggctgggcccccc

SEQ ID NO: 1717 - DNA, 20 bp, Homo sapiens
ccctggggctgggcccccca

SEQ ID NO: 1718 - DNA, 20 bp, Homo sapiens
cctggggctgggccccccag

SEQ ID NO: 1719 - DNA, 20 bp, Homo sapiens
ctggggctgggccccccagc

SEQ ID NO: 1720 - DNA, 20 bp, Homo sapiens
tggggctgggccccccagct

SEQ ID NO: 1721 - DNA, 20 bp, Homo sapiens
ggggctgggccccccagctc

SEQ ID NO: 1722 - DNA, 20 bp, Homo sapiens
gggctgggccccccagctca

SEQ ID NO: 1723 - DNA, 20 bp, Homo sapiens
ggctgggccccccagctcag

SEQ ID NO: 1724 - DNA, 20 bp, Homo sapiens
gctgggccccccagctcagg

SEQ ID NO: 1725 - DNA, 20 bp, Homo sapiens
ctgggccccccagctcaggg

SEQ ID NO: 1726 - DNA, 20 bp, Homo sapiens
tgggccccccagctcagggg

SEQ ID NO: 1727 - DNA, 20 bp, Homo sapiens
gggccccccagctcaggggc

SEQ ID NO: 1728 - DNA, 20 bp, Homo sapiens
ggccccccagctcaggggca

SEQ ID NO: 1729 - DNA, 20 bp, Homo sapiens
gccccccagctcaggggcag

SEQ ID NO: 1730 - DNA, 20 bp, Homo sapiens
ccccccagctcaggggcagc

SEQ ID NO: 1731 - DNA, 20 bp, Homo sapiens
cccccagctcaggggcagct

SEQ ID NO: 1732 - DNA, 20 bp, Homo sapiens
ccccagctcaggggcagctc

SEQ ID NO: 1733 - DNA, 20 bp, Homo sapiens
cccagctcaggggcagctca

SEQ ID NO: 1734 - DNA, 20 bp, Homo sapiens
ccagctcaggggcagctcat

SEQ ID NO: 1735 - DNA, 20 bp, Homo sapiens
cagctcaggggcagctcata

SEQ ID NO: 1736 - DNA, 20 bp, Homo sapiens
agctcaggggcagctcatag

SEQ ID NO: 1737 - DNA, 20 bp, Homo sapiens
gctcaggggcagctcataga

SEQ ID NO: 1738 - DNA, 20 bp, Homo sapiens
ctcaggggcagctcatagag

SEQ ID NO: 1739 - DNA, 20 bp, Homo sapiens
tcaggggcagctcatagagt

SEQ ID NO: 1740 - DNA, 20 bp, Homo sapiens
caggggcagctcatagagtc

SEQ ID NO: 1741 - DNA, 20 bp, Homo sapiens
aggggcagctcatagagtcc

SEQ ID NO: 1742 - DNA, 20 bp, Homo sapiens
ggggcagctcatagagtccc

SEQ ID NO: 1743 - DNA, 20 bp, Homo sapiens
gggcagctcatagagtcccc

SEQ ID NO: 1744 - DNA, 20 bp, Homo sapiens
ggcagctcatagagtccccc

SEQ ID NO: 1745 - DNA, 21 bp, Homo sapiens
gcagctcatagagtcccccct

SEQ ID NO: 1746 - DNA, 21 bp, Homo sapiens
cagctcatagagtcccccctc

SEQ ID NO: 1747 - DNA, 20 bp, Homo sapiens
agctcatagagtcccccctc

SEQ ID NO: 1748 - DNA, 20 bp, Homo sapiens
gctcatagagtcccccctcc

SEQ ID NO: 1749 - DNA, 20 bp, Homo sapiens
ctcatagagtcccccctccc

SEQ ID NO: 1750 - DNA, 20 bp, Homo sapiens
tcatagagtcccccctccca

SEQ ID NO: 1751 - DNA, 20 bp, Homo sapiens
catagagtcccccctcccac

SEQ ID NO: 1752 - DNA, 20 bp, Homo sapiens
atagagtcccccctcccacc

SEQ ID NO: 1753 - DNA, 20 bp, Homo sapiens
tagagtcccccctcccacct

SEQ ID NO: 1754 - DNA, 20 bp, Homo sapiens
agagtcccccctcccacctc

SEQ ID NO: 1755 - DNA, 20 bp, Homo sapiens
gagtcccccctcccacctcc

SEQ ID NO: 1756 - DNA, 20 bp, Homo sapiens
agtcccccctcccacctcca

SEQ ID NO: 1757 - DNA, 20 bp, Homo sapiens
gtcccccctcccacctccag

SEQ ID NO: 1758 - DNA, 20 bp, Homo sapiens
tcccccctcccacctccagt

SEQ ID NO: 1759 - DNA, 20 bp, Homo sapiens
cccccctcccacctccagtc

SEQ ID NO: 1760 - DNA, 20 bp, Homo sapiens
ccccctcccacctccagtcc

SEQ ID NO: 1761 - DNA, 20 bp, Homo sapiens
cccctcccacctccagtccc

SEQ ID NO: 1762 - DNA, 20 bp, Homo sapiens
ccctcccacctccagtcccc

SEQ ID NO: 1763 - DNA, 20 bp, Homo sapiens
cctcccacctccagtccccc

SEQ ID NO: 1764 - DNA, 20 bp, Homo sapiens
ctcccacctccagtccccct

SEQ ID NO: 1765 - DNA, 20 bp, Homo sapiens
tcccacctccagtcccccta

SEQ ID NO: 1766 - DNA, 20 bp, Homo sapiens
cccacctccagtccccctat

SEQ ID NO: 1767 - DNA, 20 bp, Homo sapiens
ccacctccagtccccctatc

SEQ ID NO: 1768 - DNA, 20 bp, Homo sapiens
cacctccagtccccctatcc

SEQ ID NO: 1769 - DNA, 20 bp, Homo sapiens
acctccagtccccctatcct

SEQ ID NO: 1770 - DNA, 20 bp, Homo sapiens
cctccagtccccctatcctt

SEQ ID NO: 1771 - DNA, 20 bp, Homo sapiens
ctccagtccccctatccttg

SEQ ID NO: 1772 - DNA, 20 bp, Homo sapiens
tccagtccccctatccttgg

SEQ ID NO: 1773 - DNA, 20 bp, Homo sapiens
ccagtccccctatccttggc

SEQ ID NO: 1774 - DNA, 20 bp, Homo sapiens
cagtccccctatccttggca

SEQ ID NO: 1775 - DNA, 20 bp, Homo sapiens
agtccccctatccttggcac

SEQ ID NO: 1776 - DNA, 20 bp, Homo sapiens
gtccccctatccttggcacc

SEQ ID NO: 1777 - DNA, 20 bp, Homo sapiens
tccccctatccttggcacca

SEQ ID NO: 1778 - DNA, 20 bp, Homo sapiens
ccccctatccttggcaccaa

SEQ ID NO: 1779 - DNA, 20 bp, Homo sapiens
cccctatccttggcaccaaa

SEQ ID NO: 1780 - DNA, 20 bp, Homo sapiens
ccctatccttggcaccaaag

SEQ ID NO: 1781 - DNA, 20 bp, Homo sapiens
cctatccttggcaccaaaga

SEQ ID NO: 1782 - DNA, 20 bp, Homo sapiens
ctatccttggcaccaaagat

SEQ ID NO: 1783 - DNA, 20 bp, Homo sapiens
tatccttggcaccaaagatg

SEQ ID NO: 1784 - DNA, 20 bp, Homo sapiens
atccttggcaccaaagatgc

SEQ ID NO: 1785 - DNA, 20 bp, Homo sapiens
tccttggcaccaaagatgca

SEQ ID NO: 1786 - DNA, 20 bp, Homo sapiens
ccttggcaccaaagatgcag

SEQ ID NO: 1787 - DNA, 20 bp, Homo sapiens
cttggcaccaaagatgcagc

SEQ ID NO: 1788 - DNA, 20 bp, Homo sapiens
ttggcaccaaagatgcagcc

SEQ ID NO: 1789 - DNA, 20 bp, Homo sapiens
tggcaccaaagatgcagccg

SEQ ID NO: 1790 - DNA, 20 bp, Homo sapiens
ggcaccaaagatgcagccgc

SEQ ID NO: 1791 - DNA, 20 bp, Homo sapiens
gcaccaaagatgcagccgcc

SEQ ID NO: 1792 - DNA, 20 bp, Homo sapiens
caccaaagatgcagccgcct

SEQ ID NO: 1793 - DNA, 20 bp, Homo sapiens
accaaagatgcagccgcctt

SEQ ID NO: 1794 - DNA, 20 bp, Homo sapiens
ccaaagatgcagccgccttc

SEQ ID NO: 1795 - DNA, 20 bp, Homo sapiens
caaagatgcagccgccttcc

SEQ ID NO: 1796 - DNA, 20 bp, Homo sapiens
aaagatgcagccgccttcct

SEQ ID NO: 1797 - DNA, 20 bp, Homo sapiens
aagatgcagccgccttcctt

SEQ ID NO: 1798 - DNA, 20 bp, Homo sapiens
agatgcagccgccttccttg

SEQ ID NO: 1799 - DNA, 20 bp, Homo sapiens
gatgcagccgccttccttga

SEQ ID NO: 1800 - DNA, 20 bp, Homo sapiens
atgcagccgccttccttgac

SEQ ID NO: 1801 - DNA, 20 bp, Homo sapiens
tgcagccgccttccttgacc

SEQ ID NO: 1802 - DNA, 20 bp, Homo sapiens
gcagccgccttccttgacct

SEQ ID NO: 1803 - DNA, 20 bp, Homo sapiens
cagccgccttccttgacctt

SEQ ID NO: 1804 - DNA, 20 bp, Homo sapiens
agccgccttccttgaccttc

SEQ ID NO: 1805 - DNA, 20 bp, Homo sapiens
gccgccttccttgaccttcc

SEQ ID NO: 1806 - DNA, 20 bp, Homo sapiens
ccgccttccttgaccttcct

SEQ ID NO: 1807 - DNA, 20 bp, Homo sapiens
cgccttccttgaccttcctc

SEQ ID NO: 1808 - DNA, 20 bp, Homo sapiens
gccttccttgaccttcctct

SEQ ID NO: 1809 - DNA, 20 bp, Homo sapiens
ccttccttgaccttcctctg

SEQ ID NO: 1810 - DNA, 20 bp, Homo sapiens
cttccttgaccttcctctgg

SEQ ID NO: 1811 - DNA, 20 bp, Homo sapiens
ttccttgaccttcctctggg

SEQ ID NO: 1812 - DNA, 20 bp, Homo sapiens
tccttgaccttcctctgggg

SEQ ID NO: 1813 - DNA, 20 bp, Homo sapiens
ccttgaccttcctctggggc

SEQ ID NO: 1814 - DNA, 20 bp, Homo sapiens
cttgaccttcctctggggct

SEQ ID NO: 1815 - DNA, 20 bp, Homo sapiens
ttgaccttcctctggggctc

SEQ ID NO: 1816 - DNA, 20 bp, Homo sapiens
tgaccttcctctggggctct

SEQ ID NO: 1817 - DNA, 20 bp, Homo sapiens
gaccttcctctggggctcta

SEQ ID NO: 1818 - DNA, 20 bp, Homo sapiens
accttcctctggggctctag

SEQ ID NO: 1819 - DNA, 20 bp, Homo sapiens
ccttcctctggggctctagg

SEQ ID NO: 1820 - DNA, 20 bp, Homo sapiens
cttcctctggggctctaggg

SEQ ID NO: 1821 - DNA, 20 bp, Homo sapiens
ttcctctggggctctagggt

SEQ ID NO: 1822 - DNA, 20 bp, Homo sapiens
tcctctggggctctagggtt

SEQ ID NO: 1823 - DNA, 20 bp, Homo sapiens
cctctggggctctagggttg

SEQ ID NO: 1824 - DNA, 20 bp, Homo sapiens
ctctggggctctagggttgc

SEQ ID NO: 1825 - DNA, 20 bp, Homo sapiens
tctggggctctagggttgct

SEQ ID NO: 1826 - DNA, 20 bp, Homo sapiens
ctggggctctagggttgctg

SEQ ID NO: 1827 - DNA, 20 bp, Homo sapiens
tggggctctagggttgctgg

SEQ ID NO: 1828 - DNA, 20 bp, Homo sapiens
ggggctctagggttgctgga

SEQ ID NO: 1829 - DNA, 20 bp, Homo sapiens
gggctctagggttgctggag

SEQ ID NO: 1830 - DNA, 20 bp, Homo sapiens
ggctctagggttgctggagc

SEQ ID NO: 1831 - DNA, 20 bp, Homo sapiens
gctctagggttgctggagcc

SEQ ID NO: 1832 - DNA, 20 bp, Homo sapiens
ctctagggttgctggagcct

SEQ ID NO: 1833 - DNA, 20 bp, Homo sapiens
tctagggttgctggagcctg

SEQ ID NO: 1834 - DNA, 20 bp, Homo sapiens
ctagggttgctggagcctga

SEQ ID NO: 1835 - DNA, 20 bp, Homo sapiens
tagggttgctggagcctgag

SEQ ID NO: 1836 - DNA, 20 bp, Homo sapiens
agggttgctggagcctgagt

SEQ ID NO: 1837 - DNA, 20 bp, Homo sapiens
gggttgctggagcctgagtc

SEQ ID NO: 1838 - DNA, 20 bp, Homo sapiens
ggttgctggagcctgagtca

SEQ ID NO: 1839 - DNA, 20 bp, Homo sapiens
gttgctggagcctgagtcag

SEQ ID NO: 1840 - DNA, 20 bp, Homo sapiens
ttgctggagcctgagtcagg

SEQ ID NO: 1841 - DNA, 20 bp, Homo sapiens
tgctggagcctgagtcaggg

SEQ ID NO: 1842 - DNA, 20 bp, Homo sapiens
gctggagcctgagtcagggc

SEQ ID NO: 1843 - DNA, 20 bp, Homo sapiens
ctggagcctgagtcagggcc

SEQ ID NO: 1844 - DNA, 20 bp, Homo sapiens
tggagcctgagtcagggccc

SEQ ID NO: 1845 - DNA, 20 bp, Homo sapiens
ggagcctgagtcagggccca

SEQ ID NO: 1846 - DNA, 20 bp, Homo sapiens
gagcctgagtcagggcccag

SEQ ID NO: 1847 - DNA, 20 bp, Homo sapiens
agcctgagtcagggcccaga

SEQ ID NO: 1848 - DNA, 20 bp, Homo sapiens
gcctgagtcagggcccagag

SEQ ID NO: 1849 - DNA, 20 bp, Homo sapiens
cctgagtcagggcccagagg

SEQ ID NO: 1850 - DNA, 20 bp, Homo sapiens
ctgagtcagggcccagaggc

SEQ ID NO: 1851 - DNA, 20 bp, Homo sapiens
tgagtcagggcccagaggct

SEQ ID NO: 1852 - DNA, 20 bp, Homo sapiens
gagtcagggcccagaggctg

SEQ ID NO: 1853 - DNA, 20 bp, Homo sapiens
agtcagggcccagaggctga

SEQ ID NO: 1854 - DNA, 20 bp, Homo sapiens
gtcagggcccagaggctgag

SEQ ID NO: 1855 - DNA, 20 bp, Homo sapiens
tcagggcccagaggctgagt

SEQ ID NO: 1856 - DNA, 20 bp, Homo sapiens
cagggcccagaggctgagtt

SEQ ID NO: 1857 - DNA, 20 bp, Homo sapiens
agggcccagaggctgagttt

SEQ ID NO: 1858 - DNA, 20 bp, Homo sapiens
gggcccagaggctgagtttt

SEQ ID NO: 1859 - DNA, 20 bp, Homo sapiens
ggcccagaggctgagttttc

SEQ ID NO: 1860 - DNA, 20 bp, Homo sapiens
gcccagaggctgagttttct

SEQ ID NO: 1861 - DNA, 20 bp, Homo sapiens
cccagaggctgagttttctc

SEQ ID NO: 1862 - DNA, 20 bp, Homo sapiens
ccagaggctgagttttctct

SEQ ID NO: 1863 - DNA, 20 bp, Homo sapiens
cagaggctgagttttctctt

SEQ ID NO: 1864 - DNA, 20 bp, Homo sapiens
agaggctgagttttctcttt

SEQ ID NO: 1865 - DNA, 20 bp, Homo sapiens
gaggctgagttttctctttg

SEQ ID NO: 1866 - DNA, 20 bp, Homo sapiens
aggctgagttttctctttgt

SEQ ID NO: 1867 - DNA, 20 bp, Homo sapiens
ggctgagttttctctttgtg

SEQ ID NO: 1868 - DNA, 20 bp, Homo sapiens
gctgagttttctctttgtgg

SEQ ID NO: 1869 - DNA, 20 bp, Homo sapiens
ctgagttttctctttgtggg

SEQ ID NO: 1870 - DNA, 20 bp, Homo sapiens
tgagttttctctttgtgggg

SEQ ID NO: 1871 - DNA, 20 bp, Homo sapiens
gagttttctctttgtggggc

SEQ ID NO: 1872 - DNA, 20 bp, Homo sapiens
agttttctctttgtggggct

SEQ ID NO: 1873 - DNA, 20 bp, Homo sapiens
gttttctctttgtggggctt

SEQ ID NO: 1874 - DNA, 20 bp, Homo sapiens
ttttctctttgtggggcttg

SEQ ID NO: 1875 - DNA, 20 bp, Homo sapiens
tttctctttgtggggcttgg

SEQ ID NO: 1876 - DNA, 20 bp, Homo sapiens
ttctctttgtggggcttggc

SEQ ID NO: 1877 - DNA, 20 bp, Homo sapiens
tctctttgtggggcttggcg

SEQ ID NO: 1878 - DNA, 20 bp, Homo sapiens
ctctttgtggggcttggcgt

SEQ ID NO: 1879 - DNA, 20 bp, Homo sapiens
tctttgtggggcttggcgtg

SEQ ID NO: 1880 - DNA, 20 bp, Homo sapiens
ctttgtggggcttggcgtgg

SEQ ID NO: 1881 - DNA, 20 bp, Homo sapiens
tttgtggggcttggcgtgga

SEQ ID NO: 1882 - DNA, 20 bp, Homo sapiens
ttgtggggcttggcgtggag

SEQ ID NO: 1883 - DNA, 20 bp, Homo sapiens
tgtggggcttggcgtggagc

SEQ ID NO: 1884 - DNA, 20 bp, Homo sapiens
gtggggcttggcgtggagca

SEQ ID NO: 1885 - DNA, 20 bp, Homo sapiens
tggggcttggcgtggagcag

SEQ ID NO: 1886 - DNA, 20 bp, Homo sapiens
ggggcttggcgtggagcagg

SEQ ID NO: 1887 - DNA, 20 bp, Homo sapiens
gggcttggcgtggagcaggc

SEQ ID NO: 1888 - DNA, 20 bp, Homo sapiens
ggcttggcgtggagcaggcg

SEQ ID NO: 1889 - DNA, 20 bp, Homo sapiens
gcttggcgtggagcaggcgg

SEQ ID NO: 1890 - DNA, 20 bp, Homo sapiens
cttggcgtggagcaggcggt

SEQ ID NO: 1891 - DNA, 20 bp, Homo sapiens
ttggcgtggagcaggcggtg

SEQ ID NO: 1892 - DNA, 20 bp, Homo sapiens
tggcgtggagcaggcggtgg

SEQ ID NO: 1893 - DNA, 20 bp, Homo sapiens
ggcgtggagcaggcggtggg

SEQ ID NO: 1894 - DNA, 20 bp, Homo sapiens
gcgtggagcaggcggtgggg

SEQ ID NO: 1895 - DNA, 20 bp, Homo sapiens
cgtggagcaggcggtgggga

SEQ ID NO: 1896 - DNA, 20 bp, Homo sapiens
gtggagcaggcggtggggag

SEQ ID NO: 1897 - DNA, 20 bp, Homo sapiens
tggagcaggcggtggggaga

SEQ ID NO: 1898 - DNA, 20 bp, Homo sapiens
ggagcaggcggtggggagag

SEQ ID NO: 1899 - DNA, 20 bp, Homo sapiens
gagcaggcggtggggagaga

SEQ ID NO: 1900 - DNA, 20 bp, Homo sapiens
agcaggcggtggggagagat

SEQ ID NO: 1901 - DNA, 20 bp, Homo sapiens
gcaggcggtggggagagatg

SEQ ID NO: 1902 - DNA, 20 bp, Homo sapiens
caggcggtggggagagatgg

SEQ ID NO: 1903 - DNA, 20 bp, Homo sapiens
aggcggtggggagagatgga

SEQ ID NO: 1904 - DNA, 20 bp, Homo sapiens
ggcggtggggagagatggac

SEQ ID NO: 1905 - DNA, 20 bp, Homo sapiens
gcggtggggagagatggaca

SEQ ID NO: 1906 - DNA, 20 bp, Homo sapiens
cggtggggagagatggacag

SEQ ID NO: 1907 - DNA, 20 bp, Homo sapiens
ggtggggagagatggacagt

SEQ ID NO: 1908 - DNA, 20 bp, Homo sapiens
gtggggagagatggacagtt

SEQ ID NO: 1909 - DNA, 20 bp, Homo sapiens
tggggagagatggacagttc

SEQ ID NO: 1910 - DNA, 20 bp, Homo sapiens
ggggagagatggacagttca

SEQ ID NO: 1911 - DNA, 20 bp, Homo sapiens
gggagagatggacagttcac

SEQ ID NO: 1912 - DNA, 20 bp, Homo sapiens
ggagagatggacagttcaca

SEQ ID NO: 1913 - DNA, 20 bp, Homo sapiens
gagagatggacagttcacac

SEQ ID NO: 1914 - DNA, 20 bp, Homo sapiens
agagatggacagttcacacc

SEQ ID NO: 1915 - DNA, 20 bp, Homo sapiens
gagatggacagttcacaccc

SEQ ID NO: 1916 - DNA, 20 bp, Homo sapiens
agatggacagttcacaccct

SEQ ID NO: 1917 - DNA, 20 bp, Homo sapiens
gatggacagttcacaccctg

SEQ ID NO: 1918 - DNA, 20 bp, Homo sapiens
atggacagttcacaccctgc

SEQ ID NO: 1919 - DNA, 20 bp, Homo sapiens
tggacagttcacaccctgca

SEQ ID NO: 1920 - DNA, 20 bp, Homo sapiens
ggacagttcacaccctgcaa

SEQ ID NO: 1921 - DNA, 20 bp, Homo sapiens
gacagttcacaccctgcaag

SEQ ID NO: 1922 - DNA, 20 bp, Homo sapiens
acagttcacaccctgcaagg

SEQ ID NO: 1923 - DNA, 20 bp, Homo sapiens
cagttcacaccctgcaaggc

SEQ ID NO: 1924 - DNA, 20 bp, Homo sapiens
agttcacaccctgcaaggcc

SEQ ID NO: 1925 - DNA, 20 bp, Homo sapiens
gttcacaccctgcaaggccc

SEQ ID NO: 1926 - DNA, 20 bp, Homo sapiens
ttcacaccctgcaaggccca

SEQ ID NO: 1927 - DNA, 20 bp, Homo sapiens
tcacaccctgcaaggcccac

SEQ ID NO: 1928 - DNA, 20 bp, Homo sapiens
cacaccctgcaaggcccaca

SEQ ID NO: 1929 - DNA, 20 bp, Homo sapiens
acaccctgcaaggcccacag

SEQ ID NO: 1930 - DNA, 20 bp, Homo sapiens
caccctgcaaggcccacagg

SEQ ID NO: 1931 - DNA, 20 bp, Homo sapiens
accctgcaaggcccacagga

SEQ ID NO: 1932 - DNA, 20 bp, Homo sapiens
ccctgcaaggcccacaggag

SEQ ID NO: 1933 - DNA, 20 bp, Homo sapiens
cctgcaaggcccacaggagg

SEQ ID NO: 1934 - DNA, 20 bp, Homo sapiens
ctgcaaggcccacaggaggc

SEQ ID NO: 1935 - DNA, 20 bp, Homo sapiens
tgcaaggcccacaggaggca

SEQ ID NO: 1936 - DNA, 20 bp, Homo sapiens
gcaaggcccacaggaggcaa

SEQ ID NO: 1937 - DNA, 20 bp, Homo sapiens
caaggcccacaggaggcaag

SEQ ID NO: 1938 - DNA, 20 bp, Homo sapiens
aaggcccacaggaggcaagc

SEQ ID NO: 1939 - DNA, 20 bp, Homo sapiens
aggcccacaggaggcaagca

SEQ ID NO: 1940 - DNA, 20 bp, Homo sapiens
ggcccacaggaggcaagcaa

SEQ ID NO: 1941 - DNA, 20 bp, Homo sapiens
gcccacaggaggcaagcaag

SEQ ID NO: 1942 - DNA, 20 bp, Homo sapiens
cccacaggaggcaagcaagc

SEQ ID NO: 1943 - DNA, 20 bp, Homo sapiens
ccacaggaggcaagcaagct

SEQ ID NO: 1944 - DNA, 20 bp, Homo sapiens
cacaggaggcaagcaagctc

SEQ ID NO: 1945 - DNA, 20 bp, Homo sapiens
acaggaggcaagcaagctct

SEQ ID NO: 1946 - DNA, 20 bp, Homo sapiens
caggaggcaagcaagctctc

SEQ ID NO: 1947 - DNA, 20 bp, Homo sapiens
aggaggcaagcaagctctct

SEQ ID NO: 1948 - DNA, 20 bp, Homo sapiens
ggaggcaagcaagctctctt

SEQ ID NO: 1949 - DNA, 20 bp, Homo sapiens
gaggcaagcaagctctcttg

SEQ ID NO: 1950 - DNA, 20 bp, Homo sapiens
aggcaagcaagctctcttgc

SEQ ID NO: 1951 - DNA, 20 bp, Homo sapiens
ggcaagcaagctctcttgcc

SEQ ID NO: 1952 - DNA, 20 bp, Homo sapiens
gcaagcaagctctcttgccg

SEQ ID NO: 1953 - DNA, 20 bp, Homo sapiens
caagcaagctctcttgccga

SEQ ID NO: 1954 - DNA, 20 bp, Homo sapiens
aagcaagctctcttgccgag

SEQ ID NO: 1955 - DNA, 20 bp, Homo sapiens
agcaagctctcttgccgagg

SEQ ID NO: 1956 - DNA, 20 bp, Homo sapiens
gcaagctctcttgccgagga

SEQ ID NO: 1957 - DNA, 20 bp, Homo sapiens
caagctctcttgccgaggag

SEQ ID NO: 1958 - DNA, 20 bp, Homo sapiens
aagctctcttgccgaggagc

SEQ ID NO: 1959 - DNA, 20 bp, Homo sapiens
agctctcttgccgaggagcc

SEQ ID NO: 1960 - DNA, 20 bp, Homo sapiens
gctctcttgccgaggagcca

SEQ ID NO: 1961 - DNA, 20 bp, Homo sapiens
ctctcttgccgaggagccag

SEQ ID NO: 1962 - DNA, 20 bp, Homo sapiens
tctcttgccgaggagccagg

SEQ ID NO: 1963 - DNA, 20 bp, Homo sapiens
ctcttgccgaggagccaggc

SEQ ID NO: 1964 - DNA, 20 bp, Homo sapiens
tcttgccgaggagccaggca

SEQ ID NO: 1965 - DNA, 20 bp, Homo sapiens
cttgccgaggagccaggcaa

SEQ ID NO: 1966 - DNA, 20 bp, Homo sapiens
ttgccgaggagccaggcaac

SEQ ID NO: 1967 - DNA, 20 bp, Homo sapiens
tgccgaggagccaggcaact

SEQ ID NO: 1968 - DNA, 20 bp, Homo sapiens
gccgaggagccaggcaactt

SEQ ID NO: 1969 - DNA, 20 bp, Homo sapiens
ccgaggagccaggcaacttc

SEQ ID NO: 1970 - DNA, 20 bp, Homo sapiens
cgaggagccaggcaacttca

SEQ ID NO: 1971 - DNA, 20 bp, Homo sapiens
gaggagccaggcaacttcag

SEQ ID NO: 1972 - DNA, 20 bp, Homo sapiens
aggagccaggcaacttcagt

SEQ ID NO: 1973 - DNA, 20 bp, Homo sapiens
ggagccaggcaacttcagtc

SEQ ID NO: 1974 - DNA, 20 bp, Homo sapiens
gagccaggcaacttcagtcc

SEQ ID NO: 1975 - DNA, 20 bp, Homo sapiens
agccaggcaacttcagtcct

SEQ ID NO: 1976 - DNA, 20 bp, Homo sapiens
gccaggcaacttcagtcctg

SEQ ID NO: 1977 - DNA, 20 bp, Homo sapiens
ccaggcaacttcagtcctgg

SEQ ID NO: 1978 - DNA, 20 bp, Homo sapiens
caggcaacttcagtcctggg

SEQ ID NO: 1979 - DNA, 20 bp, Homo sapiens
aggcaacttcagtcctggga

SEQ ID NO: 1980 - DNA, 20 bp, Homo sapiens
ggcaacttcagtcctgggag

SEQ ID NO: 1981 - DNA, 20 bp, Homo sapiens
gcaacttcagtcctgggaga

SEQ ID NO: 1982 - DNA, 20 bp, Homo sapiens
caacttcagtcctgggagac

SEQ ID NO: 1983 - DNA, 20 bp, Homo sapiens
aacttcagtcctgggagacc

SEQ ID NO: 1984 - DNA, 20 bp, Homo sapiens
acttcagtcctgggagaccc

SEQ ID NO: 1985 - DNA, 20 bp, Homo sapiens
cttcagtcctgggagaccca

SEQ ID NO: 1986 - DNA, 20 bp, Homo sapiens
ttcagtcctgggagacccat

SEQ ID NO: 1987 - DNA, 20 bp, Homo sapiens
tcagtcctgggagacccatg

SEQ ID NO: 1988 - DNA, 20 bp, Homo sapiens
cagtcctgggagacccatgt

SEQ ID NO: 1989 - DNA, 20 bp, Homo sapiens
agtcctgggagacccatgta

SEQ ID NO: 1990 - DNA, 20 bp, Homo sapiens
gtcctgggagacccatgtaa

SEQ ID NO: 1991 - DNA, 20 bp, Homo sapiens
tcctgggagacccatgtaaa

SEQ ID NO: 1992 - DNA, 20 bp, Homo sapiens
cctgggagacccatgtaaat

SEQ ID NO: 1993 - DNA, 20 bp, Homo sapiens
ctgggagacccatgtaaata

SEQ ID NO: 1994 - DNA, 20 bp, Homo sapiens
tgggagacccatgtaaatac

SEQ ID NO: 1995 - DNA, 20 bp, Homo sapiens
gggagacccatgtaaatacc

SEQ ID NO: 1996 - DNA, 20 bp, Homo sapiens
ggagacccatgtaaatacca

SEQ ID NO: 1997 - DNA, 20 bp, Homo sapiens
gagacccatgtaaataccag

SEQ ID NO: 1998 - DNA, 20 bp, Homo sapiens
agacccatgtaaataccaga

SEQ ID NO: 1999 - DNA, 20 bp, Homo sapiens
gacccatgtaaataccagac

SEQ ID NO: 2000 - DNA, 20 bp, Homo sapiens
acccatgtaaataccagact

SEQ ID NO: 2001 - DNA, 20 bp, Homo sapiens
cccatgtaaataccagactg

SEQ ID NO: 2002 - DNA, 20 bp, Homo sapiens
ccatgtaaataccagactgc

SEQ ID NO: 2003 - DNA, 20 bp, Homo sapiens
catgtaaataccagactgca

SEQ ID NO: 2004 - DNA, 20 bp, Homo sapiens
atgtaaataccagactgcag

SEQ ID NO: 2005 - DNA, 20 bp, Homo sapiens
tgtaaataccagactgcagg

SEQ ID NO: 2006 - DNA, 20 bp, Homo sapiens
gtaaataccagactgcaggt

SEQ ID NO: 2007 - DNA, 20 bp, Homo sapiens
taaataccagactgcaggtt

SEQ ID NO: 2008 - DNA, 20 bp, Homo sapiens
taaataccagactgcaggtt

SEQ ID NO: 2009 - DNA, 20 bp, Homo sapiens
aataccagactgcaggttgg

SEQ ID NO: 2010 - DNA, 20 bp, Homo sapiens
ataccagactgcaggttgga

SEQ ID NO: 2011 - DNA, 20 bp, Homo sapiens
taccagactgcaggttggac

SEQ ID NO: 2012 - DNA, 20 bp, Homo sapiens
accagactgcaggttggacc

SEQ ID NO: 2013 - DNA, 20 bp, Homo sapiens
ccagactgcaggttggaccc

SEQ ID NO: 2014 - DNA, 20 bp, Homo sapiens
cagactgcaggttggacccc

SEQ ID NO: 2015 - DNA, 20 bp, Homo sapiens
agactgcaggttggacccca

SEQ ID NO: 2016 - DNA, 20 bp, Homo sapiens
gactgcaggttggaccccag

SEQ ID NO: 2017 - DNA, 20 bp, Homo sapiens
actgcaggttggaccccaga

SEQ ID NO: 2018 - DNA, 20 bp, Homo sapiens
ctgcaggttggaccccagag

SEQ ID NO: 2019 - DNA, 20 bp, Homo sapiens
tgcaggttggaccccagaga

SEQ ID NO: 2020 - DNA, 20 bp, Homo sapiens
gcaggttggaccccagagat

SEQ ID NO: 2021 - DNA, 20 bp, Homo sapiens
caggttggaccccagagatt

SEQ ID NO: 2022 - DNA, 20 bp, Homo sapiens
aggttggaccccagagattc

SEQ ID NO: 2023 - DNA, 20 bp, Homo sapiens
ggttggaccccagagattcc

SEQ ID NO: 2024 - DNA, 20 bp, Homo sapiens
gttggaccccagagattccc

SEQ ID NO: 2025 - DNA, 20 bp, Homo sapiens
ttggaccccagagattccca

SEQ ID NO: 2026 - DNA, 20 bp, Homo sapiens
tggaccccagagattcccaa

SEQ ID NO: 2027 - DNA, 20 bp, Homo sapiens
ggaccccagagattcccaag

SEQ ID NO: 2028 - DNA, 20 bp, Homo sapiens
gaccccagagattcccaagc

SEQ ID NO: 2029 - DNA, 20 bp, Homo sapiens
accccagagattcccaagcc

SEQ ID NO: 2030 - DNA, 20 bp, Homo sapiens
ccccagagattcccaagcca

SEQ ID NO: 2031 - DNA, 20 bp, Homo sapiens
cccagagattcccaagccaa

SEQ ID NO: 2032 - DNA, 20 bp, Homo sapiens
ccagagattcccaagccaaa

SEQ ID NO: 2033 - DNA, 20 bp, Homo sapiens
cagagattcccaagccaaaa

SEQ ID NO: 2034 - DNA, 20 bp, Homo sapiens
agagattcccaagccaaaaa

SEQ ID NO: 2035 - DNA, 20 bp, Homo sapiens
gagattcccaagccaaaaac

SEQ ID NO: 2036 - DNA, 20 bp, Homo sapiens
agattcccaagccaaaaacc

SEQ ID NO: 2037 - DNA, 20 bp, Homo sapiens
gattcccaagccaaaaacct

SEQ ID NO: 2038 - DNA, 20 bp, Homo sapiens
attcccaagccaaaaacctt

SEQ ID NO: 2039 - DNA, 20 bp, Homo sapiens
ttcccaagccaaaaacctta

SEQ ID NO: 2040 - DNA, 20 bp, Homo sapiens
tcccaagccaaaaaccttag

SEQ ID NO: 2041 - DNA, 20 bp, Homo sapiens
cccaagccaaaaaccttagc

SEQ ID NO: 2042 - DNA, 20 bp, Homo sapiens
ccaagccaaaaaccttagct

SEQ ID NO: 2043 - DNA, 20 bp, Homo sapiens
caagccaaaaaccttagctc

SEQ ID NO: 2044 - DNA, 20 bp, Homo sapiens
aagccaaaaaccttagctcc

SEQ ID NO: 2045 - DNA, 20 bp, Homo sapiens
agccaaaaaccttagctccc

SEQ ID NO: 2046 - DNA, 20 bp, Homo sapiens
gccaaaaaccttagctccct

SEQ ID NO: 2047 - DNA, 20 bp, Homo sapiens
ccaaaaaccttagctccctc

SEQ ID NO: 2048 - DNA, 20 bp, Homo sapiens
caaaaaccttagctccctcc

SEQ ID NO: 2049 - DNA, 20 bp, Homo sapiens
aaaaaccttagctccctccc

SEQ ID NO: 2050 - DNA, 20 bp, Homo sapiens
aaaaccttagctccctcccg

SEQ ID NO: 2051 - DNA, 20 bp, Homo sapiens
aaaccttagctccctcccgc

SEQ ID NO: 2052 - DNA, 20 bp, Homo sapiens
aaccttagctccctcccgca

SEQ ID NO: 2053 - DNA, 20 bp, Homo sapiens
accttagctccctcccgcac

SEQ ID NO: 2054 - DNA, 20 bp, Homo sapiens
ccttagctccctcccgcacc

SEQ ID NO: 2055 - DNA, 20 bp, Homo sapiens
cttagctccctcccgcaccc

SEQ ID NO: 2056 - DNA, 20 bp, Homo sapiens
ttagctccctcccgcacccc

SEQ ID NO: 2057 - DNA, 20 bp, Homo sapiens
tagctccctcccgcaccccg

SEQ ID NO: 2058 - DNA, 20 bp, Homo sapiens
agctccctcccgcaccccga

SEQ ID NO: 2059 - DNA, 20 bp, Homo sapiens
gctccctcccgcaccccgat

SEQ ID NO: 2060 - DNA, 20 bp, Homo sapiens
ctccctcccgcaccccgatg

SEQ ID NO: 2061 - DNA, 20 bp, Homo sapiens
tccctcccgcaccccgatgt

SEQ ID NO: 2062 - DNA, 20 bp, Homo sapiens
ccctcccgcaccccgatgtg

SEQ ID NO: 2063 - DNA, 20 bp, Homo sapiens
cctcccgcaccccgatgtgg

SEQ ID NO: 2064 - DNA, 20 bp, Homo sapiens
ctcccgcaccccgatgtgga

SEQ ID NO: 2065 - DNA, 20 bp, Homo sapiens
tcccgcaccccgatgtggac

SEQ ID NO: 2066 - DNA, 20 bp, Homo sapiens
cccgcaccccgatgtggacc

SEQ ID NO: 2067 - DNA, 20 bp, Homo sapiens
ccgcaccccgatgtggacct

SEQ ID NO: 2068 - DNA, 21 bp, Homo sapiens
cgcaccccgatgtggacctct

SEQ ID NO: 2069 - DNA, 20 bp, Homo sapiens
caccccgatgtggacctcta

SEQ ID NO: 2070 - DNA, 20 bp, Homo sapiens
accccgatgtggacctctac

SEQ ID NO: 2071 - DNA, 20 bp, Homo sapiens
ccccgatgtggacctctact

SEQ ID NO: 2072 - DNA, 20 bp, Homo sapiens
cccgatgtggacctctactt

SEQ ID NO: 2073 - DNA, 20 bp, Homo sapiens
ccgatgtggacctctacttt

SEQ ID NO: 2074 - DNA, 20 bp, Homo sapiens
cgatgtggacctctactttc

SEQ ID NO: 2075 - DNA, 20 bp, Homo sapiens
gatgtggacctctactttcc

SEQ ID NO: 2076 - DNA, 20 bp, Homo sapiens
atgtggacctctactttcca

SEQ ID NO: 2077 - DNA, 20 bp, Homo sapiens
tgtggacctctactttccag

SEQ ID NO: 2078 - DNA, 20 bp, Homo sapiens
gtggacctctactttccagg

SEQ ID NO: 2079 - DNA, 20 bp, Homo sapiens
tggacctctactttccaggc

SEQ ID NO: 2080 - DNA, 20 bp, Homo sapiens
ggacctctactttccaggct

SEQ ID NO: 2081 - DNA, 20 bp, Homo sapiens
gacctctactttccaggcta

SEQ ID NO: 2082 - DNA, 20 bp, Homo sapiens
acctctactttccaggctag

SEQ ID NO: 2083 - DNA, 20 bp, Homo sapiens
cctctactttccaggctagt

SEQ ID NO: 2084 - DNA, 20 bp, Homo sapiens
ctctactttccaggctagtc

SEQ ID NO: 2085 - DNA, 20 bp, Homo sapiens
tctactttccaggctagtcc

SEQ ID NO: 2086 - DNA, 20 bp, Homo sapiens
ctactttccaggctagtccg

SEQ ID NO: 2087 - DNA, 20 bp, Homo sapiens
tactttccaggctagtccgg

SEQ ID NO: 2088 - DNA, 20 bp, Homo sapiens
actttccaggctagtccgga

SEQ ID NO: 2089 - DNA, 20 bp, Homo sapiens
ctttccaggctagtccggac

SEQ ID NO: 2090 - DNA, 20 bp, Homo sapiens
tttccaggctagtccggacc

SEQ ID NO: 2091 - DNA, 20 bp, Homo sapiens
ttccaggctagtccggaccc

SEQ ID NO: 2092 - DNA, 20 bp, Homo sapiens
tccaggctagtccggaccca

SEQ ID NO: 2093 - DNA, 20 bp, Homo sapiens
ccaggctagtccggacccac

SEQ ID NO: 2094 - DNA, 20 bp, Homo sapiens
caggctagtccggacccacc

SEQ ID NO: 2095 - DNA, 20 bp, Homo sapiens
aggctagtccggacccacct

SEQ ID NO: 2096 - DNA, 20 bp, Homo sapiens
ggctagtccggacccacctc

SEQ ID NO: 2097 - DNA, 20 bp, Homo sapiens
gctagtccggacccacctca

SEQ ID NO: 2098 - DNA, 20 bp, Homo sapiens
ctagtccggacccacctcac

SEQ ID NO: 2099 - DNA, 20 bp, Homo sapiens
tagtccggacccacctcacc

SEQ ID NO: 2100 - DNA, 20 bp, Homo sapiens
agtccggacccacctcaccc

SEQ ID NO: 2101 - DNA, 20 bp, Homo sapiens
gtccggacccacctcacccc

SEQ ID NO: 2102 - DNA, 20 bp, Homo sapiens
tccggacccacctcaccccg

SEQ ID NO: 2103 - DNA, 20 bp, Homo sapiens
ccggacccacctcaccccgt

SEQ ID NO: 2104 - DNA, 20 bp, Homo sapiens
cggacccacctcaccccgtt

SEQ ID NO: 2105 - DNA, 20 bp, Homo sapiens
gacccacctcaccccgttac

SEQ ID NO: 2106 - DNA, 20 bp, Homo sapiens
acccacctcaccccgttaca

SEQ ID NO: 2107 - DNA, 20 bp, Homo sapiens
cccacctcaccccgttacag

SEQ ID NO: 2108 - DNA, 20 bp, Homo sapiens
ccacctcaccccgttacagc

SEQ ID NO: 2109 - DNA, 20 bp, Homo sapiens
cacctcaccccgttacagct

SEQ ID NO: 2110 - DNA, 20 bp, Homo sapiens
acctcaccccgttacagctc

SEQ ID NO: 2111 - DNA, 20 bp, Homo sapiens
cctcaccccgttacagctcc

SEQ ID NO: 2112 - DNA, 20 bp, Homo sapiens
ctcaccccgttacagctccc

SEQ ID NO: 2113 - DNA, 20 bp, Homo sapiens
tcaccccgttacagctcccc

SEQ ID NO: 2114 - DNA, 20 bp, Homo sapiens
caccccgttacagctcccca

SEQ ID NO: 2115 - DNA, 20 bp, Homo sapiens
accccgttacagctccccaa

SEQ ID NO: 2116 - DNA, 20 bp, Homo sapiens
ccccgttacagctccccaag

SEQ ID NO: 2117 - DNA, 20 bp, Homo sapiens
cccgttacagctccccaagt

SEQ ID NO: 2118 - DNA, 20 bp, Homo sapiens
ccgttacagctccccaagtg

SEQ ID NO: 2119 - DNA, 20 bp, Homo sapiens
cgttacagctccccaagtgg

SEQ ID NO: 2120 - DNA, 20 bp, Homo sapiens
gttacagctccccaagtggt

SEQ ID NO: 2121 - DNA, 20 bp, Homo sapiens
ttacagctccccaagtggtt

SEQ ID NO: 2122 - DNA, 20 bp, Homo sapiens
tacagctccccaagtggttt

SEQ ID NO: 2123 - DNA, 20 bp, Homo sapiens
acagctccccaagtggtttc

SEQ ID NO: 2124 - DNA, 20 bp, Homo sapiens
cagctccccaagtggtttcc

SEQ ID NO: 2125 - DNA, 20 bp, Homo sapiens
agctccccaagtggtttcca

SEQ ID NO: 2126 - DNA, 20 bp, Homo sapiens
gctccccaagtggtttccac

SEQ ID NO: 2127 - DNA, 20 bp, Homo sapiens
ctccccaagtggtttccaca

SEQ ID NO: 2128 - DNA, 20 bp, Homo sapiens
tccccaagtggtttccacat

SEQ ID NO: 2129 - DNA, 20 bp, Homo sapiens
ccccaagtggtttccacatg

SEQ ID NO: 2130 - DNA, 20 bp, Homo sapiens
cccaagtggtttccacatgc

SEQ ID NO: 2131 - DNA, 20 bp, Homo sapiens
ccaagtggtttccacatgct

SEQ ID NO: 2132 - DNA, 20 bp, Homo sapiens
caagtggtttccacatgctc

SEQ ID NO: 2133 - DNA, 20 bp, Homo sapiens
aagtggtttccacatgctct

SEQ ID NO: 2134 - DNA, 20 bp, Homo sapiens
agtggtttccacatgctctg

SEQ ID NO: 2135 - DNA, 20 bp, Homo sapiens
gtggtttccacatgctctga

SEQ ID NO: 2136 - DNA, 20 bp, Homo sapiens
tggtttccacatgctctgag

SEQ ID NO: 2137 - DNA, 20 bp, Homo sapiens
ggtttccacatgctctgaga

SEQ ID NO: 2138 - DNA, 20 bp, Homo sapiens
gtttccacatgctctgagaa

SEQ ID NO: 2139 - DNA, 20 bp, Homo sapiens
tttccacatgctctgagaag

SEQ ID NO: 2140 - DNA, 20 bp, Homo sapiens
ttccacatgctctgagaaga

SEQ ID NO: 2141 - DNA, 20 bp, Homo sapiens
tccacatgctctgagaagag

SEQ ID NO: 2142 - DNA, 20 bp, Homo sapiens
ccacatgctctgagaagagg

SEQ ID NO: 2143 - DNA, 20 bp, Homo sapiens
cacatgctctgagaagagga

SEQ ID NO: 2144 - DNA, 20 bp, Homo sapiens
acatgctctgagaagaggag

SEQ ID NO: 2145 - DNA, 20 bp, Homo sapiens
catgctctgagaagaggagc

SEQ ID NO: 2146 - DNA, 20 bp, Homo sapiens
atgctctgagaagaggagcc

SEQ ID NO: 2147 - DNA, 20 bp, Homo sapiens
tgctctgagaagaggagccc

SEQ ID NO: 2148 - DNA, 20 bp, Homo sapiens
gctctgagaagaggagccct

SEQ ID NO: 2149 - DNA, 20 bp, Homo sapiens
ctctgagaagaggagccctc

SEQ ID NO: 2150 - DNA, 20 bp, Homo sapiens
tctgagaagaggagccctca

SEQ ID NO: 2151 - DNA, 20 bp, Homo sapiens
ctgagaagaggagccctcat

SEQ ID NO: 2152 - DNA, 20 bp, Homo sapiens
tgagaagaggagccctcatc

SEQ ID NO: 2153 - DNA, 20 bp, Homo sapiens
gagaagaggagccctcatct

SEQ ID NO: 2154 - DNA, 20 bp, Homo sapiens
agaagaggagccctcatctt

SEQ ID NO: 2155 - DNA, 20 bp, Homo sapiens
gaagaggagccctcatcttg

SEQ ID NO: 2156 - DNA, 20 bp, Homo sapiens
aagaggagccctcatcttga

SEQ ID NO: 2157 - DNA, 20 bp, Homo sapiens
agaggagccctcatcttgaa

SEQ ID NO: 2158 - DNA, 20 bp, Homo sapiens
gaggagccctcatcttgaag

SEQ ID NO: 2159 - DNA, 20 bp, Homo sapiens
aggagccctcatcttgaagg

SEQ ID NO: 2160 - DNA, 20 bp, Homo sapiens
ggagccctcatcttgaaggg

SEQ ID NO: 2161 - DNA, 20 bp, Homo sapiens
gagccctcatcttgaagggc

SEQ ID NO: 2162 - DNA, 20 bp, Homo sapiens
agccctcatcttgaagggcc

SEQ ID NO: 2163 - DNA, 20 bp, Homo sapiens
gccctcatcttgaagggccc

SEQ ID NO: 2164 - DNA, 20 bp, Homo sapiens
ccctcatcttgaagggccca

SEQ ID NO: 2165 - DNA, 20 bp, Homo sapiens
cctcatcttgaagggcccag

SEQ ID NO: 2166 - DNA, 20 bp, Homo sapiens
ctcatcttgaagggcccagg

SEQ ID NO: 2167 - DNA, 20 bp, Homo sapiens
tcatcttgaagggcccagga

SEQ ID NO: 2168 - DNA, 20 bp, Homo sapiens
catcttgaagggcccaggag

SEQ ID NO: 2169 - DNA, 20 bp, Homo sapiens
atcttgaagggcccaggagg

SEQ ID NO: 2170 - DNA, 20 bp, Homo sapiens
tcttgaagggcccaggaggg

SEQ ID NO: 2171 - DNA, 20 bp, Homo sapiens
cttgaagggcccaggagggt

SEQ ID NO: 2172 - DNA, 20 bp, Homo sapiens
ttgaagggcccaggagggtc

SEQ ID NO: 2173 - DNA, 20 bp, Homo sapiens
tgaagggcccaggagggtct

SEQ ID NO: 2174 - DNA, 20 bp, Homo sapiens
gaagggcccaggagggtcta

SEQ ID NO: 2175 - DNA, 20 bp, Homo sapiens
aagggcccaggagggtctat

SEQ ID NO: 2176 - DNA, 20 bp, Homo sapiens
agggcccaggagggtctatg

SEQ ID NO: 2177 - DNA, 20 bp, Homo sapiens
gggcccaggagggtctatgg

SEQ ID NO: 2178 - DNA, 20 bp, Homo sapiens
ggcccaggagggtctatggg

SEQ ID NO: 2179 - DNA, 20 bp, Homo sapiens
gcccaggagggtctatgggg

SEQ ID NO: 2180 - DNA, 20 bp, Homo sapiens
cccaggagggtctatgggga

SEQ ID NO: 2181 - DNA, 20 bp, Homo sapiens
ccaggagggtctatggggag

SEQ ID NO: 2182 - DNA, 20 bp, Homo sapiens
caggagggtctatggggaga

SEQ ID NO: 2183 - DNA, 20 bp, Homo sapiens
aggagggtctatggggagag

SEQ ID NO: 2184 - DNA, 20 bp, Homo sapiens
ggagggtctatggggagagg

SEQ ID NO: 2185 - DNA, 20 bp, Homo sapiens
gagggtctatggggagagga

SEQ ID NO: 2186 - DNA, 20 bp, Homo sapiens
agggtctatggggagaggaa

SEQ ID NO: 2187 - DNA, 20 bp, Homo sapiens
gggtctatggggagaggaac

SEQ ID NO: 2188 - DNA, 20 bp, Homo sapiens
ggtctatggggagaggaact

SEQ ID NO: 2189 - DNA, 20 bp, Homo sapiens
gtctatggggagaggaactc

SEQ ID NO: 2190 - DNA, 20 bp, Homo sapiens
tctatggggagaggaactcc

SEQ ID NO: 2191 - DNA, 20 bp, Homo sapiens
ctatggggagaggaactcct

SEQ ID NO: 2192 - DNA, 20 bp, Homo sapiens
tatggggagaggaactcctt

SEQ ID NO: 2193 - DNA, 20 bp, Homo sapiens
atggggagaggaactccttg

SEQ ID NO: 2194 - DNA, 20 bp, Homo sapiens
tggggagaggaactccttgg

SEQ ID NO: 2195 - DNA, 20 bp, Homo sapiens
ggggagaggaactccttggc

SEQ ID NO: 2196 - DNA, 20 bp, Homo sapiens
gggagaggaactccttggcc

SEQ ID NO: 2197 - DNA, 20 bp, Homo sapiens
ggagaggaactccttggcct

SEQ ID NO: 2198 - DNA, 20 bp, Homo sapiens
gagaggaactccttggccta

SEQ ID NO: 2199 - DNA, 20 bp, Homo sapiens
agaggaactccttggcctag

SEQ ID NO: 2200 - DNA, 20 bp, Homo sapiens
gaggaactccttggcctagc

SEQ ID NO: 2201 - DNA, 20 bp, Homo sapiens
aggaactccttggcctagcc

SEQ ID NO: 2202 - DNA, 20 bp, Homo sapiens
ggaactccttggcctagccc

SEQ ID NO: 2203 - DNA, 20 bp, Homo sapiens
gaactccttggcctagccca

SEQ ID NO: 2204 - DNA, 20 bp, Homo sapiens
aactccttggcctagcccac

SEQ ID NO: 2205 - DNA, 20 bp, Homo sapiens
actccttggcctagcccacc

SEQ ID NO: 2206 - DNA, 20 bp, Homo sapiens
ctccttggcctagcccaccc

SEQ ID NO: 2207 - DNA, 20 bp, Homo sapiens
tccttggcctagcccaccct

SEQ ID NO: 2208 - DNA, 20 bp, Homo sapiens
ccttggcctagcccaccctg

SEQ ID NO: 2209 - DNA, 20 bp, Homo sapiens
cttggcctagcccaccctgc

SEQ ID NO: 2210 - DNA, 20 bp, Homo sapiens
ttggcctagcccaccctgct

SEQ ID NO: 2211 - DNA, 20 bp, Homo sapiens
tggcctagcccaccctgctg

SEQ ID NO: 2212 - DNA, 20 bp, Homo sapiens
ggcctagcccaccctgctgc

SEQ ID NO: 2213 - DNA, 20 bp, Homo sapiens
gcctagcccaccctgctgcc

SEQ ID NO: 2214 - DNA, 20 bp, Homo sapiens
cctagcccaccctgctgcct

SEQ ID NO: 2215 - DNA, 20 bp, Homo sapiens
ctagcccaccctgctgcctt

SEQ ID NO: 2216 - DNA, 20 bp, Homo sapiens
tagcccaccctgctgccttc

SEQ ID NO: 2217 - DNA, 20 bp, Homo sapiens
agcccaccctgctgccttct

SEQ ID NO: 2218 - DNA, 20 bp, Homo sapiens
gcccaccctgctgccttctg

SEQ ID NO: 2219 - DNA, 20 bp, Homo sapiens
cccaccctgctgccttctga

SEQ ID NO: 2220 - DNA, 20 bp, Homo sapiens
ccaccctgctgccttctgac

SEQ ID NO: 2221 - DNA, 20 bp, Homo sapiens
caccctgctgccttctgacg

SEQ ID NO: 2222 - DNA, 20 bp, Homo sapiens
accctgctgccttctgacgg

SEQ ID NO: 2223 - DNA, 20 bp, Homo sapiens
ccctgctgccttctgacggc

SEQ ID NO: 2224 - DNA, 20 bp, Homo sapiens
cctgctgccttctgacggcc

SEQ ID NO: 2225 - DNA, 20 bp, Homo sapiens
ctgctgccttctgacggccc

SEQ ID NO: 2226 - DNA, 20 bp, Homo sapiens
tgctgccttctgacggccct

SEQ ID NO: 2227 - DNA, 20 bp, Homo sapiens
gctgccttctgacggccctg

SEQ ID NO: 2228 - DNA, 20 bp, Homo sapiens
ctgccttctgacggccctgc

SEQ ID NO: 2229 - DNA, 20 bp, Homo sapiens
tgccttctgacggccctgca

SEQ ID NO: 2230 - DNA, 20 bp, Homo sapiens
gccttctgacggccctgcaa

SEQ ID NO: 2231 - DNA, 20 bp, Homo sapiens
ccttctgacggccctgcaat

SEQ ID NO: 2232 - DNA, 20 bp, Homo sapiens
cttctgacggccctgcaatg

SEQ ID NO: 2233 - DNA, 20 bp, Homo sapiens
ttctgacggccctgcaatgt

SEQ ID NO: 2234 - DNA, 20 bp, Homo sapiens
tctgacggccctgcaatgta

SEQ ID NO: 2235 - DNA, 20 bp, Homo sapiens
ctgacggccctgcaatgtat

SEQ ID NO: 2236 - DNA, 20 bp, Homo sapiens
tgacggccctgcaatgtatc

SEQ ID NO: 2237 - DNA, 20 bp, Homo sapiens
gacggccctgcaatgtatcc

SEQ ID NO: 2238 - DNA, 20 bp, Homo sapiens
acggccctgcaatgtatccc

SEQ ID NO: 2239 - DNA, 20 bp, Homo sapiens
cggccctgcaatgtatccct

SEQ ID NO: 2240 - DNA, 20 bp, Homo sapiens
ggccctgcaatgtatccctt

SEQ ID NO: 2241 - DNA, 20 bp, Homo sapiens
gccctgcaatgtatcccttc

SEQ ID NO: 2242 - DNA, 20 bp, Homo sapiens
ccctgcaatgtatcccttct

SEQ ID NO: 2243 - DNA, 20 bp, Homo sapiens
cctgcaatgtatcccttctc

SEQ ID NO: 2244 - DNA, 20 bp, Homo sapiens
ctgcaatgtatcccttctca

SEQ ID NO: 2245 - DNA, 20 bp, Homo sapiens
tgcaatgtatcccttctcac

SEQ ID NO: 2246 - DNA, 20 bp, Homo sapiens
gcaatgtatcccttctcaca

SEQ ID NO: 2247 - DNA, 20 bp, Homo sapiens
caatgtatcccttctcacag

SEQ ID NO: 2248 - DNA, 20 bp, Homo sapiens
aatgtatcccttctcacagc

SEQ ID NO: 2249 - DNA, 20 bp, Homo sapiens
atgtatcccttctcacagca

SEQ ID NO: 2250 - DNA, 20 bp, Homo sapiens
tgtatcccttctcacagcac

SEQ ID NO: 2251 - DNA, 20 bp, Homo sapiens
gtatcccttctcacagcaca

SEQ ID NO: 2252 - DNA, 20 bp, Homo sapiens
tatcccttctcacagcacat

SEQ ID NO: 2253 - DNA, 20 bp, Homo sapiens
atcccttctcacagcacatg

SEQ ID NO: 2254 - DNA, 20 bp, Homo sapiens
tcccttctcacagcacatgc

SEQ ID NO: 2255 - DNA, 20 bp, Homo sapiens
cccttctcacagcacatgct

SEQ ID NO: 2256 - DNA, 20 bp, Homo sapiens
ccttctcacagcacatgctg

SEQ ID NO: 2257 - DNA, 20 bp, Homo sapiens
cttctcacagcacatgctgg

SEQ ID NO: 2258 - DNA, 20 bp, Homo sapiens
ttctcacagcacatgctggc

SEQ ID NO: 2259 - DNA, 20 bp, Homo sapiens
tctcacagcacatgctggcc

SEQ ID NO: 2260 - DNA, 20 bp, Homo sapiens
ctcacagcacatgctggcca

SEQ ID NO: 2261 - DNA, 20 bp, Homo sapiens
tcacagcacatgctggccag

SEQ ID NO: 2262 - DNA, 20 bp, Homo sapiens
cacagcacatgctggccagc

SEQ ID NO: 2263 - DNA, 20 bp, Homo sapiens
acagcacatgctggccagcc

SEQ ID NO: 2264 - DNA, 20 bp, Homo sapiens
cagcacatgctggccagcct

SEQ ID NO: 2265 - DNA, 20 bp, Homo sapiens
agcacatgctggccagcctg

SEQ ID NO: 2266 - DNA, 20 bp, Homo sapiens
gcacatgctggccagcctgg

SEQ ID NO: 2267 - DNA, 20 bp, Homo sapiens
cacatgctggccagcctggg

SEQ ID NO: 2268 - DNA, 20 bp, Homo sapiens
acatgctggccagcctgggg

SEQ ID NO: 2269 - DNA, 20 bp, Homo sapiens
catgctggccagcctggggc

SEQ ID NO: 2270 - DNA, 20 bp, Homo sapiens
atgctggccagcctggggcc

SEQ ID NO: 2271 - DNA, 20 bp, Homo sapiens
tgctggccagcctggggcct

SEQ ID NO: 2272 - DNA, 20 bp, Homo sapiens
gctggccagcctggggcctg

SEQ ID NO: 2273 - DNA, 20 bp, Homo sapiens
ctggccagcctggggcctgg

SEQ ID NO: 2274 - DNA, 20 bp, Homo sapiens
tggccagcctggggcctggc

SEQ ID NO: 2275 - DNA, 20 bp, Homo sapiens
ggccagcctggggcctggca

SEQ ID NO: 2276 - DNA, 20 bp, Homo sapiens
gccagcctggggcctggcag

SEQ ID NO: 2277 - DNA, 20 bp, Homo sapiens
ccagcctggggcctggcagg

SEQ ID NO: 2278 - DNA, 20 bp, Homo sapiens
cagcctggggcctggcaggg

SEQ ID NO: 2279 - DNA, 20 bp, Homo sapiens
agcctggggcctggcaggga

SEQ ID NO: 2280 - DNA, 20 bp, Homo sapiens
gcctggggcctggcagggag

SEQ ID NO: 2281 - DNA, 20 bp, Homo sapiens
cctggggcctggcagggagg

SEQ ID NO: 2282 - DNA, 20 bp, Homo sapiens
ctggggcctggcagggaggt

SEQ ID NO: 2283 - DNA, 20 bp, Homo sapiens
tggggcctggcagggaggtc

SEQ ID NO: 2284 - DNA, 20 bp, Homo sapiens
ggggcctggcagggaggtca

SEQ ID NO: 2285 - DNA, 20 bp, Homo sapiens
gggcctggcagggaggtcag

SEQ ID NO: 2286 - DNA, 20 bp, Homo sapiens
ggcctggcagggaggtcagg

SEQ ID NO: 2287 - DNA, 20 bp, Homo sapiens
gcctggcagggaggtcaggc

SEQ ID NO: 2288 - DNA, 20 bp, Homo sapiens
cctggcagggaggtcaggcc

SEQ ID NO: 2289 - DNA, 20 bp, Homo sapiens
ctggcagggaggtcaggccc

SEQ ID NO: 2290 - DNA, 20 bp, Homo sapiens
tggcagggaggtcaggccct

SEQ ID NO: 2291 - DNA, 20 bp, Homo sapiens
ggcagggaggtcaggccctg

SEQ ID NO: 2292 - DNA, 20 bp, Homo sapiens
gcagggaggtcaggccctgg

SEQ ID NO: 2293 - DNA, 20 bp, Homo sapiens
cagggaggtcaggccctgga

SEQ ID NO: 2294 - DNA, 20 bp, Homo sapiens
agggaggtcaggccctggaa

SEQ ID NO: 2295 - DNA, 20 bp, Homo sapiens
gggaggtcaggccctggaac

SEQ ID NO: 2296 - DNA, 20 bp, Homo sapiens
ggaggtcaggccctggaact

SEQ ID NO: 2297 - DNA, 20 bp, Homo sapiens
gaggtcaggccctggaactc

SEQ ID NO: 2298 - DNA, 20 bp, Homo sapiens
aggtcaggccctggaactct

SEQ ID NO: 2299 - DNA, 20 bp, Homo sapiens
ggtcaggccctggaactcta

SEQ ID NO: 2300 - DNA, 20 bp, Homo sapiens
gtcaggccctggaactctat

SEQ ID NO: 2301 - DNA, 20 bp, Homo sapiens
tcaggccctggaactctatc

SEQ ID NO: 2302 - DNA, 20 bp, Homo sapiens
caggccctggaactctatct

SEQ ID NO: 2303 - DNA, 20 bp, Homo sapiens
aggccctggaactctatctg

SEQ ID NO: 2304 - DNA, 20 bp, Homo sapiens
ggccctggaactctatctgg

SEQ ID NO: 2305 - DNA, 20 bp, Homo sapiens
gccctggaactctatctggg

SEQ ID NO: 2306 - DNA, 20 bp, Homo sapiens
ccctggaactctatctgggc

SEQ ID NO: 2307 - DNA, 20 bp, Homo sapiens
cctggaactctatctgggcc

SEQ ID NO: 2308 - DNA, 20 bp, Homo sapiens
ctggaactctatctgggcct

SEQ ID NO: 2309 - DNA, 20 bp, Homo sapiens
tggaactctatctgggcctg

SEQ ID NO: 2310 - DNA, 20 bp, Homo sapiens
ggaactctatctgggcctgg

SEQ ID NO: 2311 - DNA, 20 bp, Homo sapiens
gaactctatctgggcctggg

SEQ ID NO: 2312 - DNA, 20 bp, Homo sapiens
aactctatctgggcctgggc

SEQ ID NO: 2313 - DNA, 20 bp, Homo sapiens
actctatctgggcctgggct

SEQ ID NO: 2314 - DNA, 20 bp, Homo sapiens
ctctatctgggcctgggcta

SEQ ID NO: 2315 - DNA, 20 bp, Homo sapiens
tctatctgggcctgggctag

SEQ ID NO: 2316 - DNA, 20 bp, Homo sapiens
ctatctgggcctgggctagg

SEQ ID NO: 2317 - DNA, 20 bp, Homo sapiens
tatctgggcctgggctaggg

SEQ ID NO: 2318 - DNA, 20 bp, Homo sapiens
atctgggcctgggctagggg

SEQ ID NO: 2319 - DNA, 20 bp, Homo sapiens
tctgggcctgggctagggga

SEQ ID NO: 2320 - DNA, 20 bp, Homo sapiens
ctgggcctgggctaggggac

SEQ ID NO: 2321 - DNA, 20 bp, Homo sapiens
tgggcctgggctaggggaca

SEQ ID NO: 2322 - DNA, 20 bp, Homo sapiens
gggcctgggctaggggacat

SEQ ID NO: 2323 - DNA, 20 bp, Homo sapiens
ggcctgggctaggggacatc

SEQ ID NO: 2324 - DNA, 20 bp, Homo sapiens
gcctgggctaggggacatca

SEQ ID NO: 2325 - DNA, 20 bp, Homo sapiens
cctgggctaggggacatcag

SEQ ID NO: 2326 - DNA, 20 bp, Homo sapiens
ctgggctaggggacatcaga

SEQ ID NO: 2327 - DNA, 20 bp, Homo sapiens
tgggctaggggacatcagag

SEQ ID NO: 2328 - DNA, 20 bp, Homo sapiens
gggctaggggacatcagagg

SEQ ID NO: 2329 - DNA, 20 bp, Homo sapiens
ggctaggggacatcagaggt

SEQ ID NO: 2330 - DNA, 20 bp, Homo sapiens
gctaggggacatcagaggtt

SEQ ID NO: 2331 - DNA, 20 bp, Homo sapiens
ctaggggacatcagaggttc

SEQ ID NO: 2332 - DNA, 20 bp, Homo sapiens
taggggacatcagaggttct

SEQ ID NO: 2333 - DNA, 20 bp, Homo sapiens
aggggacatcagaggttctt

SEQ ID NO: 2334 - DNA, 20 bp, Homo sapiens
ggggacatcagaggttcttt

SEQ ID NO: 2335 - DNA, 20 bp, Homo sapiens
gggacatcagaggttctttg

SEQ ID NO: 2336 - DNA, 20 bp, Homo sapiens
ggacatcagaggttctttga

SEQ ID NO: 2337 - DNA, 20 bp, Homo sapiens
gacatcagaggttctttgag

SEQ ID NO: 2338 - DNA, 20 bp, Homo sapiens
acatcagaggttctttgagg

SEQ ID NO: 2339 - DNA, 20 bp, Homo sapiens
catcagaggttctttgaggg

SEQ ID NO: 2340 - DNA, 20 bp, Homo sapiens
atcagaggttctttgaggga

SEQ ID NO: 2341 - DNA, 20 bp, Homo sapiens
tcagaggttctttgagggac

SEQ ID NO: 2342 - DNA, 20 bp, Homo sapiens
cagaggttctttgagggact

SEQ ID NO: 2343 - DNA, 20 bp, Homo sapiens
agaggttctttgagggactg

SEQ ID NO: 2344 - DNA, 20 bp, Homo sapiens
gaggttctttgagggactgc

SEQ ID NO: 2345 - DNA, 20 bp, Homo sapiens
aggttctttgagggactgcc

SEQ ID NO: 2346 - DNA, 20 bp, Homo sapiens
ggttctttgagggactgcct

SEQ ID NO: 2347 - DNA, 20 bp, Homo sapiens
gttctttgagggactgcctc

SEQ ID NO: 2348 - DNA, 20 bp, Homo sapiens
ttctttgagggactgcctct

SEQ ID NO: 2349 - DNA, 20 bp, Homo sapiens
tctttgagggactgcctctg

SEQ ID NO: 2350 - DNA, 20 bp, Homo sapiens
ctttgagggactgcctctgc

SEQ ID NO: 2351 - DNA, 20 bp, Homo sapiens
tttgagggactgcctctgcc

SEQ ID NO: 2352 - DNA, 20 bp, Homo sapiens
ttgagggactgcctctgcca

SEQ ID NO: 2353 - DNA, 20 bp, Homo sapiens
tgagggactgcctctgccac

SEQ ID NO: 2354 - DNA, 20 bp, Homo sapiens
gagggactgcctctgccaca

SEQ ID NO: 2355 - DNA, 20 bp, Homo sapiens
agggactgcctctgccacac

SEQ ID NO: 2356 - DNA, 20 bp, Homo sapiens
gggactgcctctgccacact

SEQ ID NO: 2357 - DNA, 20 bp, Homo sapiens
ggactgcctctgccacactc

SEQ ID NO: 2358 - DNA, 20 bp, Homo sapiens
gactgcctctgccacactct

SEQ ID NO: 2359 - DNA, 20 bp, Homo sapiens
actgcctctgccacactctg

SEQ ID NO: 2360 - DNA, 20 bp, Homo sapiens
ctgcctctgccacactctga

SEQ ID NO: 2361 - DNA, 20 bp, Homo sapiens
tgcctctgccacactctgac

SEQ ID NO: 2362 - DNA, 20 bp, Homo sapiens
gcctctgccacactctgacg

SEQ ID NO: 2363 - DNA, 20 bp, Homo sapiens
cctctgccacactctgacgc

SEQ ID NO: 2364 - DNA, 20 bp, Homo sapiens
ctctgccacactctgacgca

SEQ ID NO: 2365 - DNA, 20 bp, Homo sapiens
tctgccacactctgacgcaa

SEQ ID NO: 2366 - DNA, 20 bp, Homo sapiens
ctgccacactctgacgcaaa

SEQ ID NO: 2367 - DNA, 20 bp, Homo sapiens
tgccacactctgacgcaaaa

SEQ ID NO: 2368 - DNA, 20 bp, Homo sapiens
gccacactctgacgcaaaac

SEQ ID NO: 2369 - DNA, 20 bp, Homo sapiens
ccacactctgacgcaaaacc

SEQ ID NO: 2370 - DNA, 20 bp, Homo sapiens
cacactctgacgcaaaacca

SEQ ID NO: 2371 - DNA, 20 bp, Homo sapiens
acactctgacgcaaaaccac

SEQ ID NO: 2372 - DNA, 20 bp, Homo sapiens
cactctgacgcaaaaccact

SEQ ID NO: 2373 - DNA, 20 bp, Homo sapiens
actctgacgcaaaaccactt

SEQ ID NO: 2374 - DNA, 20 bp, Homo sapiens
ctctgacgcaaaaccacttt

SEQ ID NO: 2375 - DNA, 20 bp, Homo sapiens
tctgacgcaaaaccactttc

SEQ ID NO: 2376 - DNA, 20 bp, Homo sapiens
ctgacgcaaaaccactttcc

SEQ ID NO: 2377 - DNA, 20 bp, Homo sapiens
tgacgcaaaaccactttcct

SEQ ID NO: 2378 - DNA, 20 bp, Homo sapiens
gacgcaaaaccactttcctt

SEQ ID NO: 2379 - DNA, 20 bp, Homo sapiens
acgcaaaaccactttccttt

SEQ ID NO: 2380 - DNA, 20 bp, Homo sapiens
cgcaaaaccactttcctttt

SEQ ID NO: 2381 - DNA, 20 bp, Homo sapiens
gcaaaaccactttccttttc

SEQ ID NO: 2382 - DNA, 20 bp, Homo sapiens
caaaaccactttccttttct

SEQ ID NO: 2383 - DNA, 20 bp, Homo sapiens
aaaaccactttccttttcta

SEQ ID NO: 2384 - DNA, 20 bp, Homo sapiens
aaaccactttccttttctat

SEQ ID NO: 2385 - DNA, 20 bp, Homo sapiens
aaccactttccttttctatt

SEQ ID NO: 2386 - DNA, 20 bp, Homo sapiens
accactttccttttctattc

SEQ ID NO: 2387 - DNA, 20 bp, Homo sapiens
ccactttccttttctattcc

SEQ ID NO: 2388 - DNA, 20 bp, Homo sapiens
cactttccttttctattcct

SEQ ID NO: 2389 - DNA, 20 bp, Homo sapiens
actttccttttctattcctt

SEQ ID NO: 2390 - DNA, 20 bp, Homo sapiens
ctttccttttctattccttc

SEQ ID NO: 2391 - DNA, 20 bp, Homo sapiens
tttccttttctattccttct

SEQ ID NO: 2392 - DNA, 20 bp, Homo sapiens
ttccttttctattccttctg

SEQ ID NO: 2393 - DNA, 20 bp, Homo sapiens
tccttttctattccttctgg

SEQ ID NO: 2394 - DNA, 20 bp, Homo sapiens
ccttttctattccttctggc

SEQ ID NO: 2395 - DNA, 20 bp, Homo sapiens
cttttctattccttctggcc

SEQ ID NO: 2396 - DNA, 20 bp, Homo sapiens
ttttctattccttctggcct

SEQ ID NO: 2397 - DNA, 20 bp, Homo sapiens
tttctattccttctggcctt

SEQ ID NO: 2398 - DNA, 20 bp, Homo sapiens
ttctattccttctggccttt

SEQ ID NO: 2399 - DNA, 20 bp, Homo sapiens
tctattccttctggcctttc

SEQ ID NO: 2400 - DNA, 20 bp, Homo sapiens
ctattccttctggcctttcc

SEQ ID NO: 2401 - DNA, 20 bp, Homo sapiens
tattccttctggcctttcct

SEQ ID NO: 2402 - DNA, 20 bp, Homo sapiens
attccttctggcctttcctc

SEQ ID NO: 2403 - DNA, 20 bp, Homo sapiens
ttccttctggcctttcctct

SEQ ID NO: 2404 - DNA, 20 bp, Homo sapiens
tccttctggcctttcctctc

SEQ ID NO: 2405 - DNA, 20 bp, Homo sapiens
ccttctggcctttcctctct

SEQ ID NO: 2406 - DNA, 20 bp, Homo sapiens
cttctggcctttcctctctc

SEQ ID NO: 2407 - DNA, 20 bp, Homo sapiens
ttctggcctttcctctctcc

SEQ ID NO: 2408 - DNA, 20 bp, Homo sapiens
tctggcctttcctctctcct

SEQ ID NO: 2409 - DNA, 20 bp, Homo sapiens
ctggcctttcctctctcctg

SEQ ID NO: 2410 - DNA, 20 bp, Homo sapiens
tggcctttcctctctcctgt

SEQ ID NO: 2411 - DNA, 20 bp, Homo sapiens
ggcctttcctctctcctgtt

SEQ ID NO: 2412 - DNA, 20 bp, Homo sapiens
gcctttcctctctcctgttt

SEQ ID NO: 2413 - DNA, 20 bp, Homo sapiens
cctttcctctctcctgtttc

SEQ ID NO: 2414 - DNA, 20 bp, Homo sapiens
ctttcctctctcctgtttcc

SEQ ID NO: 2415 - DNA, 20 bp, Homo sapiens
tttcctctctcctgtttccc

SEQ ID NO: 2416 - DNA, 20 bp, Homo sapiens
ttcctctctcctgtttccct

SEQ ID NO: 2417 - DNA, 20 bp, Homo sapiens
tcctctctcctgtttccctt

SEQ ID NO: 2418 - DNA, 20 bp, Homo sapiens
cctctctcctgtttcccttc

SEQ ID NO: 2419 - DNA, 20 bp, Homo sapiens
ctctctcctgtttcccttcc

SEQ ID NO: 2420 - DNA, 20 bp, Homo sapiens
tctctcctgtttcccttccc

SEQ ID NO: 2421 - DNA, 20 bp, Homo sapiens
ctctcctgtttcccttccct

SEQ ID NO: 2422 - DNA, 20 bp, Homo sapiens
tctcctgtttcccttccctt

SEQ ID NO: 2423 - DNA, 20 bp, Homo sapiens
ctcctgtttcccttcccttc

SEQ ID NO: 2424 - DNA, 20 bp, Homo sapiens
tcctgtttcccttcccttcc

SEQ ID NO: 2425 - DNA, 20 bp, Homo sapiens
cctgtttcccttcccttcca

SEQ ID NO: 2426 - DNA, 20 bp, Homo sapiens
ctgtttcccttcccttccac

SEQ ID NO: 2427 - DNA, 20 bp, Homo sapiens
tgtttcccttcccttccact

SEQ ID NO: 2428 - DNA, 20 bp, Homo sapiens
gtttcccttcccttccactg

SEQ ID NO: 2429 - DNA, 20 bp, Homo sapiens
tttcccttcccttccactgc

SEQ ID NO: 2430 - DNA, 20 bp, Homo sapiens
ttcccttcccttccactgcc

SEQ ID NO: 2431 - DNA, 20 bp, Homo sapiens
tcccttcccttccactgcct

SEQ ID NO: 2432 - DNA, 20 bp, Homo sapiens
cccttcccttccactgcctc

SEQ ID NO: 2433 - DNA, 20 bp, Homo sapiens
ccttcccttccactgcctct

SEQ ID NO: 2434 - DNA, 20 bp, Homo sapiens
cttcccttccactgcctctg

SEQ ID NO: 2435 - DNA, 20 bp, Homo sapiens
ttcccttccactgcctctgc

SEQ ID NO: 2436 - DNA, 20 bp, Homo sapiens
tcccttccactgcctctgcc

SEQ ID NO: 2437 - DNA, 20 bp, Homo sapiens
cccttccactgcctctgcct

SEQ ID NO: 2438 - DNA, 20 bp, Homo sapiens
ccttccactgcctctgcctt

SEQ ID NO: 2439 - DNA, 20 bp, Homo sapiens
cttccactgcctctgcctta

SEQ ID NO: 2440 - DNA, 20 bp, Homo sapiens
ttccactgcctctgccttag

SEQ ID NO: 2441 - DNA, 20 bp, Homo sapiens
tccactgcctctgccttaga

SEQ ID NO: 2442 - DNA, 20 bp, Homo sapiens
ccactgcctctgccttagag

SEQ ID NO: 2443 - DNA, 20 bp, Homo sapiens
cactgcctctgccttagagg

SEQ ID NO: 2444 - DNA, 20 bp, Homo sapiens
actgcctctgccttagagga

SEQ ID NO: 2445 - DNA, 20 bp, Homo sapiens
ctgcctctgccttagaggag

SEQ ID NO: 2446 - DNA, 20 bp, Homo sapiens
tgcctctgccttagaggagc

SEQ ID NO: 2447 - DNA, 20 bp, Homo sapiens
gcctctgccttagaggagcc

SEQ ID NO: 2448 - DNA, 20 bp, Homo sapiens
cctctgccttagaggagccc

SEQ ID NO: 2449 - DNA, 20 bp, Homo sapiens
ctctgccttagaggagccca

SEQ ID NO: 2450 - DNA, 20 bp, Homo sapiens
tctgccttagaggagcccac

SEQ ID NO: 2451 - DNA, 20 bp, Homo sapiens
ctgccttagaggagcccacg

SEQ ID NO: 2452 - DNA, 20 bp, Homo sapiens
tgccttagaggagcccacgg

SEQ ID NO: 2453 - DNA, 20 bp, Homo sapiens
gccttagaggagcccacggc

SEQ ID NO: 2454 - DNA, 20 bp, Homo sapiens
ccttagaggagcccacggct

SEQ ID NO: 2455 - DNA, 20 bp, Homo sapiens
cttagaggagcccacggcta

SEQ ID NO: 2456 - DNA, 20 bp, Homo sapiens
ttagaggagcccacggctaa

SEQ ID NO: 2457 - DNA, 20 bp, Homo sapiens
tagaggagcccacggctaag

SEQ ID NO: 2458 - DNA, 20 bp, Homo sapiens
agaggagcccacggctaaga

SEQ ID NO: 2459 - DNA, 20 bp, Homo sapiens
gaggagcccacggctaagag

SEQ ID NO: 2460 - DNA, 20 bp, Homo sapiens
aggagcccacggctaagagg

SEQ ID NO: 2461 - DNA, 20 bp, Homo sapiens
ggagcccacggctaagaggc

SEQ ID NO: 2462 - DNA, 20 bp, Homo sapiens
gagcccacggctaagaggct

SEQ ID NO: 2463 - DNA, 20 bp, Homo sapiens
agcccacggctaagaggctg

SEQ ID NO: 2464 - DNA, 20 bp, Homo sapiens
gcccacggctaagaggctgc

SEQ ID NO: 2465 - DNA, 20 bp, Homo sapiens
cccacggctaagaggctgct

SEQ ID NO: 2466 - DNA, 20 bp, Homo sapiens
ccacggctaagaggctgctg

SEQ ID NO: 2467 - DNA, 20 bp, Homo sapiens
cacggctaagaggctgctga

SEQ ID NO: 2468 - DNA, 20 bp, Homo sapiens
acggctaagaggctgctgaa

SEQ ID NO: 2469 - DNA, 20 bp, Homo sapiens
cggctaagaggctgctgaaa

SEQ ID NO: 2470 - DNA, 20 bp, Homo sapiens
ggctaagaggctgctgaaaa

SEQ ID NO: 2471 - DNA, 20 bp, Homo sapiens
gctaagaggctgctgaaaac

SEQ ID NO: 2472 - DNA, 20 bp, Homo sapiens
ctaagaggctgctgaaaacc

SEQ ID NO: 2473 - DNA, 20 bp, Homo sapiens
taagaggctgctgaaaacca

SEQ ID NO: 2474 - DNA, 20 bp, Homo sapiens
aagaggctgctgaaaaccat

SEQ ID NO: 2475 - DNA, 20 bp, Homo sapiens
agaggctgctgaaaaccatc

SEQ ID NO: 2476 - DNA, 20 bp, Homo sapiens
gaggctgctgaaaaccatct

SEQ ID NO: 2477 - DNA, 20 bp, Homo sapiens
aggctgctgaaaaccatctg

SEQ ID NO: 2478 - DNA, 20 bp, Homo sapiens
ggctgctgaaaaccatctgg

SEQ ID NO: 2479 - DNA, 20 bp, Homo sapiens
gctgctgaaaaccatctggc

SEQ ID NO: 2480 - DNA, 20 bp, Homo sapiens
ctgctgaaaaccatctggcc

SEQ ID NO: 2481 - DNA, 20 bp, Homo sapiens
tgctgaaaaccatctggcct

SEQ ID NO: 2482 - DNA, 20 bp, Homo sapiens
gctgaaaaccatctggcctg

SEQ ID NO: 2483 - DNA, 20 bp, Homo sapiens
ctgaaaaccatctggcctgg

SEQ ID NO: 2484 - DNA, 20 bp, Homo sapiens
tgaaaaccatctggcctggc

SEQ ID NO: 2485 - DNA, 20 bp, Homo sapiens
gaaaaccatctggcctggcc

SEQ ID NO: 2486 - DNA, 20 bp, Homo sapiens
aaaaccatctggcctggcct

SEQ ID NO: 2487 - DNA, 20 bp, Homo sapiens
aaaccatctggcctggcctg

SEQ ID NO: 2488 - DNA, 20 bp, Homo sapiens
aaccatctggcctggcctgg

SEQ ID NO: 2489 - DNA, 20 bp, Homo sapiens
accatctggcctggcctggc

SEQ ID NO: 2490 - DNA, 20 bp, Homo sapiens
ccatctggcctggcctggcc

SEQ ID NO: 2491 - DNA, 20 bp, Homo sapiens
catctggcctggcctggccc

SEQ ID NO: 2492 - DNA, 20 bp, Homo sapiens
atctggcctggcctggccct

SEQ ID NO: 2493 - DNA, 20 bp, Homo sapiens
tctggcctggcctggccctg

SEQ ID NO: 2494 - DNA, 20 bp, Homo sapiens
ctggcctggcctggccctgc

SEQ ID NO: 2495 - DNA, 20 bp, Homo sapiens
tggcctggcctggccctgcc

SEQ ID NO: 2496 - DNA, 20 bp, Homo sapiens
ggcctggcctggccctgccc

SEQ ID NO: 2497 - DNA, 20 bp, Homo sapiens
gcctggcctggccctgccct

SEQ ID NO: 2498 - DNA, 20 bp, Homo sapiens
cctggcctggccctgccctg

SEQ ID NO: 2499 - DNA, 20 bp, Homo sapiens
ctggcctggccctgccctga

SEQ ID NO: 2500 - DNA, 20 bp, Homo sapiens
tggcctggccctgccctgag

SEQ ID NO: 2501 - DNA, 20 bp, Homo sapiens
ggcctggccctgccctgagg

SEQ ID NO: 2502 - DNA, 20 bp, Homo sapiens
gcctggccctgccctgagga

SEQ ID NO: 2503 - DNA, 20 bp, Homo sapiens
cctggccctgccctgaggaa

SEQ ID NO: 2504 - DNA, 20 bp, Homo sapiens
ctggccctgccctgaggaag

SEQ ID NO: 2505 - DNA, 20 bp, Homo sapiens
tggccctgccctgaggaagg

SEQ ID NO: 2506 - DNA, 20 bp, Homo sapiens
ggccctgccctgaggaagga

SEQ ID NO: 2507 - DNA, 20 bp, Homo sapiens
gccctgccctgaggaaggag

SEQ ID NO: 2508 - DNA, 20 bp, Homo sapiens
ccctgccctgaggaaggagg

SEQ ID NO: 2509 - DNA, 20 bp, Homo sapiens
cctgccctgaggaaggaggg

SEQ ID NO: 2510 - DNA, 20 bp, Homo sapiens
ctgccctgaggaaggagggg

SEQ ID NO: 2511 - DNA, 20 bp, Homo sapiens
tgccctgaggaaggagggga

SEQ ID NO: 2512 - DNA, 20 bp, Homo sapiens
gccctgaggaaggaggggaa

SEQ ID NO: 2513 - DNA, 20 bp, Homo sapiens
ccctgaggaaggaggggaag

SEQ ID NO: 2514 - DNA, 20 bp, Homo sapiens
cctgaggaaggaggggaagc

SEQ ID NO: 2515 - DNA, 20 bp, Homo sapiens
ctgaggaaggaggggaagct

SEQ ID NO: 2516 - DNA, 20 bp, Homo sapiens
tgaggaaggaggggaagctg

SEQ ID NO: 2517 - DNA, 20 bp, Homo sapiens
gaggaaggaggggaagctgc

SEQ ID NO: 2518 - DNA, 20 bp, Homo sapiens
aggaaggaggggaagctgca

SEQ ID NO: 2519 - DNA, 20 bp, Homo sapiens
ggaaggaggggaagctgcag

SEQ ID NO: 2520 - DNA, 20 bp, Homo sapiens
gaaggaggggaagctgcagc

SEQ ID NO: 2521 - DNA, 20 bp, Homo sapiens
aaggaggggaagctgcagct

SEQ ID NO: 2522 - DNA, 20 bp, Homo sapiens
aggaggggaagctgcagctt

SEQ ID NO: 2523 - DNA, 20 bp, Homo sapiens
ggaggggaagctgcagcttg

SEQ ID NO: 2524 - DNA, 20 bp, Homo sapiens
gaggggaagctgcagcttgg

SEQ ID NO: 2525 - DNA, 20 bp, Homo sapiens
aggggaagctgcagcttggg

SEQ ID NO: 2526 - DNA, 20 bp, Homo sapiens
ggggaagctgcagcttggga

SEQ ID NO: 2527 - DNA, 20 bp, Homo sapiens
gggaagctgcagcttgggag

SEQ ID NO: 2528 - DNA, 20 bp, Homo sapiens
ggaagctgcagcttgggaga

SEQ ID NO: 2529 - DNA, 20 bp, Homo sapiens
gaagctgcagcttgggagag

SEQ ID NO: 2530 - DNA, 20 bp, Homo sapiens
aagctgcagcttgggagagc

SEQ ID NO: 2531 - DNA, 20 bp, Homo sapiens
agctgcagcttgggagagcc

SEQ ID NO: 2532 - DNA, 20 bp, Homo sapiens
gctgcagcttgggagagccc

SEQ ID NO: 2533 - DNA, 20 bp, Homo sapiens
ctgcagcttgggagagcccc

SEQ ID NO: 2534 - DNA, 20 bp, Homo sapiens
tgcagcttgggagagcccct

SEQ ID NO: 2535 - DNA, 20 bp, Homo sapiens
gcagcttgggagagcccctg

SEQ ID NO: 2536 - DNA, 20 bp, Homo sapiens
cagcttgggagagcccctgg

SEQ ID NO: 2537 - DNA, 20 bp, Homo sapiens
agcttgggagagcccctggg

SEQ ID NO: 2538 - DNA, 20 bp, Homo sapiens
gcttgggagagcccctgggg

SEQ ID NO: 2539 - DNA, 20 bp, Homo sapiens
cttgggagagcccctggggc

SEQ ID NO: 2540 - DNA, 20 bp, Homo sapiens
ttgggagagcccctggggcc

SEQ ID NO: 2541 - DNA, 20 bp, Homo sapiens
tgggagagcccctggggcct

SEQ ID NO: 2542 - DNA, 20 bp, Homo sapiens
gggagagcccctggggccta

SEQ ID NO: 2543 - DNA, 20 bp, Homo sapiens
ggagagcccctggggcctag

SEQ ID NO: 2544 - DNA, 20 bp, Homo sapiens
gagagcccctggggcctaga

SEQ ID NO: 2545 - DNA, 20 bp, Homo sapiens
agagcccctggggcctagac

SEQ ID NO: 2546 - DNA, 20 bp, Homo sapiens
gagcccctggggcctagact

SEQ ID NO: 2547 - DNA, 20 bp, Homo sapiens
agcccctggggcctagactc

SEQ ID NO: 2548 - DNA, 20 bp, Homo sapiens
gcccctggggcctagactct

SEQ ID NO: 2549 - DNA, 20 bp, Homo sapiens
cccctggggcctagactctg

SEQ ID NO: 2550 - DNA, 20 bp, Homo sapiens
ccctggggcctagactctgt

SEQ ID NO: 2551 - DNA, 20 bp, Homo sapiens
cctggggcctagactctgta

SEQ ID NO: 2552 - DNA, 20 bp, Homo sapiens
ctggggcctagactctgtaa

SEQ ID NO: 2553 - DNA, 20 bp, Homo sapiens
tggggcctagactctgtaac

SEQ ID NO: 2554 - DNA, 20 bp, Homo sapiens
ggggcctagactctgtaaca

SEQ ID NO: 2555 - DNA, 20 bp, Homo sapiens
gggcctagactctgtaacat

SEQ ID NO: 2556 - DNA, 20 bp, Homo sapiens
ggcctagactctgtaacatc

SEQ ID NO: 2557 - DNA, 20 bp, Homo sapiens
gcctagactctgtaacatca

SEQ ID NO: 2558 - DNA, 20 bp, Homo sapiens
cctagactctgtaacatcac

SEQ ID NO: 2559 - DNA, 20 bp, Homo sapiens
ctagactctgtaacatcact

SEQ ID NO: 2560 - DNA, 20 bp, Homo sapiens
tagactctgtaacatcacta

SEQ ID NO: 2561 - DNA, 20 bp, Homo sapiens
agactctgtaacatcactat

SEQ ID NO: 2562 - DNA, 20 bp, Homo sapiens
gactctgtaacatcactatc

SEQ ID NO: 2563 - DNA, 20 bp, Homo sapiens
actctgtaacatcactatcc

SEQ ID NO: 2564 - DNA, 20 bp, Homo sapiens
ctctgtaacatcactatcca

SEQ ID NO: 2565 - DNA, 20 bp, Homo sapiens
tctgtaacatcactatccat

SEQ ID NO: 2566 - DNA, 20 bp, Homo sapiens
ctgtaacatcactatccatg

SEQ ID NO: 2567 - DNA, 20 bp, Homo sapiens
tgtaacatcactatccatgc

SEQ ID NO: 2568 - DNA, 20 bp, Homo sapiens
gtaacatcactatccatgca

SEQ ID NO: 2569 - DNA, 20 bp, Homo sapiens
taacatcactatccatgcac

SEQ ID NO: 2570 - DNA, 20 bp, Homo sapiens
aacatcactatccatgcacc

SEQ ID NO: 2571 - DNA, 20 bp, Homo sapiens
acatcactatccatgcacca

SEQ ID NO: 2572 - DNA, 20 bp, Homo sapiens
catcactatccatgcaccaa

SEQ ID NO: 2573 - DNA, 20 bp, Homo sapiens
atcactatccatgcaccaaa

SEQ ID NO: 2574 - DNA, 20 bp, Homo sapiens
tcactatccatgcaccaaac

SEQ ID NO: 2575 - DNA, 20 bp, Homo sapiens
cactatccatgcaccaaact

SEQ ID NO: 2576 - DNA, 20 bp, Homo sapiens
actatccatgcaccaaacta

SEQ ID NO: 2577 - DNA, 20 bp, Homo sapiens
ctatccatgcaccaaactaa

SEQ ID NO: 2578 - DNA, 20 bp, Homo sapiens
tatccatgcaccaaactaat

SEQ ID NO: 2579 - DNA, 20 bp, Homo sapiens
atccatgcaccaaactaata

SEQ ID NO: 2580 - DNA, 20 bp, Homo sapiens
tccatgcaccaaactaataa

SEQ ID NO: 2581 - DNA, 20 bp, Homo sapiens
ccatgcaccaaactaataaa

SEQ ID NO: 2582 - DNA, 20 bp, Homo sapiens
catgcaccaaactaataaaa

SEQ ID NO: 2583 - DNA, 20 bp, Homo sapiens
atgcaccaaactaataaaac

SEQ ID NO: 2584 - DNA, 20 bp, Homo sapiens
tgcaccaaactaataaaact

SEQ ID NO: 2585 - DNA, 20 bp, Homo sapiens
gcaccaaactaataaaactt

SEQ ID NO: 2586 - DNA, 20 bp, Homo sapiens
caccaaactaataaaacttt

SEQ ID NO: 2587 - DNA, 20 bp, Homo sapiens
accaaactaataaaactttg

SEQ ID NO: 2588 - DNA, 20 bp, Homo sapiens
ccaaactaataaaactttga

SEQ ID NO: 2589 - DNA, 20 bp, Homo sapiens
caaactaataaaactttgac

SEQ ID NO: 2590 - DNA, 20 bp, Homo sapiens
aaactaataaaactttgacg

SEQ ID NO: 2591 - DNA, 20 bp, Homo sapiens
aactaataaaactttgacga

SEQ ID NO: 2592 - DNA, 20 bp, Homo sapiens
actaataaaactttgacgag

SEQ ID NO: 2593 - DNA, 20 bp, Homo sapiens
ctaataaaactttgacgagt

SEQ ID NO: 2594 - DNA, 20 bp, Homo sapiens
taataaaactttgacgagtc

SEQ ID NO: 2595 - DNA, 20 bp, Homo sapiens
aataaaactttgacgagtca

SEQ ID NO: 2596 - DNA, 20 bp, Homo sapiens
ataaaactttgacgagtcac

SEQ ID NO: 2597 - DNA, 20 bp, Homo sapiens
taaaactttgacgagtcacc

SEQ ID NO: 2598 - DNA, 20 bp, Homo sapiens
aaaactttgacgagtcacct

SEQ ID NO: 2599 - DNA, 20 bp, Homo sapiens
aaactttgacgagtcacctt

SEQ ID NO: 2600 - DNA, 20 bp, Homo sapiens
aactttgacgagtcaccttc

SEQ ID NO: 2601 - DNA, 20 bp, Homo sapiens
actttgacgagtcaccttcc

SEQ ID NO: 2602 - DNA, 20 bp, Homo sapiens
ctttgacgagtcaccttcca

SEQ ID NO: 2603 - DNA, 20 bp, Homo sapiens
tttgacgagtcaccttccag

SEQ ID NO: 2604 - DNA, 20 bp, Homo sapiens
ttgacgagtcaccttccagg

SEQ ID NO: 2605 - DNA, 20 bp, Homo sapiens
tgacgagtcaccttccagga

SEQ ID NO: 2606 - DNA, 20 bp, Homo sapiens
gacgagtcaccttccaggac

SEQ ID NO: 2607 - DNA, 20 bp, Homo sapiens
acgagtcaccttccaggacc

SEQ ID NO: 2608 - DNA, 20 bp, Homo sapiens
cgagtcaccttccaggaccc

SEQ ID NO: 2609 - DNA, 20 bp, Homo sapiens
gagtcaccttccaggacccc

SEQ ID NO: 2610 - DNA, 20 bp, Homo sapiens
agtcaccttccaggacccct

SEQ ID NO: 2611 - DNA, 20 bp, Homo sapiens
gtcaccttccaggacccctg

SEQ ID NO: 2612 - DNA, 20 bp, Homo sapiens
tcaccttccaggacccctgg

SEQ ID NO: 2613 - DNA, 20 bp, Homo sapiens
caccttccaggacccctggg

SEQ ID NO: 2614 - DNA, 20 bp, Homo sapiens
accttccaggacccctgggt

SEQ ID NO: 2615 - DNA, 20 bp, Homo sapiens
ccttccaggacccctgggta

SEQ ID NO: 2616 - DNA, 20 bp, Homo sapiens
cttccaggacccctgggtaa

SEQ ID NO: 2617 - DNA, 20 bp, Homo sapiens
ttccaggacccctgggtaaa

SEQ ID NO: 2618 - DNA, 20 bp, Homo sapiens
tccaggacccctgggtaaaa

SEQ ID NO: 2619 - DNA, 20 bp, Homo sapiens
ccaggacccctgggtaaaaa

SEQ ID NO: 2620 - DNA, 20 bp, Homo sapiens
caggacccctgggtaaaaaa

SEQ ID NO: 2621 - DNA, 20 bp, Homo sapiens
aggacccctgggtaaaaaaa

SEQ ID NO: 2622 - DNA, 20 bp, Homo sapiens
ggacccctgggtaaaaaaaa

SEQ ID NO: 2623 - DNA, 20 bp, Homo sapiens
gacccctgggtaaaaaaaaa

SEQ ID NO: 2624 - DNA, 20 bp, Homo sapiens
acccctgggtaaaaaaaaaa

SEQ ID NO: 2625 - DNA, 20 bp, Homo sapiens
cccctgggtaaaaaaaaaaa

SEQ ID NO: 2626 - DNA, 20 bp, Homo sapiens
ccctgggtaaaaaaaaaaaa

SEQ ID NO: 2627 - DNA, 20 bp, Homo sapiens
cctgggtaaaaaaaaaaaaa

SEQ ID NO: 2628 - DNA, 20 bp, Homo sapiens
ctgggtaaaaaaaaaaaaaa

SEQ ID NO: 2629 - DNA, 20 bp, Homo sapiens
tgggtaaaaaaaaaaaaaaa

SEQ ID NO: 2630 - DNA, 18 bp, Artificial Sequence
  Nucleotide forward primer
gcacgtgccaccataccc

SEQ ID NO: 2631 - DNA, 18 bp, Artificial Sequence
  Nucleotide reverse primer
tgcagagcagtcaggctg

SEQ ID NO: 2632 - DNA, 25 bp, Artificial Sequence
  Nucleotide forward primer
actggcgagcagacggtgaggaccc

SEQ ID NO: 2633 - DNA, 25 bp, Artificial Sequence
  Nucleotide reverse primer
tgcgcgcgtgaggctgccggttcgg

(19) United States
(12) Patent Application Publication
(10) Pub. No.: US 2009/0291432 A1
(43) Pub. Date: Nov. 26, 2009

(54) Genetic profiles associated with the 957C>T polymorphism in the DRD2 gene

(75) Inventors: Charles Phillip Morris, Queensland (AU); Angela Van Daal, Queensland (AU); Christopher Dean Swagell, Queensland (AU); Bruce Robert Lawford, Queensland (AU); Ross McDonald Young, Queensland (AU)

Correspondence Address: KNOBBE MARTENS OLSON & BEAR LLP, 2040 MAIN STREET, FOURTEENTH FLOOR, IRVINE, CA, 92614, US

(73) Assignees: Queensland University Of Technology, Queensland (AU); The State Of Queensland (Queensland Health), Queensland (AU)

(21) Appl. No.: 11/628,397

(22) PCT Filed: Jun. 1, 2005
(86) PCT No.: PCT/AU2005/000775

§ 371 Date: Aug. 14, 2008

(30) Foreign Application Priority Data
Jun. 1, 2004 (AU) 2004902919

(51) Int. Cl. C12Q 1/68; C07H 21/04

(52) U.S. Cl. 435/6; 536/23.5

(57) ABSTRACT

The present invention relates to a method for profiling an individual or group of individuals with respect to a neurological, psychiatric or psychological condition, phenotype or state, including a sub-threshold neurological, psychiatric or psychological condition, phenotype or state. More particularly, the present invention identifies a genetic profile associated with the 957C>T polymorphysm within the dopamine receptor D2 (DRD2), indicating a predisposition to schizophrenia and other neurological diseases.

### BACKGROUND OF THE INVENTION

1. Field of the Invention

The present invention relates generally to a method for profiling an individual or group of individuals with respect to a neurological, psychiatric or psychological condition, phenotype or state, including a sub-threshold neurological, psychiatric or psychological condition, phenotype or state. More particularly, the present invention identifies genetic profiles associated with a neurological, psychiatric or psychological condition, phenotype or state which permit the development of agents useful in diagnosing the presence of a neurological, psychiatric or psychological condition, phenotype or state or a risk or likelihood of development of same. The present invention further contemplates methods for the treatment or prophylaxis of a neurological, psychiatric or psychological condition, phenotype or state in an individual, including implementing behavioral modification protocols, to ameliorate the risk of developing an adverse neurological, psychiatric or psychological condition, phenotype or state.

2. Description of the Prior Art

Reference to any prior art in this specification is not, and should not be taken as, an acknowledgment or any form of suggestion that this prior art forms a part of the common general knowledge in any country.

Psychological “disorders” are endemic in any society. Reference to “disorders” in this context means that an individual exhibits behavioral patterns which are inconsistent with society norms. Many psychological phenotypes have a physiological basis while others result from or are compounded by environmental conditioning. The difficulty for clinical psychiatrists, neurologists and psychologists is to diagnose a physiologically-based condition for which therapeutic intervention is possible.

One particularly complex psychological phenotype is schizophrenia. Schizophrenia is a common, chronic, disabling illness with an incidence of 15 new cases per 100,000 population per year (Kelly et al., Ir. J. Med. Sci. 172:37-40, 2003). Additionally, “unaffected” first degree relatives show both childhood (Niendam et. al., Am. J. Psychiatry. 160:2060-2062, 2003) and adulthood (MacDonald et al., Arch. Gen. Psychiatry. 60:57-65, 2003) deficits in cognitive functioning. Siblings of schizophrenic patients also exhibit an abnormal MRI response in the dorsolateral prefrontal cortex implicating inefficient information processing (Callicott et. al., Am. J. Psychiatry. 160:709-719, 2003). Furthermore, both schizophrenic subjects and their unaffected siblings show both reductions in hippocampal volume and hippocampal shape deformity (Tepest et al., Biol, Psychiatry. 54:1234-1240, 2003). Decreased temporoparietal P300 amplitude and increased frontal P300 amplitude are found in both schizophrenic patients and their siblings (Winterer et. al., Arch. Gen. Psychiatry. 60:1158-1167, 2003). Taken together, these findings indicate that the underlying pathophysiological state of schizophrenia is considerably more widespread in the general population than prevalence figures for schizophrenia would suggest and that a considerable genetic vulnerability for this disorder exists. Consequently, schizophrenia is a complex condition having a wide variety of manifestations some of which may have a pathophysiological origin whereas others may originate from environmental conditioning including substance abuse. This makes the treatment and diagnosis of schizophrenia difficult for clinicians.

The apparent high genetic risk for schizophrenia has led to considerable research efforts aimed at the identification of susceptibility genes. This has resulted in linkages or associations with regions 6p21-22, 1q21-22 and 13q32-34 with single studies reporting significance at P<0.05 (Owen et al., Mol. Psychiatry. 9:14-27, 2004) although a recent large multicenter linkage study of schizophrenia loci on chromosome 22q failed to find any evidence for linkage or association to schizophrenia (Mowry B J et. al., Mol. Psychiatry. 2004). Other regions that may be implicated include 8p21-22, 6q21-25, 5q21-q33, 10p15-p11 and 1q42 (Owen et al. 2004 supra). Despite this limited progress, the conclusive identification of specific molecular genetic etiological factors in the pathogenesis of schizophrenia has not occurred (Miyamoto et al., Mol. Intervent. 3:27-39, 2004).

Several lines of evidence have implicated the dopamine 2 receptor (DRD2) gene as a candidate gene for schizophrenia genetic susceptibility. For example, all anti-psychotic medications are either antagonists or partial agonists of DRD2. DRD2 receptor has been repeatedly demonstrated to be the primary site of action for these medications (Seeman and Kapur Proc. Natl. Acad. Sci. USA 97:7673-7675, 2000) indicating that schizophrenic symptoms are ameliorated by a reduction in DRD2 function. Additionally, recent evidence strongly suggests that schizophrenic patients have increased brain DRD2 density (Abi-Dargham et al., Proc. Natl. Acad. Sci. 97:8104-8109, 2000). However, the absence of a clear genetic link between the DRD2 and schizophrenia have hampered the development of appropriate therapeutic and diagnostic protocols.

### SUMMARY OF THE INVENTION

The present invention now identifies a genetic link between DRD2 and a neurological, psychiatric or psychological condition, phenotype or state. In particular, a population of individuals having related pathopsychological symptoms and behavioral patterns are shown to exhibit a particular polymorphism with the genetic region encoding the DRD2 receptor. Even more particularly, the present invention identifies a polymorphism in or near the DRD2 genetic locus which is prevalent in individuals with schizophrenia, alcoholism or related neurological, psychiatric or psychological conditions including addictions including smoking and drug abuse.

Accordingly, the present invention contemplates a method for identifying a genetic profile associated with a neurological, psychiatric or psychological condition, phenotype or state including a sub-threshold neurological, psychiatric or psychological condition, phenotype or state in an individual or a group of individuals, said method comprising screening individuals for a polymorphism including a mutation in a genetic locus comprising the DRD2 gene, including its 5′ and 3′ terminal regions, promoter, introns and exons which has a statistically significant linkage or association to symptoms or behavior characterizing the neurological, psychiatric or psychological condition, phenotype or state or sub-threshold forms thereof.

Neurological, psychiatric or psychological conditions, phenotypes and states include, but are not limited to, Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder, Smoking Dependence and Social Phobia. It should be noted, however, that a person considered not to suffer any symptom associated with the above disorders still falls within the scope of a “norma” or a non-symptomatic or non-pathogenic neurological, psychiatric or psychological condition, phenotype or state.

Schizophrenia and alcoholism are particularly exemplified herein associated with a polymorphism in the genetic locus comprising the DRD2 gene including its 5′ and 3′ terminal regions, promoter, introns and exons. A list of potential polymorphisms in the DRD2 gene are shown in Table 2.

In one embodiment polymorphism having a linkage or association to schizophrenia or alcoholism is a thymine (T) to cytosine (C) substitution at nucleotide position 957 (957C>T) using the numbering system from the cDNA sequence. This is represented as 957C>T. The nucleotide position is calculated using the cDNA sequence, wherein the numbering is calculated from the “A” of the AGT encoding the methionine being at position +1, encoding DRD2 (see SEQ ID NO:1). However, the present invention extends to any polymorphism or other mutation in the DRD2 genetic locus and which is linked to a neurological, psychiatric or psychological condition, phenotype or state such as schizophrenia or alcoholism.

The present invention enables clinicians to make a genetic-based diagnosis of a neurological, psychiatric or psychological condition, phenotype or state or a risk or likelihood that an individual will develop such a neurological, psychiatric or psychological condition, phenotype or state and can thereby implement treatment or preventative or symptom-ameliorating or controlling protocols including therapeutic intervention and/or behavioral modification protocols to reduce the adverse consequences of the neurological, psychiatric or psychological condition, phenotype or state.

In addition, the identification of a polymorphism including a mutation in the DRD2 genetic locus enables agents to be identified which mask the physiological impact or consequences of the genetic profile. For example, it is proposed that 957C>T results in decreased translation and stability of D2 mRNA. Consequently, agents which cause reduced levels of DRD2, such as DRD2 antagonists maybe useful in the treatment of schizophrenia or alcoholism or other neurological, psychiatric or psychological conditions, phenotypes or states.

The present invention further contemplates combinations of two or more polymorphisms such as 957C>T and Taq1A. The combinations may be at the same allele (i.e. haplotypes) or at different alleles.

Throughout the specification, unless the context requires otherwise, the word “comprise” or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence list is provided following the claims.

A summary of the sequence identifiers used throughout the subject specification is provided in Table 1.

TABLE 1
Summary of the sequence identifiers

| SEQUENCE | |
|---|---|
| ID NO: | DESCRIPTION |
| 1 | cDNA sequence corresponding to the dopamine D2 |
| | receptor (DRD2) with 5′- and 3′-non translated sequences. |
| 2 | cDNA sequence corresponding to the DRD2 cDNA |
| | sequence containing a C > T polymorphism at nucleotide |
| | position 957 |
| 3 | genomic DRD2 sequence with promoter and 5′- and 3′- |
| | untranslated sequence |
| 4 | sequence specific primer for the “C” allele of DRD2 |
| 5 | sequence specific primer for the “T” allele of DRD2 |
| 6 | common reverse primer for DRD2 |
| 7 | amino acid sequence for DRD2 |
| 8-2629 | Oligomeric sequences from the DRD2 gene |
| 2630 | Taq1A PCR forward primer |
| 2631 | Taq1A PCR reverse primer |
| 2632 | −141delC PCR forward primer |
| 2633 | −141delC PCR reverse primer |

### DETAILED DESCRIPTION OF THE PREFERRED EMBODIMENTS

The present invention is predicated in part on the identification of genetic profiles having a statistically significant association with a neurological, psychiatric or psychological condition, phenotype or state including a sub-threshold neurological, psychiatric or psychological condition, phenotype or state. By “genetic profiles” is meant that groups of individuals exhibiting a particular neurological, psychiatric or psychological condition, phenotype or state or sub-threshold forms thereof or who are at the risk of developing same exhibit a common polymorphism at or within the DRD2 genetic locus including its 5′ or 3′ terminal regions, promoter, exons or introns. The genetic profile may be a single polymorphism or multiple polymorphisms, that is two or more polymorphisms that are statistically significantly linked to a neurological, psychiatric or psychological condition, phenotype or state or sub-threshold forms thereof. Reference to a polymorphism in this context includes a mutation.

The singular forms “a”, “an”, and “the” include single and plural aspects unless the context clearly indicates otherwise. Thus, for example, reference to a “polymorphism” includes a single polymorphism, as well as two or more polymorphisms; reference to a psychological phenotype includes a single psychological phenotype, as well as two or more psychological phenotypes.

Accordingly, one aspect of the present invention contemplates a method for identifying a genetic profile associated with a neurological, psychiatric or psychological condition, phenotype or state including a sub-threshold neurological, psychiatric or psychological condition, phenotype or state in an individual or a group of individuals, said method comprising screening individuals for a polymorphism in a genetic locus comprising the DRD2 gene including its 5′ and 3′ terminal regions, promoter, introns and exons which has statistically significant linkage or association to symptoms or behavior characterizing the neurological, psychiatric or psychological condition, phenotype or state or sub-threshold forms thereof.

The genetic locus comprising the DRD2 gene may be referred to as the “DRD2 gene”, “DRD2 nucleic acid”, “DRD2 locus”, “DRD2 genetic locus” or “DRD2 polynucleotide”. Each refers to polynucleotides, all of which, are in the DRD2 region including its 5′ or 3′ terminal regions, promoter, introns or exons. Accordingly, the DRD2 locus is intended to include coding sequences, intervening sequences and regulatory elements controlling transcription and/or translation. The DRD2 genetic locus is intended to include all allelic variations of the DNA sequence on either or both chromosomes. Consequently, homozygous and heterozygous variations of the DRD2 locus are contemplated herein.

As indicated above, the DRD2 locus comprises different profiles for different neurological, psychiatric or psychological conditions, phenotypes or states or sub-threshold forms thereof. Such profiles include polymorphisms, although any nucleotide substitution, addition, deletion or insertion or other mutation in the DRD2 genetic locus is encompassed by the present invention when associated with a neurological, psychiatric or psychological condition, phenotype or state. Accordingly, the present invention extends to rare mutations which although not present in larger numbers of individuals in a population, when the mutation is present, it leads to a very high likelihood of development of a pathopsychological disorder.

The term “polymorphism” or “mutation” refers to a difference in a DNA or RNA sequence or sequences among individuals, groups or populations which give rise to a statistically significant phenotype or physiological condition. Examples of genetic polymorphisms include mutations that result by chance or are induced by external features. These polymorphisms or mutations may be indicative of a disease or disorder and may arise following a genetic disease, a chromosomal abnormality, a genetic predisposition, a viral infection, a fungal infection, a bacterial infection or a protist infection or following chemotherapy, radiation therapy or substance abuse including alcohol or drug abuse. The polymorphisms may also dictate or contribute to symptoms with a psychological phenotype. In a preferred aspect, the polymorphisms of the present invention are indicative of a neurological, psychiatric or psychological condition, phenotype or state or sub-threshold condition, phenotype or state thereof. As used herein, polymorphisms including mutations may refer to one or more changes in a DNA or RNA sequence which are present in a group of individuals having a particular neurological, psychiatric or psychological condition, phenotype or state or sub-threshold forms thereof or are at risk of developing same.

Examples of nucleotide changes contemplated herein include single nucleotide polymorphisms (SNPs), multiple nucleotide polymorphisms (MNPs), frame shift mutations, including insertions and deletions (also called deletion insertion polymorphisms or DIPS), nucleotide substitutions and nonsense mutations. Two or more polymorphisms may also be used either at the same allele (i.e. haplotypes) or at different alleles.

Examples of a neurological, psychiatric or psychological condition, phenotype or state contemplated by the present invention and which may be directly or indirectly linked to a genetic profile such as a polymorphism or mutation to the DRD2 genetic locus include but are not limited to Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder and Social Phobia.

The most exemplified conditions herein up to the present time are schizophrenia and alcoholism. Reference herein to “schizophrenia” includes conditions which have symptoms similar to schizophrenia and hence are regard as schizophrenia-related conditions. Such symptoms of schizophrenia include behavioral and physiological conditions. Due to the composition of schizophrenia and related conditions, the ability to identify a genetic profile to assist in defining Schizophrenia is of significant importance. The present invention now provides this genetic profile.

Reference hereto to “alcoholism” refers to a disorder characterized by dependence on alcohol, repeated excessive use of alcoholic beverages, development of withdrawal symptoms on reducing or ceasing alcohol intake, morbidity that may include cirrhosis of the liver, and decreased ability to function socially and vocationally. It is characterized by a cluster of behavioral, cognitive, and physiological phenomena that develop after repeated substance misuse and that typically include a strong desire to take the alcohol, difficulties in controlling its use, persisting in its use despite harmful consequences, a higher priority given to its use than other activities and obligations, increased tolerance, and sometimes a physical withdrawal state.

Any number of methods may be used to calculate the statistical significance of a polymorphism and its association with a neurological, psychiatric or psychological condition. Particular statistical analysis methods which may be used are described in Fisher and vanBelle, “Biostatistics: A Methodology for the Health Sciences” Wiley-Intersciences (New York) 1993. This analysis may also include a regression calculation of which polymorphic sites in the DRD2 gene give the most significant contribution to the differences in phenotype. One regression model useful in the invention starts with a model of the form

r=r₀+(S×d)

where r is the response, r₀ is a constant called the “intercept”, S is the slope and d is the dose. To determine the dose, the most-common and least common nucleotides at the polymorphic site are first defined. Then, for each individual in the trial population, one calculates a “dose” as the number of least-common nucleotides the individual has at the polymorphic site of interest. This value can be 0 (homozygous for the least-common nucleotide), 1 (heterozygous), or 2 (homozygous for the most common nucleotide). An individual's “response” is the value of the clinical measurement. Standard linear regression methods are then used to fit all the individuals' doses and responses to a single model (see e.g. Fisher and vanBelle, supra, Ch 9). The outputs of the regression calculation are the intercept r₀, the slope S, and the variance (which measures how well the data fits this simple linear model). The Students t-test value and the level of significance can then be calculated for each of the polymorphic sites.

In relation to the genetic profile associated with schizophrenia, alcoholism or a related condition or other neurological, psychiatric or psychological condition, phenotype or state, the present invention encompasses any polymorphism or mutation within or proximal to the DRD2 genetic locus including its 5′ or 3′ terminal regions, promoter, introns and exons. Examples of possible polymorphisms or mutations are given in Table 2.

TABLE 2

| Nucleotide | | | | | | |
|---|---|---|---|---|---|---|
| position | Nucleotide | g | c | t | a | del |
| 1 | g | * | — | — | — | — |
| 2 | g | * | — | — | — | — |
| 3 | a | — | — | — | * | — |
| 4 | t | — | — | * | — | — |
| 5 | c | — | * | — | — | — |
| 6 | c | — | * | — | — | — |
| 7 | g | * | — | — | — | — |
| 8 | g | * | — | — | — | — |
| 9 | g | * | — | — | — | — |
| 10 | a | — | — | — | * | — |
| 11 | t | — | — | * | — | — |
| 12 | a | — | — | — | * | — |
| 13 | t | — | — | * | — | — |
| 14 | g | * | — | — | — | — |
| 15 | g | * | — | — | — | — |
| 16 | t | — | — | * | — | — |
| 17 | g | * | — | — | — | — |
| 18 | a | — | — | — | * | — |
| 19 | t | — | — | * | — | — |
| 20 | t | — | — | * | — | — |
| 21 | t | — | — | * | — | — |
| 22 | g | * | — | — | — | — |
| 23 | t | — | — | * | — | — |
| 24 | t | — | — | * | — | — |
| 25 | g | * | — | — | — | — |
| 26 | t | — | — | * | — | — |
| 27 | t | — | — | * | — | — |
| 28 | t | — | — | * | — | — |
| 29 | c | — | * | — | — | — |
| 30 | g | * | — | — | — | — |
| 31 | c | — | * | — | — | — |
| 32 | t | — | — | * | — | — |
| 33 | g | * | — | — | — | — |
| 34 | c | — | * | — | — | — |
| 35 | a | — | — | — | * | — |
| 36 | c | — | * | — | — | — |
| 37 | c | — | * | — | — | — |
| 38 | a | — | — | — | * | — |
| 39 | c | — | * | — | — | — |
| 40 | t | — | — | * | — | — |
| 41 | t | — | — | * | — | — |
| 42 | c | — | * | — | — | — |
| 43 | t | — | — | * | — | — |
| 44 | g | * | — | — | — | — |
| 45 | t | — | — | * | — | — |
| 46 | c | — | * | — | — | — |
| 47 | c | — | * | — | — | — |
| 48 | t | — | — | * | — | — |
| 49 | c | — | * | — | — | — |
| 50 | t | — | — | * | — | — |
| 51 | t | — | — | * | — | — |
| 52 | c | — | * | — | — | — |
| 53 | c | — | * | — | — | — |
| 54 | t | — | — | * | — | — |
| 55 | a | — | — | — | * | — |
| 56 | t | — | — | * | — | — |
| 57 | a | — | — | — | * | — |
| 58 | c | — | * | — | — | — |
| 59 | t | — | — | * | — | — |
| 60 | g | * | — | — | — | — |
| 61 | t | — | — | * | — | — |
| 62 | a | — | — | — | * | — |
| 63 | g | * | — | — | — | — |
| 64 | a | — | — | — | * | — |
| 65 | a | — | — | — | * | — |
| 66 | t | — | — | * | — | — |
| 67 | c | — | * | — | — | — |
| 68 | c | — | * | — | — | — |
| 69 | c | — | * | — | — | — |
| 70 | t | — | — | * | — | — |
| 71 | g | * | — | — | — | — |
| 72 | t | — | — | * | — | — |
| 73 | g | * | — | — | — | — |
| 74 | t | — | — | * | — | — |
| 75 | g | * | — | — | — | — |
| 76 | g | * | — | — | — | — |
| 77 | g | * | — | — | — | — |
| 78 | t | — | — | * | — | — |
| 79 | c | — | * | — | — | — |
| 80 | t | — | — | * | — | — |
| 81 | t | — | — | * | — | — |
| 82 | g | * | — | — | — | — |
| 83 | g | * | — | — | — | — |
| 84 | t | — | — | * | — | — |
| 85 | g | * | — | — | — | — |
| 86 | g | * | — | — | — | — |
| 87 | c | — | * | — | — | — |
| 88 | a | — | — | — | * | — |
| 89 | g | * | — | — | — | — |
| 90 | g | * | — | — | — | — |
| 91 | c | — | * | — | — | — |
| 92 | a | — | — | — | * | — |
| 93 | g | * | — | — | — | — |
| 94 | a | — | — | — | * | — |
| 95 | g | * | — | — | — | — |
| 96 | c | — | * | — | — | — |
| 97 | c | — | * | — | — | — |
| 98 | c | — | * | — | — | — |
| 99 | t | — | — | * | — | — |
| 100 | c | — | * | — | — | — |
| 101 | a | — | — | — | * | — |
| 102 | c | — | * | — | — | — |
| 103 | c | — | * | — | — | — |
| 104 | c | — | * | — | — | — |
| 105 | c | — | * | — | — | — |
| 106 | a | — | — | — | * | — |
| 107 | c | — | * | — | — | — |
| 108 | t | — | — | * | — | — |
| 109 | g | * | — | — | — | — |
| 110 | a | — | — | — | * | — |
| 111 | a | — | — | — | * | — |
| 112 | a | — | — | — | * | — |
| 113 | g | * | — | — | — | — |
| 114 | g | * | — | — | — | — |
| 115 | t | — | — | * | — | — |
| 116 | g | * | — | — | — | — |
| 117 | c | — | * | — | — | — |
| 118 | a | — | — | — | * | — |
| 119 | g | * | — | — | — | — |
| 120 | c | — | * | — | — | — |
| 121 | t | — | — | * | — | — |
| 122 | c | — | * | — | — | — |
| 123 | a | — | — | — | * | — |
| 124 | t | — | — | * | — | — |
| 125 | a | — | — | — | * | — |
| 126 | g | * | — | — | — | — |
| 127 | c | — | * | — | — | — |
| 128 | c | — | * | — | — | — |
| 129 | c | — | * | — | — | — |
| 130 | t | — | — | * | — | — |
| 131 | g | * | — | — | — | — |
| 132 | c | — | * | — | — | — |
| 133 | t | — | — | * | — | — |
| 134 | c | — | * | — | — | — |
| 135 | a | — | — | — | * | — |
| 136 | a | — | — | — | * | — |
| 137 | c | — | * | — | — | — |
| 138 | c | — | * | — | — | — |
| 139 | a | — | — | — | * | — |
| 140 | a | — | — | — | * | — |
| 141 | t | — | — | * | — | — |
| 142 | g | * | — | — | — | — |
| 143 | a | — | — | — | * | — |
| 144 | g | * | — | — | — | — |
| 145 | t | — | — | * | — | — |
| 146 | g | * | — | — | — | — |
| 147 | g | * | — | — | — | — |
| 148 | c | — | * | — | — | — |
| 149 | a | — | — | — | * | — |
| 150 | g | * | — | — | — | — |
| 151 | c | — | * | — | — | |
| 152 | t | — | — | * | — | |
| 153 | g | * | — | — | — | |
| 154 | c | — | * | — | — | |
| 155 | c | — | * | — | — | |
| 156 | t | — | — | * | — | |
| 157 | c | — | * | — | — | |
| 158 | a | — | — | — | * | |
| 159 | g | * | — | — | — | |
| 160 | a | — | — | — | * | |
| 161 | t | — | — | * | — | |
| 162 | t | — | — | * | — | |
| 163 | t | — | — | * | — | |
| 164 | t | — | — | * | — | |
| 165 | g | * | — | — | — | |
| 166 | a | — | — | — | * | |
| 167 | c | — | * | — | — | |
| 168 | t | — | — | * | — | |
| 169 | g | * | — | — | — | |
| 170 | a | — | — | — | * | |
| 171 | g | * | — | — | — | |
| 172 | g | * | — | — | — | |
| 173 | a | — | — | — | * | |
| 174 | g | * | — | — | — | |
| 175 | c | — | * | — | — | |
| 176 | t | — | — | * | — | |
| 177 | a | — | — | — | * | |
| 178 | g | * | — | — | — | |
| 179 | t | — | — | * | — | |
| 180 | g | * | — | — | — | |
| 181 | a | — | — | — | * | |
| 182 | c | — | * | — | — | |
| 183 | t | — | — | * | — | |
| 184 | c | — | * | — | — | |
| 185 | a | — | — | — | * | |
| 186 | g | * | — | — | — | |
| 187 | a | — | — | — | * | |
| 188 | g | * | — | — | — | |
| 189 | a | — | — | — | * | |
| 190 | a | — | — | — | * | |
| 191 | g | * | — | — | — | |
| 192 | c | — | * | — | — | |
| 193 | a | — | — | — | * | |
| 194 | a | — | — | — | * | |
| 195 | g | * | — | — | — | |
| 196 | g | * | — | — | — | |
| 197 | a | — | — | — | * | |
| 198 | c | — | * | — | — | |
| 199 | t | — | — | * | — | |
| 200 | c | — | * | — | — | |
| 201 | t | — | — | * | — | |
| 202 | g | * | — | — | — | |
| 203 | g | * | — | — | — | |
| 204 | a | — | — | — | * | |
| 205 | g | * | — | — | — | |
| 206 | a | — | — | — | * | |
| 207 | a | — | — | — | * | |
| 208 | t | — | — | * | — | |
| 209 | c | — | * | — | — | |
| 210 | a | — | — | — | * | |
| 211 | a | — | — | — | * | |
| 212 | t | — | — | * | — | |
| 213 | c | — | * | — | — | |
| 214 | c | — | * | — | — | |
| 215 | t | — | — | * | — | |
| 216 | a | — | — | — | * | |
| 217 | g | * | — | — | — | |
| 218 | a | — | — | — | * | |
| 219 | g | * | — | — | — | |
| 220 | g | * | — | — | — | |
| 221 | t | — | — | * | — | |
| 222 | a | — | — | — | * | |
| 223 | g | * | — | — | — | |
| 224 | t | — | — | * | — | |
| 225 | a | — | — | — | * | |
| 226 | c | — | * | — | — | |
| 227 | c | — | * | — | — | |
| 228 | a | — | — | — | * | |
| 229 | g | * | — | — | — | |
| 230 | t | — | — | * | — | |
| 231 | a | — | — | — | * | |
| 232 | g | * | — | — | — | |
| 233 | c | — | * | — | — | |
| 234 | c | — | * | — | — | |
| 235 | a | — | — | — | * | |
| 236 | c | — | * | — | — | |
| 237 | a | — | — | — | * | |
| 238 | a | — | — | — | * | |
| 239 | c | — | * | — | — | |
| 240 | t | — | — | * | — | |
| 241 | t | — | — | * | — | |
| 242 | t | — | — | * | — | |
| 243 | c | — | * | — | — | |
| 244 | a | — | — | — | * | |
| 245 | g | * | — | — | — | |
| 246 | t | — | — | * | — | |
| 247 | t | — | — | * | — | |
| 248 | t | — | — | * | — | |
| 249 | t | — | — | * | — | |
| 250 | c | — | * | — | — | |
| 251 | c | — | * | — | — | |
| 252 | a | — | — | — | * | |
| 253 | g | * | — | — | — | |
| 254 | g | * | — | — | — | |
| 255 | a | — | — | — | * | |
| 256 | a | — | — | — | * | |
| 257 | c | — | * | — | — | |
| 258 | a | — | — | — | * | |
| 259 | g | * | — | — | — | |
| 260 | c | — | * | — | — | |
| 261 | a | — | — | — | * | |
| 262 | t | — | — | * | — | |
| 263 | a | — | — | — | * | |
| 264 | a | — | — | — | * | |
| 265 | c | — | * | — | — | |
| 266 | a | — | — | — | * | |
| 267 | g | * | — | — | — | |
| 268 | g | * | — | — | — | |
| 269 | g | * | — | — | — | |
| 270 | t | — | — | * | — | |
| 271 | g | * | — | — | — | |
| 272 | c | — | * | — | — | |
| 273 | c | — | * | — | — | |
| 274 | a | — | — | — | * | |
| 275 | g | * | — | — | — | |
| 276 | a | — | — | — | * | |
| 277 | g | * | — | — | — | |
| 278 | g | * | — | — | — | |
| 279 | g | * | — | — | — | |
| 280 | t | — | — | * | — | |
| 281 | t | — | — | * | — | |
| 282 | g | * | — | — | — | |
| 283 | c | — | * | — | — | |
| 284 | c | — | * | — | — | |
| 285 | a | — | — | — | * | |
| 286 | g | * | — | — | — | |
| 287 | t | — | — | * | — | |
| 288 | g | * | — | — | — | |
| 289 | t | — | — | * | — | |
| 290 | c | — | * | — | — | |
| 291 | t | — | — | * | — | |
| 292 | g | * | — | — | — | |
| 293 | g | * | — | — | — | |
| 294 | t | — | — | * | — | |
| 295 | a | — | — | — | * | |
| 296 | c | — | * | — | — | |
| 297 | c | — | * | — | — | |
| 298 | a | — | — | — | * | |
| 299 | g | * | — | — | — | |
| 300 | c | — | * | — | — | |
| 301 | c | — | * | — | — | |
| 302 | a | — | — | — | * | |
| 303 | a | — | — | — | * | |
| 304 | g | * | — | — | — | |
| 305 | g | * | — | — | — | |
| 306 | t | — | — | * | — | |
| 307 | g | * | — | — | — | |
| 308 | g | * | — | — | — | |
| 309 | a | — | — | — | * | |
| 310 | g | * | — | — | — | |
| 311 | a | — | — | — | * | |
| 312 | t | — | — | * | — | |
| 313 | g | * | — | — | — | |
| 314 | c | — | * | — | — | |
| 315 | c | — | * | — | — | |
| 316 | a | — | — | — | * | |
| 317 | t | — | — | * | — | |
| 318 | c | — | * | — | — | |
| 319 | t | — | — | * | — | |
| 320 | g | * | — | — | — | |
| 321 | g | * | — | — | — | |
| 322 | t | — | — | * | — | |
| 323 | g | * | — | — | — | |
| 324 | c | — | * | — | — | |
| 325 | c | — | * | — | — | |
| 326 | c | — | * | — | — | |
| 327 | t | — | — | * | — | |
| 328 | g | * | — | — | — | |
| 329 | t | — | — | * | — | |
| 330 | a | — | — | — | * | |
| 331 | t | — | — | * | — | |
| 332 | t | — | — | * | — | |
| 333 | t | — | — | * | — | |
| 334 | a | — | — | — | * | |
| 335 | g | * | — | — | — | |
| 336 | c | — | * | — | — | |
| 337 | t | — | — | * | — | |
| 338 | a | — | — | — | * | |
| 339 | t | — | — | * | — | |
| 340 | g | * | — | — | — | |
| 341 | a | — | — | — | * | |
| 342 | t | — | — | * | — | |
| 343 | a | — | — | — | * | |
| 344 | g | * | — | — | — | |
| 345 | c | — | * | — | — | |
| 346 | t | — | — | * | — | |
| 347 | a | — | — | — | * | |
| 348 | a | — | — | — | * | |
| 349 | g | * | — | — | — | |
| 350 | t | — | — | * | — | |
| 351 | t | — | — | * | — | |
| 352 | c | — | * | — | — | |
| 353 | t | — | — | * | — | |
| 354 | c | — | * | — | — | |
| 355 | a | — | — | — | * | |
| 356 | c | — | * | — | — | |
| 357 | t | — | — | * | — | |
| 358 | g | * | — | — | — | |
| 359 | g | * | — | — | — | |
| 360 | a | — | — | — | * | |
| 361 | c | — | * | — | — | |
| 362 | t | — | — | * | — | |
| 363 | g | * | — | — | — | |
| 364 | c | — | * | — | — | |
| 365 | t | — | — | * | — | |
| 366 | g | * | — | — | — | |
| 367 | c | — | * | — | — | |
| 368 | t | — | — | * | — | |
| 369 | c | — | * | — | — | |
| 370 | c | — | * | — | — | |
| 371 | a | — | — | — | * | |
| 372 | g | * | — | — | — | |
| 373 | c | — | * | — | — | |
| 374 | t | — | — | * | — | |
| 375 | t | — | — | * | — | |
| 376 | g | * | — | — | — | |
| 377 | a | — | — | — | * | |
| 378 | t | — | — | * | — | |
| 379 | t | — | — | * | — | |
| 380 | t | — | — | * | — | |
| 381 | t | — | — | * | — | |
| 382 | g | * | — | — | — | |
| 383 | g | * | — | — | — | |
| 384 | t | — | — | * | — | |
| 385 | t | — | — | * | — | |
| 386 | g | * | — | — | — | |
| 387 | a | — | — | — | * | |
| 388 | a | — | — | — | * | |
| 389 | a | — | — | — | * | |
| 390 | g | * | — | — | — | |
| 391 | c | — | * | — | — | |
| 392 | c | — | * | — | — | |
| 393 | t | — | — | * | — | |
| 394 | a | — | — | — | * | |
| 395 | a | — | — | — | * | |
| 396 | c | — | * | — | — | |
| 397 | a | — | — | — | * | |
| 398 | g | * | — | — | — | |
| 399 | t | — | — | * | — | |
| 400 | a | — | — | — | * | |
| 401 | g | * | — | — | — | |
| 402 | c | — | * | — | — | |
| 403 | t | — | — | * | — | |
| 404 | a | — | — | — | * | |
| 405 | g | * | — | — | — | |
| 406 | c | — | * | — | — | |
| 407 | t | — | — | * | — | |
| 408 | t | — | — | * | — | |
| 409 | t | — | — | * | — | |
| 410 | a | — | — | — | * | |
| 411 | t | — | — | * | — | |
| 412 | t | — | — | * | — | |
| 413 | c | — | * | — | — | |
| 414 | t | — | — | * | — | |
| 415 | c | — | * | — | — | |
| 416 | c | — | * | — | — | |
| 417 | c | — | * | — | — | |
| 418 | t | — | — | * | — | |
| 419 | g | * | — | — | — | |
| 420 | c | — | * | — | — | |
| 421 | c | — | * | — | — | |
| 422 | t | — | — | * | — | |
| 423 | g | * | — | — | — | |
| 424 | t | — | — | * | — | |
| 425 | t | — | — | * | — | |
| 426 | t | — | — | * | — | |
| 427 | t | — | — | * | — | |
| 428 | a | — | — | — | * | |
| 429 | c | — | * | — | — | |
| 430 | a | — | — | — | * | |
| 431 | a | — | — | — | * | |
| 432 | g | * | — | — | — | |
| 433 | t | — | — | * | — | |
| 434 | c | — | * | — | — | |
| 435 | t | — | — | * | — | |
| 436 | g | * | — | — | — | |
| 437 | g | * | — | — | — | |
| 438 | t | — | — | * | — | |
| 439 | t | — | — | * | — | |
| 440 | c | — | * | — | — | |
| 441 | t | — | — | * | — | |
| 442 | c | — | * | — | — | |
| 443 | t | — | — | * | — | |
| 444 | a | — | — | — | * | |
| 445 | a | — | — | — | * | |
| 446 | t | — | — | * | — | |
| 447 | c | — | * | — | — | |
| 448 | t | — | — | * | — | |
| 449 | c | — | * | — | — | |
| 450 | t | — | — | * | — | |
| 451 | a | — | — | — | * | |
| 452 | t | — | — | * | — | |
| 453 | g | * | — | — | — | |
| 454 | a | — | — | — | * | |
| 455 | t | — | — | * | — | |
| 456 | t | — | — | * | — | |
| 457 | c | — | * | — | — | |
| 458 | t | — | — | * | — | |
| 459 | g | * | — | — | — | |
| 460 | t | — | — | * | — | |
| 461 | g | * | — | — | — | |
| 462 | a | — | — | — | * | |
| 463 | g | * | — | — | — | |
| 464 | c | — | * | — | — | |
| 465 | t | — | — | * | — | |
| 466 | a | — | — | — | * | |
| 467 | c | — | * | — | — | |
| 468 | t | — | — | * | — | |
| 469 | a | — | — | — | * | |
| 470 | c | — | * | — | — | |
| 471 | a | — | — | — | * | |
| 472 | a | — | — | — | * | |
| 473 | t | — | — | * | — | |
| 474 | a | — | — | — | * | |
| 475 | t | — | — | * | — | |
| 476 | t | — | — | * | — | |
| 477 | t | — | — | * | — | |
| 478 | t | — | — | * | — | |
| 479 | c | — | * | — | — | |
| 480 | c | — | * | — | — | |
| 481 | a | — | — | — | * | |
| 482 | t | — | — | * | — | |
| 483 | g | * | — | — | — | |
| 484 | a | — | — | — | * | |
| 485 | a | — | — | — | * | |
| 486 | t | — | — | * | — | |
| 487 | t | — | — | * | — | |
| 488 | c | — | * | — | — | |
| 489 | c | — | * | — | — | |
| 490 | t | — | — | * | — | |
| 491 | t | — | — | * | — | |
| 492 | t | — | — | * | — | |
| 493 | t | — | — | * | — | |
| 494 | c | — | * | — | — | |
| 495 | t | — | — | * | — | |
| 496 | g | * | — | — | — | |
| 497 | c | — | * | — | — | |
| 498 | t | — | — | * | — | |
| 499 | t | — | — | * | — | |
| 500 | a | — | — | — | * | |
| 501 | a | — | — | — | * | |
| 502 | a | — | — | — | * | |
| 503 | c | — | * | — | — | |
| 504 | t | — | — | * | — | |
| 505 | g | * | — | — | — | |
| 506 | t | — | — | * | — | |
| 507 | c | — | * | — | — | |
| 508 | a | — | — | — | * | |
| 509 | g | * | — | — | — | |
| 510 | a | — | — | — | * | |
| 511 | a | — | — | — | * | |
| 512 | t | — | — | * | — | |
| 513 | t | — | — | * | — | |
| 514 | g | * | — | — | — | |
| 515 | t | — | — | * | — | |
| 516 | t | — | — | * | — | |
| 517 | t | — | — | * | — | |
| 518 | g | * | — | — | — | |
| 519 | t | — | — | * | — | |
| 520 | t | — | — | * | — | |
| 521 | t | — | — | * | — | |
| 522 | c | — | * | — | — | |
| 523 | t | — | — | * | — | |
| 524 | c | — | * | — | — | |
| 525 | a | — | — | — | * | |
| 526 | c | — | * | — | — | |
| 527 | a | — | — | — | * | |
| 528 | t | — | — | * | — | |
| 529 | t | — | — | * | — | |
| 530 | c | — | * | — | — | |
| 531 | g | * | — | — | — | |
| 532 | t | — | — | * | — | |
| 533 | g | * | — | — | — | |
| 534 | t | — | — | * | — | |
| 535 | t | — | — | * | — | |
| 536 | c | — | * | — | — | |
| 537 | c | — | * | — | — | |
| 538 | c | — | * | — | — | |
| 539 | a | — | — | — | * | |
| 540 | a | — | — | — | * | |
| 541 | c | — | * | — | — | |
| 542 | t | — | — | * | — | |
| 543 | g | * | — | — | — | |
| 544 | c | — | * | — | — | |
| 545 | t | — | — | * | — | |
| 546 | a | — | — | — | * | |
| 547 | c | — | * | — | — | |
| 548 | c | — | * | — | — | |
| 549 | c | — | * | — | — | |
| 550 | t | — | — | * | — | |
| 551 | g | * | — | — | — | |
| 552 | a | — | — | — | * | |
| 553 | t | — | — | * | — | |
| 554 | t | — | — | * | — | |
| 555 | g | * | — | — | — | |
| 556 | t | — | — | * | — | |
| 557 | g | * | — | — | — | |
| 558 | t | — | — | * | — | |
| 559 | t | — | — | * | — | |
| 560 | g | * | — | — | — | |
| 561 | g | * | — | — | — | |
| 562 | a | — | — | — | * | |
| 563 | g | * | — | — | — | |
| 564 | g | * | — | — | — | |
| 565 | a | — | — | — | * | |
| 566 | a | — | — | — | * | |
| 567 | g | * | — | — | — | |
| 568 | a | — | — | — | * | |
| 569 | g | * | — | — | — | |
| 570 | g | * | — | — | — | |
| 571 | g | * | — | — | — | |
| 572 | g | * | — | — | — | |
| 573 | g | * | — | — | — | |
| 574 | t | — | — | * | — | |
| 575 | a | — | — | — | * | |
| 576 | t | — | — | * | — | |
| 577 | g | * | — | — | — | |
| 578 | a | — | — | — | * | |
| 579 | a | — | — | — | * | |
| 580 | c | — | * | — | — | |
| 581 | a | — | — | — | * | |
| 582 | g | * | — | — | — | |
| 583 | t | — | — | * | — | |
| 584 | t | — | — | * | — | |
| 585 | g | * | — | — | — | |
| 586 | g | * | — | — | — | |
| 587 | a | — | — | — | * | |
| 588 | t | — | — | * | — | |
| 589 | g | * | — | — | — | |
| 590 | g | * | — | — | — | |
| 591 | t | — | — | * | — | |
| 592 | g | * | — | — | — | |
| 593 | t | — | — | * | — | |
| 594 | g | * | — | — | — | |
| 595 | g | * | — | — | — | |
| 596 | t | — | — | * | — | |
| 597 | c | — | * | — | — | |
| 598 | c | — | * | — | — | |
| 599 | t | — | — | * | — | |
| 600 | c | — | * | — | — | |
| 601 | a | — | — | — | * | |
| 602 | g | * | — | — | — | |
| 603 | a | — | — | — | * | |
| 604 | g | * | — | — | — | |
| 605 | a | — | — | — | * | |
| 606 | t | — | — | * | — | |
| 607 | g | * | — | — | — | |
| 608 | a | — | — | — | * | |
| 609 | g | * | — | — | — | |
| 610 | a | — | — | — | * | |
| 611 | a | — | — | — | * | |
| 612 | a | — | — | — | * | |
| 613 | c | — | * | — | — | |
| 614 | a | — | — | — | * | |
| 615 | c | — | * | — | — | |
| 616 | a | — | — | — | * | |
| 617 | g | * | — | — | — | |
| 618 | c | — | * | — | — | |
| 619 | c | — | * | — | — | |
| 620 | a | — | — | — | * | |
| 621 | g | * | — | — | — | |
| 622 | a | — | — | — | * | |
| 623 | g | * | — | — | — | |
| 624 | g | * | — | — | — | |
| 625 | g | * | — | — | — | |
| 626 | t | — | — | * | — | |
| 627 | c | — | * | — | — | |
| 628 | a | — | — | — | * | |
| 629 | g | * | — | — | — | |
| 630 | g | * | — | — | — | |
| 631 | t | — | — | * | — | |
| 632 | g | * | — | — | — | |
| 633 | t | — | — | * | — | |
| 634 | g | * | — | — | — | |
| 635 | a | — | — | — | * | |
| 636 | g | * | — | — | — | |
| 637 | c | — | * | — | — | |
| 638 | a | — | — | — | * | |
| 639 | g | * | — | — | — | |
| 640 | a | — | — | — | * | |
| 641 | a | — | — | — | * | |
| 642 | g | * | — | — | — | |
| 643 | a | — | — | — | * | |
| 644 | a | — | — | — | * | |
| 645 | c | — | * | — | — | |
| 646 | c | — | * | — | — | |
| 647 | c | — | * | — | — | |
| 648 | c | — | * | — | — | |
| 649 | a | — | — | — | * | |
| 650 | a | — | — | — | * | |
| 651 | g | * | — | — | — | |
| 652 | t | — | — | * | — | |
| 653 | g | * | — | — | — | |
| 654 | c | — | * | — | — | |
| 655 | t | — | — | * | — | |
| 656 | g | * | — | — | — | |
| 657 | c | — | * | — | — | |
| 658 | t | — | — | * | — | |
| 659 | g | * | — | — | — | |
| 660 | g | * | — | — | — | |
| 661 | t | — | — | * | — | |
| 662 | t | — | — | * | — | |
| 663 | a | — | — | — | * | |
| 664 | t | — | — | * | — | |
| 665 | a | — | — | — | * | |
| 666 | g | * | — | — | — | |
| 667 | a | — | — | — | * | |
| 668 | c | — | * | — | — | |
| 669 | t | — | — | * | — | |
| 670 | a | — | — | — | * | |
| 671 | a | — | — | — | * | |
| 672 | a | — | — | — | * | |
| 673 | a | — | — | — | * | |
| 674 | a | — | — | — | * | |
| 675 | t | — | — | * | — | |
| 676 | g | * | — | — | — | |
| 677 | c | — | * | — | — | |
| 678 | c | — | * | — | — | |
| 679 | t | — | — | * | — | |
| 680 | t | — | — | * | — | |
| 681 | t | — | — | * | — | |
| 682 | g | * | — | — | — | |
| 683 | c | — | * | — | — | |
| 684 | t | — | — | * | — | |
| 685 | c | — | * | — | — | |
| 686 | c | — | * | — | — | |
| 687 | t | — | — | * | — | |
| 688 | t | — | — | * | — | |
| 689 | a | — | — | — | * | |
| 690 | t | — | — | * | — | |
| 691 | t | — | — | * | — | |
| 692 | c | — | * | — | — | |
| 693 | a | — | — | — | * | |
| 694 | c | — | * | — | — | |
| 695 | t | — | — | * | — | |
| 696 | c | — | * | — | — | |
| 697 | a | — | — | — | * | |
| 698 | c | — | * | — | — | |
| 699 | c | — | * | — | — | |
| 700 | c | — | * | — | — | |
| 701 | t | — | — | * | — | |
| 702 | c | — | * | — | — | |
| 703 | a | — | — | — | * | |
| 704 | a | — | — | — | * | |
| 705 | t | — | — | * | — | |
| 706 | a | — | — | — | * | |
| 707 | a | — | — | — | * | |
| 708 | a | — | — | — | * | |
| 709 | g | * | — | — | — | |
| 710 | a | — | — | — | * | |
| 711 | a | — | — | — | * | |
| 712 | g | * | — | — | — | |
| 713 | g | * | — | — | — | |
| 714 | a | — | — | — | * | |
| 715 | g | * | — | — | — | |
| 716 | c | — | * | — | — | |
| 717 | c | — | * | — | — | |
| 718 | g | * | — | — | — | |
| 719 | a | — | — | — | * | |
| 720 | g | * | — | — | — | |
| 721 | a | — | — | — | * | |
| 722 | c | — | * | — | — | |
| 723 | t | — | — | * | — | |
| 724 | g | * | — | — | — | |
| 725 | a | — | — | — | * | |
| 726 | g | * | — | — | — | |
| 727 | t | — | — | * | — | |
| 728 | t | — | — | * | — | |
| 729 | t | — | — | * | — | |
| 730 | t | — | — | * | — | |
| 731 | t | — | — | * | — | |
| 732 | c | — | * | — | — | |
| 733 | t | — | — | * | — | |
| 734 | t | — | — | * | — | |
| 735 | c | — | * | — | — | |
| 736 | t | — | — | * | — | |
| 737 | c | — | * | — | — | |
| 738 | c | — | * | — | — | |
| 739 | a | — | — | — | * | |
| 740 | t | — | — | * | — | |
| 741 | t | — | — | * | — | |
| 742 | g | * | — | — | — | |
| 743 | a | — | — | — | * | |
| 744 | g | * | — | — | — | |
| 745 | a | — | — | — | * | |
| 746 | g | * | — | — | — | |
| 747 | c | — | * | — | — | |
| 748 | t | — | — | * | — | |
| 749 | t | — | — | * | — | |
| 750 | t | — | — | * | — | |
| 751 | c | — | * | — | — | |
| 752 | t | — | — | * | — | |
| 753 | t | — | — | * | — | |
| 754 | t | — | — | * | — | |
| 755 | g | * | — | — | — | |
| 756 | a | — | — | — | * | |
| 757 | a | — | — | — | * | |
| 758 | c | — | * | — | — | |
| 759 | t | — | — | * | — | |
| 760 | g | * | — | — | — | |
| 761 | c | — | * | — | — | |
| 762 | a | — | — | — | * | |
| 763 | c | — | * | — | — | |
| 764 | a | — | — | — | * | |
| 765 | g | * | — | — | — | |
| 766 | c | — | * | — | — | |
| 767 | t | — | — | * | — | |
| 768 | c | — | * | — | — | |
| 769 | c | — | * | — | — | |
| 770 | a | — | — | — | * | |
| 771 | t | — | — | * | — | |
| 772 | t | — | — | * | — | |
| 773 | c | — | * | — | — | |
| 774 | a | — | — | — | * | |
| 775 | g | * | — | — | — | |
| 776 | a | — | — | — | * | |
| 777 | a | — | — | — | * | |
| 778 | a | — | — | — | * | |
| 779 | c | — | * | — | — | |
| 780 | t | — | — | * | — | |
| 781 | g | * | — | — | — | |
| 782 | a | — | — | — | * | |
| 783 | a | — | — | — | * | |
| 784 | t | — | — | * | — | |
| 785 | g | * | — | — | — | |
| 786 | g | * | — | — | — | |
| 787 | t | — | — | * | — | |
| 788 | t | — | — | * | — | |
| 789 | g | * | — | — | — | |
| 790 | a | — | — | — | * | |
| 791 | a | — | — | — | * | |
| 792 | t | — | — | * | — | |
| 793 | g | * | — | — | — | |
| 794 | g | * | — | — | — | |
| 795 | t | — | — | * | — | |
| 796 | c | — | * | — | — | |
| 797 | c | — | * | — | — | |
| 798 | t | — | — | * | — | |
| 799 | t | — | — | * | — | |
| 800 | g | * | — | — | — | |
| 801 | c | — | * | — | — | |
| 802 | a | — | — | — | * | |
| 803 | t | — | — | * | — | |
| 804 | t | — | — | * | — | |
| 805 | t | — | — | * | — | |
| 806 | g | * | — | — | — | |
| 807 | c | — | * | — | — | |
| 808 | a | — | — | — | * | |
| 809 | g | * | — | — | — | |
| 810 | a | — | — | — | * | |
| 811 | c | — | * | — | — | |
| 812 | t | — | — | * | — | |
| 813 | t | — | — | * | — | |
| 814 | t | — | — | * | — | |
| 815 | c | — | * | — | — | |
| 816 | t | — | — | * | — | |
| 817 | g | * | — | — | — | |
| 818 | a | — | — | — | * | |
| 819 | a | — | — | — | * | |
| 820 | a | — | — | — | * | |
| 821 | t | — | — | * | — | |
| 822 | c | — | * | — | — | |
| 823 | t | — | — | * | — | |
| 824 | a | — | — | — | * | |
| 825 | a | — | — | — | * | |
| 826 | c | — | * | — | — | |
| 827 | a | — | — | — | * | |
| 828 | t | — | — | * | — | |
| 829 | c | — | * | — | — | |
| 830 | c | — | * | — | — | |
| 831 | t | — | — | * | — | |
| 832 | a | — | — | — | * | |
| 833 | g | * | — | — | — | |
| 834 | t | — | — | * | — | |
| 835 | t | — | — | * | — | |
| 836 | t | — | — | * | — | |
| 837 | c | — | * | — | — | |
| 838 | a | — | — | — | * | |
| 839 | t | — | — | * | — | |
| 840 | t | — | — | * | — | |
| 841 | g | * | — | — | — | |
| 842 | c | — | * | — | — | |
| 843 | c | — | * | — | — | |
| 844 | t | — | — | * | — | |
| 845 | g | * | — | — | — | |
| 846 | c | — | * | — | — | |
| 847 | t | — | — | * | — | |
| 848 | c | — | * | — | — | |
| 849 | t | — | — | * | — | |
| 850 | c | — | * | — | — | |
| 851 | a | — | — | — | * | |
| 852 | t | — | — | * | — | |
| 853 | c | — | * | — | — | |
| 854 | c | — | * | — | — | |
| 855 | t | — | — | * | — | |
| 856 | c | — | * | — | — | |
| 857 | a | — | — | — | * | |
| 858 | t | — | — | * | — | |
| 859 | a | — | — | — | * | |
| 860 | a | — | — | — | * | |
| 861 | t | — | — | * | — | |
| 862 | c | — | * | — | — | |
| 863 | t | — | — | * | — | |
| 864 | t | — | — | * | — | |
| 865 | c | — | * | — | — | |
| 866 | c | — | * | — | — | |
| 867 | a | — | — | — | * | |
| 868 | a | — | — | — | * | |
| 869 | g | * | — | — | — | |
| 870 | t | — | — | * | — | |
| 871 | g | * | — | — | — | |
| 872 | c | — | * | — | — | |
| 873 | t | — | — | * | — | |
| 874 | c | — | * | — | — | |
| 875 | t | — | — | * | — | |
| 876 | c | — | * | — | — | |
| 877 | c | — | * | — | — | |
| 878 | c | — | * | — | — | |
| 879 | c | — | * | — | — | |
| 880 | a | — | — | — | * | |
| 881 | a | — | — | — | * | |
| 882 | a | — | — | — | * | |
| 883 | t | — | — | * | — | |
| 884 | t | — | — | * | — | |
| 885 | a | — | — | — | * | |
| 886 | a | — | — | — | * | |
| 887 | c | — | * | — | — | |
| 888 | a | — | — | — | * | |
| 889 | a | — | — | — | * | |
| 890 | c | — | * | — | — | |
| 891 | a | — | — | — | * | |
| 892 | t | — | — | * | — | |
| 893 | t | — | — | * | — | |
| 894 | c | — | * | — | — | |
| 895 | t | — | — | * | — | |
| 896 | t | — | — | * | — | |
| 897 | g | * | — | — | — | |
| 898 | c | — | * | — | — | |
| 899 | c | — | * | — | — | |
| 900 | a | — | — | — | * | |
| 901 | t | — | — | * | — | |
| 902 | c | — | * | — | — | |
| 903 | a | — | — | — | * | |
| 904 | c | — | * | — | — | |
| 905 | a | — | — | — | * | |
| 906 | a | — | — | — | * | |
| 907 | g | * | — | — | — | |
| 908 | a | — | — | — | * | |
| 909 | c | — | * | — | — | |
| 910 | t | — | — | * | — | |
| 911 | a | — | — | — | * | |
| 912 | t | — | — | * | — | |
| 913 | t | — | — | * | — | |
| 914 | g | * | — | — | — | |
| 915 | g | * | — | — | — | |
| 916 | a | — | — | — | * | |
| 917 | a | — | — | — | * | |
| 918 | g | * | — | — | — | |
| 919 | g | * | — | — | — | |
| 920 | t | — | — | * | — | |
| 921 | a | — | — | — | * | |
| 922 | a | — | — | — | * | |
| 923 | a | — | — | — | * | |
| 924 | t | — | — | * | — | |
| 925 | a | — | — | — | * | |
| 926 | a | — | — | — | * | |
| 927 | g | * | — | — | — | |
| 928 | a | — | — | — | * | |
| 929 | t | — | — | * | — | |
| 930 | c | — | * | — | — | |
| 931 | a | — | — | — | * | |
| 932 | c | — | * | — | — | |
| 933 | a | — | — | — | * | |
| 934 | g | * | — | — | — | |
| 935 | a | — | — | — | * | |
| 936 | t | — | — | * | — | |
| 937 | g | * | — | — | — | |
| 938 | a | — | — | — | * | |
| 939 | g | * | — | — | — | |
| 940 | a | — | — | — | * | |
| 941 | a | — | — | — | * | |
| 942 | a | — | — | — | * | |
| 943 | g | * | — | — | — | |
| 944 | t | — | — | * | — | |
| 945 | g | * | — | — | — | |
| 946 | c | — | * | — | — | |
| 947 | t | — | — | * | — | |
| 948 | t | — | — | * | — | |
| 949 | t | — | — | * | — | |
| 950 | g | * | — | — | — | |
| 951 | t | — | — | * | — | |
| 952 | c | — | * | — | — | |
| 953 | a | — | — | — | * | |
| 954 | t | — | — | * | — | |
| 955 | c | — | * | — | — | |
| 956 | t | — | — | * | — | |
| 957 | a | — | — | — | * | |
| 958 | t | — | — | * | — | |
| 959 | g | * | — | — | — | |
| 960 | g | * | — | — | — | |
| 961 | a | — | — | — | * | |
| 962 | a | — | — | — | * | |
| 963 | a | — | — | — | * | |
| 964 | g | * | — | — | — | |
| 965 | g | * | — | — | — | |
| 966 | a | — | — | — | * | |
| 967 | c | — | * | — | — | |
| 968 | t | — | — | * | — | |
| 969 | g | * | — | — | — | |
| 970 | a | — | — | — | * | |
| 971 | a | — | — | — | * | |
| 972 | g | * | — | — | — | |
| 973 | a | — | — | — | * | |
| 974 | a | — | — | — | * | |
| 975 | c | — | * | — | — | |
| 976 | t | — | — | * | — | |
| 977 | t | — | — | * | — | |
| 978 | c | — | * | — | — | |
| 979 | a | — | — | — | * | |
| 980 | a | — | — | — | * | |
| 981 | g | * | — | — | — | |
| 982 | g | * | — | — | — | |
| 983 | c | — | * | — | — | |
| 984 | a | — | — | — | * | |
| 985 | g | * | — | — | — | |
| 986 | t | — | — | * | — | |
| 987 | a | — | — | — | * | |
| 988 | g | * | — | — | — | |
| 989 | a | — | — | — | * | |
| 990 | t | — | — | * | — | |
| 991 | t | — | — | * | — | |
| 992 | g | * | — | — | — | |
| 993 | t | — | — | * | — | |
| 994 | a | — | — | — | * | |
| 995 | c | — | * | — | — | |
| 996 | t | — | — | * | — | |
| 997 | t | — | — | * | — | |
| 998 | a | — | — | — | * | |
| 999 | c | — | * | — | — | |
| 1000 | c | — | * | — | — | |
| 1001 | t | — | — | * | — | |
| 1002 | a | — | — | — | * | |
| 1003 | a | — | — | — | * | |
| 1004 | a | — | — | — | * | |
| 1005 | t | — | — | * | — | |
| 1006 | a | — | — | — | * | |
| 1007 | c | — | * | — | — | |
| 1008 | a | — | — | — | * | |
| 1009 | a | — | — | — | * | |
| 1010 | c | — | * | — | — | |
| 1011 | t | — | — | * | — | |
| 1012 | t | — | — | * | — | |
| 1013 | c | — | * | — | — | |
| 1014 | a | — | — | — | * | |
| 1015 | g | * | — | — | — | |
| 1016 | a | — | — | — | * | |
| 1017 | a | — | — | — | * | |
| 1018 | g | * | — | — | — | |
| 1019 | t | — | — | * | — | |
| 1020 | t | — | — | * | — | |
| 1021 | a | — | — | — | * | |
| 1022 | t | — | — | * | — | |
| 1023 | a | — | — | — | * | |
| 1024 | t | — | — | * | — | |
| 1025 | g | * | — | — | — | |
| 1026 | t | — | — | * | — | |
| 1027 | a | — | — | — | * | |
| 1028 | g | * | — | — | — | |
| 1029 | a | — | — | — | * | |
| 1030 | t | — | — | * | — | |
| 1031 | t | — | — | * | — | |
| 1032 | g | * | — | — | — | |
| 1033 | t | — | — | * | — | |
| 1034 | t | — | — | * | — | |
| 1035 | t | — | — | * | — | |
| 1036 | t | — | — | * | — | |
| 1037 | c | — | * | — | — | |
| 1038 | t | — | — | * | — | |
| 1039 | a | — | — | — | * | |
| 1040 | a | — | — | — | * | |
| 1041 | c | — | * | — | — | |
| 1042 | t | — | — | * | — | |
| 1043 | t | — | — | * | — | |
| 1044 | t | — | — | * | — | |
| 1045 | g | * | — | — | — | |
| 1046 | a | — | — | — | * | |
| 1047 | t | — | — | * | — | |
| 1048 | c | — | * | — | — | |
| 1049 | a | — | — | — | * | |
| 1050 | c | — | * | — | — | |
| 1051 | t | — | — | * | — | |
| 1052 | t | — | — | * | — | |
| 1053 | t | — | — | * | — | |
| 1054 | c | — | * | — | — | |
| 1055 | c | — | * | — | — | |
| 1056 | c | — | * | — | — | |
| 1057 | a | — | — | — | * | |
| 1058 | c | — | * | — | — | |
| 1059 | c | — | * | — | — | |
| 1060 | t | — | — | * | — | |
| 1061 | a | — | — | — | * | |
| 1062 | t | — | — | * | — | |
| 1063 | t | — | — | * | — | |
| 1064 | t | — | — | * | — | |
| 1065 | a | — | — | — | * | |
| 1066 | c | — | * | — | — | |
| 1067 | t | — | — | * | — | |
| 1068 | t | — | — | * | — | |
| 1069 | g | * | — | — | — | |
| 1070 | g | * | — | — | — | |
| 1071 | t | — | — | * | — | |
| 1072 | a | — | — | — | * | |
| 1073 | g | * | — | — | — | |
| 1074 | t | — | — | * | — | |
| 1075 | t | — | — | * | — | |
| 1076 | t | — | — | * | — | |
| 1077 | a | — | — | — | * | |
| 1078 | c | — | * | — | — | |
| 1079 | g | * | — | — | — | |
| 1080 | a | — | — | — | * | |
| 1081 | g | * | — | — | — | |
| 1082 | a | — | — | — | * | |
| 1083 | t | — | — | * | — | |
| 1084 | g | * | — | — | — | |
| 1085 | t | — | — | * | — | |
| 1086 | t | — | — | * | — | |
| 1087 | t | — | — | * | — | |
| 1088 | t | — | — | * | — | |
| 1089 | a | — | — | — | * | |
| 1090 | c | — | * | — | — | |
| 1091 | t | — | — | * | — | |
| 1092 | t | — | — | * | — | |
| 1093 | t | — | — | * | — | |
| 1094 | g | * | — | — | — | |
| 1095 | t | — | — | * | — | |
| 1096 | t | — | — | * | — | |
| 1097 | t | — | — | * | — | |
| 1098 | t | — | — | * | — | |
| 1099 | c | — | * | — | — | |
| 1100 | a | — | — | — | * | |
| 1101 | c | — | * | — | — | |
| 1102 | a | — | — | — | * | |
| 1103 | t | — | — | * | — | |
| 1104 | a | — | — | — | * | |
| 1105 | g | * | — | — | — | |
| 1106 | a | — | — | — | * | |
| 1107 | t | — | — | * | — | |
| 1108 | t | — | — | * | — | |
| 1109 | g | * | — | — | — | |
| 1110 | g | * | — | — | — | |
| 1111 | c | — | * | — | — | |
| 1112 | a | — | — | — | * | |
| 1113 | a | — | — | — | * | |
| 1114 | c | — | * | — | — | |
| 1115 | t | — | — | * | — | |
| 1116 | g | * | — | — | — | |
| 1117 | c | — | * | — | — | |
| 1118 | a | — | — | — | * | |
| 1119 | g | * | — | — | — | |
| 1120 | t | — | — | * | — | |
| 1121 | a | — | — | — | * | |
| 1122 | a | — | — | — | * | |
| 1123 | c | — | * | — | — | |
| 1124 | c | — | * | — | — | |
| 1125 | a | — | — | — | * | |
| 1126 | c | — | * | — | — | |
| 1127 | t | — | — | * | — | |
| 1128 | g | * | — | — | — | |
| 1129 | a | — | — | — | * | |
| 1130 | a | — | — | — | * | |
| 1131 | a | — | — | — | * | |
| 1132 | c | — | * | — | — | |
| 1133 | a | — | — | — | * | |
| 1134 | c | — | * | — | — | |
| 1135 | a | — | — | — | * | |
| 1136 | a | — | — | — | * | |
| 1137 | g | * | — | — | — | |
| 1138 | a | — | — | — | * | |
| 1139 | t | — | — | * | — | |
| 1140 | t | — | — | * | — | |
| 1141 | t | — | — | * | — | |
| 1142 | t | — | — | * | — | |
| 1143 | t | — | — | * | — | |
| 1144 | t | — | — | * | — | |
| 1145 | t | — | — | * | — | |
| 1146 | t | — | — | * | — | |
| 1147 | t | — | — | * | — | |
| 1148 | t | — | — | * | — | |
| 1149 | t | — | — | * | — | |
| 1150 | t | — | — | * | — | |
| 1151 | t | — | — | * | — | |
| 1152 | t | — | — | * | — | |
| 1153 | t | — | — | * | — | |
| 1154 | t | — | — | * | — | |
| 1155 | t | — | — | * | — | |
| 1156 | t | — | — | * | — | |
| 1157 | t | — | — | * | — | |
| 1158 | t | — | — | * | — | |
| 1159 | t | — | — | * | — | |
| 1160 | t | — | — | * | — | |
| 1161 | t | — | — | * | — | |
| 1162 | t | — | — | * | — | |
| 1163 | t | — | — | * | — | |
| 1164 | g | * | — | — | — | |
| 1165 | t | — | — | * | — | |
| 1166 | g | * | — | — | — | |
| 1167 | a | — | — | — | * | |
| 1168 | c | — | * | — | — | |
| 1169 | a | — | — | — | * | |
| 1170 | g | * | — | — | — | |
| 1171 | a | — | — | — | * | |
| 1172 | a | — | — | — | * | |
| 1173 | t | — | — | * | — | |
| 1174 | t | — | — | * | — | |
| 1175 | t | — | — | * | — | |
| 1176 | c | — | * | — | — | |
| 1177 | a | — | — | — | * | |
| 1178 | c | — | * | — | — | |
| 1179 | t | — | — | * | — | |
| 1180 | c | — | * | — | — | |
| 1181 | t | — | — | * | — | |
| 1182 | t | — | — | * | — | |
| 1183 | g | * | — | — | — | |
| 1184 | t | — | — | * | — | |
| 1185 | t | — | — | * | — | |
| 1186 | g | * | — | — | — | |
| 1187 | c | — | * | — | — | |
| 1188 | c | — | * | — | — | |
| 1189 | c | — | * | — | — | |
| 1190 | a | — | — | — | * | |
| 1191 | g | * | — | — | — | |
| 1192 | g | * | — | — | — | |
| 1193 | c | — | * | — | — | |
| 1194 | t | — | — | * | — | |
| 1195 | g | * | — | — | — | |
| 1196 | a | — | — | — | * | |
| 1197 | a | — | — | — | * | |
| 1198 | g | * | — | — | — | |
| 1199 | t | — | — | * | — | |
| 1200 | g | * | — | — | — | |
| 1201 | c | — | * | — | — | |
| 1202 | a | — | — | — | * | |
| 1203 | a | — | — | — | * | |
| 1204 | t | — | — | * | — | |
| 1205 | g | * | — | — | — | |
| 1206 | g | * | — | — | — | |
| 1207 | c | — | * | — | — | |
| 1208 | a | — | — | — | * | |
| 1209 | c | — | * | — | — | |
| 1210 | a | — | — | — | * | |
| 1211 | g | * | — | — | — | |
| 1212 | t | — | — | * | — | |
| 1213 | c | — | * | — | — | |
| 1214 | t | — | — | * | — | |
| 1215 | c | — | * | — | — | |
| 1216 | g | * | — | — | — | |
| 1217 | g | * | — | — | — | |
| 1218 | c | — | * | — | — | |
| 1219 | t | — | — | * | — | |
| 1220 | c | — | * | — | — | |
| 1221 | a | — | — | — | * | |
| 1222 | c | — | * | — | — | |
| 1223 | t | — | — | * | — | |
| 1224 | g | * | — | — | — | |
| 1225 | c | — | * | — | — | |
| 1226 | a | — | — | — | * | |
| 1227 | a | — | — | — | * | |
| 1228 | c | — | * | — | — | |
| 1229 | c | — | * | — | — | |
| 1230 | t | — | — | * | — | |
| 1231 | c | — | * | — | — | |
| 1232 | c | — | * | — | — | |
| 1233 | a | — | — | — | * | |
| 1234 | c | — | * | — | — | |
| 1235 | t | — | — | * | — | |
| 1236 | t | — | — | * | — | |
| 1237 | c | — | * | — | — | |
| 1238 | c | — | * | — | — | |
| 1239 | c | — | * | — | — | |
| 1240 | g | * | — | — | — | |
| 1241 | g | * | — | — | — | |
| 1242 | t | — | — | * | — | |
| 1243 | t | — | — | * | — | |
| 1244 | c | — | * | — | — | |
| 1245 | a | — | — | — | * | |
| 1246 | a | — | — | — | * | |
| 1247 | g | * | — | — | — | |
| 1248 | t | — | — | * | — | |
| 1249 | g | * | — | — | — | |
| 1250 | a | — | — | — | * | |
| 1251 | t | — | — | * | — | |
| 1252 | c | — | * | — | — | |
| 1253 | c | — | * | — | — | |
| 1254 | t | — | — | * | — | |
| 1255 | c | — | * | — | — | |
| 1256 | c | — | * | — | — | |
| 1257 | t | — | — | * | — | |
| 1258 | g | * | — | — | — | |
| 1259 | c | — | * | — | — | |
| 1260 | c | — | * | — | — | |
| 1261 | t | — | — | * | — | |
| 1262 | c | — | * | — | — | |
| 1263 | a | — | — | — | * | |
| 1264 | g | * | — | — | — | |
| 1265 | c | — | * | — | — | |
| 1266 | c | — | * | — | — | |
| 1267 | t | — | — | * | — | |
| 1268 | c | — | * | — | — | |
| 1269 | c | — | * | — | — | |
| 1270 | c | — | * | — | — | |
| 1271 | a | — | — | — | * | |
| 1272 | a | — | — | — | * | |
| 1273 | c | — | * | — | — | |
| 1274 | t | — | — | * | — | |
| 1275 | a | — | — | — | * | |
| 1276 | g | * | — | — | — | |
| 1277 | c | — | * | — | — | |
| 1278 | t | — | — | * | — | |
| 1279 | g | * | — | — | — | |
| 1280 | g | * | — | — | — | |
| 1281 | g | * | — | — | — | |
| 1282 | a | — | — | — | * | |
| 1283 | t | — | — | * | — | |
| 1284 | t | — | — | * | — | |
| 1285 | a | — | — | — | * | |
| 1286 | c | — | * | — | — | |
| 1287 | a | — | — | — | * | |
| 1288 | g | * | — | — | — | |
| 1289 | g | * | — | — | — | |
| 1290 | a | — | — | — | * | |
| 1291 | g | * | — | — | — | |
| 1292 | c | — | * | — | — | |
| 1293 | c | — | * | — | — | |
| 1294 | c | — | * | — | — | |
| 1295 | a | — | — | — | * | |
| 1296 | c | — | * | — | — | |
| 1297 | c | — | * | — | — | |
| 1298 | a | — | — | — | * | |
| 1299 | c | — | * | — | — | |
| 1300 | c | — | * | — | — | |
| 1301 | a | — | — | — | * | |
| 1302 | t | — | — | * | — | |
| 1303 | g | * | — | — | — | |
| 1304 | c | — | * | — | — | |
| 1305 | c | — | * | — | — | |
| 1306 | c | — | * | — | — | |
| 1307 | g | * | — | — | — | |
| 1308 | g | * | — | — | — | |
| 1309 | c | — | * | — | — | |
| 1310 | t | — | — | * | — | |
| 1311 | a | — | — | — | * | |
| 1312 | a | — | — | — | * | |
| 1313 | t | — | — | * | — | |
| 1314 | t | — | — | * | — | |
| 1315 | t | — | — | * | — | |
| 1316 | a | — | — | — | * | |
| 1317 | t | — | — | * | — | |
| 1318 | t | — | — | * | — | |
| 1319 | t | — | — | * | — | |
| 1320 | t | — | — | * | — | |
| 1321 | t | — | — | * | — | |
| 1322 | a | — | — | — | * | |
| 1323 | g | * | — | — | — | |
| 1324 | t | — | — | * | — | |
| 1325 | g | * | — | — | — | |
| 1326 | g | * | — | — | — | |
| 1327 | a | — | — | — | * | |
| 1328 | g | * | — | — | — | |
| 1329 | a | — | — | — | * | |
| 1330 | t | — | — | * | — | |
| 1331 | g | * | — | — | — | |
| 1332 | g | * | — | — | — | |
| 1333 | g | * | — | — | — | |
| 1334 | g | * | — | — | — | |
| 1335 | t | — | — | * | — | |
| 1336 | t | — | — | * | — | |
| 1337 | t | — | — | * | — | |
| 1338 | c | — | * | — | — | |
| 1339 | a | — | — | — | * | |
| 1340 | c | — | * | — | — | |
| 1341 | c | — | * | — | — | |
| 1342 | a | — | — | — | * | |
| 1343 | t | — | — | * | — | |
| 1344 | g | * | — | — | — | |
| 1345 | t | — | — | * | — | |
| 1346 | t | — | — | * | — | |
| 1347 | g | * | — | — | — | |
| 1348 | g | * | — | — | — | |
| 1349 | c | — | * | — | — | |
| 1350 | c | — | * | — | — | |
| 1351 | a | — | — | — | * | |
| 1352 | g | * | — | — | — | |
| 1353 | g | * | — | — | — | |
| 1354 | c | — | * | — | — | |
| 1355 | t | — | — | * | — | |
| 1356 | g | * | — | — | — | |
| 1357 | g | * | — | — | — | |
| 1358 | t | — | — | * | — | |
| 1359 | c | — | * | — | — | |
| 1360 | t | — | — | * | — | |
| 1361 | c | — | * | — | — | |
| 1362 | g | * | — | — | — | |
| 1363 | a | — | — | — | * | |
| 1364 | a | — | — | — | * | |
| 1365 | c | — | * | — | — | |
| 1366 | t | — | — | * | — | |
| 1367 | c | — | * | — | — | |
| 1368 | c | — | * | — | — | |
| 1369 | t | — | — | * | — | |
| 1370 | g | * | — | — | — | |
| 1371 | a | — | — | — | * | |
| 1372 | c | — | * | — | — | |
| 1373 | c | — | * | — | — | |
| 1374 | t | — | — | * | — | |
| 1375 | c | — | * | — | — | |
| 1376 | a | — | — | — | * | |
| 1377 | g | * | — | — | — | |
| 1378 | g | * | — | — | — | |
| 1379 | t | — | — | * | — | |
| 1380 | g | * | — | — | — | |
| 1381 | a | — | — | — | * | |
| 1382 | t | — | — | * | — | |
| 1383 | c | — | * | — | — | |
| 1384 | c | — | * | — | — | |
| 1385 | a | — | — | — | * | |
| 1386 | c | — | * | — | — | |
| 1387 | c | — | * | — | — | |
| 1388 | c | — | * | — | — | |
| 1389 | a | — | — | — | * | |
| 1390 | c | — | * | — | — | |
| 1391 | c | — | * | — | — | |
| 1392 | t | — | — | * | — | |
| 1393 | c | — | * | — | — | |
| 1394 | g | * | — | — | — | |
| 1395 | g | * | — | — | — | |
| 1396 | c | — | * | — | — | |
| 1397 | c | — | * | — | — | |
| 1398 | t | — | — | * | — | |
| 1399 | t | — | — | * | — | |
| 1400 | c | — | * | — | — | |
| 1401 | c | — | * | — | — | |
| 1402 | a | — | — | — | * | |
| 1403 | a | — | — | — | * | |
| 1404 | a | — | — | — | * | |
| 1405 | g | * | — | — | — | |
| 1406 | t | — | — | * | — | |
| 1407 | g | * | — | — | — | |
| 1408 | c | — | * | — | — | |
| 1409 | t | — | — | * | — | |
| 1410 | g | * | — | — | — | |
| 1411 | g | * | — | — | — | |
| 1412 | g | * | — | — | — | |
| 1413 | a | — | — | — | * | |
| 1414 | t | — | — | * | — | |
| 1415 | t | — | — | * | — | |
| 1416 | a | — | — | — | * | |
| 1417 | c | — | * | — | — | |
| 1418 | a | — | — | — | * | |
| 1419 | a | — | — | — | * | |
| 1420 | g | * | — | — | — | |
| 1421 | t | — | — | * | — | |
| 1422 | a | — | — | — | * | |
| 1423 | t | — | — | * | — | |
| 1424 | g | * | — | — | — | |
| 1425 | a | — | — | — | * | |
| 1426 | g | * | — | — | — | |
| 1427 | c | — | * | — | — | |
| 1428 | c | — | * | — | — | |
| 1429 | a | — | — | — | * | |
| 1430 | c | — | * | — | — | |
| 1431 | t | — | — | * | — | |
| 1432 | g | * | — | — | — | |
| 1433 | c | — | * | — | — | |
| 1434 | g | * | — | — | — | |
| 1435 | c | — | * | — | — | |
| 1436 | a | — | — | — | * | |
| 1437 | t | — | — | * | — | |
| 1438 | t | — | — | * | — | |
| 1439 | t | — | — | * | — | |
| 1440 | t | — | — | * | — | |
| 1441 | a | — | — | — | * | |
| 1442 | a | — | — | — | * | |
| 1443 | g | * | — | — | — | |
| 1444 | t | — | — | * | — | |
| 1445 | g | * | — | — | — | |
| 1446 | c | — | * | — | — | |
| 1447 | a | — | — | — | * | |
| 1448 | t | — | — | * | — | |
| 1449 | a | — | — | — | * | |
| 1450 | g | * | — | — | — | |
| 1451 | t | — | — | * | — | |
| 1452 | t | — | — | * | — | |
| 1453 | c | — | * | — | — | |
| 1454 | a | — | — | — | * | |
| 1455 | g | * | — | — | — | |
| 1456 | t | — | — | * | — | |
| 1457 | a | — | — | — | * | |
| 1458 | g | * | — | — | — | |
| 1459 | g | * | — | — | — | |
| 1460 | a | — | — | — | * | |
| 1461 | t | — | — | * | — | |
| 1462 | t | — | — | * | — | |
| 1463 | a | — | — | — | * | |
| 1464 | a | — | — | — | * | |
| 1465 | g | * | — | — | — | |
| 1466 | t | — | — | * | — | |
| 1467 | a | — | — | — | * | |
| 1468 | c | — | * | — | — | |
| 1469 | a | — | — | — | * | |
| 1470 | t | — | — | * | — | |
| 1471 | t | — | — | * | — | |
| 1472 | c | — | * | — | — | |
| 1473 | a | — | — | — | * | |
| 1474 | t | — | — | * | — | |
| 1475 | g | * | — | — | — | |
| 1476 | t | — | — | * | — | |
| 1477 | t | — | — | * | — | |
| 1478 | g | * | — | — | — | |
| 1479 | t | — | — | * | — | |
| 1480 | g | * | — | — | — | |
| 1481 | t | — | — | * | — | |
| 1482 | a | — | — | — | * | |
| 1483 | a | — | — | — | * | |
| 1484 | g | * | — | — | — | |
| 1485 | c | — | * | — | — | |
| 1486 | a | — | — | — | * | |
| 1487 | t | — | — | * | — | |
| 1488 | c | — | * | — | — | |
| 1489 | a | — | — | — | * | |
| 1490 | c | — | * | — | — | |
| 1491 | t | — | — | * | — | |
| 1492 | a | — | — | — | * | |
| 1493 | c | — | * | — | — | |
| 1494 | c | — | * | — | — | |
| 1495 | g | * | — | — | — | |
| 1496 | c | — | * | — | — | |
| 1497 | t | — | — | * | — | |
| 1498 | c | — | * | — | — | |
| 1499 | c | — | * | — | — | |
| 1500 | a | — | — | — | * | |
| 1501 | c | — | * | — | — | |
| 1502 | t | — | — | * | — | |
| 1503 | t | — | — | * | — | |
| 1504 | t | — | — | * | — | |
| 1505 | a | — | — | — | * | |
| 1506 | g | * | — | — | — | |
| 1507 | a | — | — | — | * | |
| 1508 | g | * | — | — | — | |
| 1509 | c | — | * | — | — | |
| 1510 | c | — | * | — | — | |
| 1511 | a | — | — | — | * | |
| 1512 | g | * | — | — | — | |
| 1513 | c | — | * | — | — | |
| 1514 | c | — | * | — | — | |
| 1515 | t | — | — | * | — | |
| 1516 | c | — | * | — | — | |
| 1517 | g | * | — | — | — | |
| 1518 | a | — | — | — | * | |
| 1519 | t | — | — | * | — | |
| 1520 | c | — | * | — | — | |
| 1521 | t | — | — | * | — | |
| 1522 | t | — | — | * | — | |
| 1523 | c | — | * | — | — | |
| 1524 | c | — | * | — | — | |
| 1525 | a | — | — | — | * | |
| 1526 | a | — | — | — | * | |
| 1527 | a | — | — | — | * | |
| 1528 | g | * | — | — | — | |
| 1529 | t | — | — | * | — | |
| 1530 | a | — | — | — | * | |
| 1531 | c | — | * | — | — | |
| 1532 | c | — | * | — | — | |
| 1533 | g | * | — | — | — | |
| 1534 | g | * | — | — | — | |
| 1535 | g | * | — | — | — | |
| 1536 | a | — | — | — | * | |
| 1537 | t | — | — | * | — | |
| 1538 | t | — | — | * | — | |
| 1539 | a | — | — | — | * | |
| 1540 | c | — | * | — | — | |
| 1541 | a | — | — | — | * | |
| 1542 | g | * | — | — | — | |
| 1543 | g | * | — | — | — | |
| 1544 | c | — | * | — | — | |
| 1545 | g | * | — | — | — | |
| 1546 | t | — | — | * | — | |
| 1547 | g | * | — | — | — | |
| 1548 | a | — | — | — | * | |
| 1549 | a | — | — | — | * | |
| 1550 | c | — | * | — | — | |
| 1551 | c | — | * | — | — | |
| 1552 | a | — | — | — | * | |
| 1553 | c | — | * | — | — | |
| 1554 | c | — | * | — | — | |
| 1555 | a | — | — | — | * | |
| 1556 | c | — | * | — | — | |
| 1557 | g | * | — | — | — | |
| 1558 | c | — | * | — | — | |
| 1559 | a | — | — | — | * | |
| 1560 | t | — | — | * | — | |
| 1561 | t | — | — | * | — | |
| 1562 | t | — | — | * | — | |
| 1563 | t | — | — | * | — | |
| 1564 | a | — | — | — | * | |
| 1565 | a | — | — | — | * | |
| 1566 | g | * | — | — | — | |
| 1567 | t | — | — | * | — | |
| 1568 | g | * | — | — | — | |
| 1569 | c | — | * | — | — | |
| 1570 | a | — | — | — | * | |
| 1571 | t | — | — | * | — | |
| 1572 | a | — | — | — | * | |
| 1573 | g | * | — | — | — | |
| 1574 | t | — | — | * | — | |
| 1575 | t | — | — | * | — | |
| 1576 | c | — | * | — | — | |
| 1577 | a | — | — | — | * | |
| 1578 | g | * | — | — | — | |
| 1579 | t | — | — | * | — | |
| 1580 | a | — | — | — | * | |
| 1581 | g | * | — | — | — | |
| 1582 | c | — | * | — | — | |
| 1583 | a | — | — | — | * | |
| 1584 | t | — | — | * | — | |
| 1585 | t | — | — | * | — | |
| 1586 | a | — | — | — | * | |
| 1587 | a | — | — | — | * | |
| 1588 | g | * | — | — | — | |
| 1589 | t | — | — | * | — | |
| 1590 | a | — | — | — | * | |
| 1591 | c | — | * | — | — | |
| 1592 | a | — | — | — | * | |
| 1593 | t | — | — | * | — | |
| 1594 | t | — | — | * | — | |
| 1595 | c | — | * | — | — | |
| 1596 | a | — | — | — | * | |
| 1597 | t | — | — | * | — | |
| 1598 | a | — | — | — | * | |
| 1599 | t | — | — | * | — | |
| 1600 | t | — | — | * | — | |
| 1601 | g | * | — | — | — | |
| 1602 | t | — | — | * | — | |
| 1603 | t | — | — | * | — | |
| 1604 | g | * | — | — | — | |
| 1605 | t | — | — | * | — | |
| 1606 | g | * | — | — | — | |
| 1607 | t | — | — | * | — | |
| 1608 | a | — | — | — | * | |
| 1609 | a | — | — | — | * | |
| 1610 | g | * | — | — | — | |
| 1611 | c | — | * | — | — | |
| 1612 | a | — | — | — | * | |
| 1613 | t | — | — | * | — | |
| 1614 | c | — | * | — | — | |
| 1615 | a | — | — | — | * | |
| 1616 | c | — | * | — | — | |
| 1617 | c | — | * | — | — | |
| 1618 | a | — | — | — | * | |
| 1619 | c | — | * | — | — | |
| 1620 | c | — | * | — | — | |
| 1621 | a | — | — | — | * | |
| 1622 | c | — | * | — | — | |
| 1623 | t | — | — | * | — | |
| 1624 | c | — | * | — | — | |
| 1625 | c | — | * | — | — | |
| 1626 | a | — | — | — | * | |
| 1627 | c | — | * | — | — | |
| 1628 | t | — | — | * | — | |
| 1629 | t | — | — | * | — | |
| 1630 | t | — | — | * | — | |
| 1631 | a | — | — | — | * | |
| 1632 | g | * | — | — | — | |
| 1633 | a | — | — | — | * | |
| 1634 | a | — | — | — | * | |
| 1635 | c | — | * | — | — | |
| 1636 | a | — | — | — | * | |
| 1637 | t | — | — | * | — | |
| 1638 | t | — | — | * | — | |
| 1639 | c | — | * | — | — | |
| 1640 | t | — | — | * | — | |
| 1641 | t | — | — | * | — | |
| 1642 | c | — | * | — | — | |
| 1643 | a | — | — | — | * | |
| 1644 | t | — | — | * | — | |
| 1645 | g | * | — | — | — | |
| 1646 | t | — | — | * | — | |
| 1647 | t | — | — | * | — | |
| 1648 | c | — | * | — | — | |
| 1649 | t | — | — | * | — | |
| 1650 | c | — | * | — | — | |
| 1651 | a | — | — | — | * | |
| 1652 | t | — | — | * | — | |
| 1653 | a | — | — | — | * | |
| 1654 | c | — | * | — | — | |
| 1655 | c | — | * | — | — | |
| 1656 | g | * | — | — | — | |
| 1657 | a | — | — | — | * | |
| 1658 | a | — | — | — | * | |
| 1659 | a | — | — | — | * | |
| 1660 | c | — | * | — | — | |
| 1661 | c | — | * | — | — | |
| 1662 | c | — | * | — | — | |
| 1663 | c | — | * | — | — | |
| 1664 | a | — | — | — | * | |
| 1665 | t | — | — | * | — | |
| 1666 | a | — | — | — | * | |
| 1667 | c | — | * | — | — | |
| 1668 | c | — | * | — | — | |
| 1669 | c | — | * | — | — | |
| 1670 | a | — | — | — | * | |
| 1671 | t | — | — | * | — | |
| 1672 | c | — | * | — | — | |
| 1673 | a | — | — | — | * | |
| 1674 | a | — | — | — | * | |
| 1675 | a | — | — | — | * | |
| 1676 | t | — | — | * | — | |
| 1677 | a | — | — | — | * | |
| 1678 | t | — | — | * | — | |
| 1679 | g | * | — | — | — | |
| 1680 | t | — | — | * | — | |
| 1681 | c | — | * | — | — | |
| 1682 | c | — | * | — | — | |
| 1683 | c | — | * | — | — | |
| 1684 | c | — | * | — | — | |
| 1685 | a | — | — | — | * | |
| 1686 | t | — | — | * | — | |
| 1687 | t | — | — | * | — | |
| 1688 | t | — | — | * | — | |
| 1689 | g | * | — | — | — | |
| 1690 | t | — | — | * | — | |
| 1691 | c | — | * | — | — | |
| 1692 | c | — | * | — | — | |
| 1693 | c | — | * | — | — | |
| 1694 | t | — | — | * | — | |
| 1695 | g | * | — | — | — | |
| 1696 | c | — | * | — | — | |
| 1697 | c | — | * | — | — | |
| 1698 | t | — | — | * | — | |
| 1699 | c | — | * | — | — | |
| 1700 | c | — | * | — | — | |
| 1701 | a | — | — | — | * | |
| 1702 | g | * | — | — | — | |
| 1703 | c | — | * | — | — | |
| 1704 | c | — | * | — | — | |
| 1705 | t | — | — | * | — | |
| 1706 | c | — | * | — | — | |
| 1707 | t | — | — | * | — | |
| 1708 | g | * | — | — | — | |
| 1709 | g | * | — | — | — | |
| 1710 | c | — | * | — | — | |
| 1711 | c | — | * | — | — | |
| 1712 | a | — | — | — | * | |
| 1713 | c | — | * | — | — | |
| 1714 | c | — | * | — | — | |
| 1715 | a | — | — | — | * | |
| 1716 | c | — | * | — | — | |
| 1717 | c | — | * | — | — | |
| 1718 | a | — | — | — | * | |
| 1719 | a | — | — | — | * | |
| 1720 | t | — | — | * | — | |
| 1721 | t | — | — | * | — | |
| 1722 | c | — | * | — | — | |
| 1723 | a | — | — | — | * | |
| 1724 | c | — | * | — | — | |
| 1725 | t | — | — | * | — | |
| 1726 | c | — | * | — | — | |
| 1727 | t | — | — | * | — | |
| 1728 | c | — | * | — | — | |
| 1729 | t | — | — | * | — | |
| 1730 | g | * | — | — | — | |
| 1731 | t | — | — | * | — | |
| 1732 | c | — | * | — | — | |
| 1733 | t | — | — | * | — | |
| 1734 | c | — | * | — | — | |
| 1735 | t | — | — | * | — | |
| 1736 | g | * | — | — | — | |
| 1737 | t | — | — | * | — | |
| 1738 | a | — | — | — | * | |
| 1739 | g | * | — | — | — | |
| 1740 | a | — | — | — | * | |
| 1741 | t | — | — | * | — | |
| 1742 | t | — | — | * | — | |
| 1743 | g | * | — | — | — | |
| 1744 | a | — | — | — | * | |
| 1745 | c | — | * | — | — | |
| 1746 | t | — | — | * | — | |
| 1747 | c | — | * | — | — | |
| 1748 | t | — | — | * | — | |
| 1749 | a | — | — | — | * | |
| 1750 | g | * | — | — | — | |
| 1751 | a | — | — | — | * | |
| 1752 | t | — | — | * | — | |
| 1753 | a | — | — | — | * | |
| 1754 | c | — | * | — | — | |
| 1755 | c | — | * | — | — | |
| 1756 | t | — | — | * | — | |
| 1757 | c | — | * | — | — | |
| 1758 | a | — | — | — | * | |
| 1759 | t | — | — | * | — | |
| 1760 | a | — | — | — | * | |
| 1761 | t | — | — | * | — | |
| 1762 | a | — | — | — | * | |
| 1763 | a | — | — | — | * | |
| 1764 | g | * | — | — | — | |
| 1765 | t | — | — | * | — | |
| 1766 | g | * | — | — | — | |
| 1767 | g | * | — | — | — | |
| 1768 | c | — | * | — | — | |
| 1769 | a | — | — | — | * | |
| 1770 | t | — | — | * | — | |
| 1771 | t | — | — | * | — | |
| 1772 | g | * | — | — | — | |
| 1773 | t | — | — | * | — | |
| 1774 | a | — | — | — | * | |
| 1775 | c | — | * | — | — | |
| 1776 | a | — | — | — | * | |
| 1777 | g | * | — | — | — | |
| 1778 | t | — | — | * | — | |
| 1779 | a | — | — | — | * | |
| 1780 | t | — | — | * | — | |
| 1781 | a | — | — | — | * | |
| 1782 | t | — | — | * | — | |
| 1783 | g | * | — | — | — | |
| 1784 | t | — | — | * | — | |
| 1785 | c | — | * | — | — | |
| 1786 | c | — | * | — | — | |
| 1787 | t | — | — | * | — | |
| 1788 | t | — | — | * | — | |
| 1789 | t | — | — | * | — | |
| 1790 | t | — | — | * | — | |
| 1791 | g | * | — | — | — | |
| 1792 | t | — | — | * | — | |
| 1793 | a | — | — | — | * | |
| 1794 | a | — | — | — | * | |
| 1795 | c | — | * | — | — | |
| 1796 | t | — | — | * | — | |
| 1797 | g | * | — | — | — | |
| 1798 | g | * | — | — | — | |
| 1799 | c | — | * | — | — | |
| 1800 | t | — | — | * | — | |
| 1801 | t | — | — | * | — | |
| 1802 | a | — | — | — | * | |
| 1803 | t | — | — | * | — | |
| 1804 | t | — | — | * | — | |
| 1805 | t | — | — | * | — | |
| 1806 | c | — | * | — | — | |
| 1807 | a | — | — | — | * | |
| 1808 | c | — | * | — | — | |
| 1809 | t | — | — | * | — | |
| 1810 | t | — | — | * | — | |
| 1811 | g | * | — | — | — | |
| 1812 | g | * | — | — | — | |
| 1813 | c | — | * | — | — | |
| 1814 | a | — | — | — | * | |
| 1815 | c | — | * | — | — | |
| 1816 | a | — | — | — | * | |
| 1817 | a | — | — | — | * | |
| 1818 | t | — | — | * | — | |
| 1819 | g | * | — | — | — | |
| 1820 | t | — | — | * | — | |
| 1821 | c | — | * | — | — | |
| 1822 | t | — | — | * | — | |
| 1823 | t | — | — | * | — | |
| 1824 | c | — | * | — | — | |
| 1825 | c | — | * | — | — | |
| 1826 | a | — | — | — | * | |
| 1827 | g | * | — | — | — | |
| 1828 | a | — | — | — | * | |
| 1829 | t | — | — | * | — | |
| 1830 | t | — | — | * | — | |
| 1831 | c | — | * | — | — | |
| 1832 | c | — | * | — | — | |
| 1833 | a | — | — | — | * | |
| 1834 | t | — | — | * | — | |
| 1835 | g | * | — | — | — | |
| 1836 | a | — | — | — | * | |
| 1837 | g | * | — | — | — | |
| 1838 | g | * | — | — | — | |
| 1839 | t | — | — | * | — | |
| 1840 | a | — | — | — | * | |
| 1841 | g | * | — | — | — | |
| 1842 | c | — | * | — | — | |
| 1843 | a | — | — | — | * | |
| 1844 | g | * | — | — | — | |
| 1845 | g | * | — | — | — | |
| 1846 | t | — | — | * | — | |
| 1847 | g | * | — | — | — | |
| 1848 | t | — | — | * | — | |
| 1849 | g | * | — | — | — | |
| 1850 | a | — | — | — | * | |
| 1851 | g | * | — | — | — | |
| 1852 | a | — | — | — | * | |
| 1853 | a | — | — | — | * | |
| 1854 | t | — | — | * | — | |
| 1855 | t | — | — | * | — | |
| 1856 | t | — | — | * | — | |
| 1857 | c | — | * | — | — | |
| 1858 | t | — | — | * | — | |
| 1859 | c | — | * | — | — | |
| 1860 | t | — | — | * | — | |
| 1861 | c | — | * | — | — | |
| 1862 | c | — | * | — | — | |
| 1863 | t | — | — | * | — | |
| 1864 | t | — | — | * | — | |
| 1865 | t | — | — | * | — | |
| 1866 | g | * | — | — | — | |
| 1867 | t | — | — | * | — | |
| 1868 | t | — | — | * | — | |
| 1869 | a | — | — | — | * | |
| 1870 | a | — | — | — | * | |
| 1871 | g | * | — | — | — | |
| 1872 | a | — | — | — | * | |
| 1873 | c | — | * | — | — | |
| 1874 | c | — | * | — | — | |
| 1875 | t | — | — | * | — | |
| 1876 | a | — | — | — | * | |
| 1877 | a | — | — | — | * | |
| 1878 | t | — | — | * | — | |
| 1879 | a | — | — | — | * | |
| 1880 | a | — | — | — | * | |
| 1881 | t | — | — | * | — | |
| 1882 | a | — | — | — | * | |
| 1883 | t | — | — | * | — | |
| 1884 | t | — | — | * | — | |
| 1885 | c | — | * | — | — | |
| 1886 | c | — | * | — | — | |
| 1887 | t | — | — | * | — | |
| 1888 | t | — | — | * | — | |
| 1889 | t | — | — | * | — | |
| 1890 | g | * | — | — | — | |
| 1891 | c | — | * | — | — | |
| 1892 | a | — | — | — | * | |
| 1893 | t | — | — | * | — | |
| 1894 | g | * | — | — | — | |
| 1895 | t | — | — | * | — | |
| 1896 | a | — | — | — | * | |
| 1897 | a | — | — | — | * | |
| 1898 | a | — | — | — | * | |
| 1899 | t | — | — | * | — | |
| 1900 | a | — | — | — | * | |
| 1901 | c | — | * | — | — | |
| 1902 | c | — | * | — | — | |
| 1903 | a | — | — | — | * | |
| 1904 | c | — | * | — | — | |
| 1905 | a | — | — | — | * | |
| 1906 | t | — | — | * | — | |
| 1907 | c | — | * | — | — | |
| 1908 | t | — | — | * | — | |
| 1909 | t | — | — | * | — | |
| 1910 | g | * | — | — | — | |
| 1911 | t | — | — | * | — | |
| 1912 | t | — | — | * | — | |
| 1913 | t | — | — | * | — | |
| 1914 | a | — | — | — | * | |
| 1915 | t | — | — | * | — | |
| 1916 | c | — | * | — | — | |
| 1917 | c | — | * | — | — | |
| 1918 | a | — | — | — | * | |
| 1919 | t | — | — | * | — | |
| 1920 | g | * | — | — | — | |
| 1921 | t | — | — | * | — | |
| 1922 | t | — | — | * | — | |
| 1923 | g | * | — | — | — | |
| 1924 | a | — | — | — | * | |
| 1925 | t | — | — | * | — | |
| 1926 | g | * | — | — | — | |
| 1927 | g | * | — | — | — | |
| 1928 | a | — | — | — | * | |
| 1929 | c | — | * | — | — | |
| 1930 | a | — | — | — | * | |
| 1931 | c | — | * | — | — | |
| 1932 | a | — | — | — | * | |
| 1933 | t | — | — | * | — | |
| 1934 | g | * | — | — | — | |
| 1935 | g | * | — | — | — | |
| 1936 | c | — | * | — | — | |
| 1937 | t | — | — | * | — | |
| 1938 | t | — | — | * | — | |
| 1939 | g | * | — | — | — | |
| 1940 | t | — | — | * | — | |
| 1941 | t | — | — | * | — | |
| 1942 | t | — | — | * | — | |
| 1943 | c | — | * | — | — | |
| 1944 | c | — | * | — | — | |
| 1945 | a | — | — | — | * | |
| 1946 | c | — | * | — | — | |
| 1947 | c | — | * | — | — | |
| 1948 | t | — | — | * | — | |
| 1949 | t | — | — | * | — | |
| 1950 | t | — | — | * | — | |
| 1951 | t | — | — | * | — | |
| 1952 | g | * | — | — | — | |
| 1953 | g | * | — | — | — | |
| 1954 | c | — | * | — | — | |
| 1955 | t | — | — | * | — | |
| 1956 | a | — | — | — | * | |
| 1957 | t | — | — | * | — | |
| 1958 | t | — | — | * | — | |
| 1959 | g | * | — | — | — | |
| 1960 | t | — | — | * | — | |
| 1961 | g | * | — | — | — | |
| 1962 | c | — | * | — | — | |
| 1963 | a | — | — | — | * | |
| 1964 | t | — | — | * | — | |
| 1965 | a | — | — | — | * | |
| 1966 | g | * | — | — | — | |
| 1967 | t | — | — | * | — | |
| 1968 | g | * | — | — | — | |
| 1969 | c | — | * | — | — | |
| 1970 | t | — | — | * | — | |
| 1971 | g | * | — | — | — | |
| 1972 | c | — | * | — | — | |
| 1973 | t | — | — | * | — | |
| 1974 | a | — | — | — | * | |
| 1975 | t | — | — | * | — | |
| 1976 | g | * | — | — | — | |
| 1977 | a | — | — | — | * | |
| 1978 | a | — | — | — | * | |
| 1979 | c | — | * | — | — | |
| 1980 | a | — | — | — | * | |
| 1981 | t | — | — | * | — | |
| 1982 | a | — | — | — | * | |
| 1983 | g | * | — | — | — | |
| 1984 | g | * | — | — | — | |
| 1985 | t | — | — | * | — | |
| 1986 | g | * | — | — | — | |
| 1987 | t | — | — | * | — | |
| 1988 | g | * | — | — | — | |
| 1989 | g | * | — | — | — | |
| 1990 | a | — | — | — | * | |
| 1991 | a | — | — | — | * | |
| 1992 | a | — | — | — | * | |
| 1993 | t | — | — | * | — | |
| 1994 | g | * | — | — | — | |
| 1995 | a | — | — | — | * | |
| 1996 | g | * | — | — | — | |
| 1997 | g | * | — | — | — | |
| 1998 | t | — | — | * | — | |
| 1999 | g | * | — | — | — | |
| 2000 | t | — | — | * | — | |
| 2001 | a | — | — | — | * | |
| 2002 | t | — | — | * | — | |
| 2003 | a | — | — | — | * | |
| 2004 | t | — | — | * | — | |
| 2005 | t | — | — | * | — | |
| 2006 | t | — | — | * | — | |
| 2007 | t | — | — | * | — | |
| 2008 | t | — | — | * | — | |
| 2009 | a | — | — | — | * | |
| 2010 | t | — | — | * | — | |
| 2011 | g | * | — | — | — | |
| 2012 | a | — | — | — | * | |
| 2013 | c | — | * | — | — | |
| 2014 | c | — | * | — | — | |
| 2015 | t | — | — | * | — | |
| 2016 | c | — | * | — | — | |
| 2017 | t | — | — | * | — | |
| 2018 | t | — | — | * | — | |
| 2019 | c | — | * | — | — | |
| 2020 | c | — | * | — | — | |
| 2021 | c | — | * | — | — | |
| 2022 | a | — | — | — | * | |
| 2023 | t | — | — | * | — | |
| 2024 | c | — | * | — | — | |
| 2025 | a | — | — | — | * | |
| 2026 | a | — | — | — | * | |
| 2027 | g | * | — | — | — | |
| 2028 | c | — | * | — | — | |
| 2029 | a | — | — | — | * | |
| 2030 | a | — | — | — | * | |
| 2031 | t | — | — | * | — | |
| 2032 | t | — | — | * | — | |
| 2033 | a | — | — | — | * | |
| 2034 | a | — | — | — | * | |
| 2035 | a | — | — | — | * | |
| 2036 | t | — | — | * | — | |
| 2037 | t | — | — | * | — | |
| 2038 | t | — | — | * | — | |
| 2039 | c | — | * | — | — | |
| 2040 | t | — | — | * | — | |
| 2041 | a | — | — | — | * | |
| 2042 | a | — | — | — | * | |
| 2043 | t | — | — | * | — | |
| 2044 | a | — | — | — | * | |
| 2045 | t | — | — | * | — | |
| 2046 | g | * | — | — | — | |
| 2047 | a | — | — | — | * | |
| 2048 | a | — | — | — | * | |
| 2049 | t | — | — | * | — | |
| 2050 | a | — | — | — | * | |
| 2051 | a | — | — | — | * | |
| 2052 | t | — | — | * | — | |
| 2053 | t | — | — | * | — | |
| 2054 | g | * | — | — | — | |
| 2055 | c | — | * | — | — | |
| 2056 | a | — | — | — | * | |
| 2057 | c | — | * | — | — | |
| 2058 | t | — | — | * | — | |
| 2059 | c | — | * | — | — | |
| 2060 | t | — | — | * | — | |
| 2061 | g | * | — | — | — | |
| 2062 | a | — | — | — | * | |
| 2063 | g | * | — | — | — | |
| 2064 | t | — | — | * | — | |
| 2065 | t | — | — | * | — | |
| 2066 | a | — | — | — | * | |
| 2067 | t | — | — | * | — | |
| 2068 | c | — | * | — | — | |
| 2069 | t | — | — | * | — | |
| 2070 | g | * | — | — | — | |
| 2071 | t | — | — | * | — | |
| 2072 | t | — | — | * | — | |
| 2073 | c | — | * | — | — | |
| 2074 | t | — | — | * | — | |
| 2075 | g | * | — | — | — | |
| 2076 | t | — | — | * | — | |
| 2077 | a | — | — | — | * | |
| 2078 | a | — | — | — | * | |
| 2079 | c | — | * | — | — | |
| 2080 | t | — | — | * | — | |
| 2081 | c | — | * | — | — | |
| 2082 | t | — | — | * | — | |
| 2083 | g | * | — | — | — | |
| 2084 | a | — | — | — | * | |
| 2085 | a | — | — | — | * | |
| 2086 | g | * | — | — | — | |
| 2087 | a | — | — | — | * | |
| 2088 | a | — | — | — | * | |
| 2089 | c | — | * | — | — | |
| 2090 | t | — | — | * | — | |
| 2091 | g | * | — | — | — | |
| 2092 | t | — | — | * | — | |
| 2093 | g | * | — | — | — | |
| 2094 | t | — | — | * | — | |
| 2095 | a | — | — | — | * | |
| 2096 | a | — | — | — | * | |
| 2097 | c | — | * | — | — | |
| 2098 | a | — | — | — | * | |
| 2099 | g | * | — | — | — | |
| 2100 | a | — | — | — | * | |
| 2101 | a | — | — | — | * | |
| 2102 | t | — | — | * | — | |
| 2103 | t | — | — | * | — | |
| 2104 | t | — | — | * | — | |
| 2105 | c | — | * | — | — | |
| 2106 | t | — | — | * | — | |
| 2107 | t | — | — | * | — | |
| 2108 | a | — | — | — | * | |
| 2109 | a | — | — | — | * | |
| 2110 | a | — | — | — | * | |
| 2111 | g | * | — | — | — | |
| 2112 | t | — | — | * | — | |
| 2113 | g | * | — | — | — | |
| 2114 | a | — | — | — | * | |
| 2115 | g | * | — | — | — | |
| 2116 | t | — | — | * | — | |
| 2117 | c | — | * | — | — | |
| 2118 | a | — | — | — | * | |
| 2119 | t | — | — | * | — | |
| 2120 | c | — | * | — | — | |
| 2121 | c | — | * | — | — | |
| 2122 | c | — | * | — | — | |
| 2123 | t | — | — | * | — | |
| 2124 | g | * | — | — | — | |
| 2125 | t | — | — | * | — | |
| 2126 | a | — | — | — | * | |
| 2127 | t | — | — | * | — | |
| 2128 | t | — | — | * | — | |
| 2129 | c | — | * | — | — | |
| 2130 | t | — | — | * | — | |
| 2131 | c | — | * | — | — | |
| 2132 | t | — | — | * | — | |
| 2133 | t | — | — | * | — | |
| 2134 | t | — | — | * | — | |
| 2135 | a | — | — | — | * | |
| 2136 | c | — | * | — | — | |
| 2137 | t | — | — | * | — | |
| 2138 | c | — | * | — | — | |
| 2139 | t | — | — | * | — | |
| 2140 | t | — | — | * | — | |
| 2141 | t | — | — | * | — | |
| 2142 | c | — | * | — | — | |
| 2143 | a | — | — | — | * | |
| 2144 | a | — | — | — | * | |
| 2145 | a | — | — | — | * | |
| 2146 | a | — | — | — | * | |
| 2147 | t | — | — | * | — | |
| 2148 | a | — | — | — | * | |
| 2149 | g | * | — | — | — | |
| 2150 | g | * | — | — | — | |
| 2151 | a | — | — | — | * | |
| 2152 | g | * | — | — | — | |
| 2153 | g | * | — | — | — | |
| 2154 | c | — | * | — | — | |
| 2155 | a | — | — | — | * | |
| 2156 | g | * | — | — | — | |
| 2157 | c | — | * | — | — | |
| 2158 | a | — | — | — | * | |
| 2159 | a | — | — | — | * | |
| 2160 | a | — | — | — | * | |
| 2161 | g | * | — | — | — | |
| 2162 | a | — | — | — | * | |
| 2163 | a | — | — | — | * | |
| 2164 | c | — | * | — | — | |
| 2165 | a | — | — | — | * | |
| 2166 | a | — | — | — | * | |
| 2167 | a | — | — | — | * | |
| 2168 | t | — | — | * | — | |
| 2169 | g | * | — | — | — | |
| 2170 | t | — | — | * | — | |
| 2171 | c | — | * | — | — | |
| 2172 | c | — | * | — | — | |
| 2173 | a | — | — | — | * | |
| 2174 | t | — | — | * | — | |
| 2175 | t | — | — | * | — | |
| 2176 | t | — | — | * | — | |
| 2177 | c | — | * | — | — | |
| 2178 | a | — | — | — | * | |
| 2179 | c | — | * | — | — | |
| 2180 | a | — | — | — | * | |
| 2181 | c | — | * | — | — | |
| 2182 | c | — | * | — | — | |
| 2183 | c | — | * | — | — | |
| 2184 | a | — | — | — | * | |
| 2185 | g | * | — | — | — | |
| 2186 | g | * | — | — | — | |
| 2187 | t | — | — | * | — | |
| 2188 | g | * | — | — | — | |
| 2189 | g | * | — | — | — | |
| 2190 | a | — | — | — | * | |
| 2191 | a | — | — | — | * | |
| 2192 | a | — | — | — | * | |
| 2193 | g | * | — | — | — | |
| 2194 | g | * | — | — | — | |
| 2195 | c | — | * | — | — | |
| 2196 | c | — | * | — | — | |
| 2197 | c | — | * | — | — | |
| 2198 | a | — | — | — | * | |
| 2199 | t | — | — | * | — | |
| 2200 | t | — | — | * | — | |
| 2201 | a | — | — | — | * | |
| 2202 | g | * | — | — | — | |
| 2203 | a | — | — | — | * | |
| 2204 | g | * | — | — | — | |
| 2205 | t | — | — | * | — | |
| 2206 | c | — | * | — | — | |
| 2207 | t | — | — | * | — | |
| 2208 | a | — | — | — | * | |
| 2209 | c | — | * | — | — | |
| 2210 | c | — | * | — | — | |
| 2211 | a | — | — | — | * | |
| 2212 | g | * | — | — | — | |
| 2213 | t | — | — | * | — | |
| 2214 | g | * | — | — | — | |
| 2215 | a | — | — | — | * | |
| 2216 | g | * | — | — | — | |
| 2217 | g | * | — | — | — | |
| 2218 | a | — | — | — | * | |
| 2219 | a | — | — | — | * | |
| 2220 | a | — | — | — | * | |
| 2221 | t | — | — | * | — | |
| 2222 | c | — | * | — | — | |
| 2223 | a | — | — | — | * | |
| 2224 | t | — | — | * | — | |
| 2225 | t | — | — | * | — | |
| 2226 | t | — | — | * | — | |
| 2227 | t | — | — | * | — | |
| 2228 | c | — | * | — | — | |
| 2229 | t | — | — | * | — | |
| 2230 | a | — | — | — | * | |
| 2231 | a | — | — | — | * | |
| 2232 | g | * | — | — | — | |
| 2233 | g | * | — | — | — | |
| 2234 | g | * | — | — | — | |
| 2235 | t | — | — | * | — | |
| 2236 | g | * | — | — | — | |
| 2237 | a | — | — | — | * | |
| 2238 | t | — | — | * | — | |
| 2239 | t | — | — | * | — | |
| 2240 | a | — | — | — | * | |
| 2241 | c | — | * | — | — | |
| 2242 | a | — | — | — | * | |
| 2243 | c | — | * | — | — | |
| 2244 | a | — | — | — | * | |
| 2245 | c | — | * | — | — | |
| 2246 | g | * | — | — | — | |
| 2247 | c | — | * | — | — | |
| 2248 | a | — | — | — | * | |
| 2249 | g | * | — | — | — | |
| 2250 | a | — | — | — | * | |
| 2251 | t | — | — | * | — | |
| 2252 | g | * | — | — | — | |
| 2253 | t | — | — | * | — | |
| 2254 | c | — | * | — | — | |
| 2255 | a | — | — | — | * | |
| 2256 | a | — | — | — | * | |
| 2257 | a | — | — | — | * | |
| 2258 | a | — | — | — | * | |
| 2259 | c | — | * | — | — | |
| 2260 | c | — | * | — | — | |
| 2261 | c | — | * | — | — | |
| 2262 | t | — | — | * | — | |
| 2263 | c | — | * | — | — | |
| 2264 | a | — | — | — | * | |
| 2265 | g | * | — | — | — | |
| 2266 | g | * | — | — | — | |
| 2267 | t | — | — | * | — | |
| 2268 | a | — | — | — | * | |
| 2269 | a | — | — | — | * | |
| 2270 | c | — | * | — | — | |
| 2271 | t | — | — | * | — | |
| 2272 | c | — | * | — | — | |
| 2273 | t | — | — | * | — | |
| 2274 | c | — | * | — | — | |
| 2275 | t | — | — | * | — | |
| 2276 | g | * | — | — | — | |
| 2277 | c | — | * | — | — | |
| 2278 | t | — | — | * | — | |
| 2279 | c | — | * | — | — | |
| 2280 | t | — | — | * | — | |
| 2281 | c | — | * | — | — | |
| 2282 | c | — | * | — | — | |
| 2283 | t | — | — | * | — | |
| 2284 | g | * | — | — | — | |
| 2285 | c | — | * | — | — | |
| 2286 | c | — | * | — | — | |
| 2287 | t | — | — | * | — | |
| 2288 | t | — | — | * | — | |
| 2289 | g | * | — | — | — | |
| 2290 | g | * | — | — | — | |
| 2291 | t | — | — | * | — | |
| 2292 | t | — | — | * | — | |
| 2293 | g | * | — | — | — | |
| 2294 | g | * | — | — | — | |
| 2295 | a | — | — | — | * | |
| 2296 | c | — | * | — | — | |
| 2297 | a | — | — | — | * | |
| 2298 | g | * | — | — | — | |
| 2299 | t | — | — | * | — | |
| 2300 | g | * | — | — | — | |
| 2301 | c | — | * | — | — | |
| 2302 | c | — | * | — | — | |
| 2303 | c | — | * | — | — | |
| 2304 | t | — | — | * | — | |
| 2305 | g | * | — | — | — | |
| 2306 | a | — | — | — | * | |
| 2307 | a | — | — | — | * | |
| 2308 | c | — | * | — | — | |
| 2309 | t | — | — | * | — | |
| 2310 | t | — | — | * | — | |
| 2311 | c | — | * | — | — | |
| 2312 | t | — | — | * | — | |
| 2313 | g | * | — | — | — | |
| 2314 | t | — | — | * | — | |
| 2315 | c | — | * | — | — | |
| 2316 | c | — | * | — | — | |
| 2317 | t | — | — | * | — | |
| 2318 | t | — | — | * | — | |
| 2319 | c | — | * | — | — | |
| 2320 | t | — | — | * | — | |
| 2321 | c | — | * | — | — | |
| 2322 | a | — | — | — | * | |
| 2323 | g | * | — | — | — | |
| 2324 | a | — | — | — | * | |
| 2325 | a | — | — | — | * | |
| 2326 | g | * | — | — | — | |
| 2327 | a | — | — | — | * | |
| 2328 | t | — | — | * | — | |
| 2329 | t | — | — | * | — | |
| 2330 | c | — | * | — | — | |
| 2331 | t | — | — | * | — | |
| 2332 | c | — | * | — | — | |
| 2333 | c | — | * | — | — | |
| 2334 | c | — | * | — | — | |
| 2335 | c | — | * | — | — | |
| 2336 | t | — | — | * | — | |
| 2337 | t | — | — | * | — | |
| 2338 | g | * | — | — | — | |
| 2339 | c | — | * | — | — | |
| 2340 | c | — | * | — | — | |
| 2341 | c | — | * | — | — | |
| 2342 | a | — | — | — | * | |
| 2343 | c | — | * | — | — | |
| 2344 | c | — | * | — | — | |
| 2345 | c | — | * | — | — | |
| 2346 | t | — | — | * | — | |
| 2347 | t | — | — | * | — | |
| 2348 | c | — | * | — | — | |
| 2349 | t | — | — | * | — | |
| 2350 | c | — | * | — | — | |
| 2351 | t | — | — | * | — | |
| 2352 | g | * | — | — | — | |
| 2353 | g | * | — | — | — | |
| 2354 | g | * | — | — | — | |
| 2355 | g | * | — | — | — | |
| 2356 | t | — | — | * | — | |
| 2357 | c | — | * | — | — | |
| 2358 | t | — | — | * | — | |
| 2359 | a | — | — | — | * | |
| 2360 | c | — | * | — | — | |
| 2361 | c | — | * | — | — | |
| 2362 | a | — | — | — | * | |
| 2363 | t | — | — | * | — | |
| 2364 | c | — | * | — | — | |
| 2365 | t | — | — | * | — | |
| 2366 | c | — | * | — | — | |
| 2367 | g | * | — | — | — | |
| 2368 | a | — | — | — | * | |
| 2369 | c | — | * | — | — | |
| 2370 | t | — | — | * | — | |
| 2371 | a | — | — | — | * | |
| 2372 | c | — | * | — | — | |
| 2373 | c | — | * | — | — | |
| 2374 | t | — | — | * | — | |
| 2375 | t | — | — | * | — | |
| 2376 | c | — | * | — | — | |
| 2377 | a | — | — | — | * | |
| 2378 | g | * | — | — | — | |
| 2379 | t | — | — | * | — | |
| 2380 | c | — | * | — | — | |
| 2381 | c | — | * | — | — | |
| 2382 | c | — | * | — | — | |
| 2383 | t | — | — | * | — | |
| 2384 | t | — | — | * | — | |
| 2385 | c | — | * | — | — | |
| 2386 | c | — | * | — | — | |
| 2387 | c | — | * | — | — | |
| 2388 | t | — | — | * | — | |
| 2389 | a | — | — | — | * | |
| 2390 | g | * | — | — | — | |
| 2391 | a | — | — | — | * | |
| 2392 | t | — | — | * | — | |
| 2393 | t | — | — | * | — | |
| 2394 | a | — | — | — | * | |
| 2395 | t | — | — | * | — | |
| 2396 | c | — | * | — | — | |
| 2397 | c | — | * | — | — | |
| 2398 | t | — | — | * | — | |
| 2399 | a | — | — | — | * | |
| 2400 | c | — | * | — | — | |
| 2401 | a | — | — | — | * | |
| 2402 | t | — | — | * | — | |
| 2403 | g | * | — | — | — | |
| 2404 | g | * | — | — | — | |
| 2405 | g | * | — | — | — | |
| 2406 | g | * | — | — | — | |
| 2407 | c | — | * | — | — | |
| 2408 | t | — | — | * | — | |
| 2409 | g | * | — | — | — | |
| 2410 | t | — | — | * | — | |
| 2411 | a | — | — | — | * | |
| 2412 | g | * | — | — | — | |
| 2413 | a | — | — | — | * | |
| 2414 | t | — | — | * | — | |
| 2415 | c | — | * | — | — | |
| 2416 | c | — | * | — | — | |
| 2417 | c | — | * | — | — | |
| 2418 | t | — | — | * | — | |
| 2419 | c | — | * | — | — | |
| 2420 | a | — | — | — | * | |
| 2421 | a | — | — | — | * | |
| 2422 | t | — | — | * | — | |
| 2423 | a | — | — | — | * | |
| 2424 | a | — | — | — | * | |
| 2425 | c | — | * | — | — | |
| 2426 | a | — | — | — | * | |
| 2427 | a | — | — | — | * | |
| 2428 | c | — | * | — | — | |
| 2429 | a | — | — | — | * | |
| 2430 | a | — | — | — | * | |
| 2431 | t | — | — | * | — | |
| 2432 | a | — | — | — | * | |
| 2433 | t | — | — | * | — | |
| 2434 | c | — | * | — | — | |
| 2435 | c | — | * | — | — | |
| 2436 | a | — | — | — | * | |
| 2437 | a | — | — | — | * | |
| 2438 | g | * | — | — | — | |
| 2439 | a | — | — | — | * | |
| 2440 | a | — | — | — | * | |
| 2441 | t | — | — | * | — | |
| 2442 | t | — | — | * | — | |
| 2443 | c | — | * | — | — | |
| 2444 | c | — | * | — | — | |
| 2445 | a | — | — | — | * | |
| 2446 | c | — | * | — | — | |
| 2447 | a | — | — | — | * | |
| 2448 | c | — | * | — | — | |
| 2449 | c | — | * | — | — | |
| 2450 | t | — | — | * | — | |
| 2451 | c | — | * | — | — | |
| 2452 | t | — | — | * | — | |
| 2453 | c | — | * | — | — | |
| 2454 | t | — | — | * | — | |
| 2455 | a | — | — | — | * | |
| 2456 | a | — | — | — | * | |
| 2457 | g | * | — | — | — | |
| 2458 | g | * | — | — | — | |
| 2459 | t | — | — | * | — | |
| 2460 | t | — | — | * | — | |
| 2461 | t | — | — | * | — | |
| 2462 | t | — | — | * | — | |
| 2463 | c | — | * | — | — | |
| 2464 | t | — | — | * | — | |
| 2465 | g | * | — | — | — | |
| 2466 | c | — | * | — | — | |
| 2467 | a | — | — | — | * | |
| 2468 | t | — | — | * | — | |
| 2469 | t | — | — | * | — | |
| 2470 | t | — | — | * | — | |
| 2471 | g | * | — | — | — | |
| 2472 | a | — | — | — | * | |
| 2473 | g | * | — | — | — | |
| 2474 | g | * | — | — | — | |
| 2475 | g | * | — | — | — | |
| 2476 | a | — | — | — | * | |
| 2477 | t | — | — | * | — | |
| 2478 | g | * | — | — | — | |
| 2479 | t | — | — | * | — | |
| 2480 | a | — | — | — | * | |
| 2481 | a | — | — | — | * | |
| 2482 | t | — | — | * | — | |
| 2483 | a | — | — | — | * | |
| 2484 | a | — | — | — | * | |
| 2485 | a | — | — | — | * | |
| 2486 | g | * | — | — | — | |
| 2487 | t | — | — | * | — | |
| 2488 | a | — | — | — | * | |
| 2489 | c | — | * | — | — | |
| 2490 | a | — | — | — | * | |
| 2491 | a | — | — | — | * | |
| 2492 | a | — | — | — | * | |
| 2493 | a | — | — | — | * | |
| 2494 | g | * | — | — | — | |
| 2495 | a | — | — | — | * | |
| 2496 | g | * | — | — | — | |
| 2497 | a | — | — | — | * | |
| 2498 | t | — | — | * | — | |
| 2499 | g | * | — | — | — | |
| 2500 | a | — | — | — | * | |
| 2501 | t | — | — | * | — | |
| 2502 | t | — | — | * | — | |
| 2503 | t | — | — | * | — | |
| 2504 | t | — | — | * | — | |
| 2505 | t | — | — | * | — | |
| 2506 | t | — | — | * | — | |
| 2507 | t | — | — | * | — | |
| 2508 | t | — | — | * | — | |
| 2509 | t | — | — | * | — | |
| 2510 | t | — | — | * | — | |
| 2511 | g | * | — | — | — | |
| 2512 | c | — | * | — | — | |
| 2513 | c | — | * | — | — | |
| 2514 | c | — | * | — | — | |
| 2515 | c | — | * | — | — | |
| 2516 | a | — | — | — | * | |
| 2517 | t | — | — | * | — | |
| 2518 | a | — | — | — | * | |
| 2519 | c | — | * | — | — | |
| 2520 | t | — | — | * | — | |
| 2521 | t | — | — | * | — | |
| 2522 | c | — | * | — | — | |
| 2523 | c | — | * | — | — | |
| 2524 | t | — | — | * | — | |
| 2525 | t | — | — | * | — | |
| 2526 | t | — | — | * | — | |
| 2527 | g | * | — | — | — | |
| 2528 | a | — | — | — | * | |
| 2529 | c | — | * | — | — | |
| 2530 | c | — | * | — | — | |
| 2531 | c | — | * | — | — | |
| 2532 | a | — | — | — | * | |
| 2533 | g | * | — | — | — | |
| 2534 | a | — | — | — | * | |
| 2535 | t | — | — | * | — | |
| 2536 | a | — | — | — | * | |
| 2537 | g | * | — | — | — | |
| 2538 | a | — | — | — | * | |
| 2539 | t | — | — | * | — | |
| 2540 | t | — | — | * | — | |
| 2541 | g | * | — | — | — | |
| 2542 | a | — | — | — | * | |
| 2543 | t | — | — | * | — | |
| 2544 | g | * | — | — | — | |
| 2545 | t | — | — | * | — | |
| 2546 | t | — | — | * | — | |
| 2547 | c | — | * | — | — | |
| 2548 | t | — | — | * | — | |
| 2549 | a | — | — | — | * | |
| 2550 | c | — | * | — | — | |
| 2551 | t | — | — | * | — | |
| 2552 | g | * | — | — | — | |
| 2553 | c | — | * | — | — | |
| 2554 | t | — | — | * | — | |
| 2555 | c | — | * | — | — | |
| 2556 | c | — | * | — | — | |
| 2557 | c | — | * | — | — | |
| 2558 | c | — | * | — | — | |
| 2559 | a | — | — | — | * | |
| 2560 | a | — | — | — | * | |
| 2561 | g | * | — | — | — | |
| 2562 | t | — | — | * | — | |
| 2563 | c | — | * | — | — | |
| 2564 | a | — | — | — | * | |
| 2565 | t | — | — | * | — | |
| 2566 | t | — | — | * | — | |
| 2567 | t | — | — | * | — | |
| 2568 | g | * | — | — | — | |
| 2569 | c | — | * | — | — | |
| 2570 | a | — | — | — | * | |
| 2571 | a | — | — | — | * | |
| 2572 | a | — | — | — | * | |
| 2573 | t | — | — | * | — | |
| 2574 | g | * | — | — | — | |
| 2575 | g | * | — | — | — | |
| 2576 | c | — | * | — | — | |
| 2577 | a | — | — | — | * | |
| 2578 | c | — | * | — | — | |
| 2579 | a | — | — | — | * | |
| 2580 | a | — | — | — | * | |
| 2581 | g | * | — | — | — | |
| 2582 | a | — | — | — | * | |
| 2583 | a | — | — | — | * | |
| 2584 | c | — | * | — | — | |
| 2585 | t | — | — | * | — | |
| 2586 | a | — | — | — | * | |
| 2587 | a | — | — | — | * | |
| 2588 | g | * | — | — | — | |
| 2589 | a | — | — | — | * | |
| 2590 | c | — | * | — | — | |
| 2591 | a | — | — | — | * | |
| 2592 | a | — | — | — | * | |
| 2593 | a | — | — | — | * | |
| 2594 | a | — | — | — | * | |
| 2595 | g | * | — | — | — | |
| 2596 | a | — | — | — | * | |
| 2597 | a | — | — | — | * | |
| 2598 | g | * | — | — | — | |
| 2599 | a | — | — | — | * | |
| 2600 | g | * | — | — | — | |
| 2601 | g | * | — | — | — | |
| 2602 | a | — | — | — | * | |
| 2603 | t | — | — | * | — | |
| 2604 | g | * | — | — | — | |
| 2605 | a | — | — | — | * | |
| 2606 | g | * | — | — | — | |
| 2607 | a | — | — | — | * | |
| 2608 | a | — | — | — | * | |
| 2609 | a | — | — | — | * | |
| 2610 | a | — | — | — | * | |
| 2611 | g | * | — | — | — | |
| 2612 | a | — | — | — | * | |
| 2613 | g | * | — | — | — | |
| 2614 | a | — | — | — | * | |
| 2615 | a | — | — | — | * | |
| 2616 | a | — | — | — | * | |
| 2617 | t | — | — | * | — | |
| 2618 | t | — | — | * | — | |
| 2619 | c | — | * | — | — | |
| 2620 | c | — | * | — | — | |
| 2621 | a | — | — | — | * | |
| 2622 | a | — | — | — | * | |
| 2623 | g | * | — | — | — | |
| 2624 | a | — | — | — | * | |
| 2625 | g | * | — | — | — | |
| 2626 | c | — | * | — | — | |
| 2627 | t | — | — | * | — | |
| 2628 | t | — | — | * | — | |
| 2629 | g | * | — | — | — | |
| 2630 | g | * | — | — | — | |
| 2631 | a | — | — | — | * | |
| 2632 | a | — | — | — | * | |
| 2633 | a | — | — | — | * | |
| 2634 | t | — | — | * | — | |
| 2635 | g | * | — | — | — | |
| 2636 | g | * | — | — | — | |
| 2637 | g | * | — | — | — | |
| 2638 | g | * | — | — | — | |
| 2639 | g | * | — | — | — | |
| 2640 | c | — | * | — | — | |
| 2641 | t | — | — | * | — | |
| 2642 | c | — | * | — | — | |
| 2643 | a | — | — | — | * | |
| 2644 | g | * | — | — | — | |
| 2645 | t | — | — | * | — | |
| 2646 | t | — | — | * | — | |
| 2647 | t | — | — | * | — | |
| 2648 | t | — | — | * | — | |
| 2649 | t | — | — | * | — | |
| 2650 | a | — | — | — | * | |
| 2651 | t | — | — | * | — | |
| 2652 | c | — | * | — | — | |
| 2653 | t | — | — | * | — | |
| 2654 | a | — | — | — | * | |
| 2655 | c | — | * | — | — | |
| 2656 | a | — | — | — | * | |
| 2657 | a | — | — | — | * | |
| 2658 | a | — | — | — | * | |
| 2659 | g | * | — | — | — | |
| 2660 | t | — | — | * | — | |
| 2661 | g | * | — | — | — | |
| 2662 | g | * | — | — | — | |
| 2663 | g | * | — | — | — | |
| 2664 | t | — | — | * | — | |
| 2665 | t | — | — | * | — | |
| 2666 | a | — | — | — | * | |
| 2667 | a | — | — | — | * | |
| 2668 | g | * | — | — | — | |
| 2669 | a | — | — | — | * | |
| 2670 | a | — | — | — | * | |
| 2671 | a | — | — | — | * | |
| 2672 | t | — | — | * | — | |
| 2673 | g | * | — | — | — | |
| 2674 | g | * | — | — | — | |
| 2675 | a | — | — | — | * | |
| 2676 | g | * | — | — | — | |
| 2677 | a | — | — | — | * | |
| 2678 | a | — | — | — | * | |
| 2679 | c | — | * | — | — | |
| 2680 | t | — | — | * | — | |
| 2681 | g | * | — | — | — | |
| 2682 | t | — | — | * | — | |
| 2683 | a | — | — | — | * | |
| 2684 | a | — | — | — | * | |
| 2685 | c | — | * | — | — | |
| 2686 | a | — | — | — | * | |
| 2687 | c | — | * | — | — | |
| 2688 | c | — | * | — | — | |
| 2689 | a | — | — | — | * | |
| 2690 | t | — | — | * | — | |
| 2691 | a | — | — | — | * | |
| 2692 | t | — | — | * | — | |
| 2693 | a | — | — | — | * | |
| 2694 | g | * | — | — | — | |
| 2695 | a | — | — | — | * | |
| 2696 | a | — | — | — | * | |
| 2697 | g | * | — | — | — | |
| 2698 | a | — | — | — | * | |
| 2699 | t | — | — | * | — | |
| 2700 | g | * | — | — | — | |
| 2701 | t | — | — | * | — | |
| 2702 | c | — | * | — | — | |
| 2703 | t | — | — | * | — | |
| 2704 | a | — | — | — | * | |
| 2705 | t | — | — | * | — | |
| 2706 | a | — | — | — | * | |
| 2707 | t | — | — | * | — | |
| 2708 | t | — | — | * | — | |
| 2709 | t | — | — | * | — | |
| 2710 | c | — | * | — | — | |
| 2711 | c | — | * | — | — | |
| 2712 | t | — | — | * | — | |
| 2713 | g | * | — | — | — | |
| 2714 | c | — | * | — | — | |
| 2715 | g | * | — | — | — | |
| 2716 | t | — | — | * | — | |
| 2717 | c | — | * | — | — | |
| 2718 | a | — | — | — | * | |
| 2719 | c | — | * | — | — | |
| 2720 | t | — | — | * | — | |
| 2721 | t | — | — | * | — | |
| 2722 | c | — | * | — | — | |
| 2723 | t | — | — | * | — | |
| 2724 | c | — | * | — | — | |
| 2725 | c | — | * | — | — | |
| 2726 | a | — | — | — | * | |
| 2727 | g | * | — | — | — | |
| 2728 | t | — | — | * | — | |
| 2729 | t | — | — | * | — | |
| 2730 | g | * | — | — | — | |
| 2731 | a | — | — | — | * | |
| 2732 | a | — | — | — | * | |
| 2733 | g | * | — | — | — | |
| 2734 | t | — | — | * | — | |
| 2735 | t | — | — | * | — | |
| 2736 | t | — | — | * | — | |
| 2737 | t | — | — | * | — | |
| 2738 | c | — | * | — | — | |
| 2739 | t | — | — | * | — | |
| 2740 | g | * | — | — | — | |
| 2741 | t | — | — | * | — | |
| 2742 | a | — | — | — | * | |
| 2743 | a | — | — | — | * | |
| 2744 | g | * | — | — | — | |
| 2745 | g | * | — | — | — | |
| 2746 | g | * | — | — | — | |
| 2747 | g | * | — | — | — | |
| 2748 | g | * | — | — | — | |
| 2749 | g | * | — | — | — | |
| 2750 | a | — | — | — | * | |
| 2751 | a | — | — | — | * | |
| 2752 | g | * | — | — | — | |
| 2753 | a | — | — | — | * | |
| 2754 | c | — | * | — | — | |
| 2755 | c | — | * | — | — | |
| 2756 | c | — | * | — | — | |
| 2757 | a | — | — | — | * | |
| 2758 | t | — | — | * | — | |
| 2759 | g | * | — | — | — | |
| 2760 | g | * | — | — | — | |
| 2761 | a | — | — | — | * | |
| 2762 | a | — | — | — | * | |
| 2763 | t | — | — | * | — | |
| 2764 | a | — | — | — | * | |
| 2765 | t | — | — | * | — | |
| 2766 | c | — | * | — | — | |
| 2767 | a | — | — | — | * | |
| 2768 | a | — | — | — | * | |
| 2769 | t | — | — | * | — | |
| 2770 | a | — | — | — | * | |
| 2771 | t | — | — | * | — | |
| 2772 | g | * | — | — | — | |
| 2773 | t | — | — | * | — | |
| 2774 | g | * | — | — | — | |
| 2775 | t | — | — | * | — | |
| 2776 | g | * | — | — | — | |
| 2777 | g | * | — | — | — | |
| 2778 | g | * | — | — | — | |
| 2779 | c | — | * | — | — | |
| 2780 | t | — | — | * | — | |
| 2781 | g | * | — | — | — | |
| 2782 | t | — | — | * | — | |
| 2783 | c | — | * | — | — | |
| 2784 | g | * | — | — | — | |
| 2785 | t | — | — | * | — | |
| 2786 | t | — | — | * | — | |
| 2787 | c | — | * | — | — | |
| 2788 | c | — | * | — | — | |
| 2789 | c | — | * | — | — | |
| 2790 | a | — | — | — | * | |
| 2791 | c | — | * | — | — | |
| 2792 | t | — | — | * | — | |
| 2793 | t | — | — | * | — | |
| 2794 | a | — | — | — | * | |
| 2795 | t | — | — | * | — | |
| 2796 | t | — | — | * | — | |
| 2797 | c | — | * | — | — | |
| 2798 | t | — | — | * | — | |
| 2799 | t | — | — | * | — | |
| 2800 | c | — | * | — | — | |
| 2801 | a | — | — | — | * | |
| 2802 | t | — | — | * | — | |
| 2803 | g | * | — | — | — | |
| 2804 | a | — | — | — | * | |
| 2805 | t | — | — | * | — | |
| 2806 | a | — | — | — | * | |
| 2807 | g | * | — | — | — | |
| 2808 | c | — | * | — | — | |
| 2809 | a | — | — | — | * | |
| 2810 | c | — | * | — | — | |
| 2811 | t | — | — | * | — | |
| 2812 | g | * | — | — | — | |
| 2813 | c | — | * | — | — | |
| 2814 | a | — | — | — | * | |
| 2815 | a | — | — | — | * | |
| 2816 | t | — | — | * | — | |
| 2817 | g | * | — | — | — | |
| 2818 | t | — | — | * | — | |
| 2819 | g | * | — | — | — | |
| 2820 | g | * | — | — | — | |
| 2821 | c | — | * | — | — | |
| 2822 | t | — | — | * | — | |
| 2823 | c | — | * | — | — | |
| 2824 | t | — | — | * | — | |
| 2825 | c | — | * | — | — | |
| 2826 | c | — | * | — | — | |
| 2827 | a | — | — | — | * | |
| 2828 | t | — | — | * | — | |
| 2829 | c | — | * | — | — | |
| 2830 | c | — | * | — | — | |
| 2831 | c | — | * | — | — | |
| 2832 | c | — | * | — | — | |
| 2833 | a | — | — | — | * | |
| 2834 | t | — | — | * | — | |
| 2835 | t | — | — | * | — | |
| 2836 | t | — | — | * | — | |
| 2837 | c | — | * | — | — | |
| 2838 | a | — | — | — | * | |
| 2839 | c | — | * | — | — | |
| 2840 | a | — | — | — | * | |
| 2841 | a | — | — | — | * | |
| 2842 | t | — | — | * | — | |
| 2843 | c | — | * | — | — | |
| 2844 | c | — | * | — | — | |
| 2845 | a | — | — | — | * | |
| 2846 | g | * | — | — | — | |
| 2847 | a | — | — | — | * | |
| 2848 | a | — | — | — | * | |
| 2849 | a | — | — | — | * | |
| 2850 | a | — | — | — | * | |
| 2851 | c | — | * | — | — | |
| 2852 | t | — | — | * | — | |
| 2853 | g | * | — | — | — | |
| 2854 | a | — | — | — | * | |
| 2855 | g | * | — | — | — | |
| 2856 | c | — | * | — | — | |
| 2857 | c | — | * | — | — | |
| 2858 | c | — | * | — | — | |
| 2859 | c | — | * | — | — | |
| 2860 | g | * | — | — | — | |
| 2861 | g | * | — | — | — | |
| 2862 | a | — | — | — | * | |
| 2863 | g | * | — | — | — | |
| 2864 | c | — | * | — | — | |
| 2865 | c | — | * | — | — | |
| 2866 | a | — | — | — | * | |
| 2867 | t | — | — | * | — | |
| 2868 | t | — | — | * | — | |
| 2869 | c | — | * | — | — | |
| 2870 | a | — | — | — | * | |
| 2871 | g | * | — | — | — | |
| 2872 | a | — | — | — | * | |
| 2873 | a | — | — | — | * | |
| 2874 | t | — | — | * | — | |
| 2875 | t | — | — | * | — | |
| 2876 | t | — | — | * | — | |
| 2877 | c | — | * | — | — | |
| 2878 | c | — | * | — | — | |
| 2879 | c | — | * | — | — | |
| 2880 | a | — | — | — | * | |
| 2881 | a | — | — | — | * | |
| 2882 | g | * | — | — | — | |
| 2883 | g | * | — | — | — | |
| 2884 | t | — | — | * | — | |
| 2885 | t | — | — | * | — | |
| 2886 | g | * | — | — | — | |
| 2887 | t | — | — | * | — | |
| 2888 | g | * | — | — | — | |
| 2889 | g | * | — | — | — | |
| 2890 | a | — | — | — | * | |
| 2891 | g | * | — | — | — | |
| 2892 | t | — | — | * | — | |
| 2893 | t | — | — | * | — | |
| 2894 | t | — | — | * | — | |
| 2895 | a | — | — | — | * | |
| 2896 | g | * | — | — | — | |
| 2897 | a | — | — | — | * | |
| 2898 | t | — | — | * | — | |
| 2899 | g | * | — | — | — | |
| 2900 | g | * | — | — | — | |
| 2901 | g | * | — | — | — | |
| 2902 | a | — | — | — | * | |
| 2903 | a | — | — | — | * | |
| 2904 | c | — | * | — | — | |
| 2905 | a | — | — | — | * | |
| 2906 | a | — | — | — | * | |
| 2907 | a | — | — | — | * | |
| 2908 | c | — | * | — | — | |
| 2909 | t | — | — | * | — | |
| 2910 | t | — | — | * | — | |
| 2911 | c | — | * | — | — | |
| 2912 | t | — | — | * | — | |
| 2913 | g | * | — | — | — | |
| 2914 | t | — | — | * | — | |
| 2915 | g | * | — | — | — | |
| 2916 | t | — | — | * | — | |
| 2917 | g | * | — | — | — | |
| 2918 | a | — | — | — | * | |
| 2919 | c | — | * | — | — | |
| 2920 | t | — | — | * | — | |
| 2921 | c | — | * | — | — | |
| 2922 | c | — | * | — | — | |
| 2923 | a | — | — | — | * | |
| 2924 | g | * | — | — | — | |
| 2925 | t | — | — | * | — | |
| 2926 | g | * | — | — | — | |
| 2927 | t | — | — | * | — | |
| 2928 | t | — | — | * | — | |
| 2929 | g | * | — | — | — | |
| 2930 | c | — | * | — | — | |
| 2931 | t | — | — | * | — | |
| 2932 | a | — | — | — | * | |
| 2933 | c | — | * | — | — | |
| 2934 | t | — | — | * | — | |
| 2935 | g | * | — | — | — | |
| 2936 | a | — | — | — | * | |
| 2937 | c | — | * | — | — | |
| 2938 | a | — | — | — | * | |
| 2939 | t | — | — | * | — | |
| 2940 | g | * | — | — | — | |
| 2941 | a | — | — | — | * | |
| 2942 | c | — | * | — | — | |
| 2943 | c | — | * | — | — | |
| 2944 | c | — | * | — | — | |
| 2945 | g | * | — | — | — | |
| 2946 | c | — | * | — | — | |
| 2947 | t | — | — | * | — | |
| 2948 | c | — | * | — | — | |
| 2949 | t | — | — | * | — | |
| 2950 | g | * | — | — | — | |
| 2951 | c | — | * | — | — | |
| 2952 | c | — | * | — | — | |
| 2953 | t | — | — | * | — | |
| 2954 | t | — | — | * | — | |
| 2955 | c | — | * | — | — | |
| 2956 | c | — | * | — | — | |
| 2957 | c | — | * | — | — | |
| 2958 | c | — | * | — | — | |
| 2959 | a | — | — | — | * | |
| 2960 | c | — | * | — | — | |
| 2961 | c | — | * | — | — | |
| 2962 | t | — | — | * | — | |
| 2963 | t | — | — | * | — | |
| 2964 | t | — | — | * | — | |
| 2965 | c | — | * | — | — | |
| 2966 | a | — | — | — | * | |
| 2967 | g | * | — | — | — | |
| 2968 | a | — | — | — | * | |
| 2969 | t | — | — | * | — | |
| 2970 | g | * | — | — | — | |
| 2971 | a | — | — | — | * | |
| 2972 | g | * | — | — | — | |
| 2973 | a | — | — | — | * | |
| 2974 | g | * | — | — | — | |
| 2975 | c | — | * | — | — | |
| 2976 | a | — | — | — | * | |
| 2977 | t | — | — | * | — | |
| 2978 | t | — | — | * | — | |
| 2979 | t | — | — | * | — | |
| 2980 | c | — | * | — | — | |
| 2981 | c | — | * | — | — | |
| 2982 | c | — | * | — | — | |
| 2983 | t | — | — | * | — | |
| 2984 | t | — | — | * | — | |
| 2985 | g | * | — | — | — | |
| 2986 | t | — | — | * | — | |
| 2987 | t | — | — | * | — | |
| 2988 | t | — | — | * | — | |
| 2989 | t | — | — | * | — | |
| 2990 | c | — | * | — | — | |
| 2991 | t | — | — | * | — | |
| 2992 | g | * | — | — | — | |
| 2993 | t | — | — | * | — | |
| 2994 | g | * | — | — | — | |
| 2995 | a | — | — | — | * | |
| 2996 | g | * | — | — | — | |
| 2997 | a | — | — | — | * | |
| 2998 | t | — | — | * | — | |
| 2999 | g | * | — | — | — | |
| 3000 | a | — | — | — | * | |

Indicates the nucleotide present at the specific position.

A particularly important polymorphism is at nucleotide position 957 (using cDNA nucleotide numbering). The majority of individuals have a T at position 957 (the “T allele”. However, a statistically significant number of individuals presenting with symptoms of schizophrenia, alcoholism, or other neurological, psychiatric or psychological conditions, phenotypes or states have an increase in the frequency of the C allele. This is referred to as a 957C>T polymorphism. Although not intending to limit the present invention to any one theory or mode of operation, a DRD2 genetic locus carrying 957C>T results in unstable D2 translation material and hence reduced levels of DRD2.

Accordingly, the present invention provides a genetic marker for a neurological, psychiatric or psychological condition, state or phenotype in an individual said genetic marker comprising a C at nucleotide position 957 wherein the presence of a 957C polymorphism is indicative of, or a predisposition of developing a neurological, psychiatric or psychological condition, phenotype or state selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder and Social Phobia.

Preferably, the neurological, psychiatric or psychological condition, phenotype or state is schizophrenia or alcoholism or a related condition.

In a most preferred embodiment, therefore, the present invention provides a genetic marker for a neurological, psychiatric or psychological condition, phenotype or state in an individual said genetic marker comprising a C at nucleotide position 957 wherein the presence of a 957C polymorphism is indicative of or a predisposition of developing schizophrenia or alcoholism or a related condition. The identification of such a marker allows for the diagnosis of a neurological, psychiatric or psychological condition, phenotype or state in an individual and the application of pharmacogenomics, or “personalized medicine,” which involves using genomic knowledge to tailor treatments that best suit the individual patient's needs.

Reference to nucleotide position “957” is based on the cDNA sequence (SEQ ID NO:1) or its corresponding location in the genomic sequence (SEQ ID NO:3). The numbering is calculated from the “A” in the AGT encoding the methionine or initiation codon and is designated as +1. The cDNA sequence carrying a 957C>T polymorphism is shown in SEQ ID NO:2. A range of potential polymorphisms is shown in Table 2.

In a related aspect, the present invention provides a method for detecting the presence of, or the propensity to develop a neurological, psychiatric or psychological condition phenotype or state or sub-threshold form thereof, wherein the condition, phenotype or state results from or is exacerbated by any insertion or deletion in the DRD2 genetic locus including its 5′ or 3′ terminal regions, promoter, exons or introns. Insertions or deletions may involve a single nucleotide or more than one such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides within the region of interest.

In yet another aspect, the present invention provides a nonsense mutation which includes the introduction of a stop codon.

A neurological, psychiatric or psychological condition, phenotype or state or sub-threshold form thereof involving DRD2 or a risk of developing such a condition, phenotype or state may be ascertained by screening any tissue from an individual for genetic material carrying the DRD2 genetic locus for the presence of a polymorphism including a mutation which is associated with a particular neurological, psychiatric or psychological condition, phenotype or state or a sub-threshold form thereof or a pre-disposition for development of same. A 957C>T polymorphism on either or both alleles is one example of a genetic profile to be identified. Schizophrenia is an example of a particular neurological, psychiatric or psychological condition, phenotype or state. Most conveniently, blood is drawn and DNA extracted from the cells of the blood. In addition, prenatal diagnosis can be accomplished by testing fetal cells, placental cells or amniotic cells for a polymorphism in the DRD2 genetic locus.

Accordingly, another aspect of the present invention contemplates a method for diagnosing a neurological, psychiatric or psychological condition, phenotype or state in an individual, said method comprising obtaining or extracting DNA sample from cells of said individual and screening for or otherwise detecting the presence of a genetic profile in the DRD2 genetic locus including its 5′ or 3′ terminal region, promoter, intron or exons with a statistically significant association with a particular neurological, psychiatric or psychological condition, phenotype or state wherein the presence of that genetic profile is indicative of the neurological, psychiatric or psychological condition, phenotype or state or a sub-threshold form thereof or that the individual is at risk of developing same.

Generally, the genetic test is part of an overall diagnostic protocol involving psychological tests and certain behavioral analysis. Consequently, this aspect of the present invention may be considered as a confirmatory test or part of a series of tests in the final diagnosis of a neurological, psychiatric or psychological condition, phenotype or state.

Accordingly, another aspect of the present invention provides a diagnostic assay for a genetic profile predetermined to be associated with a particular neurological, psychiatric or psychological condition, phenotype or state said method comprising obtaining or extracting a DNA sample from cells of said individual and screening for or otherwise detecting the presence of a genetic profile in the DRD2 genetic locus including its 5′ or 3′ terminal region, promoter, intron or exons which has a statistically significant association with a particular neurological, psychiatric or psychological condition, phenotype or state wherein the presence of that genetic profile is indicative of the neurological, psychiatric or psychological condition, phenotype or state or a sub-threshold form thereof or that the individual is at risk of developing same.

As indicated above, the genetic profile is generally detecting a particular polymorphism or mutation within the DRD2 genetic locus or its 5′ or 3′ terminal regions, promoter, exons or introns. Any polymorphism or mutation such as those contemplated in Table 2 and which are found to be associated with a neurological, psychiatric or psychological condition, phenotype or state is encompassed by the present invention. In addition, examples of neurological, psychiatric or psychological conditions, phenotypes and states include but are not limited to Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder and Social Phobia.

Schizophrenia and alcoholism are particularly contemplated by the present invention as is the 957C>T polymorphism.

Accordingly, in a preferred embodiment, the present invention is directed to a method for diagnosing a neurological, psychiatric or psychological condition, phenotype or state including schizophrenia in an individual or a risk of development of same, said method comprising obtaining or extracting a DNA sample from cells of said individual and screening for or otherwise detecting the presence of a polymorphism at cDNA nucleotide position number 957 wherein the presence of a C at position 957 is indicative of the individual having or at risk of developing an adverse neurological, psychiatric or psychological condition, phenotype or state selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder and Social Phobia.

The method and assay of the present invention are further directed to detecting the form of the polymorphism in an individual associated with “normal” behavior. In other words, an individual which may be at risk such as through his or her genetic lines or because of substance abuse or who has behavioral tendencies which suggest a particular neurological, psychiatric or psychological condition, phenotype or state can be screened for the presence of a T at cDNA nucleotide number 957 wherein the presence of a T is at least suggestive of a non-genetic basis for any symptoms associated with the neurological, psychiatric or psychological condition, phenotype or state for which the individual first presented to a clinician.

Consequently, a “neurological, psychiatric or psychological condition, phenotype or state” may be an adverse condition or may represent “normal” behavior. The latter constitutes behavior consistent with societal “norms”.

Reference herein to an “individual” includes a human which may also be considered a subject, patient, host, recipient or target.

There are many methods which may be used to detect a DNA sequence profile. Direct DNA sequencing, either manual sequencing or automated fluorescent sequencing can detect sequence variation including a polymorphism or mutation. Another approach is the single-stranded conformation polymorphism assay (SSCP) (Orita, et al., Proc. Natl. Acad. Sci. USA. 86:2766-2770, 1989). This method does not detect all sequence changes, especially if the DNA fragment size is greater than 200 bp, but can be optimized to detect most DNA sequence variation. The reduced detection sensitivity is a disadvantage, but the increased throughput possible with SSCP makes it an attractive, viable alternative to direct sequencing for mutation detection. The fragments which have shifted mobility on SSCP gels are then sequenced to determine the exact nature of the DNA sequence variation. Other approaches based on the detection of mismatches between the two complementary DNA strands include clamped denaturing gel electrophoresis (CDGE) (Sheffield et al. Proc. Natl. Acad. Sci. USA 86:232-236, 1989), heteroduplex analysis (HA) (White et al. Genomics 12:301-306, 1992) and chemical mismatch cleavage (CMC) (Grompe et al. Proc. Natl. Acad. Sci. USA 86:5855-5892, 1989). None of the methods described above detects large deletions, duplications or insertions, nor will they detect a mutation in a regulatory region or a gene. Other methods which would detect these classes of mutations include a protein truncation assay or the asymmetric assay. A review of currently available methods of detecting DNA sequence variation can be found in Kwok (Curr Issues Mol. Biol. 5(2):43-60, 20030, Twyman and Primrose (Pharmacogenomics. 4(1):67-79, 2003), Edwards and Bartlett (Methods mol. Biol. 226:287-294, 2003) and Brennan (Am. J. Pharmacogenomics. 1(4):395-302, 2001). Once a mutation is known, an allele-specific detection approach such as allele-specific oligonucleotide (ASO) hybridization can be utilized to rapidly screen large numbers of other samples for that same mutation. Such a technique can utilize probes which are labelled with gold nanoparticles or any other reporter molecule to yield a visual color result (Elghanian et al. Science 277:1078-1081, 1997).

A rapid preliminary analysis to detect polymorphisms in DNA sequences can be performed by looking at a series of Southern blots of DNA cut with one or more restriction enzymes, preferably with a large number of restriction enzymes. Each blot contains a series of normal individuals and a series of individuals having neurologic or neuropsychiatric diseases or disorders or any other neurological, psychiatric or psychological condition, phenotype or state. Southern blots displaying hybridizing fragments (differing in length from control DNA when probed with sequences near or including the DRD2 genetic locus) indicate a possible mutation or polymorphism. If restriction enzymes which produce very large restriction fragments are used, then pulsed field gel electrophoresis (PFGE) is employed. Alternatively, the desired region of the DRD2 locus can be amplified, the resulting amplified products can be cut with a restriction enzyme and the size of fragments produced for the different polymorphisms can be determined.

Detection of point mutations may be accomplished by molecular cloning of the DRD2 alleles and sequencing the alleles using techniques well known in the art. Also, the gene or portions of the gene may be amplified, e.g., by PCR or other amplification technique, and the amplified gene or amplified portions of the gene may be sequenced.

Methods for a more complete, yet still indirect, test for confirming the presence of a susceptibility allele include: 1) single-stranded conformation analysis (SSCP) (Orita et al., 1989: supra); 2) denaturing gradient gel electrophoresis (DGGE) (Wartell et al. Nucl. Acids Res. 18:2699-2705, 1990; Sheffield et al. 1989 supra); 3) RNase protection assays (Finkelstein et al. Genomics 7:167-172, 1990; Kinszler et al. Science 251:1366-1370, 1991); 4) allele-specific oligonucleotides [ASOs] (Conner et al. Proc. Natl. Acad. Sci. USA 80:278-282, 1983); 5) the use of proteins which recognize nucleotide mismatches, such as the E. coli mutS protein (Modrich Ann. Rev. Genet. 25:229-253, 1991); 6) allele-specific PCR (Ruano and Kidd Nucl. Acids Res. 17:8392, 1989); and 7) PCR amplification of the site of the polymorphism followed by digestion using a restriction endonuclease that cuts or fails to cut when the variant allele is present.

Additionally, you could add real-time PCR such as the allele specific kinetic real-time PCR assay can be used or allele specific real-time TaqMan probes.

For allele-specific PCR, primers are used which hybridize at their 3′ ends to a particular DRD2 genetic locus polymorphism or mutation. If the particular polymorphism or mutation is not present, an amplification product is not observed. Amplification Refractory Mutation System (ARMS) can also be used, as disclosed in European Patent Application Publication No. 0332435. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening relatives of an affected individual for the presence of the mutation found in that individual. Other techniques for detecting insertions and deletions as known in the art can be used.

In SSCP, DGGE and the RNase protection assay, a electrophoretic band appears which is absent if the polymorphism or mutation is not present. SSCP detects a band which migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. In the practice of the present invention, the method involves the use of a labelled riboprobe which is complementary to the human wild-type DRD2 genetic locus. The riboprobe and either mRNA or DNA isolated from the person are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the mRNA or gene but can be a segment of either. If the riboprobe comprises only a segment of the mRNA or gene, it will be desirable to use a number of these probes to screen the whole mRNA sequence for mismatches.

In similar fashion, DNA probes can be used to detect mismatches, through enzymatic or chemical cleavage (see, for example, Cotton et al. Proc. Natl. Acad. Sci. USA 87:4033-40371988; Shenk et al. Proc. Natl. Acad. Sci. USA 72:989-993, 1975; Novack et al. Proc. Natl. Acad. Sci. USA 83:586-590, 1986). Alternatively, mismatches can be detected by shifts in the electrophoretic mobility of mismatched duplexes relative to matched duplexes (see, for example, Cariello Am. J. Human Genetics 42:726-734, 1988). With either riboprobes or DNA probes, the cellular mRNA or DNA which might contain a mutation can be amplified using PCR (see below) before hybridization. Changes in DNA of the DRD2 genetic locus can also be detected using Southern blot hybridization, especially if the changes are gross rearrangements, such as deletions and insertions.

DNA sequences of the DRD2 gene which have been amplified by use of PCR may also be screened using allele-specific probes. These probes are nucleic acid oligomers, each of which contains a region of the gene sequence harboring a known mutation. For example, one oligomer may be from about 3 to about 100 nucleotides in length such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100. An oligomer of about 20 nucleotides in length is particularly convenient. These oligomers correspond to a portion of the gene sequence. By use of a battery of such allele-specific probes, PCR amplification products can be screened to identify the presence of a previously identified mutation in the gene. Hybridization of allele-specific probes with amplified DRD2 genetic sequences can be performed, for example, on a nylon filter. Hybridization to a particular probe under high stringency hybridization conditions indicates the presence of the same mutation in the tissue as in the allele-specific probe.

Once the site containing the polymorphisms has been amplified, the SNPs can also be detected by primer extension. Here a primer is annealed immediately adjacent to the variant site, and the 5′ end is extended a single base pair by incubation with di-deoxytrinucleotides. Whether the extended base was a A, T, G or C can then be determined by mass spectrometry (MALDI-TOF) or fluorescent flow cytometric analysis (Taylor et al. Biotechniques 30:661-669, 2001) or other techniques.

Nucleic acid analysis via microchip technology is also applicable to the present invention. In this technique, thousands of distinct oligonucleotide probes are built up in an array on a silicon chip. Nucleic acids to be analyzed are fluorescently labeled and hybridized to the probes on the chip. It is also possible to study nucleic acid-protein interactions using these nucleic acid microchips. Using this technique, one can determine the presence of mutations or even sequence the nucleic acid being analyzed or one can measure expression levels of a gene of interest. The method is one of parallel processing of many, including thousands, of probes at once and can tremendously increase the rate of analysis.

The most definitive test for mutations in the DRD2 genetic locus is to directly compare genomic DRD2 sequences from patients with those from a control population. Alternatively, one can sequence mRNA after amplification, e.g., by PCR, thereby eliminating the necessity of determining the exon structure of the candidate gene.

Mutations falling outside the coding region of DRD2 can be detected by examining the non-coding regions, such as introns and regulatory sequences near or within the genes. An early indication that mutations in non-coding regions are important may come from Northern blot experiments that reveal messenger RNA molecules of abnormal size or abundance in patients as compared to those of control individuals.

Alteration of mRNA expression from the DRD2 genetic locus can be detected by any techniques known in the art. These include Northern blot analysis, PCR amplification and RNase protection. Diminished mRNA expression indicates an alteration of the wild-type gene. Alteration of wild-type genes can also be detected by screening for alteration of wild-type protein. For example, monoclonal antibodies immunoreactive with DRD2 can be used to screen a tissue. Lack of cognate antigen or a reduction in the levels of antigen would indicate a mutation. Antibodies specific for products of mutant alleles could also be used to detect mutant gene product. Such immunological assays can be done in any convenient formats known in the art. These include Western blots, immunohistochemical assays and ELISA assays. Any means for detecting an altered protein can be used to detect alteration of the wild-type DRD2 gene. Functional assays, such as protein binding determinations, can be used. In addition, assays can be used which detect DRD2 biochemical function. Finding a mutant DRD2 gene product indicates alteration of a wild-type DRD2 gene.

A mutant DRD2 gene or corresponding gene products can also be detected in other human body samples which contain DNA, such as serum, stool, urine and sputum. The same techniques discussed above for detection of mutant genes or gene products in tissues can be applied to other body samples. By screening such body samples, an early diagnosis can be achieved for subjects at risk of developing a particular neurological, psychiatric or psychological condition, phenotype or state or sub-threshold forms thereof.

The primer pairs of the present invention are useful for determination of the nucleotide sequence of a particular DRD2 allele using PCR. The pairs of single-stranded DNA primers can be annealed to sequences within or surrounding the gene in order to prime amplifying DNA synthesis of the gene itself. A complete set of these primers allows synthesis of all of the nucleotides of the gene coding sequences, i.e., the exons. The set of primers preferably allows synthesis of both intron and exon sequences. Allele-specific primers can also be used. Such primers anneal only to particular DRD2 polymorphic or mutant alleles, and thus will only amplify a product in the presence of the polymorphic or mutant allele as a template.

In order to facilitate subsequent cloning of amplified sequences, primers may have restriction enzyme site sequences appended to their 5′ ends. Thus, all nucleotides of the primers are derived from the gene sequence or sequences adjacent the gene, except for the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Given the sequence of each gene and polymorphisms described herein, design of particular primers is well within the skill of the art. The present invention adds to this by presenting data on the intron/exon boundaries thereby allowing one to design primers to amplify and sequence all of the exonic regions completely.

The nucleic acid probes provided by the present invention are useful for a number of purposes. They can be used in Southern blot hybridization to genomic DNA and in the RNase protection method for detecting point mutations already discussed above. The probes can be used to detect PCR amplification products. They may also be used to detect mismatches with the DRD2 gene or mRNA using other techniques.

The present invention identifies the presence of an altered (or a mutant) DRD2 genetic locus associated with a neurological, psychiatric or psychological condition, phenotype or state, including schizophrenia or a sub-threshold form thereof or an individual of risk of developing same. In order to detect a DRD2 gene polymorphism or mutation, a biological sample is prepared and analyzed for a difference between the sequence of the allele being analyzed and the sequence of the “wild-type” allele. In this context, a “wild-type” allele includes the nucleotide at a given position most commonly represented in the population and for which there is not direct evidence for these individuals having the neurological, psychiatric or psychological condition, phenotype or state under investigation. Polymorphic or mutant alleles can be initially identified by any of the techniques described above. The polymorphic or mutant alleles may then be sequenced to identify the specific polymorphism or mutation of the particular allele. Alternatively, polymorphic or mutant alleles can be initially identified by identifying polymorphic or mutant (altered) proteins, using conventional techniques. The polymorphisms or mutations, especially those statistically associated with a neurological, psychiatric or psychological condition, phenotype or state or a sub-threshold form thereof are then used for the diagnostic and prognostic methods of the present invention.

As used herein, the phrase “amplifying” refers to increasing the content of a specific genetic region of interest within a sample. The amplification of the genetic region of interest may be performed using any method of amplification known to those of skill in the relevant art. In a preferred aspect, the present method for detecting a polymorphism utilizes PCR as the amplification step.

PCR amplification utilizes primers to amplify a genetic region of interest. Reference herein to a “primer” is not to be taken as any limitation to structure, size or function. Reference to primers herein, includes reference to a sequence of deoxyribonucleotides comprising at least 3 nucleotides. Generally, the primers comprises from about 3 to about 100 nucleotides, preferably from about 5 to about 50 nucleotides and even more preferably from about 10 to about 25 nucleotides such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. The primers of the present invention may be synthetically produced by, for example, the stepwise addition of nucleotides or may be fragments, parts or portions or extension products of other nucleic acid molecules. The term “primer” is used in its most general sense to include any length of nucleotides which, when used for amplification purposes, can provide free 3′ hydroxyl group for the initiation of DNA synthesis by a DNA polymerase. DNA synthesis results in the extension of the primer to produce a primer extension product complementary to the nucleic acid strand to which the primer has annealed or hybridized.

Accordingly, the present invention extends to an isolated oligonucleotide which comprises from about 3 to about 100 consecutive nucleotides from the DRD2 genetic locus and which encompass at least one polymorphism or mutation associated with or otherwise likely to be found in individuals with a particular neurological, psychiatric or psychological condition, phenotype or state such as those selected from normal behavior, Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder and Social Phobia.

Schizophrenia and alcoholism are considered as particular examples of a neurological, psychiatric or psychological condition, phenotype or state. Insofar as the oligonucleotide primers seek to identify a polymorphism at position 957 (using cDNA numbering) of the DRD2 gene, then the preferred oligonucleotides are defined by SEQ ID NO:4 (C957) or SEQ ID NO:5 (T957). A convenient reverse primer includes SEQ ID NO:6.

However, the present invention extends to any oligomeric which encompasses a polymorphism within the DRD2 genetic locus.

Examples of these from the DRD2 cDNA include the following or their complementary forms:

| | |
|---|---|
| (SEQ ID NO: 8) | ggcagccgtc cggggccgcc |
| (SEQ ID NO: 9) | gcagccgtc cggggccgcc a |
| (SEQ ID NO: 10) | cagccgtc cggggccgcc ac |
| (SEQ ID NO: 11) | agccgtc cggggccgcc act |
| (SEQ ID NO: 12) | gccgtc cggggccgcc actc |
| (SEQ ID NO: 13) | ccgtc cggggccgcc actct |
| (SEQ ID NO: 14) | cgtc cggggccgcc actctc |
| (SEQ ID NO: 15) | gtc cggggccgcc actctcc |
| (SEQ ID NO: 16) | tc cggggccgcc actctcct |
| (SEQ ID NO: 17) | c cggggccgcc actctcctc |
| (SEQ ID NO: 18) | cggggccgcc actctcctcg |
| (SEQ ID NO: 19) | ggggccgcc actctcctcg g |
| (SEQ ID NO: 20) | gggccgcc actctcctcg gc |
| (SEQ ID NO: 21) | ggccgcc actctcctcg gcc |
| (SEQ ID NO: 22) | gccgcc actctcctcg gccg |
| (SEQ ID NO: 23) | ccgcc actctcctcg gccgg |
| (SEQ ID NO: 24) | gcc actctcctcg gccggtc |
| (SEQ ID NO: 25) | cc actctcctcg gccggtcc |
| (SEQ ID NO: 26) | c actctcctcg gccggtccc |
| (SEQ ID NO: 27) | actctcctcg gccggtccct |
| (SEQ ID NO: 28) | ctctcctcg gccggtccct g |
| (SEQ ID NO: 29) | tctcctcg gccggtccct gg |
| (SEQ ID NO: 30) | ctcctcg gccggtccct ggc |
| (SEQ ID NO: 31) | tcctcg gccggtccct ggct |
| (SEQ ID NO: 32) | cctcg gccggtccct ggctc |
| (SEQ ID NO: 33) | ctcg gccggtccct ggctcc |
| (SEQ ID NO: 34) | tcg gccggtccct ggctccc |
| (SEQ ID NO: 35) | cg gccggtccct ggctcccg |
| (SEQ ID NO: 36) | g gccggtccct ggctcccgg |
| (SEQ ID NO: 37) | gccggtccct ggctcccgga |
| (SEQ ID NO: 38) | ccggtccct ggctcccgga g |
| (SEQ ID NO: 39) | cggtccct ggctcccgga gg |
| (SEQ ID NO: 40) | ggtccct ggctcccgga ggc |
| (SEQ ID NO: 41) | gtccct ggctcccgga ggcg |
| (SEQ ID NO: 42) | tccct ggctcccgga ggcgg |
| (SEQ ID NO: 43) | ccct ggctcccgga ggcggc |
| (SEQ ID NO: 44) | cct ggctcccgga ggcggcc |
| (SEQ ID NO: 45) | ct ggctcccgga ggcggccg |
| (SEQ ID NO: 46) | t ggctcccgga ggcggccgc |
| (SEQ ID NO: 47) | ggctcccgga ggcggccgcg |
| (SEQ ID NO: 48) | gctcccgga ggcggccgcg c |
| (SEQ ID NO: 49) | ctcccgga ggcggccgcg cg |
| (SEQ ID NO: 50) | tcccgga ggcggccgcg cgt |
| (SEQ ID NO: 51) | cccgga ggcggccgcg cgtg |
| (SEQ ID NO: 52) | ccgga ggcggccgcg cgtgg |
| (SEQ ID NO: 53) | cgga ggcggccgcg cgtgga |
| (SEQ ID NO: 54) | gga ggcggccgcg cgtggat |
| (SEQ ID NO: 55) | ga ggcggccgcg cgtggatg |
| (SEQ ID NO: 56) | a ggcggccgcg cgtggatgc |
| (SEQ ID NO: 57) | ggcggccgcg cgtggatgcg |
| (SEQ ID NO: 58) | gcggccgcg cgtggatgcg g |
| (SEQ ID NO: 59) | cggccgcg cgtggatgcg gc |
| (SEQ ID NO: 60) | ggccgcg cgtggatgcg gcg |
| (SEQ ID NO: 61) | gccgcg cgtggatgcg gcgg |
| (SEQ ID NO: 62) | ccgcg cgtggatgcg gcggg |
| (SEQ ID NO: 63) | cgcg cgtggatgcg gcggga |
| (SEQ ID NO: 64) | gcg cgtggatgcg gcgggag |
| (SEQ ID NO: 65) | cg cgtggatgcg gcgggagc |
| (SEQ ID NO: 66) | g cgtggatgcg gcgggagct |
| (SEQ ID NO: 67) | cgtggatgcg gcgggagctg |
| (SEQ ID NO: 68) | gtggatgcg gcgggagctg g |
| (SEQ ID NO: 69) | tggatgcg gcgggagctg ga |
| (SEQ ID NO: 70) | ggatgcg gcgggagctg gaa |
| (SEQ ID NO: 71) | gatgcg gcgggagctg gaag |
| (SEQ ID NO: 72) | atgcg gcgggagctg gaagc |
| (SEQ ID NO: 73) | tgcg gcgggagctg gaagcc |
| (SEQ ID NO: 74) | gcg gcgggagctg gaagcct |
| (SEQ ID NO: 75) | cg gcgggagctg gaagcctc |
| (SEQ ID NO: 76) | g gcgggagctg gaagcctca |
| (SEQ ID NO: 77) | gcgggagctg gaagcctcaa |
| (SEQ ID NO: 78) | cgggagctg gaagcctcaa g |
| (SEQ ID NO: 79) | gggagctg gaagcctcaa gc |
| (SEQ ID NO: 80) | ggagctg gaagcctcaa gca |
| (SEQ ID NO: 81) | gagctg gaagcctcaa gcag |
| (SEQ ID NO: 82) | agctg gaagcctcaa gcagc |
| (SEQ ID NO: 83) | gctg gaagcctcaa gcagcc |
| (SEQ ID NO: 84) | ctg gaagcctcaa gcagccg |
| (SEQ ID NO: 85) | tg gaagcctcaa gcagccgg |
| (SEQ ID NO: 86) | g gaagcctcaa gcagccggc |
| (SEQ ID NO: 87) | gaagcctcaa gcagccggcg |
| (SEQ ID NO: 88) | aagcctcaa gcagccggcg c |
| (SEQ ID NO: 89) | agcctcaa gcagccggcg cc |
| (SEQ ID NO: 90) | gcctcaa gcagccggcg ccg |
| (SEQ ID NO: 91) | cctcaa gcagccggcg ccgt |
| (SEQ ID NO: 92) | ctcaa gcagccggcg ccgtc |
| (SEQ ID NO: 93) | tcaa gcagccggcg ccgtct |
| (SEQ ID NO: 94) | caa gcagccggcg ccgtctc |
| (SEQ ID NO: 95) | aa gcagccggcg ccgtctct |
| (SEQ ID NO: 96) | a gcagccggcg ccgtctctg |
| (SEQ ID NO: 97) | gcagccggcg ccgtctctgc |
| (SEQ ID NO: 98) | cagccggcg ccgtctctgc c |
| (SEQ ID NO: 99) | agccggcg ccgtctctgc cc |
| (SEQ ID NO: 100) | gccggcg ccgtctctgc ccc |
| (SEQ ID NO: 101) | ccggcg ccgtctctgc cccg |
| (SEQ ID NO: 102) | cggcg ccgtctctgc cccgg |
| (SEQ ID NO: 103) | ggcg ccgtctctgc cccggg |
| (SEQ ID NO: 104) | gcg ccgtctctgc cccgggg |
| (SEQ ID NO: 105) | cg ccgtctctgc cccggggc |
| (SEQ ID NO: 106) | g ccgtctctgc cccggggcg |
| (SEQ ID NO: 107) | ccgtctctgc cccggggcgc |
| (SEQ ID NO: 108) | cgtctctgc cccggggcgc c |
| (SEQ ID NO: 109) | gtctctgc cccggggcgc cc |
| (SEQ ID NO: 110) | tctctgc cccggggcgc cct |
| (SEQ ID NO: 111) | ctctgc cccggggcgc ccta |
| (SEQ ID NO: 112) | tctgc cccggggcgc cctat |
| (SEQ ID NO: 113) | ctgc cccggggcgc cctatg |
| (SEQ ID NO: 114) | tgc cccggggcgc cctatgg |
| (SEQ ID NO: 115) | gc cccggggcgc cctatggc |
| (SEQ ID NO: 116) | c cccggggcgc cctatggct |
| (SEQ ID NO: 117) | cccggggcgc cctatggctt |
| (SEQ ID NO: 118) | ccggggcgc cctatggctt g |
| (SEQ ID NO: 119) | cggggcgc cctatggctt ga |
| (SEQ ID NO: 120) | ggggcgc cctatggctt gaa |
| (SEQ ID NO: 121) | gggcgc cctatggctt gaag |
| (SEQ ID NO: 122) | ggcgc cctatggctt gaaga |
| (SEQ ID NO: 123) | gcgc cctatggctt gaagag |
| (SEQ ID NO: 124) | cgc cctatggctt gaagagc |
| (SEQ ID NO: 125) | gc cctatggctt gaagagcc |
| (SEQ ID NO: 126) | c cctatggctt gaagagcct |
| (SEQ ID NO: 127) | cctatggctt gaagagcctg |
| (SEQ ID NO: 128) | ctatggctt gaagagcctg g |
| (SEQ ID NO: 129) | tatggctt gaagagcctg gc |
| (SEQ ID NO: 130) | atggctt gaagagcctg gcc |
| (SEQ ID NO: 131) | tggctt gaagagcctg gcca |
| (SEQ ID NO: 132) | ggctt gaagagcctg gccac |
| (SEQ ID NO: 133) | gctt gaagagcctg gccacc |
| (SEQ ID NO: 134) | ctt gaagagcctg gccaccc |
| (SEQ ID NO: 135) | tt gaagagcctg gccaccca |
| (SEQ ID NO: 136) | t gaagagcctg gccacccag |
| (SEQ ID NO: 137) | gaagagcctg gccacccagt |
| (SEQ ID NO: 138) | aagagcctg gccacccagt g |
| (SEQ ID NO: 139) | agagcctg gccacccagt gg |
| (SEQ ID NO: 140) | gagcctg gccacccagt ggc |
| (SEQ ID NO: 141) | gcctg gccacccagt ggct |
| (SEQ ID NO: 142) | cctg gccacccagt ggctc |
| (SEQ ID NO: 143) | ctg gccacccagt ggctcc |
| (SEQ ID NO: 144) | tg gccacccagt ggctccac |
| (SEQ ID NO: 145) | g gccacccagt ggctccacc |
| (SEQ ID NO: 146) | gccacccagt ggctccaccg |
| (SEQ ID NO: 147) | ccacccagt ggctccaccg c |
| (SEQ ID NO: 148) | cacccagt ggctccaccg cc |
| (SEQ ID NO: 149) | acccagt ggctccaccg ccc |
| (SEQ ID NO: 150) | cccagt ggctccaccg ccct |
| (SEQ ID NO: 151) | ccagt ggctccaccg ccctg |
| (SEQ ID NO: 152) | cagt ggctccaccg ccctga |
| (SEQ ID NO: 153) | agt ggctccaccg ccctgat |
| (SEQ ID NO: 154) | gt ggctccaccg ccctgatg |
| (SEQ ID NO: 155) | t ggctccaccg ccctgatgg |
| (SEQ ID NO: 156) | ggctccaccg ccctgatgga |
| (SEQ ID NO: 157) | gctccaccg ccctgatgga t |
| (SEQ ID NO: 158) | ctccaccg ccctgatgga tc |
| (SEQ ID NO: 159) | tccaccg ccctgatgga tcc |
| (SEQ ID NO: 160) | ccaccg ccctgatgga tcca |
| (SEQ ID NO: 161) | caccg ccctgatgga tccac |
| (SEQ ID NO: 162) | accg ccctgatgga tccact |
| (SEQ ID NO: 163) | ccg ccctgatgga tccactg |
| (SEQ ID NO: 164) | cg ccctgatgga tccactga |
| (SEQ ID NO: 165) | g ccctgatgga tccactgaa |
| (SEQ ID NO: 166) | ccctgatgga tccactgaat |
| (SEQ ID NO: 167) | cctgatgga tccactgaat c |
| (SEQ ID NO: 168) | ctgatgga tccactgaat ct |
| (SEQ ID NO: 169) | tgatgga tccactgaat ctg |
| (SEQ ID NO: 170) | gatgga tccactgaat ctgt |
| (SEQ ID NO: 171) | atgga tccactgaat ctgtc |
| (SEQ ID NO: 172) | tgga tccactgaat ctgtcc |
| (SEQ ID NO: 173) | gga tccactgaat ctgtcct |
| (SEQ ID NO: 174) | ga tccactgaat ctgtcctg |
| (SEQ ID NO: 175) | a tccactgaat ctgtcctgg |
| (SEQ ID NO: 176) | tccactgaat ctgtcctggt |
| (SEQ ID NO: 177) | ccactgaat ctgtcctggt a |
| (SEQ ID NO: 178) | cactgaat ctgtcctggt at |
| (SEQ ID NO: 179) | actgaat ctgtcctggt atg |
| (SEQ ID NO: 180) | ctgaat ctgtcctggt atga |
| (SEQ ID NO: 181) | tgaat ctgtcctggt atgat |
| (SEQ ID NO: 182) | gaat ctgtcctggt atgatg |
| (SEQ ID NO: 183) | aat ctgtcctggt atgatga |
| (SEQ ID NO: 184) | at ctgtcctggt atgatgat |
| (SEQ ID NO: 185) | t ctgtcctggt atgatgatg |
| (SEQ ID NO: 186) | ctgtcctggt atgatgatga |
| (SEQ ID NO: 187) | tgtcctggt atgatgatga t |
| (SEQ ID NO: 188) | gtcctggt atgatgatga tc |
| (SEQ ID NO: 189) | tcctggt atgatgatga tct |
| (SEQ ID NO: 190) | cctggt atgatgatga tctg |
| (SEQ ID NO: 191) | ctggt atgatgatga tctgg |
| (SEQ ID NO: 192) | tggt atgatgatga tctgga |
| (SEQ ID NO: 193) | ggt atgatgatga tctggag |
| (SEQ ID NO: 194) | gt atgatgatga tctggaga |
| (SEQ ID NO: 195) | t atgatgatga tctggagag |
| (SEQ ID NO: 196) | atgatgatga tctggagagg |
| (SEQ ID NO: 197) | tgatgatga tctggagagg c |
| (SEQ ID NO: 198) | gatgatga tctggagagg ca |
| (SEQ ID NO: 199) | atgatga tctggagagg cag |
| (SEQ ID NO: 200) | tgatga tctggagagg caga |
| (SEQ ID NO: 201) | gatga tctggagagg cagaa |
| (SEQ ID NO: 202) | atga tctggagagg cagaac |
| (SEQ ID NO: 203) | tga tctggagagg cagaact |
| (SEQ ID NO: 204) | ga tctggagagg cagaactg |
| (SEQ ID NO: 205) | a tctggagagg cagaactgg |
| (SEQ ID NO: 206) | tctggagagg cagaactgga |
| (SEQ ID NO: 207) | ctggagagg cagaactgga g |
| (SEQ ID NO: 208) | tggagagg cagaactgga gc |
| (SEQ ID NO: 209) | ggagagg cagaactgga gcc |
| (SEQ ID NO: 210) | gagagg cagaactgga gccg |
| (SEQ ID NO: 211) | agagg cagaactgga gccgg |
| (SEQ ID NO: 212) | gagg cagaactgga gccggc |
| (SEQ ID NO: 213) | agg cagaactgga gccggcc |
| (SEQ ID NO: 214) | gg cagaactgga gccggccc |
| (SEQ ID NO: 215) | g cagaactgga gccggccct |
| (SEQ ID NO: 216) | cagaactgga gccggccctt |
| (SEQ ID NO: 217) | agaactgga gccggccctt c |
| (SEQ ID NO: 218) | gaactgga gccggccctt ca |
| (SEQ ID NO: 219) | aactgga gccggccctt caa |
| (SEQ ID NO: 220) | actgga gccggccctt caac |
| (SEQ ID NO: 221) | ctgga gccggccctt caacg |
| (SEQ ID NO: 222) | tgga gccggccctt caacgg |
| (SEQ ID NO: 223) | gga gccggccctt caacggg |
| (SEQ ID NO: 224) | ga gccggccctt caacgggt |
| (SEQ ID NO: 225) | a gccggccctt caacgggtc |
| (SEQ ID NO: 226) | gccggccctt caacgggtca |
| (SEQ ID NO: 227) | ccggccctt caacgggtca g |
| (SEQ ID NO: 228) | cggccctt caacgggtca ga |
| (SEQ ID NO: 229) | ggccctt caacgggtca gac |
| (SEQ ID NO: 230) | gccctt caacgggtca gacg |
| (SEQ ID NO: 231) | ccctt caacgggtca gacgg |
| (SEQ ID NO: 232) | cctt caacgggtca gacggg |
| (SEQ ID NO: 233) | ctt caacgggtca gacggga |
| (SEQ ID NO: 234) | tt caacgggtca gacgggaa |
| (SEQ ID NO: 235) | t caacgggtca gacgggaag |
| (SEQ ID NO: 236) | caacgggtca gacgggaagg |
| (SEQ ID NO: 237) | aacgggtca gacgggaagg c |
| (SEQ ID NO: 238) | acgggtca gacgggaagg cg |
| (SEQ ID NO: 239) | cgggtca gacgggaagg cgg |
| (SEQ ID NO: 240) | gggtca gacgggaagg cgga |
| (SEQ ID NO: 241) | ggtca gacgggaagg cggac |
| (SEQ ID NO: 242) | gtca gacgggaagg cggaca |
| (SEQ ID NO: 243) | tca gacgggaagg cggacag |
| (SEQ ID NO: 244) | ca gacgggaagg cggacaga |
| (SEQ ID NO: 245) | a gacgggaagg cggacagac |
| (SEQ ID NO: 246) | gacgggaagg cggacagacc |
| (SEQ ID NO: 247) | acgggaagg cggacagacc c |
| (SEQ ID NO: 248) | cgggaagg cggacagacc cc |
| (SEQ ID NO: 249) | gggaagg cggacagacc cca |
| (SEQ ID NO: 250) | ggaagg cggacagacc ccac |
| (SEQ ID NO: 251) | gaagg cggacagacc ccact |
| (SEQ ID NO: 252) | aagg cggacagacc ccacta |
| (SEQ ID NO: 253) | agg cggacagacc ccactac |
| (SEQ ID NO: 254) | gg cggacagacc ccactaca |
| (SEQ ID NO: 255) | g cggacagacc ccactacaa |
| (SEQ ID NO: 256) | cggacagacc ccactacaac |
| (SEQ ID NO: 257) | ggacagacc ccactacaac t |
| (SEQ ID NO: 258) | gacagacc ccactacaac ta |
| (SEQ ID NO: 259) | acagacc ccactacaac tac |
| (SEQ ID NO: 260) | cagacc ccactacaac tact |
| (SEQ ID NO: 261) | agacc ccactacaac tacta |
| (SEQ ID NO: 262) | gacc ccactacaac tactat |
| (SEQ ID NO: 263) | acc ccactacaac tactatg |
| (SEQ ID NO: 264) | cc ccactacaac tactatgc |
| (SEQ ID NO: 265) | c ccactacaac tactatgcc |
| (SEQ ID NO: 266) | ccactacaac tactatgcca |
| (SEQ ID NO: 267) | cactacaac tactatgcca c |
| (SEQ ID NO: 268) | actacaac tactatgcca ca |
| (SEQ ID NO: 269) | ctacaac tactatgcca cac |
| (SEQ ID NO: 270) | tacaac tactatgcca cact |
| (SEQ ID NO: 271) | acaac tactatgcca cactg |
| (SEQ ID NO: 272) | caac tactatgcca cactgc |
| (SEQ ID NO: 273) | aac tactatgcca cactgct |
| (SEQ ID NO: 274) | ac tactatgcca cactgctc |
| (SEQ ID NO: 275) | c tactatgcca cactgctca |
| (SEQ ID NO: 276) | tactatgcca cactgctcac |
| (SEQ ID NO: 277) | actatgcca cactgctcac c |
| (SEQ ID NO: 278) | ctatgcca cactgctcac cc |
| (SEQ ID NO: 279) | tatgcca cactgctcac cct |
| (SEQ ID NO: 280) | atgcca cactgctcac cctg |
| (SEQ ID NO: 281) | tgcca cactgctcac cctgc |
| (SEQ ID NO: 282) | gcca cactgctcac cctgct |
| (SEQ ID NO: 283) | cca cactgctcac cctgctc |
| (SEQ ID NO: 284) | ca cactgctcac cctgctca |
| (SEQ ID NO: 285) | a cactgctcac cctgctcatc |
| (SEQ ID NO: 286) | cactgctcac cctgctcatc |
| (SEQ ID NO: 287) | actgctcac cctgctcatc g |
| (SEQ ID NO: 288) | ctgctcac cctgctcatc gc |
| (SEQ ID NO: 289) | tgctcac cctgctcatc gct |
| (SEQ ID NO: 290) | gctcac cctgctcatc gctg |
| (SEQ ID NO: 291) | ctcac cctgctcatc gctgt |
| (SEQ ID NO: 292) | tcac cctgctcatc gctgtc |
| (SEQ ID NO: 293) | cac cctgctcatc gctgtca |
| (SEQ ID NO: 294) | ac cctgctcatc gctgtcat |
| (SEQ ID NO: 295) | c cctgctcatc gctgtcatc |
| (SEQ ID NO: 296) | cctgctcatc gctgtcatcg |
| (SEQ ID NO: 297) | ctgctcatc gctgtcatcg t |
| (SEQ ID NO: 298) | tgctcatc gctgtcatcg tc |
| (SEQ ID NO: 299) | gctcatc gctgtcatcg tct |
| (SEQ ID NO: 300) | ctcatc gctgtcatcg tctt |
| (SEQ ID NO: 301) | tcatc gctgtcatcg tcttc |
| (SEQ ID NO: 302) | catc gctgtcatcg tcttcg |
| (SEQ ID NO: 303) | atc gctgtcatcg tcttcgg |
| (SEQ ID NO: 304) | tc gctgtcatcg tcttcggc |
| (SEQ ID NO: 305) | c gctgtcatcg tcttcggca |
| (SEQ ID NO: 306) | gctgtcatcg tcttcggcaa |
| (SEQ ID NO: 307) | ctgtcatcg tcttcggcaa c |
| (SEQ ID NO: 308) | tgtcatcg tcttcggcaa cg |
| (SEQ ID NO: 309) | gtcatcg tcttcggcaa cgt |
| (SEQ ID NO: 310) | tcatcg tcttcggcaa cgtg |
| (SEQ ID NO: 311) | catcg tcttcggcaa cgtgc |
| (SEQ ID NO: 312) | atcg tcttcggcaa cgtgct |
| (SEQ ID NO: 313) | tcg tcttcggcaa cgtgctg |
| (SEQ ID NO: 314) | cg tcttcggcaa cgtgctgg |
| (SEQ ID NO: 315) | g tcttcggcaa cgtgctggt |
| (SEQ ID NO: 316) | tcttcggcaa cgtgctggtg |
| (SEQ ID NO: 317) | cttcggcaa cgtgctggtg t |
| (SEQ ID NO: 318) | ttcggcaa cgtgctggtg tg |
| (SEQ ID NO: 319) | tcggcaa cgtgctggtg tgc |
| (SEQ ID NO: 320) | cggcaa cgtgctggtg tgca |
| (SEQ ID NO: 321) | ggcaa cgtgctggtg tgcat |
| (SEQ ID NO: 322) | gcaa cgtgctggtg tgcatg |
| (SEQ ID NO: 323) | caa cgtgctggtg tgcatgg |
| (SEQ ID NO: 324) | aa cgtgctggtg tgcatggc |
| (SEQ ID NO: 325) | a cgtgctggtg tgcatggct |
| (SEQ ID NO: 326) | cgtgctggtg tgcatggctg |
| (SEQ ID NO: 327) | tgctggtg tgcatggctg t |
| (SEQ ID NO: 328) | tgctggtg tgcatggctg tg |
| (SEQ ID NO: 329) | gctggtg tgcatggctg tgt |
| (SEQ ID NO: 330) | ctggtg tgcatggctg tgtc |
| (SEQ ID NO: 331) | tggtg tgcatggctg tgtcc |
| (SEQ ID NO: 332) | ggtg tgcatggctg tgtccc |
| (SEQ ID NO: 333) | gtg tgcatggctg tgtcccg |
| (SEQ ID NO: 334) | tg tgcatggctg tgtcccgc |
| (SEQ ID NO: 335) | g tgcatggctg tgtcccgcg |
| (SEQ ID NO: 336) | tgcatggctg tgtcccgcga |
| (SEQ ID NO: 337) | gcatggctg tgtcccgcga g |
| (SEQ ID NO: 338) | catggctg tgtcccgcga ga |
| (SEQ ID NO: 339) | atggctg tgtcccgcga gaa |
| (SEQ ID NO: 340) | tggctg tgtcccgcga gaag |
| (SEQ ID NO: 341) | ggctg tgtcccgcga gaagg |
| (SEQ ID NO: 342) | gctg tgtcccgcga gaaggc |
| (SEQ ID NO: 343) | ctg tgtcccgcga gaaggcg |
| (SEQ ID NO: 344) | tg tgtcccgcga gaaggcgc |
| (SEQ ID NO: 345) | g tgtcccgcga gaaggcgct |
| (SEQ ID NO: 346) | tgtcccgcga gaaggcgctg |
| (SEQ ID NO: 347) | gtcccgcga gaaggcgctg c |
| (SEQ ID NO: 348) | tcccgcga gaaggcgctg ca |
| (SEQ ID NO: 349) | cccgcga gaaggcgctg cag |
| (SEQ ID NO: 350) | ccgcga gaaggcgctg caga |
| (SEQ ID NO: 351) | cgcga gaaggcgctg cagac |
| (SEQ ID NO: 352) | gcga gaaggcgctg cagacc |
| (SEQ ID NO: 353) | cga gaaggcgctg cagacca |
| (SEQ ID NO: 354) | ga gaaggcgctg cagaccac |
| (SEQ ID NO: 355) | a gaaggcgctg cagaccacc |
| (SEQ ID NO: 356) | gaaggcgctg cagaccacca |
| (SEQ ID NO: 357) | aaggcgctg cagaccacca c |
| (SEQ ID NO: 358) | aggcgctg cagaccacca cc |
| (SEQ ID NO: 359) | ggcgctg cagaccacca cca |
| (SEQ ID NO: 360) | gcgatg cagaccacca ccaa |
| (SEQ ID NO: 361) | cgctg cagaccacca ccaac |
| (SEQ ID NO: 362) | gctg cagaccacca ccaact |
| (SEQ ID NO: 363) | ctg cagaccacca ccaacta |
| (SEQ ID NO: 364) | tg cagaccacca ccaactac |
| (SEQ ID NO: 365) | g cagaccacca ccaactacc |
| (SEQ ID NO: 366) | cagaccacca ccaactacct |
| (SEQ ID NO: 367) | agaccacca ccaactacct g |
| (SEQ ID NO: 368) | gaccacca ccaactacct ga |
| (SEQ ID NO: 369) | accacca ccaactacct gat |
| (SEQ ID NO: 370) | ccacca ccaactacct gatc |
| (SEQ ID NO: 371) | cacca ccaactacct gatcg |
| (SEQ ID NO: 372) | acca ccaactacct gatcgt |
| (SEQ ID NO: 373) | cca ccaactacct gatcgtc |
| (SEQ ID NO: 374) | ca ccaactacct gatcgtca |
| (SEQ ID NO: 375) | a ccaactacct gatcgtcag |
| (SEQ ID NO: 376) | ccaactacct gatcgtcagc |
| (SEQ ID NO: 377) | caactacct gatcgtcagc c |
| (SEQ ID NO: 378) | aactacct gatcgtcagc ct |
| (SEQ ID NO: 379) | actacct gatcgtcagc ctc |
| (SEQ ID NO: 380) | ctacct gatcgtcagc ctcg |
| (SEQ ID NO: 381) | tacct gatcgtcagc ctcgc |
| (SEQ ID NO: 382) | acct gatcgtcagc ctcgca |
| (SEQ ID NO: 383) | cct gatcgtcagc ctcgcag |
| (SEQ ID NO: 384) | ct gatcgtcagc ctcgcagt |
| (SEQ ID NO: 385) | t gatcgtcagc ctcgcagtg |
| (SEQ ID NO: 386) | gatcgtcagc ctcgcagtgg |
| (SEQ ID NO: 387) | atcgtcagc ctcgcagtgg c |
| (SEQ ID NO: 388) | tcgtcagc ctcgcagtgg cc |
| (SEQ ID NO: 389) | cgtcagc ctcgcagtgg ccg |
| (SEQ ID NO: 390) | gtcagc ctcgcagtgg ccga |
| (SEQ ID NO: 391) | tcagc ctcgcagtgg ccgac |
| (SEQ ID NO: 392) | cagc ctcgcagtgg ccgacc |
| (SEQ ID NO: 393) | agc ctcgcagtgg ccgacct |
| (SEQ ID NO: 394) | gc ctcgcagtgg ccgacctc |
| (SEQ ID NO: 395) | c ctcgcagtgg ccgacctcc |
| (SEQ ID NO: 396) | ctcgcagtgg ccgacctcct |
| (SEQ ID NO: 397) | tcgcagtgg ccgacctcct c |
| (SEQ ID NO: 398) | cgcagtgg ccgacctcct cg |
| (SEQ ID NO: 399) | gcagtgg ccgacctcct cgt |
| (SEQ ID NO: 400) | cagtgg ccgacctcct cgtc |
| (SEQ ID NO: 401) | agtgg ccgacctcct cgtcg |
| (SEQ ID NO: 402) | gtgg ccgacctcct cgtcgc |
| (SEQ ID NO: 403) | tgg ccgacctcct cgtcgcc |
| (SEQ ID NO: 404) | gg ccgacctcct cgtcgcca |
| (SEQ ID NO: 405) | g ccgacctcct cgtcgccac |
| (SEQ ID NO: 406) | ccgacctcct cgtcgccaca |
| (SEQ ID NO: 407) | cgacctcct cgtcgccaca c |
| (SEQ ID NO: 408) | gacctcct cgtcgccaca ct |
| (SEQ ID NO: 409) | acctcct cgtcgccaca ctg |
| (SEQ ID NO: 410) | cctcct cgtcgccaca ctgg |
| (SEQ ID NO: 411) | ctcct cgtcgccaca ctggt |
| (SEQ ID NO: 412) | tcct cgtcgccaca ctggtc |
| (SEQ ID NO: 413) | cct cgtcgccaca ctggtca |
| (SEQ ID NO: 414) | ct cgtcgccaca ctggtcat |
| (SEQ ID NO: 415) | t cgtcgccaca ctggtcatg |
| (SEQ ID NO: 416) | cgtcgccaca ctggtcatgc |
| (SEQ ID NO: 417) | gtcgccaca ctggtcatgc c |
| (SEQ ID NO: 418) | tcgccaca ctggtcatgc cc |
| (SEQ ID NO: 419) | cgccaca ctggtcatgc cct |
| (SEQ ID NO: 420) | gccaca ctggtcatgc cctg |
| (SEQ ID NO: 421) | ccaca ctggtcatgc cctgg |
| (SEQ ID NO: 422) | caca ctggtcatgc cctggg |
| (SEQ ID NO: 423) | aca ctggtcatgc cctgggt |
| (SEQ ID NO: 424) | ca ctggtcatgc cctgggtt |
| (SEQ ID NO: 425) | a ctggtcatgc cctgggttg |
| (SEQ ID NO: 426) | ctggtcatgc cctgggttgt |
| (SEQ ID NO: 427) | tggtcatgc cctgggttgt c |
| (SEQ ID NO: 428) | ggtcatgc cctgggttgt ct |
| (SEQ ID NO: 429) | gtcatgc cctgggttgt cta |
| (SEQ ID NO: 430) | tcatgc cctgggttgt ctac |
| (SEQ ID NO: 431) | catgc cctgggttgt ctac |
| (SEQ ID NO: 432) | atgc cctgggttgt ctacc |
| (SEQ ID NO: 433) | tgc cctgggttgt ctacct |
| (SEQ ID NO: 434) | gc cctgggttgt ctacctg |
| (SEQ ID NO: 435) | c cctgggttgt ctacctgg |
| (SEQ ID NO: 436) | cctgggttgt ctacctggag |
| (SEQ ID NO: 437) | ctgggttgt ctacctggag g |
| (SEQ ID NO: 438) | tgggttgt ctacctggag gt |
| (SEQ ID NO: 439) | gggttgt ctacctggag gtg |
| (SEQ ID NO: 440) | ggttgt ctacctggag gtgg |
| (SEQ ID NO: 441) | gttgt ctacctggag gtggt |
| (SEQ ID NO: 442) | ttgt ctacctggag gtggta |
| (SEQ ID NO: 443) | tgt ctacctggag gtggtag |
| (SEQ ID NO: 444) | gt ctacctggag gtggtagg |
| (SEQ ID NO: 445) | t ctacctggag gtggtaggt |
| (SEQ ID NO: 446) | ctacctggag gtggtaggtg |
| (SEQ ID NO: 447) | tacctggag gtggtaggtg a |
| (SEQ ID NO: 448) | acctggag gtggtaggtg ag |
| (SEQ ID NO: 449) | cctggag gtggtaggtg agt |
| (SEQ ID NO: 450) | ctggag gtggtaggtg agtg |
| (SEQ ID NO: 451) | tggag gtggtaggtg agtgg |
| (SEQ ID NO: 452) | ggag gtggtaggtg agtgga |
| (SEQ ID NO: 453) | gag gtggtaggtg agtggaa |
| (SEQ ID NO: 454) | ag gtggtaggtg agtggaaa |
| (SEQ ID NO: 455) | g gtggtaggtg agtggaaat |
| (SEQ ID NO: 456) | gtggtaggtg agtggaaatt |
| (SEQ ID NO: 457) | tggtaggtg agtggaaatt c |
| (SEQ ID NO: 458) | ggtaggtg agtggaaatt ca |
| (SEQ ID NO: 459) | gtaggtg agtggaaatt cag |
| (SEQ ID NO: 460) | taggtg agtggaaatt cagc |
| (SEQ ID NO: 461) | aggtg agtggaaatt cagca |
| (SEQ ID NO: 462) | ggtg agtggaaatt cagcag |
| (SEQ ID NO: 463) | gtg agtggaaatt cagcagg |
| (SEQ ID NO: 464) | tg agtggaaatt cagcagga |
| (SEQ ID NO: 465) | g agtggaaatt cagcaggat |
| (SEQ ID NO: 466) | agtggaaatt cagcaggatt |
| (SEQ ID NO: 467) | gtggaaatt cagcaggatt c |
| (SEQ ID NO: 468) | tggaaatt cagcaggatt ca |
| (SEQ ID NO: 469) | ggaaatt cagcaggatt cac |
| (SEQ ID NO: 470) | gaaatt cagcaggatt cact |
| (SEQ ID NO: 471) | aaatt cagcaggatt cactg |
| (SEQ ID NO: 472) | aatt cagcaggatt cactgt |
| (SEQ ID NO: 473) | att cagcaggatt cactgtg |
| (SEQ ID NO: 474) | tt cagcaggatt cactgtga |
| (SEQ ID NO: 475) | t cagcaggatt cactgtgac |
| (SEQ ID NO: 476) | cagcaggatt cactgtgaca |
| (SEQ ID NO: 477) | agcaggatt cactgtgaca t |
| (SEQ ID NO: 478) | gcaggatt cactgtgaca tc |
| (SEQ ID NO: 479) | caggatt cactgtgaca tct |
| (SEQ ID NO: 480) | aggatt cactgtgaca tctt |
| (SEQ ID NO: 481) | ggatt cactgtgaca tcttc |
| (SEQ ID NO: 482) | gatt cactgtgaca tcttcg |
| (SEQ ID NO: 483) | att cactgtgaca tcttcgt |
| (SEQ ID NO: 484) | tt cactgtgaca tcttcgtc |
| (SEQ ID NO: 485) | t cactgtgaca tcttcgtca |
| (SEQ ID NO: 486) | cactgtgaca tcttcgtcac |
| (SEQ ID NO: 487) | actgtgaca tcttcgtcac t |
| (SEQ ID NO: 488) | ctgtgaca tcttcgtcac tc |
| (SEQ ID NO: 489) | tgtgaca tcttcgtcac tct |
| (SEQ ID NO: 490) | gtgaca tcttcgtcac tctg |
| (SEQ ID NO: 491) | tgaca tcttcgtcac tctgg |
| (SEQ ID NO: 492) | gaca tcttcgtcac tctgga |
| (SEQ ID NO: 493) | aca tcttcgtcac tctggac |
| (SEQ ID NO: 494) | ca tcttcgtcac tctggacg |
| (SEQ ID NO: 495) | a tcttcgtcac tctggacgt |
| (SEQ ID NO: 496) | tcttcgtcac tctggacgtc |
| (SEQ ID NO: 497) | cttcgtcac tctggacgtc a |
| (SEQ ID NO: 498) | ttcgtcac tctggacgtc at |
| (SEQ ID NO: 499) | tcgtcac tctggacgtc atg |
| (SEQ ID NO: 500) | cgtcac tctggacgtc atga |
| (SEQ ID NO: 501) | gtcac tctggacgtc atgat |
| (SEQ ID NO: 502) | tcac tctggacgtc atgatg |
| (SEQ ID NO: 503) | cac tctggacgtc atgatgt |
| (SEQ ID NO: 504) | ac tctggacgtc atgatgtg |
| (SEQ ID NO: 505) | c tctggacgtc atgatgtgc |
| (SEQ ID NO: 506) | tctggacgtc atgatgtgca |
| (SEQ ID NO: 507) | ctggacgtc atgatgtgca c |
| (SEQ ID NO: 508) | tggacgtc atgatgtgca cg |
| (SEQ ID NO: 509) | ggacgtc atgatgtgca cgg |
| (SEQ ID NO: 510) | gacgtc atgatgtgca cggc |
| (SEQ ID NO: 511) | acgtc atgatgtgca cggcg |
| (SEQ ID NO: 512) | cgtc atgatgtgca cggcga |
| (SEQ ID NO: 513) | gtc atgatgtgca cggcgag |
| (SEQ ID NO: 514) | tc atgatgtgca cggcgagc |
| (SEQ ID NO: 515) | c atgatgtgca cggcgagca |
| (SEQ ID NO: 516) | atgatgtgca cggcgagcat |
| (SEQ ID NO: 517) | tgatgtgca cggcgagcat c |
| (SEQ ID NO: 518) | gatgtgca cggcgagcat cc |
| (SEQ ID NO: 519) | atgtgca cggcgagcat cct |
| (SEQ ID NO: 520) | tgtgca cggcgagcat cctg |
| (SEQ ID NO: 521) | gtgca cggcgagcat cctga |
| (SEQ ID NO: 522) | tgca cggcgagcat cctgaa |
| (SEQ ID NO: 523) | gca cggcgagcat cctgaac |
| (SEQ ID NO: 524) | ca cggcgagcat cctgaact |
| (SEQ ID NO: 525) | a cggcgagcat cctgaactt |
| (SEQ ID NO: 526) | cggcgagcat cctgaacttg |
| (SEQ ID NO: 527) | ggcgagcat cctgaacttg t |
| (SEQ ID NO: 528) | gcgagcat cctgaacttg tg |
| (SEQ ID NO: 529) | cgagcat cctgaacttg tgt |
| (SEQ ID NO: 530) | gagcat cctgaacttg tgtg |
| (SEQ ID NO: 531) | agcat cctgaacttg tgtgc |
| (SEQ ID NO: 532) | gcat cctgaacttg tgtgcc |
| (SEQ ID NO: 533) | cat cctgaacttg tgtgcca |
| (SEQ ID NO: 534) | at cctgaacttg tgtgccat |
| (SEQ ID NO: 535) | t cctgaacttg tgtgccatc |
| (SEQ ID NO: 536) | cctgaacttg tgtgccatca |
| (SEQ ID NO: 537) | ctgaacttg tgtgccatca g |
| (SEQ ID NO: 538) | tgaacttg tgtgccatca gc |
| (SEQ ID NO: 539) | gaacttg tgtgccatca gca |
| (SEQ ID NO: 540) | aacttg tgtgccatca gcat |
| (SEQ ID NO: 541) | acttg tgtgccatca gcatc |
| (SEQ ID NO: 542) | cttg tgtgccatca gcatcg |
| (SEQ ID NO: 543) | ttg tgtgccatca gcatcga |
| (SEQ ID NO: 544) | tg tgtgccatca gcatcgac |
| (SEQ ID NO: 545) | g tgtgccatca gcatcgaca |
| (SEQ ID NO: 546) | tgtgccatca gcatcgacag |
| (SEQ ID NO: 547) | gtgccatca gcatcgacag g |
| (SEQ ID NO: 548) | tgccatca gcatcgacag gt |
| (SEQ ID NO: 549) | gccatca gcatcgacag gta |
| (SEQ ID NO: 550) | ccatca gcatcgacag gtac |
| (SEQ ID NO: 551) | catca gcatcgacag gtaca |
| (SEQ ID NO: 552) | atca gcatcgacag gtacac |
| (SEQ ID NO: 553) | tca gcatcgacag gtacaca |
| (SEQ ID NO: 554) | ca gcatcgacag gtacacag |
| (SEQ ID NO: 555) | a gcatcgacag gtacacagc |
| (SEQ ID NO: 556) | gcatcgacag gtacacagct |
| (SEQ ID NO: 557) | catcgacag gtacacagct g |
| (SEQ ID NO: 558) | atcgacag gtacacagct gt |
| (SEQ ID NO: 559) | tcgacag gtacacagct gtg |
| (SEQ ID NO: 560) | cgacag gtacacagct gtgg |
| (SEQ ID NO: 561) | gacag gtacacagct gtggc |
| (SEQ ID NO: 562) | acag gtacacagct gtggcc |
| (SEQ ID NO: 563) | cag gtacacagct gtggcca |
| (SEQ ID NO: 564) | ag gtacacagct gtggccat |
| (SEQ ID NO: 565) | g gtacacagct gtggccatg |
| (SEQ ID NO: 566) | g gtacacagct gtggccatg |
| (SEQ ID NO: 567) | tacacagct gtggccatgc c |
| (SEQ ID NO: 568) | acacagct gtggccatgc cc |
| (SEQ ID NO: 569) | cacagct gtggccatgc cca |
| (SEQ ID NO: 570) | acagct gtggccatgc ccat |
| (SEQ ID NO: 571) | cagct gtggccatgc ccatg |
| (SEQ ID NO: 572) | agct gtggccatgc ccatgc |
| (SEQ ID NO: 573) | gct gtggccatgc ccatgct |
| (SEQ ID NO: 574) | ct gtggccatgc ccatgctg |
| (SEQ ID NO: 575) | t gtggccatgc ccatgctgt |
| (SEQ ID NO: 576) | gtggccatgc ccatgctgta |
| (SEQ ID NO: 577) | tggccatgc ccatgctgta c |
| (SEQ ID NO: 578) | ggccatgc ccatgctgta ca |
| (SEQ ID NO: 579) | gccatgc ccatgctgta caa |
| (SEQ ID NO: 580) | ccatgc ccatgctgta caat |
| (SEQ ID NO: 581) | catgc ccatgctgta caata |
| (SEQ ID NO: 582) | atgc ccatgctgta caatac |
| (SEQ ID NO: 583) | tgc ccatgctgta caatacg |
| (SEQ ID NO: 584) | gc ccatgctgta caatacgc |
| (SEQ ID NO: 585) | c ccatgctgta caatacgcg |
| (SEQ ID NO: 586) | ccatgctgta caatacgcgc |
| (SEQ ID NO: 587) | catgctgta caatacgcgc t |
| (SEQ ID NO: 588) | atgctgta caatacgcgc ta |
| (SEQ ID NO: 589) | tgctgta caatacgcgc tac |
| (SEQ ID NO: 590) | gctgta caatacgcgc taca |
| (SEQ ID NO: 591) | ctgta caatacgcgc tacag |
| (SEQ ID NO: 592) | tgta caatacgcgc tacagc |
| (SEQ ID NO: 593) | gta caatacgcgc tacagct |
| (SEQ ID NO: 594) | ta caatacgcgc tacagctc |
| (SEQ ID NO: 595) | a caatacgcgc tacagctcc |
| (SEQ ID NO: 596) | caatacgcgc tacagctcca |
| (SEQ ID NO: 597) | aatacgcgc tacagctcca a |
| (SEQ ID NO: 598) | atacgcgc tacagctcca ag |
| (SEQ ID NO: 599) | tacgcgc tacagctcca agc |
| (SEQ ID NO: 600) | acgcgc tacagctcca agcg |
| (SEQ ID NO: 601) | cgcgc tacagctcca agcgc |
| (SEQ ID NO: 602) | gcgc tacagctcca agcgcc |
| (SEQ ID NO: 603) | cgc tacagctcca agcgccg |
| (SEQ ID NO: 604) | gc tacagctcca agcgccgg |
| (SEQ ID NO: 605) | c tacagctcca agcgccggg |
| (SEQ ID NO: 606) | tacagctcca agcgccgggt |
| (SEQ ID NO: 607) | acagctcca agcgccgggt c |
| (SEQ ID NO: 608) | cagctcca agcgccgggt ca |
| (SEQ ID NO: 609) | agctcca agcgccgggt cac |
| (SEQ ID NO: 610) | gctcca agcgccgggt cacc |
| (SEQ ID NO: 611) | ctcca agcgccgggt caccg |
| (SEQ ID NO: 612) | tcca agcgccgggt caccgt |
| (SEQ ID NO: 613) | cca agcgccgggt caccgtc |
| (SEQ ID NO: 614) | ca agcgccgggt caccgtca |
| (SEQ ID NO: 615) | a agcgccgggt caccgtcat |
| (SEQ ID NO: 616) | agcgccgggt caccgtcatg |
| (SEQ ID NO: 617) | gcgccgggt caccgtcatg a |
| (SEQ ID NO: 618) | cgccgggt caccgtcatg at |
| (SEQ ID NO: 619) | gccgggt caccgtcatg atc |
| (SEQ ID NO: 620) | ccgggt caccgtcatg atct |
| (SEQ ID NO: 621) | cgggt caccgtcatg atctc |
| (SEQ ID NO: 622) | gggt caccgtcatg atctcc |
| (SEQ ID NO: 623) | ggt caccgtcatg atctcca |
| (SEQ ID NO: 624) | gt caccgtcatg atctccat |
| (SEQ ID NO: 625) | t caccgtcatg atctccatc |
| (SEQ ID NO: 626) | caccgtcatg atctccatcg |
| (SEQ ID NO: 627) | accgtcatg atctccatcg t |
| (SEQ ID NO: 628) | ccgtcatg atctccatcg tc |
| (SEQ ID NO: 629) | cgtcatg atctccatcg tct |
| (SEQ ID NO: 630) | gtcatg atctccatcg tctg |
| (SEQ ID NO: 631) | tcatg atctccatcg tctgg |
| (SEQ ID NO: 632) | catg atctccatcg tctggg |
| (SEQ ID NO: 633) | atg atctccatcg tctgggt |
| (SEQ ID NO: 634) | tg atctccatcg tctgggtc |
| (SEQ ID NO: 635) | g atctccatcg tctgggtcc |
| (SEQ ID NO: 636) | atctccatcg tctgggtcct |
| (SEQ ID NO: 637) | tctccatcg tctgggtcct g |
| (SEQ ID NO: 638) | ctccatcg tctgggtcct gt |
| (SEQ ID NO: 639) | tccatcg tctgggtcct gtc |
| (SEQ ID NO: 640) | ccatcg tctgggtcct gtcc |
| (SEQ ID NO: 641) | catcg tctgggtcct gtcct |
| (SEQ ID NO: 642) | atcg tctgggtcct gtcctt |
| (SEQ ID NO: 643) | tcg tctgggtcct gtccttc |
| (SEQ ID NO: 644) | cg tctgggtcct gtccttca |
| (SEQ ID NO: 645) | g tctgggtcct gtccttcac |
| (SEQ ID NO: 646) | tctgggtcct gtccttcacc |
| (SEQ ID NO: 647) | ctgggtcct gtccttcacc a |
| (SEQ ID NO: 648) | tgggtcct gtccttcacc at |
| (SEQ ID NO: 649) | gggtcct gtccttcacc atc |
| (SEQ ID NO: 650) | ggtcct gtccttcacc atct |
| (SEQ ID NO: 651) | gtcct gtccttcacc atctc |
| (SEQ ID NO: 652) | tcct gtccttcacc atctcc |
| (SEQ ID NO: 653) | cct gtccttcacc atctcct |
| (SEQ ID NO: 654) | ct gtccttcacc atctcctg |
| (SEQ ID NO: 655) | t gtccttcacc atctcctgc |
| (SEQ ID NO: 656) | gtccttcacc atctcctgcc |
| (SEQ ID NO: 657) | tccttcacc atctcctgcc c |
| (SEQ ID NO: 658) | ccttcacc atctcctgcc ca |
| (SEQ ID NO: 659) | cttcacc atctcctgcc cac |
| (SEQ ID NO: 660) | ttcacc atctcctgcc cact |
| (SEQ ID NO: 661) | tcacc atctcctgcc cactc |
| (SEQ ID NO: 662) | cacc atctcctgcc cactcc |
| (SEQ ID NO: 663) | acc atctcctgcc cactcct |
| (SEQ ID NO: 664) | cc atctcctgcc cactcctc |
| (SEQ ID NO: 665) | c atctcctgcc cactcctct |
| (SEQ ID NO: 666) | atctcctgcc cactcctctt |
| (SEQ ID NO: 667) | tctcctgcc cactcctctt c |
| (SEQ ID NO: 668) | ctcctgcc cactcctctt cg |
| (SEQ ID NO: 669) | tcctgcc cactcctctt cgg |
| (SEQ ID NO: 670) | cctgcc cactcctctt cgga |
| (SEQ ID NO: 671) | ctgcc cactcctctt cggac |
| (SEQ ID NO: 672) | tgcc cactcctctt cggact |
| (SEQ ID NO: 673) | gcc cactcctctt cggactc |
| (SEQ ID NO: 674) | cc cactcctctt cggactca |
| (SEQ ID NO: 675) | c cactcctctt cggactcaa |
| (SEQ ID NO: 676) | cactcctctt cggactcaat |
| (SEQ ID NO: 677) | actcctctt cggactcaat a |
| (SEQ ID NO: 678) | ctcctctt cggactcaat aa |
| (SEQ ID NO: 679) | tcctctt cggactcaat aac |
| (SEQ ID NO: 680) | cctctt cggactcaat aacg |
| (SEQ ID NO: 681) | ctctt cggactcaat aacgc |
| (SEQ ID NO: 682) | tctt cggactcaat aacgca |
| (SEQ ID NO: 683) | ctt cggactcaat aacgcag |
| (SEQ ID NO: 684) | tt cggactcaat aacgcaga |
| (SEQ ID NO: 685) | t cggactcaat aacgcagac |
| (SEQ ID NO: 686) | cggactcaat aacgcagacc |
| (SEQ ID NO: 687) | ggactcaat aacgcagacc a |
| (SEQ ID NO: 688) | gactcaat aacgcagacc ag |
| (SEQ ID NO: 689) | actcaat aacgcagacc aga |
| (SEQ ID NO: 690) | ctcaat aacgcagacc agaa |
| (SEQ ID NO: 691) | tcaat aacgcagacc agaac |
| (SEQ ID NO: 692) | caat aacgcagacc agaacg |
| (SEQ ID NO: 693) | aat aacgcagacc agaacga |
| (SEQ ID NO: 694) | at aacgcagacc agaacgag |
| (SEQ ID NO: 695) | t aacgcagacc agaacgagt |
| (SEQ ID NO: 696) | aacgcagacc agaacgagtg |
| (SEQ ID NO: 697) | acgcagacc agaacgagtg c |
| (SEQ ID NO: 698) | cgcagacc agaacgagtg ca |
| (SEQ ID NO: 699) | gcagacc agaacgagtg cat |
| (SEQ ID NO: 700) | cagacc agaacgagtg catc |
| (SEQ ID NO: 701) | agacc agaacgagtg catca |
| (SEQ ID NO: 702) | gacc agaacgagtg catcat |
| (SEQ ID NO: 703) | acc agaacgagtg catcatt |
| (SEQ ID NO: 704) | cc agaacgagtg catcattg |
| (SEQ ID NO: 705) | c agaacgagtg catcattgc |
| (SEQ ID NO: 706) | agaacgagtg catcattgcc |
| (SEQ ID NO: 707) | gaacgagtg catcattgcc a |
| (SEQ ID NO: 708) | aacgagtg catcattgcc aa |
| (SEQ ID NO: 709) | acgagtg catcattgcc aac |
| (SEQ ID NO: 710) | cgagtg catcattgcc aacc |
| (SEQ ID NO: 711) | gagtg catcattgcc aaccc |
| (SEQ ID NO: 712) | agtg catcattgcc aacccg |
| (SEQ ID NO: 713) | gtg catcattgcc aacccgg |
| (SEQ ID NO: 714) | tg catcattgcc aacccggc |
| (SEQ ID NO: 715) | g catcattgcc aacccggcc |
| (SEQ ID NO: 716) | catcattgcc aacccggcct |
| (SEQ ID NO: 717) | atcattgcc aacccggcct t |
| (SEQ ID NO: 718) | tcattgcc aacccggcct tc |
| (SEQ ID NO: 719) | cattgcc aacccggcct tcg |
| (SEQ ID NO: 720) | attgcc aacccggcct tcgt |
| (SEQ ID NO: 721) | ttgcc aacccggcct tcgtg |
| (SEQ ID NO: 722) | tgcc aacccggcct tcgtgg |
| (SEQ ID NO: 723) | gcc aacccggcct tcgtggt |
| (SEQ ID NO: 724) | cc aacccggcct tcgtggtc |
| (SEQ ID NO: 725) | c aacccggcct tcgtggtct |
| (SEQ ID NO: 726) | aacccggcct tcgtggtcta |
| (SEQ ID NO: 727) | acccggcct tcgtggtcta c |
| (SEQ ID NO: 728) | cccggcct tcgtggtcta ct |
| (SEQ ID NO: 729) | ccggcct tcgtggtcta ctc |
| (SEQ ID NO: 730) | cggcct tcgtggtcta ctcc |
| (SEQ ID NO: 731) | ggcct tcgtggtcta ctcct |
| (SEQ ID NO: 732) | gcct tcgtggtcta ctcctc |
| (SEQ ID NO: 733) | cct tcgtggtcta ctcctcc |
| (SEQ ID NO: 734) | ct tcgtggtcta ctcctcca |
| (SEQ ID NO: 735) | t tcgtggtcta ctcctccat |
| (SEQ ID NO: 736) | tcgtggtcta ctcctccatc |
| (SEQ ID NO: 737) | cgtggtcta ctcctccatc g |
| (SEQ ID NO: 738) | gtggtcta ctcctccatc gt |
| (SEQ ID NO: 739) | tggtcta ctcctccatc gtc |
| (SEQ ID NO: 740) | ggtcta ctcctccatc gtct |
| (SEQ ID NO: 741) | gtcta ctcctccatc gtctc |
| (SEQ ID NO: 742) | tcta ctcctccatc gtctcc |
| (SEQ ID NO: 743) | cta ctcctccatc gtctcct |
| (SEQ ID NO: 744) | ta ctcctccatc gtctcctt |
| (SEQ ID NO: 745) | a ctcctccatc gtctccttc |
| (SEQ ID NO: 746) | ctcctccatc gtctccttct |
| (SEQ ID NO: 747) | tcctccatc gtctccttct a |
| (SEQ ID NO: 748) | cctccatc gtctccttct ac |
| (SEQ ID NO: 749) | ctccatc gtctccttct acg |
| (SEQ ID NO: 750) | tccatc gtctccttct acgt |
| (SEQ ID NO: 751) | ccatc gtctccttct acgtg |
| (SEQ ID NO: 752) | catc gtctccttct acgtgc |
| (SEQ ID NO: 753) | atc gtctccttct acgtgcc |
| (SEQ ID NO: 754) | tc gtctccttct acgtgccc |
| (SEQ ID NO: 755) | c gtctccttct acgtgccct |
| (SEQ ID NO: 756) | gtctccttct acgtgtccctt |
| (SEQ ID NO: 757) | tctccttct acgtgccctt c |
| (SEQ ID NO: 758) | ctccttct acgtgccctt ca |
| (SEQ ID NO: 759) | tccttct acgtgccctt cat |
| (SEQ ID NO: 760) | ccttct acgtgccctt catt |
| (SEQ ID NO: 761) | cttct acgtgccctt cattg |
| (SEQ ID NO: 762) | ttct acgtgccctt cattgt |
| (SEQ ID NO: 763) | tct acgtgccctt cattgtc |
| (SEQ ID NO: 764) | ct acgtgccctt cattgtca |
| (SEQ ID NO: 765) | t acgtgccctt cattgtcac |
| (SEQ ID NO: 766) | acgtgccctt cattgtcacc |
| (SEQ ID NO: 767) | cgtgccctt cattgtcacc c |
| (SEQ ID NO: 768) | gtgccctt cattgtcacc ct |
| (SEQ ID NO: 769) | tgccctt cattgtcacc ctg |
| (SEQ ID NO: 770) | gccctt cattgtcacc ctgc |
| (SEQ ID NO: 771) | ccctt cattgtcacc ctgct |
| (SEQ ID NO: 772) | cctt cattgtcacc ctgctg |
| (SEQ ID NO: 773) | ctt cattgtcacc ctgctgg |
| (SEQ ID NO: 774) | tt cattgtcacc ctgctggt |
| (SEQ ID NO: 775) | t cattgtcacc ctgctggtc |
| (SEQ ID NO: 776) | cattgtcacc ctgctggtct |
| (SEQ ID NO: 777) | attgtcacc ctgctggtct a |
| (SEQ ID NO: 778) | ttgtcacc ctgctggtct ac |
| (SEQ ID NO: 779) | tgtcacc ctgctggtct aca |
| (SEQ ID NO: 780) | gtcacc ctgctggtct acat |
| (SEQ ID NO: 781) | tcacc ctgctggtct acatc |
| (SEQ ID NO: 782) | cacc ctgctggtct acatca |
| (SEQ ID NO: 783) | acc ctgctggtct acatcaa |
| (SEQ ID NO: 784) | cc ctgctggtct acatcaag |
| (SEQ ID NO: 785) | c ctgctggtct acatcaaga |
| (SEQ ID NO: 786) | ctgctggtct acatcaagat |
| (SEQ ID NO: 787) | tgctggtct acatcaagat c |
| (SEQ ID NO: 788) | gctggtct acatcaagat ct |
| (SEQ ID NO: 789) | ctggtct acatcaagat cta |
| (SEQ ID NO: 790) | tggtct acatcaagat ctac |
| (SEQ ID NO: 791) | ggtct acatcaagat ctaca |
| (SEQ ID NO: 792) | gtct acatcaagat ctacat |
| (SEQ ID NO: 793) | tct acatcaagat ctacatt |
| (SEQ ID NO: 794) | ct acatcaagat ctacattg |
| (SEQ ID NO: 795) | t acatcaagat ctacattgt |
| (SEQ ID NO: 796) | acatcaagat ctacattgtc |
| (SEQ ID NO: 797) | catcaagat ctacattgtc c |
| (SEQ ID NO: 798) | atcaagat ctacattgtc ct |
| (SEQ ID NO: 799) | tcaagat ctacattgtc ctc |
| (SEQ ID NO: 800) | caagat ctacattgtc ctcc |
| (SEQ ID NO: 801) | aagat ctacattgtc ctccg |
| (SEQ ID NO: 802) | agat ctacattgtc ctccgc |
| (SEQ ID NO: 803) | gat ctacattgtc ctccgca |
| (SEQ ID NO: 804) | at ctacattgtc ctccgcag |
| (SEQ ID NO: 805) | t ctacattgtc ctccgcaga |
| (SEQ ID NO: 806) | ctacattgtc ctccgcagac |
| (SEQ ID NO: 807) | tacattgtc ctccgcagac g |
| (SEQ ID NO: 808) | acattgtc ctccgcagac gc |
| (SEQ ID NO: 809) | cattgtc ctccgcagac gcc |
| (SEQ ID NO: 810) | attgtc ctccgcagac gccg |
| (SEQ ID NO: 811) | ttgtc ctccgcagac gccgc |
| (SEQ ID NO: 812) | tgtc ctccgcagac gccgca |
| (SEQ ID NO: 813) | gtc ctccgcagac gccgcaa |
| (SEQ ID NO: 814) | gtc ctccgcagac gccgcaag |
| (SEQ ID NO: 815) | tc ctccgcagac gccgcaag |
| (SEQ ID NO: 816) | c ctccgcagac gccgcaagc |
| (SEQ ID NO: 817) | ctccgcagac gccgcaagcg |
| (SEQ ID NO: 818) | tccgcagac gccgcaagcg a |
| (SEQ ID NO: 819) | ccgcagac gccgcaagcg ag |
| (SEQ ID NO: 820) | cgcagac gccgcaagcg agt |
| (SEQ ID NO: 821) | gcagac gccgcaagcg agtc |
| (SEQ ID NO: 822) | cagac gccgcaagcg agtca |
| (SEQ ID NO: 823) | agac gccgcaagcg agtcaa |
| (SEQ ID NO: 824) | gac gccgcaagcg agtcaac |
| (SEQ ID NO: 825) | ac gccgcaagcg agtcaaca |
| (SEQ ID NO: 826) | c gccgcaagcg agtcaacac |
| (SEQ ID NO: 827) | gccgcaagcg agtcaacacc |
| (SEQ ID NO: 828) | ccgcaagcg agtcaacacc a |
| (SEQ ID NO: 829) | cgcaagcg agtcaacacc aa |
| (SEQ ID NO: 830) | gcaagcg agtcaacacc aaa |
| (SEQ ID NO: 831) | caagcg agtcaacacc aaac |
| (SEQ ID NO: 832) | aagcg agtcaacacc aaacg |
| (SEQ ID NO: 833) | agcg agtcaacacc aaacgc |
| (SEQ ID NO: 834) | gcg agtcaacacc aaacgca |
| (SEQ ID NO: 835) | cg agtcaacacc aaacgcag |
| (SEQ ID NO: 836) | g agtcaacacc aaacgcagc |
| (SEQ ID NO: 837) | agtcaacacc aaacgcagca |
| (SEQ ID NO: 838) | gtcaacacc aaacgcagca g |
| (SEQ ID NO: 839) | tcaacacc aaacgcagca gc |
| (SEQ ID NO: 840) | caacacc aaacgcagca gcc |
| (SEQ ID NO: 841) | aacacc aaacgcagca gccg |
| (SEQ ID NO: 842) | acacc aaacgcagca gccga |
| (SEQ ID NO: 843) | cacc aaacgcagca gccgag |
| (SEQ ID NO: 844) | acc aaacgcagca gccgagc |
| (SEQ ID NO: 845) | cc aaacgcagca gccgagct |
| (SEQ ID NO: 846) | c aaacgcagca gccgagctt |
| (SEQ ID NO: 847) | aaacgcagca gccgagcttt |
| (SEQ ID NO: 848) | aacgcagca gccgagcttt c |
| (SEQ ID NO: 849) | acgcagca gccgagcttt ca |
| (SEQ ID NO: 850) | cgcagca gccgagcttt cag |
| (SEQ ID NO: 851) | gcagca gccgagcttt cagg |
| (SEQ ID NO: 852) | cagca gccgagcttt caggg |
| (SEQ ID NO: 853) | agca gccgagcttt cagggc |
| (SEQ ID NO: 854) | gca gccgagcttt cagggcc |
| (SEQ ID NO: 855) | ca gccgagcttt cagggccc |
| (SEQ ID NO: 856) | a gccgagcttt cagggccca |
| (SEQ ID NO: 857) | gccgagcttt cagggcccac |
| (SEQ ID NO: 858) | ccgagcttt cagggcccac c |
| (SEQ ID NO: 859) | cgagcttt cagggcccac ct |
| (SEQ ID NO: 860) | gagcttt cagggcccac ctg |
| (SEQ ID NO: 861) | agcttt cagggcccac ctga |
| (SEQ ID NO: 862) | gcttt cagggcccac ctgag |
| (SEQ ID NO: 863) | cttt cagggcccac ctgagg |
| (SEQ ID NO: 864) | ttt cagggcccac ctgaggg |
| (SEQ ID NO: 865) | tt cagggcccac ctgagggc |
| (SEQ ID NO: 866) | t cagggcccac ctgagggct |
| (SEQ ID NO: 867) | cagggcccac ctgagggctc |
| (SEQ ID NO: 868) | agggcccac ctgagggctc c |
| (SEQ ID NO: 869) | gggcccac ctgagggctc ca |
| (SEQ ID NO: 870) | ggcccac ctgagggctc cac |
| (SEQ ID NO: 871) | gcccac ctgagggctc cact |
| (SEQ ID NO: 872) | cccac ctgagggctc cacta |
| (SEQ ID NO: 873) | ccac ctgagggctc cactaa |
| (SEQ ID NO: 874) | cac ctgagggctc cactaaa |
| (SEQ ID NO: 875) | ac ctgagggctc cactaaag |
| (SEQ ID NO: 876) | c ctgagggctc cactaaagg |
| (SEQ ID NO: 877) | ctgagggctc cactaaaggg |
| (SEQ ID NO: 878) | tgagggctc cactaaaggg c |
| (SEQ ID NO: 879) | gagggctc cactaaaggg ca |
| (SEQ ID NO: 880) | agggctc cactaaaggg caa |
| (SEQ ID NO: 881) | gggctc cactaaaggg caac |
| (SEQ ID NO: 882) | ggctc cactaaaggg caact |
| (SEQ ID NO: 883) | gctc cactaaaggg caactg |
| (SEQ ID NO: 884) | ctc cactaaaggg caactgt |
| (SEQ ID NO: 885) | tc cactaaaggg caactgta |
| (SEQ ID NO: 886) | c cactaaaggg caactgtac |
| (SEQ ID NO: 887) | cactaaaggg caactgtact |
| (SEQ ID NO: 888) | actaaaggg caactgtact c |
| (SEQ ID NO: 889) | ctaaaggg caactgtact ca |
| (SEQ ID NO: 890) | taaaggg caactgtact cac |
| (SEQ ID NO: 891) | aaaggg caactgtact cacc |
| (SEQ ID NO: 892) | aaggg caactgtact caccc |
| (SEQ ID NO: 893) | aggg caactgtact cacccc |
| (SEQ ID NO: 894) | ggg caactgtact caccccg |
| (SEQ ID NO: 895) | gg caactgtact caccccga |
| (SEQ ID NO: 896) | g caactgtact caccccgag |
| (SEQ ID NO: 897) | caactgtact caccccgagg |
| (SEQ ID NO: 898) | aactgtact caccccgagg a |
| (SEQ ID NO: 899) | actgtact caccccgagg ac |
| (SEQ ID NO: 900) | ctgtact caccccgagg aca |
| (SEQ ID NO: 901) | tgtact caccccgagg acat |
| (SEQ ID NO: 902) | gtact caccccgagg acatg |
| (SEQ ID NO: 903) | tact caccccgagg acatga |
| (SEQ ID NO: 904) | act caccccgagg acatgaa |
| (SEQ ID NO: 905) | ct caccccgagg acatgaaa |
| (SEQ ID NO: 906) | t caccccgagg acatgaaac |
| (SEQ ID NO: 907) | caccccgagg acatgaaact |
| (SEQ ID NO: 908) | accccgagg acatgaaact c |
| (SEQ ID NO: 909) | ccccgagg acatgaaact ct |
| (SEQ ID NO: 910) | cccgagg acatgaaact ctg |
| (SEQ ID NO: 911) | ccgagg acatgaaact ctgc |
| (SEQ ID NO: 912) | cgagg acatgaaact ctgca |
| (SEQ ID NO: 913) | gagg acatgaaact ctgcac |
| (SEQ ID NO: 914) | agg acatgaaact ctgcacc |
| (SEQ ID NO: 915) | gg acatgaaact ctgcaccg |
| (SEQ ID NO: 916) | g acatgaaact ctgcaccgt |
| (SEQ ID NO: 917) | acatgaaact ctgcaccgtt |
| (SEQ ID NO: 918) | catgaaact ctgcaccgtt a |
| (SEQ ID NO: 919) | atgaaact ctgcaccgtt at |
| (SEQ ID NO: 920) | tgaaact ctgcaccgtt atc |
| (SEQ ID NO: 921) | tgaaact ctgcaccgtt atc |
| (SEQ ID NO: 922) | gaaact ctgcaccgtt atca |
| (SEQ ID NO: 923) | aaact ctgcaccgtt atcat |
| (SEQ ID NO: 924) | aact ctgcaccgtt atcatg |
| (SEQ ID NO: 925) | act ctgcaccgtt atcatga |
| (SEQ ID NO: 926) | act ctgcaccgtt atcatga |
| (SEQ ID NO: 927) | t ctgcaccgtt atcatgaag |
| (SEQ ID NO: 928) | ctgcaccgtt atcatgaagt |
| (SEQ ID NO: 929) | tgcaccgtt atcatgaagt c |
| (SEQ ID NO: 930) | gcaccgtt atcatgaagt ct |
| (SEQ ID NO: 931) | caccgtt atcatgaagt cta |
| (SEQ ID NO: 932) | accgtt atcatgaagt ctaa |
| (SEQ ID NO: 933) | ccgtt atcatgaagt ctaat |
| (SEQ ID NO: 934) | cgtt atcatgaagt ctaatg |
| (SEQ ID NO: 935) | gtt atcatgaagt ctaatgg |
| (SEQ ID NO: 936) | tt atcatgaagt ctaatggg |
| (SEQ ID NO: 937) | t atcatgaagt ctaatggga |
| (SEQ ID NO: 938) | atcatgaagt ctaatgggag |
| (SEQ ID NO: 939) | tcatgaagt ctaatgggag t |
| (SEQ ID NO: 940) | catgaagt ctaatgggag tt |
| (SEQ ID NO: 941) | atgaagt ctaatgggag ttt |
| (SEQ ID NO: 942) | tgaagt ctaatgggag tttc |
| (SEQ ID NO: 943) | gaagt ctaatgggag tttcc |
| (SEQ ID NO: 944) | aagt ctaatgggag tttccc |
| (SEQ ID NO: 945) | agt ctaatgggag tttccca |
| (SEQ ID NO: 946) | gt ctaatgggag tttcccag |
| (SEQ ID NO: 947) | t ctaatgggag tttcccagt |
| (SEQ ID NO: 948) | ctaatgggag tttcccagtg |
| (SEQ ID NO: 949) | taatgggag tttcccagtg a |
| (SEQ ID NO: 950) | aatgggag tttcccagtg aa |
| (SEQ ID NO: 951) | atgggag tttcccagtg aac |
| (SEQ ID NO: 952) | tgggag tttcccagtg aaca |
| (SEQ ID NO: 953) | gggag tttcccagtg aacag |
| (SEQ ID NO: 954) | ggag tttcccagtg aacagg |
| (SEQ ID NO: 955) | gag tttcccagtg aacaggc |
| (SEQ ID NO: 956) | ag tttcccagtg aacaggcg |
| (SEQ ID NO: 957) | g tttcccagtg aacaggcgg |
| (SEQ ID NO: 958) | tttcccagtg aacaggcgga |
| (SEQ ID NO: 959) | ttcccagtg aacaggcgga g |
| (SEQ ID NO: 960) | tcccagtg aacaggcgga ga |
| (SEQ ID NO: 961) | cccagtg aacaggcgga gag |
| (SEQ ID NO: 962) | ccagtg aacaggcgga gagt |
| (SEQ ID NO: 963) | cagtg aacaggcgga gagtg |
| (SEQ ID NO: 964) | agtg aacaggcgga gagtgg |
| (SEQ ID NO: 965) | gtg aacaggcgga gagtgga |
| (SEQ ID NO: 966) | tg aacaggcgga gagtggag |
| (SEQ ID NO: 967) | g aacaggcgga gagtggagg |
| (SEQ ID NO: 968) | aacaggcgga gagtggaggc |
| (SEQ ID NO: 969) | acaggcgga gagtggaggc t |
| (SEQ ID NO: 970) | caggcgga gagtggaggc tg |
| (SEQ ID NO: 971) | aggcgga gagtggaggc tgc |
| (SEQ ID NO: 972) | ggcgga gagtggaggc tgcc |
| (SEQ ID NO: 973) | gcgga gagtggaggc tgccc |
| (SEQ ID NO: 974) | cgga gagtggaggc tgcccg |
| (SEQ ID NO: 975) | gga gagtggaggc tgcccgg |
| (SEQ ID NO: 976) | ga gagtggaggc tgcccggc |
| (SEQ ID NO: 977) | a gagtggaggc tgcccggcg |
| (SEQ ID NO: 978) | gagtggaggc tgcccggcga |
| (SEQ ID NO: 979) | agtggaggc tgcccggcga g |
| (SEQ ID NO: 980) | gtggaggc tgcccggcga gc |
| (SEQ ID NO: 981) | tggaggc tgcccggcga gcc |
| (SEQ ID NO: 982) | ggaggc tgcccggcga gccc |
| (SEQ ID NO: 983) | gaggc tgcccggcga gccca |
| (SEQ ID NO: 984) | aggc tgcccggcga gcccag |
| (SEQ ID NO: 985) | ggc tgcccggcga gcccagg |
| (SEQ ID NO: 986) | gc tgcccggcga gcccagga |
| (SEQ ID NO: 987) | c tgcccggcga gcccaggag |
| (SEQ ID NO: 988) | tgcccggcga gcccaggagac |
| (SEQ ID NO: 989) | gcccggcga gcccaggagc t |
| (SEQ ID NO: 990) | cccggcga gcccaggagc tg |
| (SEQ ID NO: 991) | ccggcga gcccaggagc tgg |
| (SEQ ID NO: 992) | cggaga gcccaggagc tgga |
| (SEQ ID NO: 993) | ggcga gcccaggagc tggag |
| (SEQ ID NO: 994) | gcga gcccaggagc tggaga |
| (SEQ ID NO: 995) | cga gcccaggagc tggagat |
| (SEQ ID NO: 996) | ga gcccaggagc tggagatg |
| (SEQ ID NO: 997) | a gcccaggagc tggagatgg |
| (SEQ ID NO: 998) | gcccaggagc tggagatgga |
| (SEQ ID NO: 999) | cccaggagc tggagatgga g |
| (SEQ ID NO: 1000) | ccaggagc tggagatgga ga |
| (SEQ ID NO: 1001) | caggagc tggagatgga gat |
| (SEQ ID NO: 1002) | aggagc tggagatgga gatg |
| (SEQ ID NO: 1003) | ggagc tggagatgga gatgc |
| (SEQ ID NO: 1004) | gaga tggagatgga gatgct |
| (SEQ ID NO: 1005) | agc tggagatgga gatgctc |
| (SEQ ID NO: 1006) | gc tggagatgga gatgctct |
| (SEQ ID NO: 1007) | c tggagatgga gatgctctc |
| (SEQ ID NO: 1008) | tggagatgga gatgctctcc |
| (SEQ ID NO: 1009) | ggagatgga gatgctctcc a |
| (SEQ ID NO: 1010) | gagatgga gatgctctcc ag |
| (SEQ ID NO: 1011) | agatgga gatgctctcc agc |
| (SEQ ID NO: 1012) | gatgga gatgctctcc agca |
| (SEQ ID NO: 1013) | atgga gatgctctcc agcac |
| (SEQ ID NO: 1014) | tgga gatgctctcc agcacc |
| (SEQ ID NO: 1015) | gga gatgctctcc agcacca |
| (SEQ ID NO: 1016) | ga gatgctctcc agcaccag |
| (SEQ ID NO: 1017) | a gatgctctcc agcaccagc |
| (SEQ ID NO: 1018) | gatgctctcc agcaccagcc |
| (SEQ ID NO: 1019) | atgctctcc agcaccagcc c |
| (SEQ ID NO: 1020) | tgctctcc agcaccagcc ca |
| (SEQ ID NO: 1021) | gctctcc agcaccagcc cac |
| (SEQ ID NO: 1022) | ctctcc agcaccagcc cacc |
| (SEQ ID NO: 1023) | tctcc agcaccagcc caccc |
| (SEQ ID NO: 1024) | ctcc agcaccagcc cacccg |
| (SEQ ID NO: 1025) | tcc agcaccagcc cacccga |
| (SEQ ID NO: 1026) | cc agcaccagcc cacccgag |
| (SEQ ID NO: 1027) | c agcaccagcc cacccgaga |
| (SEQ ID NO: 1028) | agcaccagcc cacccgagag |
| (SEQ ID NO: 1029) | gcaccagcc cacccgagag g |
| (SEQ ID NO: 1030) | caccagcc cacccgagag ga |
| (SEQ ID NO: 1031) | accagcc cacccgagag gac |
| (SEQ ID NO: 1032) | ccagcc cacccgagag gacc |
| (SEQ ID NO: 1033) | cagcc cacccgagag gaccc |
| (SEQ ID NO: 1034) | agcc cacccgagag gacccg |
| (SEQ ID NO: 1035) | gcc cacccgagag gacccgg |
| (SEQ ID NO: 1036) | cc cacccgagag gacccggt |
| (SEQ ID NO: 1037) | c cacccgagag gacccggta |
| (SEQ ID NO: 1038) | cacccgagag gacccggtac |
| (SEQ ID NO: 1039) | acccgagag gacccggtac a |
| (SEQ ID NO: 1040) | cccgagag gacccggtac ag |
| (SEQ ID NO: 1041) | ccgagag gacccggtac agc |
| (SEQ ID NO: 1042) | cgagag gacccggtac agcc |
| (SEQ ID NO: 1043) | gagag gacccggtac agccc |
| (SEQ ID NO: 1044) | agag gacccggtac agcccc |
| (SEQ ID NO: 1045) | gag gacccggtac agcccca |
| (SEQ ID NO: 1046) | ag gacccggtac agccccat |
| (SEQ ID NO: 1047) | g gacccggtac agccccatc |
| (SEQ ID NO: 1048) | gacccggtac agccccatcc |
| (SEQ ID NO: 1049) | acccggtac agccccatcc c |
| (SEQ ID NO: 1050) | cccggtac agccccatcc ca |
| (SEQ ID NO: 1051) | ccggtac agccccatcc cac |
| (SEQ ID NO: 1052) | cggtac agccccatcc cacc |
| (SEQ ID NO: 1053) | ggtac agccccatcc caccc |
| (SEQ ID NO: 1054) | gtac agccccatcc caccca |
| (SEQ ID NO: 1055) | tac agccccatcc cacccag |
| (SEQ ID NO: 1056) | ac agccccatcc cacccagc |
| (SEQ ID NO: 1057) | c agccccatcc cacccagcc |
| (SEQ ID NO: 1058) | agccccatcc cacccagcca |
| (SEQ ID NO: 1059) | gccccatcc cacccagcca c |
| (SEQ ID NO: 1060) | ccccatcc cacccagcca cc |
| (SEQ ID NO: 1061) | cccatcc cacccagcca cca |
| (SEQ ID NO: 1062) | ccatcc cacccagcca ccac |
| (SEQ ID NO: 1063) | catcc cacccagcca ccacc |
| (SEQ ID NO: 1064) | atcc cacccagcca ccacca |
| (SEQ ID NO: 1065) | tcc cacccagcca ccaccag |
| (SEQ ID NO: 1066) | cc cacccagcca ccaccagc |
| (SEQ ID NO: 1067) | c cacccagcca ccaccagct |
| (SEQ ID NO: 1068) | cacccagcca ccaccagctg |
| (SEQ ID NO: 1069) | acccagcca ccaccagctg a |
| (SEQ ID NO: 1070) | cccagcca ccaccagctg ac |
| (SEQ ID NO: 1071) | ccagcca ccaccagctg act |
| (SEQ ID NO: 1072) | cagcca ccaccagctg actc |
| (SEQ ID NO: 1073) | agcca ccaccagctg actct |
| (SEQ ID NO: 1074) | gcca ccaccagctg actctc |
| (SEQ ID NO: 1075) | cca ccaccagctg actctcc |
| (SEQ ID NO: 1076) | ca ccaccagctg actctccc |
| (SEQ ID NO: 1077) | a ccaccagctg actctcccc |
| (SEQ ID NO: 1078) | ccaccagctg actctccccg |
| (SEQ ID NO: 1079) | caccagctg actctccccg a |
| (SEQ ID NO: 1080) | accagctg actctccccg ac |
| (SEQ ID NO: 1081) | ccagctg actctccccg acc |
| (SEQ ID NO: 1082) | cagctg actctccccg accc |
| (SEQ ID NO: 1083) | agctg actctccccg acccg |
| (SEQ ID NO: 1084) | gctg actctccccg acccgt |
| (SEQ ID NO: 1085) | ctg actctccccg acccgtc |
| (SEQ ID NO: 1086) | tg actctccccg acccgtcc |
| (SEQ ID NO: 1087) | g actctccccg acccgtccc |
| (SEQ ID NO: 1088) | actctccccg acccgtccca |
| (SEQ ID NO: 1089) | ctctccccg acccgtccca c |
| (SEQ ID NO: 1090) | tctccccg acccgtccca cc |
| (SEQ ID NO: 1091) | ctccccg acccgtccca cca |
| (SEQ ID NO: 1092) | tccccg acccgtccca ccat |
| (SEQ ID NO: 1093) | ccccg acccgtccca ccatg |
| (SEQ ID NO: 1094) | cccg acccgtccca ccatgg |
| (SEQ ID NO: 1095) | ccg acccgtccca ccatggt |
| (SEQ ID NO: 1096) | cg acccgtccca ccatggtc |
| (SEQ ID NO: 1097) | g acccgtccca ccatggtct |
| (SEQ ID NO: 1098) | acccgtccca ccatggtctc |
| (SEQ ID NO: 1099) | cccgtccca ccatggtctc c |
| (SEQ ID NO: 1100) | ccgtccca ccatggtctc ca |
| (SEQ ID NO: 1101) | cgtccca ccatggtctc cac |
| (SEQ ID NO: 1102) | gtccca ccatggtctc caca |
| (SEQ ID NO: 1103) | tccca ccatggtctc cacag |
| (SEQ ID NO: 1104) | ccca ccatggtctc cacagc |
| (SEQ ID NO: 1105) | cca ccatggtctc cacagca |
| (SEQ ID NO: 1106) | ca ccatggtctc cacagcac |
| (SEQ ID NO: 1107) | a ccatggtctc cacagcact |
| (SEQ ID NO: 1108) | ccatggtctc cacagcactc |
| (SEQ ID NO: 1109) | catggtctc cacagcactc c |
| (SEQ ID NO: 1110) | atggtctc cacagcactc cc |
| (SEQ ID NO: 1111) | tggtctc cacagcactc ccg |
| (SEQ ID NO: 1112) | ggtctc cacagcactc ccga |
| (SEQ ID NO: 1113) | gtctc cacagcactc ccgac |
| (SEQ ID NO: 1114) | tctc cacagcactc ccgaca |
| (SEQ ID NO: 1115) | ctc cacagcactc ccgacag |
| (SEQ ID NO: 1116) | tc cacagcactc ccgacagc |
| (SEQ ID NO: 1117) | c cacagcactc ccgacagcc |
| (SEQ ID NO: 1118) | cacagcactc ccgacagccc |
| (SEQ ID NO: 1119) | acagcactc ccgacagccc c |
| (SEQ ID NO: 1120) | cagcactc ccgacagccc cg |
| (SEQ ID NO: 1121) | agcactc ccgacagccc cgc |
| (SEQ ID NO: 1122) | gcactc ccgacagccc cgcc |
| (SEQ ID NO: 1123) | cactc ccgacagccc cgcca |
| (SEQ ID NO: 1124) | actc ccgacagccc cgccaa |
| (SEQ ID NO: 1125) | ctc ccgacagccc cgccaaa |
| (SEQ ID NO: 1126) | tc ccgacagccc cgccaaac |
| (SEQ ID NO: 1127) | c ccgacagccc cgccaaacc |
| (SEQ ID NO: 1128) | ccgacagccc cgccaaacca |
| (SEQ ID NO: 1129) | cgacagccc cgccaaacca g |
| (SEQ ID NO: 1130) | gacagccc cgccaaacca ga |
| (SEQ ID NO: 1131) | acagccc cgccaaacca gag |
| (SEQ ID NO: 1132) | cagccc cgccaaacca gaga |
| (SEQ ID NO: 1133) | agccc cgccaaacca gagaa |
| (SEQ ID NO: 1134) | gccc cgccaaacca gagaag |
| (SEQ ID NO: 1135) | ccc cgccaaacca gagaaga |
| (SEQ ID NO: 1136) | cc cgccaaacca gagaagaa |
| (SEQ ID NO: 1137) | c cgccaaacca gagaagaat |
| (SEQ ID NO: 1138) | cgccaaacca gagaagaatg |
| (SEQ ID NO: 1139) | gccaaacca gagaagaatg g |
| (SEQ ID NO: 1140) | ccaaacca gagaagaatg gg |
| (SEQ ID NO: 1141) | caaacca gagaagaatg ggc |
| (SEQ ID NO: 1142) | aaacca gagaagaatg ggca |
| (SEQ ID NO: 1143) | aacca gagaagaatg ggcat |
| (SEQ ID NO: 1144) | acca gagaagaatg ggcatg |
| (SEQ ID NO: 1145) | cca gagaagaatg ggcatgc |
| (SEQ ID NO: 1146) | ca gagaagaatg ggcatgcc |
| (SEQ ID NO: 1147) | a gagaagaatg ggcatgcca |
| (SEQ ID NO: 1148) | gagaagaatg ggcatgccaa |
| (SEQ ID NO: 1149) | agaagaatg ggcatgccaa a |
| (SEQ ID NO: 1150) | gaagaatg ggcatgccaa ag |
| (SEQ ID NO: 1151) | aagaatg ggcatgccaa aga |
| (SEQ ID NO: 1152) | agaatg ggcatgccaa agac |
| (SEQ ID NO: 1153) | gaatg ggcatgccaa agacc |
| (SEQ ID NO: 1154) | aatg ggcatgccaa agacca |
| (SEQ ID NO: 1155) | atg ggcatgccaa agaccac |
| (SEQ ID NO: 1156) | tg ggcatgccaa agaccacc |
| (SEQ ID NO: 1157) | g ggcatgccaa agaccaccc |
| (SEQ ID NO: 1158) | ggcatgccaa agaccacccc |
| (SEQ ID NO: 1159) | gcatgccaa agaccacccc a |
| (SEQ ID NO: 1160) | catgccaa agaccacccc aa |
| (SEQ ID NO: 1161) | atgccaa agaccacccc aag |
| (SEQ ID NO: 1162) | tgccaa agaccacccc aaga |
| (SEQ ID NO: 1163) | gccaa agaccacccc aagat |
| (SEQ ID NO: 1164) | ccaa agaccacccc aagatt |
| (SEQ ID NO: 1165) | caa agaccacccc aagattg |
| (SEQ ID NO: 1166) | aa agaccacccc aagattgc |
| (SEQ ID NO: 1167) | a agaccacccc aagattgcc |
| (SEQ ID NO: 1168) | agaccacccc aagattgcca |
| (SEQ ID NO: 1169) | gaccacccc aagattgcca a |
| (SEQ ID NO: 1170) | accacccc aagattgcca ag |
| (SEQ ID NO: 1171) | ccacccc aagattgcca aga |
| (SEQ ID NO: 1172) | cacccc aagattgcca agat |
| (SEQ ID NO: 1173) | acccc aagattgcca agatc |
| (SEQ ID NO: 1174) | cccc aagattgcca agatct |
| (SEQ ID NO: 1175) | ccc aagattgcca agatctt |
| (SEQ ID NO: 1176) | cc aagattgcca agatcttt |
| (SEQ ID NO: 1177) | c aagattgcca agatctttg |
| (SEQ ID NO: 1178) | aagattgcca agatctttga |
| (SEQ ID NO: 1179) | agattgcca agatctttga g |
| (SEQ ID NO: 1180) | gattgcca agatctttga ga |
| (SEQ ID NO: 1181) | attgcca agatctttga gat |
| (SEQ ID NO: 1182) | ttgcca agatctttga gatc |
| (SEQ ID NO: 1183) | tgcca agatctttga gatcc |
| (SEQ ID NO: 1184) | gcca agatctttga gatcca |
| (SEQ ID NO: 1185) | cca agatctttga gatccag |
| (SEQ ID NO: 1186) | ca agatctttga gatccaga |
| (SEQ ID NO: 1187) | ca agatctttga gatccaga |
| (SEQ ID NO: 1188) | agatctttga gatccagacc |
| (SEQ ID NO: 1189) | gatctttga gatccagacc a |
| (SEQ ID NO: 1190) | atctttga gatccagacc at |
| (SEQ ID NO: 1191) | tctttga gatccagacc atg |
| (SEQ ID NO: 1192) | ctttga gatccagacc atgc |
| (SEQ ID NO: 1193) | tttga gatccagacc atgcc |
| (SEQ ID NO: 1194) | ttga gatccagacc atgccc |
| (SEQ ID NO: 1195) | tga gatccagacc atgccca |
| (SEQ ID NO: 1196) | ga gatccagacc atgcccaa |
| (SEQ ID NO: 1197) | a gatccagacc atgcccaat |
| (SEQ ID NO: 1198) | gatccagacc atgcccaatg |
| (SEQ ID NO: 1199) | atccagacc atgcccaatg g |
| (SEQ ID NO: 1200) | tccagacc atgcccaatg gc |
| (SEQ ID NO: 1201) | ccagacc atgcccaatg gca |
| (SEQ ID NO: 1202) | cagacc atgcccaatg gcaa |
| (SEQ ID NO: 1203) | agacc atgcccaatg gcaaa |
| (SEQ ID NO: 1204) | gacc atgcccaatg gcaaaa |
| (SEQ ID NO: 1205) | acc atgcccaatg gcaaaac |
| (SEQ ID NO: 1206) | cc atgcccaatg gcaaaacc |
| (SEQ ID NO: 1207) | c atgcccaatg gcaaaaccc |
| (SEQ ID NO: 1208) | atgcccaatg gcaaaacccg |
| (SEQ ID NO: 1209) | tgcccaatg gcaaaacccg g |
| (SEQ ID NO: 1210) | gcccaatg gcaaaacccg ga |
| (SEQ ID NO: 1211) | cccaatg gcaaaacccg gac |
| (SEQ ID NO: 1212) | ccaatg gcaaaacccg gacc |
| (SEQ ID NO: 1213) | caatg gcaaaacccg gacct |
| (SEQ ID NO: 1214) | aatg gcaaaacccg gacctc |
| (SEQ ID NO: 1215) | atg gcaaaacccg gacctcc |
| (SEQ ID NO: 1216) | tg gcaaaacccg gacctccc |
| (SEQ ID NO: 1217) | g gcaaaacccg gacctccct |
| (SEQ ID NO: 1218) | gcaaaacccg gacctccctc |
| (SEQ ID NO: 1219) | caaaacccg gacctccctc a |
| (SEQ ID NO: 1220) | aaaacccg gacctccctc aa |
| (SEQ ID NO: 1221) | aaacccg gacctccctc aag |
| (SEQ ID NO: 1222) | aacccg gacctccctc aaga |
| (SEQ ID NO: 1223) | acccg gacctccctc aagac |
| (SEQ ID NO: 1224) | cccg gacctccctc aagacc |
| (SEQ ID NO: 1225) | ccg gacctccctc aagacca |
| (SEQ ID NO: 1226) | cg gacctccctc aagaccat |
| (SEQ ID NO: 1227) | g gacctccctc aagaccatg |
| (SEQ ID NO: 1228) | gacctccctc aagaccatga |
| (SEQ ID NO: 1229) | acctccctc aagaccatga g |
| (SEQ ID NO: 1230) | cctccctc aagaccatga gc |
| (SEQ ID NO: 1231) | ctccctc aagaccatga gcc |
| (SEQ ID NO: 1232) | tccctc aagaccatga gccg |
| (SEQ ID NO: 1233) | ccctc aagaccatga gccgt |
| (SEQ ID NO: 1234) | cctc aagaccatga gccgta |
| (SEQ ID NO: 1235) | ctc aagaccatga gccgtag |
| (SEQ ID NO: 1236) | tc aagaccatga gccgtagg |
| (SEQ ID NO: 1237) | c aagaccatga gccgtagga |
| (SEQ ID NO: 1238) | aagaccatga gccgtaggaa |
| (SEQ ID NO: 1239) | agaccatga gccgtaggaa g |
| (SEQ ID NO: 1240) | gaccatga gccgtaggaa gc |
| (SEQ ID NO: 1241) | accatga gccgtaggaa gct |
| (SEQ ID NO: 1242) | ccatga gccgtaggaa gctc |
| (SEQ ID NO: 1243) | catga gccgtaggaa gctct |
| (SEQ ID NO: 1244) | atga gccgtaggaa gctctc |
| (SEQ ID NO: 1245) | tga gccgtaggaa gctctcc |
| (SEQ ID NO: 1246) | ga gccgtaggaa gctctccc |
| (SEQ ID NO: 1247) | a gccgtaggaa gctctccca |
| (SEQ ID NO: 1248) | gccgtaggaa gctctcccag |
| (SEQ ID NO: 1249) | ccgtaggaa gctctcccag c |
| (SEQ ID NO: 1250) | cgtaggaa gctctcccag ca |
| (SEQ ID NO: 1251) | gtaggaa gctctcccag cag |
| (SEQ ID NO: 1252) | taggaa gctctcccag caga |
| (SEQ ID NO: 1253) | aggaa gctctcccag cagaa |
| (SEQ ID NO: 1254) | ggaa gctctcccag cagaag |
| (SEQ ID NO: 1255) | gaa gctctcccag cagaagg |
| (SEQ ID NO: 1256) | aa gctctcccag cagaagga |
| (SEQ ID NO: 1257) | a gctctcccag cagaaggag |
| (SEQ ID NO: 1258) | gctctcccag cagaaggaga |
| (SEQ ID NO: 1259) | ctctcccag cagaaggaga a |
| (SEQ ID NO: 1260) | tctcccag cagaaggaga ag |
| (SEQ ID NO: 1261) | ctcccag cagaaggaga aga |
| (SEQ ID NO: 1262) | tcccag cagaaggaga agaa |
| (SEQ ID NO: 1263) | cccag cagaaggaga agaaa |
| (SEQ ID NO: 1264) | ccag cagaaggaga agaaag |
| (SEQ ID NO: 1265) | cag cagaaggaga agaaagc |
| (SEQ ID NO: 1266) | ag cagaaggaga agaaagcc |
| (SEQ ID NO: 1267) | g cagaaggaga agaaagcca |
| (SEQ ID NO: 1268) | cagaaggaga agaaagccac |
| (SEQ ID NO: 1269) | agaaggaga agaaagccac t |
| (SEQ ID NO: 1270) | gaaggaga agaaagccac tc |
| (SEQ ID NO: 1271) | aaggaga agaaagccac tca |
| (SEQ ID NO: 1272) | aggaga agaaagccac tcag |
| (SEQ ID NO: 1273) | ggaga agaaagccac tcaga |
| (SEQ ID NO: 1274) | gaga agaaagccac tcagat |
| (SEQ ID NO: 1275) | aga agaaagccac tcagatg |
| (SEQ ID NO: 1276) | ga agaaagccac tcagatgc |
| (SEQ ID NO: 1277) | a agaaagccac tcagatgct |
| (SEQ ID NO: 1278) | agaaagccac tcagatgctc |
| (SEQ ID NO: 1279) | gaaagccac tcagatgctc g |
| (SEQ ID NO: 1280) | aaagccac tcagatgctc gc |
| (SEQ ID NO: 1281) | aagccac tcagatgctc gcc |
| (SEQ ID NO: 1282) | agccac tcagatgctc gcca |
| (SEQ ID NO: 1283) | gccac tcagatgctc gccat |
| (SEQ ID NO: 1284) | ccac tcagatgctc gccatt |
| (SEQ ID NO: 1285) | cac tcagatgctc gccattg |
| (SEQ ID NO: 1286) | ac tcagatgctc gccattgt |
| (SEQ ID NO: 1287) | c tcagatgctc gccattgtt |
| (SEQ ID NO: 1288) | tcagatgctc gccattgttc |
| (SEQ ID NO: 1289) | cagatgctc gccattgttc t |
| (SEQ ID NO: 1290) | agatgctc gccattgttc tc |
| (SEQ ID NO: 1291) | gatgctc gccattgttc tcg |
| (SEQ ID NO: 1292) | atgctc gccattgttc tcgg |
| (SEQ ID NO: 1293) | tgctc gccattgttc tcggc |
| (SEQ ID NO: 1294) | gctc gccattgttc tcggcg |
| (SEQ ID NO: 1295) | ctc gccattgttc tcggcgt |
| (SEQ ID NO: 1296) | tc gccattgttc tcggcgtg |
| (SEQ ID NO: 1297) | c gccattgttc tcggcgtgt |
| (SEQ ID NO: 1298) | gccattgttc tcggcgtgtt |
| (SEQ ID NO: 1299) | ccattgttc tcggcgtgtt c |
| (SEQ ID NO: 1300) | cattgttc tcggcgtgtt ca |
| (SEQ ID NO: 1301) | attgttc tcggcgtgtt cat |
| (SEQ ID NO: 1302) | ttgttc tcggcgtgtt catc |
| (SEQ ID NO: 1303) | tgttc tcggcgtgtt catca |
| (SEQ ID NO: 1304) | gttc tcggcgtgtt catcat |
| (SEQ ID NO: 1305) | ttc tcggcgtgtt catcatc |
| (SEQ ID NO: 1306) | tc tcggcgtgtt catcatct |
| (SEQ ID NO: 1307) | c tcggcgtgtt catcatctg |
| (SEQ ID NO: 1308) | tcggcgtgtt catcatctgc |
| (SEQ ID NO: 1309) | tcggcgtgtt catcatctgc T |
| (SEQ ID NO: 1310) | ggcgtgtt catcatctgc tg |
| (SEQ ID NO: 1311) | gcgtgtt catcatctgc tgg |
| (SEQ ID NO: 1312) | cgtgtt catcatctgc tggc |
| (SEQ ID NO: 1313) | gtgtt catcatctgc tggct |
| (SEQ ID NO: 1314) | tgtt catcatctgc tggctg |
| (SEQ ID NO: 1315) | gtt catcatctgc tggctgc |
| (SEQ ID NO: 1316) | tt catcatctgc tggctgcc |
| (SEQ ID NO: 1317) | t catcatctgc tggctgccc |
| (SEQ ID NO: 1318) | catcatctgc tggctgccct |
| (SEQ ID NO: 1319) | atcatctgc tggctgccct t |
| (SEQ ID NO: 1320) | tcatctgc tggctgccct tc |
| (SEQ ID NO: 1321) | catctgc tggctgccct tct |
| (SEQ ID NO: 1322) | atctgc tggctgccct tctt |
| (SEQ ID NO: 1323) | tctgc tggctgccct tcttc |
| (SEQ ID NO: 1324) | ctgc tggctgccct tcttca |
| (SEQ ID NO: 1325) | tgc tggctgccct tcttcat |
| (SEQ ID NO: 1326) | gc tggctgccct tcttcatc |
| (SEQ ID NO: 1327) | c tggctgccct tcttcatca |
| (SEQ ID NO: 1328) | tggctgccct tcttcatcac |
| (SEQ ID NO: 1329) | ggctgccct tcttcatcac a |
| (SEQ ID NO: 1330) | gctgccct tcttcatcac ac |
| (SEQ ID NO: 1331) | ctgccct tcttcatcac aca |
| (SEQ ID NO: 1332) | tgccct tcttcatcac acac |
| (SEQ ID NO: 1333) | gccct tcttcatcac acaca |
| (SEQ ID NO: 1334) | ccct tcttcatcac acacat |
| (SEQ ID NO: 1335) | cct tcttcatcac acacatc |
| (SEQ ID NO: 1336) | ct tcttcatcac acacatcc |
| (SEQ ID NO: 1337) | t tcttcatcac acacatcct |
| (SEQ ID NO: 1338) | tcttcatcac acacatcctg |
| (SEQ ID NO: 1339) | cttcatcac acacatcctg a |
| (SEQ ID NO: 1340) | ttcatcac acacatcctg aa |
| (SEQ ID NO: 1341) | tcatcac acacatcctg aac |
| (SEQ ID NO: 1342) | catcac acacatcctg aaca |
| (SEQ ID NO: 1343) | atcac acacatcctg aacat |
| (SEQ ID NO: 1344) | tcac acacatcctg aacata |
| (SEQ ID NO: 1345) | cac acacatcctg aacatac |
| (SEQ ID NO: 1346) | ac acacatcctg aacataca |
| (SEQ ID NO: 1347) | c acacatcctg aacatacac |
| (SEQ ID NO: 1348) | acacatcctg aacatacact |
| (SEQ ID NO: 1349) | cacatcctg aacatacact g |
| (SEQ ID NO: 1350) | acatcctg aacatacact gt |
| (SEQ ID NO: 1351) | catcctg aacatacact gtg |
| (SEQ ID NO: 1352) | atcctg aacatacact gtga |
| (SEQ ID NO: 1353) | tcctg aacatacact gtgac |
| (SEQ ID NO: 1354) | cctg aacatacact gtgact |
| (SEQ ID NO: 1355) | ctg aacatacact gtgactg |
| (SEQ ID NO: 1356) | tg aacatacact gtgactgc |
| (SEQ ID NO: 1357) | g aacatacact gtgactgca |
| (SEQ ID NO: 1358) | aacatacact gtgactgcaa |
| (SEQ ID NO: 1359) | acatacact gtgactgcaa c |
| (SEQ ID NO: 1360) | catacact gtgactgcaa ca |
| (SEQ ID NO: 1361) | atacact gtgactgcaa cat |
| (SEQ ID NO: 1362) | tacact gtgactgcaa catc |
| (SEQ ID NO: 1363) | acact gtgactgcaa catcc |
| (SEQ ID NO: 1364) | cact gtgactgcaa catccc |
| (SEQ ID NO: 1365) | act gtgactgcaa catcccg |
| (SEQ ID NO: 1366) | ct gtgactgcaa catcccgc |
| (SEQ ID NO: 1367) | t gtgactgcaa catcccgcc |
| (SEQ ID NO: 1368) | gtgactgcaa catcccgcct |
| (SEQ ID NO: 1369) | tgactgcaa catcccgcct g |
| (SEQ ID NO: 1370) | gactgcaa catcccgcct gt |
| (SEQ ID NO: 1371) | actgcaa catcccgcct gtc |
| (SEQ ID NO: 1372) | ctgcaa catcccgcct gtcc |
| (SEQ ID NO: 1373) | tgcaa catcccgcct gtcct |
| (SEQ ID NO: 1374) | gcaa catcccgcct gtcctg |
| (SEQ ID NO: 1375) | caa catcccgcct gtcctgt |
| (SEQ ID NO: 1376) | aa catcccgcct gtcctgta |
| (SEQ ID NO: 1377) | a catcccgcct gtcctgtac |
| (SEQ ID NO: 1378) | catcccgcct gtcctgtaca |
| (SEQ ID NO: 1379) | atcccgcct gtcctgtaca g |
| (SEQ ID NO: 1380) | tcccgcct gtcctgtaca gc |
| (SEQ ID NO: 1381) | cccgcct gtcctgtaca gcg |
| (SEQ ID NO: 1382) | ccgcct gtcctgtaca gcgc |
| (SEQ ID NO: 1383) | cgcct gtcctgtaca gcgcc |
| (SEQ ID NO: 1384) | gcct gtcctgtaca gcgcct |
| (SEQ ID NO: 1385) | cct gtcctgtaca gcgcctt |
| (SEQ ID NO: 1386) | ct gtcctgtaca gcgccttc |
| (SEQ ID NO: 1387) | t gtcctgtaca gcgccttca |
| (SEQ ID NO: 1388) | gtcctgtaca gcgccttcac |
| (SEQ ID NO: 1389) | tcctgtaca gcgccttcac g |
| (SEQ ID NO: 1390) | cctgtaca gcgccttcac gt |
| (SEQ ID NO: 1391) | ctgtaca gcgccttcac gtg |
| (SEQ ID NO: 1392) | tgtaca gcgccttcac gtgg |
| (SEQ ID NO: 1393) | gtaca gcgccttcac gtggc |
| (SEQ ID NO: 1394) | taca gcgccttcac gtggct |
| (SEQ ID NO: 1395) | aca gcgccttcac gtggctg |
| (SEQ ID NO: 1396) | ca gcgccttcac gtggctgg |
| (SEQ ID NO: 1397) | a gcgccttcac gtggctggg |
| (SEQ ID NO: 1398) | gcgccttcac gtggctgggc |
| (SEQ ID NO: 1399) | cgccttcac gtggctgggc t |
| (SEQ ID NO: 1400) | gccttcac gtggctgggc ta |
| (SEQ ID NO: 1401) | ccttcac gtggctgggc tat |
| (SEQ ID NO: 1402) | cttcac gtggctgggc tatg |
| (SEQ ID NO: 1403) | ttcac gtggctgggc tatgt |
| (SEQ ID NO: 1404) | tcac gtggctgggc tatgtc |
| (SEQ ID NO: 1405) | cac gtggctgggc tatgtca |
| (SEQ ID NO: 1406) | ac gtggctgggc tatgtcaa |
| (SEQ ID NO: 1407) | c gtggctgggc tatgtcaac |
| (SEQ ID NO: 1408) | gtggctgggc tatgtcaaca |
| (SEQ ID NO: 1409) | tggctgggc tatgtcaaca g |
| (SEQ ID NO: 1410) | ggctgggc tatgtcaaca gc |
| (SEQ ID NO: 1411) | gctgggc tatgtcaaca gcg |
| (SEQ ID NO: 1412) | ctgggc tatgtcaaca gcgc |
| (SEQ ID NO: 1413) | tgggc tatgtcaaca gcgcc |
| (SEQ ID NO: 1414) | gggc tatgtcaaca gcgccg |
| (SEQ ID NO: 1415) | ggc tatgtcaaca gcgccgt |
| (SEQ ID NO: 1416) | gc tatgtcaaca gcgccgtg |
| (SEQ ID NO: 1417) | c tatgtcaaca gcgccgtga |
| (SEQ ID NO: 1418) | tatgtcaaca gcgccgtgaa |
| (SEQ ID NO: 1419) | atgtcaaca gcgccgtgaa c |
| (SEQ ID NO: 1420) | tgtcaaca gcgccgtgaa cc |
| (SEQ ID NO: 1421) | gtcaaca gcgccgtgaa ccc |
| (SEQ ID NO: 1422) | tcaaca gcgccgtgaa cccc |
| (SEQ ID NO: 1423) | caaca gcgccgtgaa cccca |
| (SEQ ID NO: 1424) | aaca gcgccgtgaa ccccat |
| (SEQ ID NO: 1425) | aca gcgccgtgaa ccccatc |
| (SEQ ID NO: 1426) | ca gcgccgtgaa ccccatca |
| (SEQ ID NO: 1427) | a gcgccgtgaa ccccatcat |
| (SEQ ID NO: 1428) | gcgccgtgaa ccccatcatc |
| (SEQ ID NO: 1429) | cgccgtgaa ccccatcatc t |
| (SEQ ID NO: 1430) | gccgtgaa ccccatcatc ta |
| (SEQ ID NO: 1431) | ccgtgaa ccccatcatc tac |
| (SEQ ID NO: 1432) | cgtgaa ccccatcatc taca |
| (SEQ ID NO: 1433) | gtgaa ccccatcatc tacac |
| (SEQ ID NO: 1434) | tgaa ccccatcatc tacacc |
| (SEQ ID NO: 1435) | gaa ccccatcatc tacacca |
| (SEQ ID NO: 1436) | aa ccccatcatc tacaccac |
| (SEQ ID NO: 1437) | a ccccatcatc tacaccacc |
| (SEQ ID NO: 1438) | ccccatcatc tacaccacct |
| (SEQ ID NO: 1439) | cccatcatc tacaccacct t |
| (SEQ ID NO: 1440) | ccatcatc tacaccacct tc |
| (SEQ ID NO: 1441) | catcatc tacaccacct tca |
| (SEQ ID NO: 1442) | atcatc tacaccacct tcaa |
| (SEQ ID NO: 1443) | tcatc tacaccacct tcaac |
| (SEQ ID NO: 1444) | catc tacaccacct tcaaca |
| (SEQ ID NO: 1445) | atc tacaccacct tcaacat |
| (SEQ ID NO: 1446) | tc tacaccacct tcaacatt |
| (SEQ ID NO: 1447) | c tacaccacct tcaacattg |
| (SEQ ID NO: 1448) | tacaccacct tcaacattga |
| (SEQ ID NO: 1449) | acaccacct tcaacattga g |
| (SEQ ID NO: 1450) | caccacct tcaacattga gt |
| (SEQ ID NO: 1451) | accacct tcaacattga gtt |
| (SEQ ID NO: 1452) | ccacct tcaacattga gttc |
| (SEQ ID NO: 1453) | cacct tcaacattga gttcc |
| (SEQ ID NO: 1454) | acct tcaacattga gttccg |
| (SEQ ID NO: 1455) | cct tcaacattga gttccgc |
| (SEQ ID NO: 1456) | ct tcaacattga gttccgca |
| (SEQ ID NO: 1457) | t tcaacattga gttccgcaa |
| (SEQ ID NO: 1458) | tcaacattga gttccgcaag |
| (SEQ ID NO: 1459) | caacattga gttccgcaag g |
| (SEQ ID NO: 1460) | aacattga gttccgcaag gc |
| (SEQ ID NO: 1461) | acattga gttccgcaag gcc |
| (SEQ ID NO: 1462) | cattga gttccgcaag gcct |
| (SEQ ID NO: 1463) | attga gttccgcaag gcctt |
| (SEQ ID NO: 1464) | ttga gttccgcaag gccttc |
| (SEQ ID NO: 1465) | tga gttccgcaag gccttcc |
| (SEQ ID NO: 1466) | ga gttccgcaag gccttcct |
| (SEQ ID NO: 1467) | a gttccgcaag gccttcctg |
| (SEQ ID NO: 1468) | gttccgcaag gccttcctga |
| (SEQ ID NO: 1469) | ttccgcaag gccttcctga a |
| (SEQ ID NO: 1470) | tccgcaag gccttcctga ag |
| (SEQ ID NO: 1471) | ccgcaag gccttcctga aga |
| (SEQ ID NO: 1472) | cgcaag gccttcctga agat |
| (SEQ ID NO: 1473) | gcaag gccttcctga agatc |
| (SEQ ID NO: 1474) | caag gccttcctga agatcc |
| (SEQ ID NO: 1475) | aag gccttcctga agatcct |
| (SEQ ID NO: 1476) | ag gccttcctga agatcctc |
| (SEQ ID NO: 1477) | g gccttcctga agatcctcc |
| (SEQ ID NO: 1478) | gccttcctga agatcctcca |
| (SEQ ID NO: 1479) | ccttcctga agatcctcca c |
| (SEQ ID NO: 1480) | cttcctga agatcctcca ct |
| (SEQ ID NO: 1481) | ttcctga agatcctcca ctg |
| (SEQ ID NO: 1482) | tcctga agatcctcca ctgc |
| (SEQ ID NO: 1483) | cctga agatcctcca ctgct |
| (SEQ ID NO: 1484) | ctga agatcctcca ctgctg |
| (SEQ ID NO: 1485) | tga agatcctcca ctgctga |
| (SEQ ID NO: 1486) | ga agatcctcca ctgctgac |
| (SEQ ID NO: 1487) | a agatcctcca ctgctgact |
| (SEQ ID NO: 1488) | agatcctcca ctgctgactc |
| (SEQ ID NO: 1489) | gatcctcca ctgctgactc t |
| (SEQ ID NO: 1490) | atcctcca ctgctgactc tg |
| (SEQ ID NO: 1491) | tcctcca ctgctgactc tgc |
| (SEQ ID NO: 1492) | cctcca ctgctgactc tgct |
| (SEQ ID NO: 1493) | ctcca ctgctgactc tgctg |
| (SEQ ID NO: 1494) | tcca ctgctgactc tgctgc |
| (SEQ ID NO: 1495) | cca ctgctgactc tgctgcc |
| (SEQ ID NO: 1496) | ca ctgctgactc tgctgcct |
| (SEQ ID NO: 1497) | a ctgctgactc tgctgcctg |
| (SEQ ID NO: 1498) | ctgctgactc tgctgcctgc |
| (SEQ ID NO: 1499) | tgctgactc tgctgcctgc c |
| (SEQ ID NO: 1500) | gctgactc tgctgcctgc cc |
| (SEQ ID NO: 1501) | ctgactc tgctgcctgc ccg |
| (SEQ ID NO: 1502) | tgactc tgctgcctgc ccgc |
| (SEQ ID NO: 1503) | gactc tgctgcctgc ccgca |
| (SEQ ID NO: 1504) | actc tgctgcctgc ccgcac |
| (SEQ ID NO: 1505) | ctc tgctgcctgc ccgcaca |
| (SEQ ID NO: 1506) | tc tgctgcctgc ccgcacag |
| (SEQ ID NO: 1507) | c tgctgcctgc ccgcacagc |
| (SEQ ID NO: 1508) | tgctgcctgc ccgcacagca |
| (SEQ ID NO: 1509) | gctgcctgc ccgcacagca g |
| (SEQ ID NO: 1510) | ctgcctgc ccgcacagca gc |
| (SEQ ID NO: 1511) | tgcctgc ccgcacagca gcc |
| (SEQ ID NO: 1512) | gcctgc ccgcacagca gcct |
| (SEQ ID NO: 1513) | cctgc ccgcacagca gcctg |
| (SEQ ID NO: 1514) | ctgc ccgcacagca gcctgc |
| (SEQ ID NO: 1515) | tgc ccgcacagca gcctgct |
| (SEQ ID NO: 1516) | gc ccgcacagca gcctgctt |
| (SEQ ID NO: 1517) | c ccgcacagca gcctgcttc |
| (SEQ ID NO: 1518) | ccgcacagca gcctgcttcc |
| (SEQ ID NO: 1519) | cgcacagca gcctgcttcc c |
| (SEQ ID NO: 1520) | gcacagca gcctgcttcc ca |
| (SEQ ID NO: 1521) | cacagca gcctgcttcc cac |
| (SEQ ID NO: 1522) | acagca gcctgcttcc cacc |
| (SEQ ID NO: 1523) | cagca gcctgcttcc cacct |
| (SEQ ID NO: 1524) | agca gcctgcttcc cacctc |
| (SEQ ID NO: 1525) | gca gcctgcttcc cacctcc |
| (SEQ ID NO: 1526) | ca gcctgcttcc cacctccc |
| (SEQ ID NO: 1527) | a gcctgcttcc cacctccct |
| (SEQ ID NO: 1528) | gcctgcttcc cacctccctg |
| (SEQ ID NO: 1529) | cctgcttcc cacctccctg c |
| (SEQ ID NO: 1530) | ctgcttcc cacctccctg cc |
| (SEQ ID NO: 1531) | tgcttcc cacctccctg ccc |
| (SEQ ID NO: 1532) | gcttcc cacctccctg ccca |
| (SEQ ID NO: 1533) | cttcc cacctccctg cccag |
| (SEQ ID NO: 1534) | ttcc cacctccctg cccagg |
| (SEQ ID NO: 1535) | tcc cacctccctg cccaggc |
| (SEQ ID NO: 1536) | cc cacctccctg cccaggcc |
| (SEQ ID NO: 1537) | c cacctccctg cccaggccg |
| (SEQ ID NO: 1538) | cacctccctg cccaggccgg |
| (SEQ ID NO: 1539) | acctccctg cccaggccgg c |
| (SEQ ID NO: 1540) | cctccctg cccaggccgg cc |
| (SEQ ID NO: 1541) | ctccctg cccaggccgg cca |
| (SEQ ID NO: 1542) | tccctg cccaggccgg ccag |
| (SEQ ID NO: 1543) | ccctg cccaggccgg ccagc |
| (SEQ ID NO: 1544) | cctg cccaggccgg ccagcc |
| (SEQ ID NO: 1545) | ctg cccaggccgg ccagcct |
| (SEQ ID NO: 1546) | tg cccaggccgg ccagcctc |
| (SEQ ID NO: 1547) | g cccaggccgg ccagcctca |
| (SEQ ID NO: 1548) | cccaggccgg ccagcctcac |
| (SEQ ID NO: 1549) | ccaggccgg ccagcctcac c |
| (SEQ ID NO: 1550) | caggccgg ccagcctcac cc |
| (SEQ ID NO: 1551) | aggccgg ccagcctcac cct |
| (SEQ ID NO: 1552) | ggccgg ccagcctcac cctt |
| (SEQ ID NO: 1553) | gccgg ccagcctcac ccttg |
| (SEQ ID NO: 1554) | ccgg ccagcctcac ccttgc |
| (SEQ ID NO: 1555) | cgg ccagcctcac ccttgcg |
| (SEQ ID NO: 1556) | gg ccagcctcac ccttgcga |
| (SEQ ID NO: 1557) | g ccagcctcac ccttgcgaa |
| (SEQ ID NO: 1558) | ccagcctcac ccttgcgaac |
| (SEQ ID NO: 1559) | cagcctcac ccttgcgaac c |
| (SEQ ID NO: 1560) | agcctcac ccttgcgaac cg |
| (SEQ ID NO: 1561) | gcctcac ccttgcgaac cgt |
| (SEQ ID NO: 1562) | cctcac ccttgcgaac cgtg |
| (SEQ ID NO: 1563) | ctcac ccttgcgaac cgtga |
| (SEQ ID NO: 1564) | tcac ccttgcgaac cgtgag |
| (SEQ ID NO: 1565) | cac ccttgcgaac cgtgagc |
| (SEQ ID NO: 1566) | ac ccttgcgaac cgtgagca |
| (SEQ ID NO: 1567) | c ccttgcgaac cgtgagcag |
| (SEQ ID NO: 1568) | ccttgcgaac cgtgagcagg |
| (SEQ ID NO: 1569) | cttgcgaac cgtgagcagg a |
| (SEQ ID NO: 1570) | ttgcgaac cgtgagcagg aa |
| (SEQ ID NO: 1571) | tgcgaac cgtgagcagg aag |
| (SEQ ID NO: 1572) | gcgaac cgtgagcagg aagg |
| (SEQ ID NO: 1573) | cgaac cgtgagcagg aaggc |
| (SEQ ID NO: 1574) | gaac cgtgagcagg aaggcc |
| (SEQ ID NO: 1575) | aac cgtgagcagg aaggcct |
| (SEQ ID NO: 1576) | ac cgtgagcagg aaggcctg |
| (SEQ ID NO: 1577) | c cgtgagcagg aaggcctgg |
| (SEQ ID NO: 1578) | cgtgagcagg aaggcctggg |
| (SEQ ID NO: 1579) | gtgagcagg aaggcctggg t |
| (SEQ ID NO: 1580) | tgagcagg aaggcctggg tg |
| (SEQ ID NO: 1581) | gagcagg aaggcctggg tgg |
| (SEQ ID NO: 1582) | agcagg aaggcctggg tgga |
| (SEQ ID NO: 1583) | gcagg aaggcctggg tggat |
| (SEQ ID NO: 1584) | cagg aaggcctggg tggatc |
| (SEQ ID NO: 1585) | agg aaggcctggg tggatcg |
| (SEQ ID NO: 1586) | gg aaggcctggg tggatcgg |
| (SEQ ID NO: 1587) | g aaggcctggg tggatcggc |
| (SEQ ID NO: 1588) | aaggcctggg tggatcggcc |
| (SEQ ID NO: 1589) | aggcctggg tggatcggcc t |
| (SEQ ID NO: 1590) | ggcctggg tggatcggcc tc |
| (SEQ ID NO: 1591) | gcctggg tggatcggcc tcc |
| (SEQ ID NO: 1592) | cctggg tggatcggcc tcct |
| (SEQ ID NO: 1593) | ctggg tggatcggcc tcctc |
| (SEQ ID NO: 1594) | tggg tggatcggcc tcctct |
| (SEQ ID NO: 1595) | ggg tggatcggcc tcctctt |
| (SEQ ID NO: 1596) | gg tggatcggcc tcctcttc |
| (SEQ ID NO: 1597) | g tggatcggcc tcctcttca |
| (SEQ ID NO: 1598) | tggatcggcc tcctcttcac |
| (SEQ ID NO: 1599) | ggatcggcc tcctcttcac c |
| (SEQ ID NO: 1600) | gatcggcc tcctcttcac cc |
| (SEQ ID NO: 1601) | atcggcc tcctcttcac ccc |
| (SEQ ID NO: 1602) | tcggcc tcctcttcac cccg |
| (SEQ ID NO: 1603) | cggcc tcctcttcac cccgg |
| (SEQ ID NO: 1604) | ggcc tcctcttcac cccggc |
| (SEQ ID NO: 1605) | gcc tcctcttcac cccggca |
| (SEQ ID NO: 1606) | cc tcctcttcac cccggcag |
| (SEQ ID NO: 1607) | c tcctcttcac cccggcagg |
| (SEQ ID NO: 1608) | tcctcttcac cccggcaggc |
| (SEQ ID NO: 1609) | cctcttcac cccggcaggc c |
| (SEQ ID NO: 1610) | ctcttcac cccggcaggc cc |
| (SEQ ID NO: 1611) | tcttcac cccggcaggc cct |
| (SEQ ID NO: 1612) | cttcac cccggcaggc cctg |
| (SEQ ID NO: 1613) | ttcac cccggcaggc cctgc |
| (SEQ ID NO: 1614) | tcac cccggcaggc cctgca |
| (SEQ ID NO: 1615) | cac cccggcaggc cctgcag |
| (SEQ ID NO: 1616) | ac cccggcaggc cctgcagt |
| (SEQ ID NO: 1617) | c cccggcaggc cctgcagtg |
| (SEQ ID NO: 1618) | cccggcaggc cctgcagtgt |
| (SEQ ID NO: 1619) | ccggcaggc cctgcagtgt t |
| (SEQ ID NO: 1620) | cggcaggc cctgcagtgt tc |
| (SEQ ID NO: 1621) | ggcaggc cctgcagtgt tcg |
| (SEQ ID NO: 1622) | gcaggc cctgcagtgt tcgc |
| (SEQ ID NO: 1623) | caggc cctgcagtgt tcgct |
| (SEQ ID NO: 1624) | aggc cctgcagtgt tcgctt |
| (SEQ ID NO: 1625) | ggc cctgcagtgt tcgcttg |
| (SEQ ID NO: 1626) | gc cctgcagtgt tcgcttgg |
| (SEQ ID NO: 1627) | c cctgcagtgt tcgcttggc |
| (SEQ ID NO: 1628) | cctgcagtgt tcgcttggct |
| (SEQ ID NO: 1629) | ctgcagtgt tcgcttggct c |
| (SEQ ID NO: 1630) | tgcagtgt tcgcttggct cc |
| (SEQ ID NO: 1631) | gcagtgt tcgcttggct cca |
| (SEQ ID NO: 1632) | cagtgt tcgcttggct ccat |
| (SEQ ID NO: 1633) | agtgt tcgcttggct ccatg |
| (SEQ ID NO: 1634) | gtgt tcgcttggct ccatgc |
| (SEQ ID NO: 1635) | tgt tcgcttggct ccatgct |
| (SEQ ID NO: 1636) | gt tcgcttggct ccatgctc |
| (SEQ ID NO: 1637) | t tcgcttggct ccatgctcc |
| (SEQ ID NO: 1638) | tcgcttggct ccatgctcct |
| (SEQ ID NO: 1639) | cgcttggct ccatgctcct c |
| (SEQ ID NO: 1640) | gcttggct ccatgctcct ca |
| (SEQ ID NO: 1641) | cttggct ccatgctcct cac |
| (SEQ ID NO: 1642) | ttggct ccatgctcct cact |
| (SEQ ID NO: 1643) | tggct ccatgctcct cactg |
| (SEQ ID NO: 1644) | ggct ccatgctcct cactgc |
| (SEQ ID NO: 1645) | gct ccatgctcct cactgcc |
| (SEQ ID NO: 1646) | ct ccatgctcct cactgccc |
| (SEQ ID NO: 1647) | t ccatgctcct cactgcccg |
| (SEQ ID NO: 1648) | ccatgctcct cactgcccgc |
| (SEQ ID NO: 1649) | catgctcct cactgcccgc a |
| (SEQ ID NO: 1650) | atgctcct cactgcccgc ac |
| (SEQ ID NO: 1651) | tgctcct cactgcccgc aca |
| (SEQ ID NO: 1652) | gctcct cactgcccgc acac |
| (SEQ ID NO: 1653) | ctcct cactgcccgc acacc |
| (SEQ ID NO: 1654) | tcct cactgcccgc acaccc |
| (SEQ ID NO: 1655) | cct cactgcccgc acaccct |
| (SEQ ID NO: 1656) | ct cactgcccgc acaccctc |
| (SEQ ID NO: 1657) | t cactgcccgc acaccctca |
| (SEQ ID NO: 1658) | cactgcccgc acaccctcac |
| (SEQ ID NO: 1659) | actgcccgc acaccctcac t |
| (SEQ ID NO: 1660) | ctgcccgc acaccctcac tc |
| (SEQ ID NO: 1661) | tgcccgc acaccctcac tct |
| (SEQ ID NO: 1662) | gcccgc acaccctcac tctg |
| (SEQ ID NO: 1663) | cccgc acaccctcac tctgc |
| (SEQ ID NO: 1664) | ccgc acaccctcac tctgcc |
| (SEQ ID NO: 1665) | cgc acaccctcac tctgcca |
| (SEQ ID NO: 1666) | gc acaccctcac tctgccag |
| (SEQ ID NO: 1667) | c acaccctcac tctgccagg |
| (SEQ ID NO: 1668) | acaccctcac tctgccaggg |
| (SEQ ID NO: 1669) | caccctcac tctgccaggg c |
| (SEQ ID NO: 1670) | accctcac tctgccaggg ca |
| (SEQ ID NO: 1671) | ccctcac tctgccaggg cag |
| (SEQ ID NO: 1672) | cctcac tctgccaggg cagt |
| (SEQ ID NO: 1673) | ctcac tctgccaggg cagtg |
| (SEQ ID NO: 1674) | tcac tctgccaggg cagtgc |
| (SEQ ID NO: 1675) | cac tctgccaggg cagtgct |
| (SEQ ID NO: 1676) | ac tctgccaggg cagtgcta |
| (SEQ ID NO: 1677) | c tctgccaggg cagtgctag |
| (SEQ ID NO: 1678) | tctgccaggg cagtgctagt |
| (SEQ ID NO: 1679) | ctgccaggg cagtgctagt g |
| (SEQ ID NO: 1680) | tgccaggg cagtgctagt ga |
| (SEQ ID NO: 1681) | gccaggg cagtgctagt gag |
| (SEQ ID NO: 1682) | ccaggg cagtgctagt gagc |
| (SEQ ID NO: 1683) | caggg cagtgctagt gagct |
| (SEQ ID NO: 1684) | aggg cagtgctagt gagctg |
| (SEQ ID NO: 1685) | ggg cagtgctagt gagctgg |
| (SEQ ID NO: 1686) | gg cagtgctagt gagctggg |
| (SEQ ID NO: 1687) | g cagtgctagt gagctgggc |
| (SEQ ID NO: 1688) | cagtgctagt gagctgggca |
| (SEQ ID NO: 1689) | agtgctagt gagctgggca t |
| (SEQ ID NO: 1690) | gtgctagt gagctgggca tg |
| (SEQ ID NO: 1691) | tgctagt gagctgggca tgg |
| (SEQ ID NO: 1692) | gctagt gagctgggca tggt |
| (SEQ ID NO: 1693) | ctagt gagctgggca tggta |
| (SEQ ID NO: 1694) | tagt gagctgggca tggtac |
| (SEQ ID NO: 1695) | agt gagctgggca tggtacc |
| (SEQ ID NO: 1696) | gt gagctgggca tggtacca |
| (SEQ ID NO: 1697) | t gagctgggca tggtaccag |
| (SEQ ID NO: 1698) | gagctgggca tggtaccagc |
| (SEQ ID NO: 1699) | agctgggca tggtaccagc c |
| (SEQ ID NO: 1700) | gctgggca tggtaccagc cc |
| (SEQ ID NO: 1701) | ctgggca tggtaccagc cct |
| (SEQ ID NO: 1702) | tgggca tggtaccagc cctg |
| (SEQ ID NO: 1703) | gggca tggtaccagc cctgg |
| (SEQ ID NO: 1704) | ggca tggtaccagc cctggg |
| (SEQ ID NO: 1705) | gca tggtaccagc cctgggg |
| (SEQ ID NO: 1706) | ca tggtaccagc cctggggc |
| (SEQ ID NO: 1707) | a tggtaccagc cctggggct |
| (SEQ ID NO: 1708) | tggtaccagc cctggggctg |
| (SEQ ID NO: 1709) | ggtaccagc cctggggctg g |
| (SEQ ID NO: 1710) | gtaccagc cctggggctg gg |
| (SEQ ID NO: 1711) | taccagc cctggggctg ggc |
| (SEQ ID NO: 1712) | accagc cctggggctg ggcc |
| (SEQ ID NO: 1713) | ccagc cctggggctg ggccc |
| (SEQ ID NO: 1714) | cagc cctggggctg ggcccc |
| (SEQ ID NO: 1715) | agc cctggggctg ggccccc |
| (SEQ ID NO: 1716) | gc cctggggctg ggcccccc |
| (SEQ ID NO: 1717) | c cctggggctg ggcccccca |
| (SEQ ID NO: 1718) | cctggggctg ggccccccag |
| (SEQ ID NO: 1719) | ctggggctg ggccccccag c |
| (SEQ ID NO: 1720) | tggggctg ggccccccag ct |
| (SEQ ID NO: 1721) | ggggctg ggccccccag ctc |
| (SEQ ID NO: 1722) | gggctg ggccccccag ctca |
| (SEQ ID NO: 1723) | ggctg ggccccccag ctcag |
| (SEQ ID NO: 1724) | gctg ggccccccag ctcagg |
| (SEQ ID NO: 1725) | ctg ggccccccag ctcaggg |
| (SEQ ID NO: 1726) | tg ggccccccag ctcagggg |
| (SEQ ID NO: 1727) | g ggccccccag ctcaggggc |
| (SEQ ID NO: 1728) | ggccccccag ctcaggggca |
| (SEQ ID NO: 1729) | gccccccag ctcaggggca g |
| (SEQ ID NO: 1730) | ccccccag ctcaggggca gc |
| (SEQ ID NO: 1731) | cccccag ctcaggggca gct |
| (SEQ ID NO: 1732) | ccccag ctcaggggca gctc |
| (SEQ ID NO: 1733) | cccag ctcaggggca gctca |
| (SEQ ID NO: 1734) | ccag ctcaggggca gctcat |
| (SEQ ID NO: 1735) | cag ctcaggggca gctcata |
| (SEQ ID NO: 1736) | ag ctcaggggca gctcatag |
| (SEQ ID NO: 1737) | g ctcaggggca gctcataga |
| (SEQ ID NO: 1738) | ctcaggggca gctcatagag |
| (SEQ ID NO: 1739) | tcaggggca gctcatagag t |
| (SEQ ID NO: 1740) | caggggca gctcatagag tc |
| (SEQ ID NO: 1741) | aggggca gctcatagag tcc |
| (SEQ ID NO: 1742) | ggggca gctcatagag tccc |
| (SEQ ID NO: 1743) | gggca gctcatagag tcccc |
| (SEQ ID NO: 1744) | ggca gctcatagag tccccc |
| (SEQ ID NO: 1745) | gca gctcatagag tcccccct |
| (SEQ ID NO: 1746) | ca gctcatagag tcccccctc |
| (SEQ ID NO: 1747) | a gctcatagag tcccccctc |
| (SEQ ID NO: 1748) | gctcatagag tcccccctcc |
| (SEQ ID NO: 1749) | ctcatagag tcccccctcc c |
| (SEQ ID NO: 1750) | tcatagag tcccccctcc ca |
| (SEQ ID NO: 1751) | catagag tcccccctcc cac |
| (SEQ ID NO: 1752) | atagag tcccccctcc cacc |
| (SEQ ID NO: 1753) | tagag tcccccctcc cacct |
| (SEQ ID NO: 1754) | agag tcccccctcc cacctc |
| (SEQ ID NO: 1755) | gag tcccccctcc cacctcc |
| (SEQ ID NO: 1756) | ag tcccccctcc cacctcca |
| (SEQ ID NO: 1757) | g tcccccctcc cacctccag |
| (SEQ ID NO: 1758) | tcccccctcc cacctccagt |
| (SEQ ID NO: 1759) | cccccctcc cacctccagt c |
| (SEQ ID NO: 1760) | ccccctcc cacctccagt cc |
| (SEQ ID NO: 1761) | cccctcc cacctccagt ccc |
| (SEQ ID NO: 1762) | ccctcc cacctccagt cccc |
| (SEQ ID NO: 1763) | cctcc cacctccagt ccccc |
| (SEQ ID NO: 1764) | ctcc cacctccagt ccccct |
| (SEQ ID NO: 1765) | tcc cacctccagt cccccta |
| (SEQ ID NO: 1766) | cc cacctccagt ccccctat |
| (SEQ ID NO: 1767) | c cacctccagt ccccctatc |
| (SEQ ID NO: 1768) | cacctccagt ccccctatcc |
| (SEQ ID NO: 1769) | acctccagt ccccctatcc t |
| (SEQ ID NO: 1770) | cctccagt ccccctatcc tt |
| (SEQ ID NO: 1771) | ctccagt ccccctatcc ttg |
| (SEQ ID NO: 1772) | tccagt ccccctatcc ttgg |
| (SEQ ID NO: 1773) | ccagt ccccctatcc ttggc |
| (SEQ ID NO: 1774) | cagt ccccctatcc ttggca |
| (SEQ ID NO: 1775) | agt ccccctatcc ttggcac |
| (SEQ ID NO: 1776) | gt ccccctatcc ttggcacc |
| (SEQ ID NO: 1777) | t ccccctatcc ttggcacca |
| (SEQ ID NO: 1778) | ccccctatcc ttggcaccaa |
| (SEQ ID NO: 1779) | cccctatcc ttggcaccaa a |
| (SEQ ID NO: 1780) | ccctatcc ttggcaccaa ag |
| (SEQ ID NO: 1781) | cctatcc ttggcaccaa aga |
| (SEQ ID NO: 1782) | ctatcc ttggcaccaa agat |
| (SEQ ID NO: 1783) | tatcc ttggcaccaa agatg |
| (SEQ ID NO: 1784) | atcc ttggcaccaa agatgc |
| (SEQ ID NO: 1785) | tcc ttggcaccaa agatgca |
| (SEQ ID NO: 1786) | cc ttggcaccaa agatgcag |
| (SEQ ID NO: 1787) | c ttggcaccaa agatgcagc |
| (SEQ ID NO: 1788) | ttggcaccaa agatgcagcc |
| (SEQ ID NO: 1789) | tggcaccaa agatgcagcc g |
| (SEQ ID NO: 1790) | ggcaccaa agatgcagcc gc |
| (SEQ ID NO: 1791) | gcaccaa agatgcagcc gcc |
| (SEQ ID NO: 1792) | caccaa agatgcagcc gcct |
| (SEQ ID NO: 1793) | accaa agatgcagcc gcctt |
| (SEQ ID NO: 1794) | ccaa agatgcagcc gccttc |
| (SEQ ID NO: 1795) | caa agatgcagcc gccttcc |
| (SEQ ID NO: 1796) | aa agatgcagcc gccttcct |
| (SEQ ID NO: 1797) | a agatgcagcc gccttcctt |
| (SEQ ID NO: 1798) | agatgcagcc gccttccttg |
| (SEQ ID NO: 1799) | gatgcagcc gccttccttg a |
| (SEQ ID NO: 1800) | atgcagcc gccttccttg ac |
| (SEQ ID NO: 1801) | tgcagcc gccttccttg acc |
| (SEQ ID NO: 1802) | gcagcc gccttccttg acct |
| (SEQ ID NO: 1803) | cagcc gccttccttg acctt |
| (SEQ ID NO: 1804) | agcc gccttccttg accttc |
| (SEQ ID NO: 1805) | gcc gccttccttg accttcc |
| (SEQ ID NO: 1806) | cc gccttccttg accttcct |
| (SEQ ID NO: 1807) | c gccttccttg accttcctc |
| (SEQ ID NO: 1808) | gccttccttg accttcctct |
| (SEQ ID NO: 1809) | ccttccttg accttcctct g |
| (SEQ ID NO: 1810) | cttccttg accttcctct gg |
| (SEQ ID NO: 1811) | ttccttg accttcctct ggg |
| (SEQ ID NO: 1812) | tccttg accttcctct gggg |
| (SEQ ID NO: 1813) | ccttg accttcctct ggggc |
| (SEQ ID NO: 1814) | cttg accttcctct ggggct |
| (SEQ ID NO: 1815) | ttg accttcctct ggggctc |
| (SEQ ID NO: 1816) | tg accttcctct ggggctct |
| (SEQ ID NO: 1817) | g accttcctct ggggctcta |
| (SEQ ID NO: 1818) | accttcctct ggggctctag |
| (SEQ ID NO: 1819) | ccttcctct ggggctctag g |
| (SEQ ID NO: 1820) | cttcctct ggggctctag gg |
| (SEQ ID NO: 1821) | ttcctct ggggctctag ggt |
| (SEQ ID NO: 1822) | tcctct ggggctctag ggtt |
| (SEQ ID NO: 1823) | cctct ggggctctag ggttg |
| (SEQ ID NO: 1824) | ctct ggggctctag ggttgc |
| (SEQ ID NO: 1825) | tct ggggctctag ggttgct |
| (SEQ ID NO: 1826) | ct ggggctctag ggttgctg |
| (SEQ ID NO: 1827) | t ggggctctag ggttgctgg |
| (SEQ ID NO: 1828) | ggggctctag ggttgctgga |
| (SEQ ID NO: 1829) | gggctctag ggttgctgga g |
| (SEQ ID NO: 1830) | ggctctag ggttgctgga gc |
| (SEQ ID NO: 1831) | gctctag ggttgctgga gcc |
| (SEQ ID NO: 1832) | ctctag ggttgctgga gcct |
| (SEQ ID NO: 1833) | tctag ggttgctgga gcctg |
| (SEQ ID NO: 1834) | ctag ggttgctgga gcctga |
| (SEQ ID NO: 1835) | tag ggttgctgga gcctgag |
| (SEQ ID NO: 1836) | ag ggttgctgga gcctgagt |
| (SEQ ID NO: 1837) | g ggttgctgga gcctgagtc |
| (SEQ ID NO: 1838) | ggttgctgga gcctgagtca |
| (SEQ ID NO: 1839) | gttgctgga gcctgagtca g |
| (SEQ ID NO: 1840) | ttgctgga gcctgagtca gg |
| (SEQ ID NO: 1841) | tgctgga gcctgagtca ggg |
| (SEQ ID NO: 1842) | gctgga gcctgagtca gggc |
| (SEQ ID NO: 1843) | ctgga gcctgagtca gggcc |
| (SEQ ID NO: 1844) | tgga gcctgagtca gggccc |
| (SEQ ID NO: 1845) | gga gcctgagtca gggccca |
| (SEQ ID NO: 1846) | ga gcctgagtca gggcccag |
| (SEQ ID NO: 1847) | a gcctgagtca gggcccaga |
| (SEQ ID NO: 1848) | gcctgagtca gggcccagag |
| (SEQ ID NO: 1849) | cctgagtca gggcccagag g |
| (SEQ ID NO: 1850) | ctgagtca gggcccagag gc |
| (SEQ ID NO: 1851) | tgagtca gggcccagag gct |
| (SEQ ID NO: 1852) | gagtca gggcccagag gctg |
| (SEQ ID NO: 1853) | agtca gggcccagag gctga |
| (SEQ ID NO: 1854) | gtca gggcccagag gctgag |
| (SEQ ID NO: 1855) | tca gggcccagag gctgagt |
| (SEQ ID NO: 1856) | ca gggcccagag gctgagtt |
| (SEQ ID NO: 1857) | a gggcccagag gctgagttt |
| (SEQ ID NO: 1858) | gggcccagag gctgagtttt |
| (SEQ ID NO: 1859) | ggcccagag gctgagtttt c |
| (SEQ ID NO: 1860) | gcccagag gctgagtttt ct |
| (SEQ ID NO: 1861) | cccagag gctgagtttt ctc |
| (SEQ ID NO: 1862) | ccagag gctgagtttt ctct |
| (SEQ ID NO: 1863) | cagag gctgagtttt ctctt |
| (SEQ ID NO: 1864) | agag gctgagtttt ctcttt |
| (SEQ ID NO: 1865) | gag gctgagtttt ctctttg |
| (SEQ ID NO: 1866) | ag gctgagtttt ctctttgt |
| (SEQ ID NO: 1867) | g gctgagtttt ctctttgtg |
| (SEQ ID NO: 1868) | gctgagtttt ctctttgtgg |
| (SEQ ID NO: 1869) | ctgagtttt ctctttgtgg g |
| (SEQ ID NO: 1870) | tgagtttt ctctttgtgg gg |
| (SEQ ID NO: 1871) | gagtttt ctctttgtgg ggc |
| (SEQ ID NO: 1872) | agtttt ctctttgtgg ggct |
| (SEQ ID NO: 1873) | gtttt ctctttgtgg ggctt |
| (SEQ ID NO: 1874) | tttt ctctttgtgg ggcttg |
| (SEQ ID NO: 1875) | ttt ctctttgtgg ggcttgg |
| (SEQ ID NO: 1876) | tt ctctttgtgg ggcttggc |
| (SEQ ID NO: 1877) | t ctctttgtgg ggcttggcg |
| (SEQ ID NO: 1878) | ctctttgtgg ggcttggcgt |
| (SEQ ID NO: 1879) | tctttgtgg ggcttggcgt g |
| (SEQ ID NO: 1880) | ctttgtgg ggcttggcgt gg |
| (SEQ ID NO: 1881) | tttgtgg ggcttggcgt gga |
| (SEQ ID NO: 1882) | ttgtgg ggcttggcgt ggag |
| (SEQ ID NO: 1883) | tgtgg ggcttggcgt ggagc |
| (SEQ ID NO: 1884) | gtgg ggcttggcgt ggagca |
| (SEQ ID NO: 1885) | tgg ggcttggcgt ggagcag |
| (SEQ ID NO: 1886) | gg ggcttggcgt ggagcagg |
| (SEQ ID NO: 1887) | g ggcttggcgt ggagcaggc |
| (SEQ ID NO: 1888) | ggcttggcgt ggagcaggcg |
| (SEQ ID NO: 1889) | gcttggcgt ggagcaggcg g |
| (SEQ ID NO: 1890) | cttggcgt ggagcaggcg gt |
| (SEQ ID NO: 1891) | ttggcgt ggagcaggcg gtg |
| (SEQ ID NO: 1892) | tggcgt ggagcaggcg gtgg |
| (SEQ ID NO: 1893) | ggcgt ggagcaggcg gtggg |
| (SEQ ID NO: 1894) | gcgt ggagcaggcg gtgggg |
| (SEQ ID NO: 1895) | cgt ggagcaggcg gtgggga |
| (SEQ ID NO: 1896) | gt ggagcaggcg gtggggag |
| (SEQ ID NO: 1897) | t ggagcaggcg gtggggaga |
| (SEQ ID NO: 1898) | ggagcaggcg gtggggagag |
| (SEQ ID NO: 1899) | gagcaggcg gtggggagag a |
| (SEQ ID NO: 1900) | agcaggcg gtggggagag at |
| (SEQ ID NO: 1901) | gcaggcg gtggggagag atg |
| (SEQ ID NO: 1902) | caggcg gtggggagag atgg |
| (SEQ ID NO: 1903) | aggcg gtggggagag atgga |
| (SEQ ID NO: 1904) | ggcg gtggggagag atggac |
| (SEQ ID NO: 1905) | gcg gtggggagag atggaca |
| (SEQ ID NO: 1906) | cg gtggggagag atggacag |
| (SEQ ID NO: 1907) | g gtggggagag atggacagt |
| (SEQ ID NO: 1908) | gtggggagag atggacagtt |
| (SEQ ID NO: 1909) | tggggagag atggacagtt c |
| (SEQ ID NO: 1910) | ggggagag atggacagtt ca |
| (SEQ ID NO: 1911) | gggagag atggacagtt cac |
| (SEQ ID NO: 1912) | ggagag atggacagtt caca |
| (SEQ ID NO: 1913) | gagag atggacagtt cacac |
| (SEQ ID NO: 1914) | agag atggacagtt cacacc |
| (SEQ ID NO: 1915) | gag atggacagtt cacaccc |
| (SEQ ID NO: 1916) | ag atggacagtt cacaccct |
| (SEQ ID NO: 1917) | g atggacagtt cacaccctg |
| (SEQ ID NO: 1918) | atggacagtt cacaccctgc |
| (SEQ ID NO: 1919) | tggacagtt cacaccctgc a |
| (SEQ ID NO: 1920) | ggacagtt cacaccctgc aa |
| (SEQ ID NO: 1921) | gacagtt cacaccctgc aag |
| (SEQ ID NO: 1922) | acagtt cacaccctgc aagg |
| (SEQ ID NO: 1923) | cagtt cacaccctgc aaggc |
| (SEQ ID NO: 1924) | agtt cacaccctgc aaggcc |
| (SEQ ID NO: 1925) | gtt cacaccctgc aaggccc |
| (SEQ ID NO: 1926) | tt cacaccctgc aaggccca |
| (SEQ ID NO: 1927) | t cacaccctgc aaggcccac |
| (SEQ ID NO: 1928) | cacaccctgc aaggcccaca |
| (SEQ ID NO: 1929) | acaccctgc aaggcccaca g |
| (SEQ ID NO: 1930) | caccctgc aaggcccaca gg |
| (SEQ ID NO: 1931) | accctgc aaggcccaca gga |
| (SEQ ID NO: 1932) | ccctgc aaggcccaca ggag |
| (SEQ ID NO: 1933) | cctgc aaggcccaca ggagg |
| (SEQ ID NO: 1934) | ctgc aaggcccaca ggaggc |
| (SEQ ID NO: 1935) | tgc aaggcccaca ggaggca |
| (SEQ ID NO: 1936) | gc aaggcccaca ggaggcaa |
| (SEQ ID NO: 1937) | c aaggcccaca ggaggcaag |
| (SEQ ID NO: 1938) | aaggcccaca ggaggcaagc |
| (SEQ ID NO: 1939) | aggcccaca ggaggcaagc a |
| (SEQ ID NO: 1940) | ggcccaca ggaggcaagc aa |
| (SEQ ID NO: 1941) | gcccaca ggaggcaagc aag |
| (SEQ ID NO: 1942) | cccaca ggaggcaagc aagc |
| (SEQ ID NO: 1943) | ccaca ggaggcaagc aagct |
| (SEQ ID NO: 1944) | caca ggaggcaagc aagctc |
| (SEQ ID NO: 1945) | aca ggaggcaagc aagctct |
| (SEQ ID NO: 1946) | ca ggaggcaagc aagctctc |
| (SEQ ID NO: 1947) | a ggaggcaagc aagctctct |
| (SEQ ID NO: 1948) | ggaggcaagc aagctctctt |
| (SEQ ID NO: 1949) | gaggcaagc aagctctctt g |
| (SEQ ID NO: 1950) | aggcaagc aagctctctt gc |
| (SEQ ID NO: 1951) | ggcaagc aagctctctt gcc |
| (SEQ ID NO: 2952) | gcaagc aagctctctt gccg |
| (SEQ ID NO: 1953) | caagc aagctctctt gccga |
| (SEQ ID NO: 1954) | aagc aagctctctt gccgag |
| (SEQ ID NO: 1955) | agc aagctctctt gccgagg |
| (SEQ ID NO: 1956) | gc aagctctctt gccgagga |
| (SEQ ID NO: 1957) | c aagctctctt gccgaggag |
| (SEQ ID NO: 1958) | aagctctctt gccgaggagc |
| (SEQ ID NO: 1959) | agctctctt gccgaggagc c |
| (SEQ ID NO: 1960) | gctctctt gccgaggagc ca |
| (SEQ ID NO: 1961) | ctctctt gccgaggagc cag |
| (SEQ ID NO: 1962) | tctctt gccgaggagc cagg |
| (SEQ ID NO: 1963) | ctctt gccgaggagc caggc |
| (SEQ ID NO: 1964) | tctt gccgaggagc caggca |
| (SEQ ID NO: 1965) | ctt gccgaggagc caggcaa |
| (SEQ ID NO: 1966) | tt gccgaggagc caggcaac |
| (SEQ ID NO: 1967) | t gccgaggagc caggcaact |
| (SEQ ID NO: 1968) | gccgaggagc caggcaactt |
| (SEQ ID NO: 1969) | ccgaggagc caggcaactt c |
| (SEQ ID NO: 1970) | cgaggagc caggcaactt ca |
| (SEQ ID NO: 1971) | gaggagc caggcaactt cag |
| (SEQ ID NO: 1972) | aggagc caggcaactt cagt |
| (SEQ ID NO: 1973) | ggagc caggcaactt cagtc |
| (SEQ ID NO: 1974) | gagc caggcaactt cagtcc |
| (SEQ ID NO: 1975) | agc caggcaactt cagtcct |
| (SEQ ID NO: 1976) | gc caggcaactt cagtcctg |
| (SEQ ID NO: 1977) | c caggcaactt cagtcctgg |
| (SEQ ID NO: 1978) | caggcaactt cagtcctggg |
| (SEQ ID NO: 1979) | aggcaactt cagtcctggg a |
| (SEQ ID NO: 1980) | ggcaactt cagtcctggg ag |
| (SEQ ID NO: 1981) | gcaactt cagtcctggg aga |
| (SEQ ID NO: 1982) | caactt cagtcctggg agac |
| (SEQ ID NO: 1983) | aactt cagtcctggg agacc |
| (SEQ ID NO: 1984) | actt cagtcctggg agaccc |
| (SEQ ID NO: 1985) | ctt cagtcctggg agaccca |
| (SEQ ID NO: 1986) | tt cagtcctggg agacccat |
| (SEQ ID NO: 1987) | t cagtcctggg agacccatg |
| (SEQ ID NO: 1988) | cagtcctggg agacccatgt |
| (SEQ ID NO: 1989) | agtcctggg agacccatgt a |
| (SEQ ID NO: 1990) | gtcctggg agacccatgt aa |
| (SEQ ID NO: 1991) | tcctggg agacccatgt aaa |
| (SEQ ID NO: 1992) | cctggg agacccatgt aaat |
| (SEQ ID NO: 1993) | ctggg agacccatgt aaata |
| (SEQ ID NO: 1994) | tggg agacccatgt aaatac |
| (SEQ ID NO: 1995) | ggg agacccatgt aaatacc |
| (SEQ ID NO: 1996) | gg agacccatgt aaatacca |
| (SEQ ID NO: 1997) | g agacccatgt aaataccag |
| (SEQ ID NO: 1998) | agacccatgt aaataccaga |
| (SEQ ID NO: 1999) | gacccatgt aaataccaga c |
| (SEQ ID NO: 2000) | acccatgt aaataccaga ct |
| (SEQ ID NO: 2001) | cccatgt aaataccaga ctg |
| (SEQ ID NO: 2002) | ccatgt aaataccaga ctgc |
| (SEQ ID NO: 2003) | catgt aaataccaga ctgca |
| (SEQ ID NO: 2004) | atgt aaataccaga ctgcag |
| (SEQ ID NO: 2005) | tgt aaataccaga ctgcagg |
| (SEQ ID NO: 2006) | gt aaataccaga ctgcaggt |
| (SEQ ID NO: 2007) | t aaataccaga ctgcaggtt |
| (SEQ ID NO: 2008) | t aaataccaga ctgcaggtt |
| (SEQ ID NO: 2009) | aataccaga ctgcaggttg g |
| (SEQ ID NO: 2010) | ataccaga ctgcaggttg ga |
| (SEQ ID NO: 2011) | taccaga ctgcaggttg gac |
| (SEQ ID NO: 2012) | accaga ctgcaggttg gacc |
| (SEQ ID NO: 2013) | ccaga ctgcaggttg gaccc |
| (SEQ ID NO: 2014) | caga ctgcaggttg gacccc |
| (SEQ ID NO: 2015) | aga ctgcaggttg gacccca |
| (SEQ ID NO: 2016) | ga ctgcaggttg gaccccag |
| (SEQ ID NO: 2017) | a ctgcaggttg gaccccaga |
| (SEQ ID NO: 2018) | ctgcaggttg gaccccagag |
| (SEQ ID NO: 2019) | tgcaggttg gaccccagag a |
| (SEQ ID NO: 2020) | gcaggttg gaccccagag at |
| (SEQ ID NO: 2021) | caggttg gaccccagag att |
| (SEQ ID NO: 2022) | aggttg gaccccagag attc |
| (SEQ ID NO: 2023) | ggttg gaccccagag attcc |
| (SEQ ID NO: 2024) | gttg gaccccagag attccc |
| (SEQ ID NO: 2025) | ttg gaccccagag attccca |
| (SEQ ID NO: 2026) | tg gaccccagag attcccaa |
| (SEQ ID NO: 2027) | g gaccccagag attcccaag |
| (SEQ ID NO: 2028) | gaccccagag attcccaagc |
| (SEQ ID NO: 2029) | accccagag attcccaagc c |
| (SEQ ID NO: 2030) | ccccagag attcccaagc ca |
| (SEQ ID NO: 2031) | cccagag attcccaagc caa |
| (SEQ ID NO: 2032) | ccagag attcccaagc caaa |
| (SEQ ID NO: 2033) | cagag attcccaagc caaaa |
| (SEQ ID NO: 2034) | agag attcccaagc caaaaa |
| (SEQ ID NO: 2035) | gag attcccaagc caaaaac |
| (SEQ ID NO: 2036) | ag attcccaagc caaaaacc |
| (SEQ ID NO: 2037) | g attcccaagc caaaaacct |
| (SEQ ID NO: 2038) | attcccaagc caaaaacctt |
| (SEQ ID NO: 2039) | ttcccaagc caaaaacctt a |
| (SEQ ID NO: 2040) | tcccaagc caaaaacctt ag |
| (SEQ ID NO: 2041) | cccaagc caaaaacctt agc |
| (SEQ ID NO: 2042) | ccaagc caaaaacctt agct |
| (SEQ ID NO: 2043) | caagc caaaaacctt agctc |
| (SEQ ID NO: 2044) | aagc caaaaacctt agctcc |
| (SEQ ID NO: 2045) | agc caaaaacctt agctccc |
| (SEQ ID NO: 2046) | gc caaaaacctt agctccct |
| (SEQ ID NO: 2047) | c caaaaacctt agctccctc |
| (SEQ ID NO: 2048) | caaaaacctt agctccctcc |
| (SEQ ID NO: 2049) | aaaaacctt agctccctcc c |
| (SEQ ID NO: 2050) | aaaacctt agctccctcc cg |
| (SEQ ID NO: 2051) | aaacctt agctccctcc cgc |
| (SEQ ID NO: 2052) | aacctt agctccctcc cgca |
| (SEQ ID NO: 2053) | acctt agctccctcc cgcac |
| (SEQ ID NO: 2054) | cctt agctccctcc cgcacc |
| (SEQ ID NO: 2055) | ctt agctccctcc cgcaccc |
| (SEQ ID NO: 2056) | tt agctccctcc cgcacccc |
| (SEQ ID NO: 2057) | t agctccctcc cgcaccccg |
| (SEQ ID NO: 2058) | agctccctcc cgcaccccga |
| (SEQ ID NO: 2059) | gctccctcc cgcaccccga t |
| (SEQ ID NO: 2060) | ctccctcc cgcaccccga tg |
| (SEQ ID NO: 2061) | tccctcc cgcaccccga tgt |
| (SEQ ID NO: 2062) | ccctcc cgcaccccga tgtg |
| (SEQ ID NO: 2063) | cctcc cgcaccccga tgtgg |
| (SEQ ID NO: 2064) | ctcc cgcaccccga tgtgga |
| (SEQ ID NO: 2065) | tcc cgcaccccga tgtggac |
| (SEQ ID NO: 2066) | cc cgcaccccga tgtggacc |
| (SEQ ID NO: 2067) | c cgcaccccga tgtggacct |
| (SEQ ID NO: 2068) | cgcaccccga tgtggacctc t |
| (SEQ ID NO: 2069) | caccccga tgtggacctc ta |
| (SEQ ID NO: 2070) | accccga tgtggacctc tac |
| (SEQ ID NO: 2071) | ccccga tgtggacctc tact |
| (SEQ ID NO: 2072) | cccga tgtggacctc tactt |
| (SEQ ID NO: 2073) | ccga tgtggacctc tacttt |
| (SEQ ID NO: 2074) | cga tgtggacctc tactttc |
| (SEQ ID NO: 2075) | ga tgtggacctc tactttcc |
| (SEQ ID NO: 2076) | a tgtggacctc tactttcca |
| (SEQ ID NO: 2077) | tgtggacctc tactttccag |
| (SEQ ID NO: 2078) | gtggacctc tactttccag g |
| (SEQ ID NO: 2079) | tggacctc tactttccag gc |
| (SEQ ID NO: 2080) | ggacctc tactttccag gct |
| (SEQ ID NO: 2081) | gacctc tactttccag gcta |
| (SEQ ID NO: 2082) | acctc tactttccag gctag |
| (SEQ ID NO: 2083) | cctc tactttccag gctagt |
| (SEQ ID NO: 2084) | ctc tactttccag gctagtc |
| (SEQ ID NO: 2085) | tc tactttccag gctagtcc |
| (SEQ ID NO: 2086) | c tactttccag gctagtccg |
| (SEQ ID NO: 2087) | tactttccag gctagtccgg |
| (SEQ ID NO: 2088) | actttccag gctagtccgg a |
| (SEQ ID NO: 2089) | ctttccag gctagtccgg ac |
| (SEQ ID NO: 2090) | tttccag gctagtccgg acc |
| (SEQ ID NO: 2091) | ttccag gctagtccgg accc |
| (SEQ ID NO: 2092) | tccag gctagtccgg accca |
| (SEQ ID NO: 2093) | ccag gctagtccgg acccac |
| (SEQ ID NO: 2094) | cag gctagtccgg acccacc |
| (SEQ ID NO: 2095) | ag gctagtccgg acccacct |
| (SEQ ID NO: 2096) | g gctagtccgg acccacctc |
| (SEQ ID NO: 2097) | gctagtccgg acccacctca |
| (SEQ ID NO: 2098) | ctagtccgg acccacctca c |
| (SEQ ID NO: 2099) | tagtccgg acccacctca cc |
| (SEQ ID NO: 2100) | agtccgg acccacctca ccc |
| (SEQ ID NO: 2101) | gtccgg acccacctca cccc |
| (SEQ ID NO: 2102) | tccgg acccacctca ccccg |
| (SEQ ID NO: 2103) | ccgg acccacctca ccccgt |
| (SEQ ID NO: 2104) | cgg acccacctca ccccgtt |
| (SEQ ID NO: 2105) | g acccacctca ccccgttac |
| (SEQ ID NO: 2106) | acccacctca ccccgttaca |
| (SEQ ID NO: 2107) | cccacctca ccccgttaca g |
| (SEQ ID NO: 2108) | ccacctca ccccgttaca gc |
| (SEQ ID NO: 2109) | cacctca ccccgttaca gct |
| (SEQ ID NO: 2110) | acctca ccccgttaca gctc |
| (SEQ ID NO: 2111) | cctca ccccgttaca gctcc |
| (SEQ ID NO: 2112) | ctca ccccgttaca gctccc |
| (SEQ ID NO: 2113) | tca ccccgttaca gctcccc |
| (SEQ ID NO: 2114) | ca ccccgttaca gctcccca |
| (SEQ ID NO: 2115) | a ccccgttaca gctccccaa |
| (SEQ ID NO: 2116) | ccccgttaca gctccccaag |
| (SEQ ID NO: 2117) | cccgttaca gctccccaag t |
| (SEQ ID NO: 2118) | ccgttaca gctccccaag tg |
| (SEQ ID NO: 2119) | cgttaca gctccccaag tgg |
| (SEQ ID NO: 2120) | gttaca gctccccaag tggt |
| (SEQ ID NO: 2121) | ttaca gctccccaag tggtt |
| (SEQ ID NO: 2122) | taca gctccccaag tggttt |
| (SEQ ID NO: 2123) | aca gctccccaag tggtttc |
| (SEQ ID NO: 2124) | ca gctccccaag tggtttcc |
| (SEQ ID NO: 2125) | a gctccccaag tggtttcca |
| (SEQ ID NO: 2126) | gctccccaag tggtttccac |
| (SEQ ID NO: 2127) | ctccccaag tggtttccac a |
| (SEQ ID NO: 2128) | tccccaag tggtttccac at |
| (SEQ ID NO: 2129) | ccccaag tggtttccac atg |
| (SEQ ID NO: 2130) | cccaag tggtttccac atgc |
| (SEQ ID NO: 2131) | ccaag tggtttccac atgct |
| (SEQ ID NO: 2132) | caag tggtttccac atgctc |
| (SEQ ID NO: 2133) | aag tggtttccac atgctct |
| (SEQ ID NO: 2134) | ag tggtttccac atgctctg |
| (SEQ ID NO: 2135) | g tggtttccac atgctctga |
| (SEQ ID NO: 2136) | tggtttccac atgctctgag |
| (SEQ ID NO: 2137) | ggtttccac atgctctgag a |
| (SEQ ID NO: 2138) | gtttccac atgctctgag aa |
| (SEQ ID NO: 2139) | tttccac atgctctgag aag |
| (SEQ ID NO: 2140) | ttccac atgctctgag aaga |
| (SEQ ID NO: 2141) | tccac atgctctgag aagag |
| (SEQ ID NO: 2142) | ccac atgctctgag aagagg |
| (SEQ ID NO: 2143) | cac atgctctgag aagagga |
| (SEQ ID NO: 2144) | ac atgctctgag aagaggag |
| (SEQ ID NO: 2145) | c atgctctgag aagaggagc |
| (SEQ ID NO: 2146) | atgctctgag aagaggagcc |
| (SEQ ID NO: 2147) | tgctctgag aagaggagcc c |
| (SEQ ID NO: 2148) | gctctgag aagaggagcc ct |
| (SEQ ID NO: 2149) | ctctgag aagaggagcc ctc |
| (SEQ ID NO: 2150) | tctgag aagaggagcc ctca |
| (SEQ ID NO: 2151) | ctgag aagaggagcc ctcat |
| (SEQ ID NO: 2152) | tgag aagaggagcc ctcatc |
| (SEQ ID NO: 2153) | gag aagaggagcc ctcatct |
| (SEQ ID NO: 2154) | ag aagaggagcc ctcatctt |
| (SEQ ID NO: 2155) | g aagaggagcc ctcatcttg |
| (SEQ ID NO: 2156) | aagaggagcc ctcatcttga |
| (SEQ ID NO: 2157) | agaggagcc ctcatcttga a |
| (SEQ ID NO: 2158) | gaggagcc ctcatcttga ag |
| (SEQ ID NO: 2159) | aggagcc ctcatcttga agg |
| (SEQ ID NO: 2160) | ggagcc ctcatcttga aggg |
| (SEQ ID NO: 2161) | gagcc ctcatcttga agggc |
| (SEQ ID NO: 2162) | agcc ctcatcttga agggcc |
| (SEQ ID NO: 2163) | gcc ctcatcttga agggccc |
| (SEQ ID NO: 2164) | cc ctcatcttga agggccca |
| (SEQ ID NO: 2165) | c ctcatcttga agggcccag |
| (SEQ ID NO: 2166) | ctcatcttga agggcccagg |
| (SEQ ID NO: 2167) | tcatcttga agggcccagg a |
| (SEQ ID NO: 2168) | catcttga agggcccagg ag |
| (SEQ ID NO: 2169) | atcttga agggcccagg agg |
| (SEQ ID NO: 2170) | tcttga agggcccagg aggg |
| (SEQ ID NO: 2171) | cttga agggcccagg agggt |
| (SEQ ID NO: 2172) | ttga agggcccagg agggtc |
| (SEQ ID NO: 2173) | tga agggcccagg agggtct |
| (SEQ ID NO: 2174) | ga agggcccagg agggtcta |
| (SEQ ID NO: 2175) | a agggcccagg agggtctat |
| (SEQ ID NO: 2176) | agggcccagg agggtctatg |
| (SEQ ID NO: 2177) | gggcccagg agggtctatg g |
| (SEQ ID NO: 2178) | ggcccagg agggtctatg gg |
| (SEQ ID NO: 2179) | gcccagg agggtctatg ggg |
| (SEQ ID NO: 2180) | cccagg agggtctatg ggga |
| (SEQ ID NO: 2181) | ccagg agggtctatg gggag |
| (SEQ ID NO: 2182) | cagg agggtctatg gggaga |
| (SEQ ID NO: 2183) | agg agggtctatg gggagag |
| (SEQ ID NO: 2184) | gg agggtctatg gggagagg |
| (SEQ ID NO: 2185) | g agggtctatg gggagagga |
| (SEQ ID NO: 2186) | agggtctatg gggagaggaa |
| (SEQ ID NO: 2187) | gggtctatg gggagaggaa c |
| (SEQ ID NO: 2188) | ggtctatg gggagaggaa ct |
| (SEQ ID NO: 2189) | gtctatg gggagaggaa ctc |
| (SEQ ID NO: 2190) | tctatg gggagaggaa ctcc |
| (SEQ ID NO: 2191) | ctatg gggagaggaa ctcct |
| (SEQ ID NO: 2192) | tatg gggagaggaa ctcctt |
| (SEQ ID NO: 2193) | atg gggagaggaa ctccttg |
| (SEQ ID NO: 2194) | tg gggagaggaa ctccttgg |
| (SEQ ID NO: 2195) | g gggagaggaa ctccttggc |
| (SEQ ID NO: 2196) | gggagaggaa ctccttggcc |
| (SEQ ID NO: 2197) | ggagaggaa ctccttggcc t |
| (SEQ ID NO: 2198) | gagaggaa ctccttggcc ta |
| (SEQ ID NO: 2199) | agaggaa ctccttggcc tag |
| (SEQ ID NO: 2200) | gaggaa ctccttggcc tagc |
| (SEQ ID NO: 2201) | aggaa ctccttggcc tagcc |
| (SEQ ID NO: 2202) | ggaa ctccttggcc tagccc |
| (SEQ ID NO: 2203) | gaa ctccttggcc tagccca |
| (SEQ ID NO: 2204) | aa ctccttggcc tagcccac |
| (SEQ ID NO: 2205) | a ctccttggcc tagcccacc |
| (SEQ ID NO: 2206) | ctccttggcc tagcccaccc |
| (SEQ ID NO: 2207) | tccttggcc tagcccaccc t |
| (SEQ ID NO: 2208) | ccttggcc tagcccaccc tg |
| (SEQ ID NO: 2209) | cttggcc tagcccaccc tgc |
| (SEQ ID NO: 2210) | ttggcc tagcccaccc tgct |
| (SEQ ID NO: 2211) | tggcc tagcccaccc tgctg |
| (SEQ ID NO: 2212) | ggcc tagcccaccc tgctgc |
| (SEQ ID NO: 2213) | gcc tagcccaccc tgctgcc |
| (SEQ ID NO: 2214) | cc tagcccaccc tgctgcct |
| (SEQ ID NO: 2215) | c tagcccaccc tgctgcctt |
| (SEQ ID NO: 2216) | tagcccaccc tgctgccttc |
| (SEQ ID NO: 2217) | agcccaccc tgctgccttc t |
| (SEQ ID NO: 2218) | gcccaccc tgctgccttc tg |
| (SEQ ID NO: 2219) | cccaccc tgctgccttc tga |
| (SEQ ID NO: 2220) | ccaccc tgctgccttc tgac |
| (SEQ ID NO: 2221) | caccc tgctgccttc tgacg |
| (SEQ ID NO: 2222) | accc tgctgccttc tgacgg |
| (SEQ ID NO: 2223) | ccc tgctgccttc tgacggc |
| (SEQ ID NO: 2224) | cc tgctgccttc tgacggcc |
| (SEQ ID NO: 2225) | c tgctgccttc tgacggccc |
| (SEQ ID NO: 2226) | tgctgccttc tgacggccct |
| (SEQ ID NO: 2227) | gctgccttc tgacggccct g |
| (SEQ ID NO: 2228) | ctgccttc tgacggccct gc |
| (SEQ ID NO: 2229) | tgccttc tgacggccct gca |
| (SEQ ID NO: 2230) | gccttc tgacggccct gcaa |
| (SEQ ID NO: 2231) | ccttc tgacggccct gcaat |
| (SEQ ID NO: 2232) | cttc tgacggccct gcaatg |
| (SEQ ID NO: 2233) | ttc tgacggccct gcaatgt |
| (SEQ ID NO: 2234) | tc tgacggccct gcaatgta |
| (SEQ ID NO: 2235) | c tgacggccct gcaatgtat |
| (SEQ ID NO: 2236) | tgacggccct gcaatgtatc |
| (SEQ ID NO: 2237) | gacggccct gcaatgtatc c |
| (SEQ ID NO: 2238) | acggccct gcaatgtatc cc |
| (SEQ ID NO: 2239) | cggccct gcaatgtatc cct |
| (SEQ ID NO: 2240) | ggccct gcaatgtatc cctt |
| (SEQ ID NO: 2241) | gccct gcaatgtatc ccttc |
| (SEQ ID NO: 2242) | ccct gcaatgtatc ccttct |
| (SEQ ID NO: 2243) | cct gcaatgtatc ccttctc |
| (SEQ ID NO: 2244) | ct gcaatgtatc ccttctca |
| (SEQ ID NO: 2245) | t gcaatgtatc ccttctcac |
| (SEQ ID NO: 2246) | gcaatgtatc ccttctcaca |
| (SEQ ID NO: 2247) | caatgtatc ccttctcaca g |
| (SEQ ID NO: 2248) | aatgtatc ccttctcaca gc |
| (SEQ ID NO: 2249) | atgtatc ccttctcaca gca |
| (SEQ ID NO: 2250) | tgtatc ccttctcaca gcac |
| (SEQ ID NO: 2251) | gtatc ccttctcaca gcaca |
| (SEQ ID NO: 2252) | tatc ccttctcaca gcacat |
| (SEQ ID NO: 2253) | atc ccttctcaca gcacatg |
| (SEQ ID NO: 2254) | tc ccttctcaca gcacatgc |
| (SEQ ID NO: 2255) | c ccttctcaca gcacatgct |
| (SEQ ID NO: 2256) | ccttctcaca gcacatgctg |
| (SEQ ID NO: 2257) | cttctcaca gcacatgctg g |
| (SEQ ID NO: 2258) | ttctcaca gcacatgctg gc |
| (SEQ ID NO: 2259) | tctcaca gcacatgctg gcc |
| (SEQ ID NO: 2260) | ctcaca gcacatgctg gcca |
| (SEQ ID NO: 2261) | tcaca gcacatgctg gccag |
| (SEQ ID NO: 2262) | caca gcacatgctg gccagc |
| (SEQ ID NO: 2263) | aca gcacatgctg gccagcc |
| (SEQ ID NO: 2264) | ca gcacatgctg gccagcct |
| (SEQ ID NO: 2265) | a gcacatgctg gccagcctg |
| (SEQ ID NO: 2266) | gcacatgctg gccagcctgg |
| (SEQ ID NO: 2267) | cacatgctg gccagcctgg g |
| (SEQ ID NO: 2268) | acatgctg gccagcctgg gg |
| (SEQ ID NO: 2269) | catgctg gccagcctgg ggc |
| (SEQ ID NO: 2270) | atgctg gccagcctgg ggcc |
| (SEQ ID NO: 2271) | tgctg gccagcctgg ggcct |
| (SEQ ID NO: 2272) | gctg gccagcctgg ggcctg |
| (SEQ ID NO: 2273) | ctg gccagcctgg ggcctgg |
| (SEQ ID NO: 2274) | tg gccagcctgg ggcctggc |
| (SEQ ID NO: 2275) | g gccagcctgg ggcctggca |
| (SEQ ID NO: 2276) | gccagcctgg ggcctggcag |
| (SEQ ID NO: 2277) | ccagcctgg ggcctggcag g |
| (SEQ ID NO: 2278) | cagcctgg ggcctggcag gg |
| (SEQ ID NO: 2279) | agcctgg ggcctggcag gga |
| (SEQ ID NO: 2280) | gcctgg ggcctggcag ggag |
| (SEQ ID NO: 2281) | cctgg ggcctggcag ggagg |
| (SEQ ID NO: 2282) | ctgg ggcctggcag ggaggt |
| (SEQ ID NO: 2283) | tgg ggcctggcag ggaggtc |
| (SEQ ID NO: 2284) | gg ggcctggcag ggaggtca |
| (SEQ ID NO: 2285) | g ggcctggcag ggaggtcag |
| (SEQ ID NO: 2286) | ggcctggcag ggaggtcagg |
| (SEQ ID NO: 2287) | gcctggcag ggaggtcagg c |
| (SEQ ID NO: 2288) | cctggcag ggaggtcagg cc |
| (SEQ ID NO: 2289) | ctggcag ggaggtcagg ccc |
| (SEQ ID NO: 2290) | tggcag ggaggtcagg ccct |
| (SEQ ID NO: 2291) | ggcag ggaggtcagg ccctg |
| (SEQ ID NO: 2292) | gcag ggaggtcagg ccctgg |
| (SEQ ID NO: 2293) | cag ggaggtcagg ccctgga |
| (SEQ ID NO: 2294) | ag ggaggtcagg ccctggaa |
| (SEQ ID NO: 2295) | g ggaggtcagg ccctggaac |
| (SEQ ID NO: 2296) | ggaggtcagg ccctggaact |
| (SEQ ID NO: 2297) | gaggtcagg ccctggaact c |
| (SEQ ID NO: 2298) | aggtcagg ccctggaact ct |
| (SEQ ID NO: 2299) | ggtcagg ccctggaact cta |
| (SEQ ID NO: 2300) | gtcagg ccctggaact ctat |
| (SEQ ID NO: 2301) | tcagg ccctggaact ctatc |
| (SEQ ID NO: 2302) | cagg ccctggaact ctatct |
| (SEQ ID NO: 2303) | agg ccctggaact ctatctg |
| (SEQ ID NO: 2304) | gg ccctggaact ctatctgg |
| (SEQ ID NO: 2305) | g ccctggaact ctatctggg |
| (SEQ ID NO: 2306) | ccctggaact ctatctgggc |
| (SEQ ID NO: 2307) | cctggaact ctatctgggc c |
| (SEQ ID NO: 2308) | ctggaact ctatctgggc ct |
| (SEQ ID NO: 2309) | tggaact ctatctgggc ctg |
| (SEQ ID NO: 2310) | ggaact ctatctgggc ctgg |
| (SEQ ID NO: 2311) | gaact ctatctgggc ctggg |
| (SEQ ID NO: 2312) | aact ctatctgggc ctgggc |
| (SEQ ID NO: 2313) | act ctatctgggc ctgggct |
| (SEQ ID NO: 2314) | ct ctatctgggc ctgggcta |
| (SEQ ID NO: 2315) | t ctatctgggc ctgggctag |
| (SEQ ID NO: 2316) | ctatctgggc ctgggctagg |
| (SEQ ID NO: 2317) | tatctgggc ctgggctagg g |
| (SEQ ID NO: 2318) | atctgggc ctgggctagg gg |
| (SEQ ID NO: 2319) | tctgggc ctgggctagg gga |
| (SEQ ID NO: 2320) | ctgggc ctgggctagg ggac |
| (SEQ ID NO: 2321) | tgggc ctgggctagg ggaca |
| (SEQ ID NO: 2322) | gggc ctgggctagg ggacat |
| (SEQ ID NO: 2323) | ggc ctgggctagg ggacatc |
| (SEQ ID NO: 2324) | gc ctgggctagg ggacatca |
| (SEQ ID NO: 2325) | c ctgggctagg ggacatcag |
| (SEQ ID NO: 2326) | ctgggctagg ggacatcaga |
| (SEQ ID NO: 2327) | tgggctagg ggacatcaga g |
| (SEQ ID NO: 2328) | gggctagg ggacatcaga gg |
| (SEQ ID NO: 2329) | ggctagg ggacatcaga ggt |
| (SEQ ID NO: 2330) | gctagg ggacatcaga ggtt |
| (SEQ ID NO: 2331) | ctagg ggacatcaga ggttc |
| (SEQ ID NO: 2332) | tagg ggacatcaga ggttct |
| (SEQ ID NO: 2333) | agg ggacatcaga ggttctt |
| (SEQ ID NO: 2334) | gg ggacatcaga ggttcttt |
| (SEQ ID NO: 2335) | g ggacatcaga ggttctttg |
| (SEQ ID NO: 2336) | ggacatcaga ggttctttga |
| (SEQ ID NO: 2337) | gacatcaga ggttctttga g |
| (SEQ ID NO: 2338) | acatcaga ggttctttga gg |
| (SEQ ID NO: 2339) | catcaga ggttctttga ggg |
| (SEQ ID NO: 2340) | atcaga ggttctttga ggga |
| (SEQ ID NO: 2341) | tcaga ggttctttga gggac |
| (SEQ ID NO: 2342) | caga ggttctttga gggact |
| (SEQ ID NO: 2343) | aga ggttctttga gggactg |
| (SEQ ID NO: 2344) | ga ggttctttga gggactgc |
| (SEQ ID NO: 2345) | a ggttctttga gggactgcc |
| (SEQ ID NO: 2346) | ggttctttga gggactgcct |
| (SEQ ID NO: 2347) | gttctttga gggactgcct c |
| (SEQ ID NO: 2348) | ttctttga gggaatgcct ct |
| (SEQ ID NO: 2349) | tctttga gggactgcct ctg |
| (SEQ ID NO: 2350) | ctttga gggactgcct ctgc |
| (SEQ ID NO: 2351) | tttga gggactgcct ctgcc |
| (SEQ ID NO: 2352) | ttga gggactgcct ctgcca |
| (SEQ ID NO: 2353) | tga gggactgcct ctgccac |
| (SEQ ID NO: 2354) | ga gggactgcct ctgccaca |
| (SEQ ID NO: 2355) | a gggactgcct ctgccacac |
| (SEQ ID NO: 2356) | gggactgcct ctgccacact |
| (SEQ ID NO: 2357) | ggactgcct ctgccacact c |
| (SEQ ID NO: 2358) | gactgcct ctgccacact ct |
| (SEQ ID NO: 2359) | actgcct ctgccacact ctg |
| (SEQ ID NO: 2360) | ctgcct ctgccacact ctga |
| (SEQ ID NO: 2361) | tgcct ctgccacact ctgac |
| (SEQ ID NO: 2362) | gcct ctgccacact ctgacg |
| (SEQ ID NO: 2363) | cct ctgccacact ctgacgc |
| (SEQ ID NO: 2364) | ct ctgccacact ctgacgca |
| (SEQ ID NO: 2365) | t ctgccacact ctgacgcaa |
| (SEQ ID NO: 2366) | ctgccacact ctgacgcaaa |
| (SEQ ID NO: 2367) | tgccacact ctgacgcaaa a |
| (SEQ ID NO: 2368) | gccacact ctgacgcaaa ac |
| (SEQ ID NO: 2369) | ccacact ctgacgcaaa acc |
| (SEQ ID NO: 2370) | cacact ctgacgcaaa acca |
| (SEQ ID NO: 2371) | acact ctgacgcaaa accac |
| (SEQ ID NO: 2372) | cact ctgacgcaaa accact |
| (SEQ ID NO: 2373) | act ctgacgcaaa accactt |
| (SEQ ID NO: 2374) | ct ctgacgcaaa accacttt |
| (SEQ ID NO: 2375) | t ctgacgcaaa accactttc |
| (SEQ ID NO: 2376) | ctgacgcaaa accactttcc |
| (SEQ ID NO: 2377) | tgacgcaaa accactttcc t |
| (SEQ ID NO: 2378) | gacgcaaa accactttcc tt |
| (SEQ ID NO: 2379) | acgcaaa accactttca ttt |
| (SEQ ID NO: 2380) | cgcaaa accactttcc tttt |
| (SEQ ID NO: 2381) | gcaaa accactttcc ttttc |
| (SEQ ID NO: 2382) | caaa accactttcc ttttct |
| (SEQ ID NO: 2383) | aaa accactttcc ttttcta |
| (SEQ ID NO: 2384) | aa accactttcc ttttctat |
| (SEQ ID NO: 2385) | a accactttcc ttttctatt |
| (SEQ ID NO: 2386) | accactttcc ttttctattc |
| (SEQ ID NO: 2387) | ccactttcc ttttctattc c |
| (SEQ ID NO: 2388) | cactttcc ttttctattc ct |
| (SEQ ID NO: 2389) | actttcc ttttctattc ctt |
| (SEQ ID NO: 2390) | ctttcc ttttctattc cttc |
| (SEQ ID NO: 2391) | tttcc ttttctattc cttct |
| (SEQ ID NO: 2392) | ttcc ttttctattc cttctg |
| (SEQ ID NO: 2393) | tcc ttttctattc cttctgg |
| (SEQ ID NO: 2394) | cc ttttctattc cttctggc |
| (SEQ ID NO: 2395) | c ttttctattc cttctggcc |
| (SEQ ID NO: 2396) | ttttctattc cttctggcct |
| (SEQ ID NO: 2397) | tttctattc cttctggcct t |
| (SEQ ID NO: 2398) | ttctattc cttctggcct tt |
| (SEQ ID NO: 2399) | tctattc cttctggcct ttc |
| (SEQ ID NO: 2400) | ctattc cttctggcct ttcc |
| (SEQ ID NO: 2401) | tattc cttctggcct ttcct |
| (SEQ ID NO: 2402) | attc cttctggcct ttcctc |
| (SEQ ID NO: 2403) | ttc cttctggcct ttcctct |
| (SEQ ID NO: 2404) | tc cttctggcct ttcctctc |
| (SEQ ID NO: 2405) | c cttctggcct ttcctctct |
| (SEQ ID NO: 2406) | cttctggcct ttcctctctc |
| (SEQ ID NO: 2407) | ttctggcct ttcctctctc c |
| (SEQ ID NO: 2408) | tctggcct ttcctctctc ct |
| (SEQ ID NO: 2409) | ctggcct ttcctctctc ctg |
| (SEQ ID NO: 2410) | tggcct ttcctctctc ctgt |
| (SEQ ID NO: 2411) | ggcct ttcctctctc ctgtt |
| (SEQ ID NO: 2412) | gcct ttcctctctc ctgttt |
| (SEQ ID NO: 2413) | cct ttcctctctc ctgtttc |
| (SEQ ID NO: 2414) | ct ttcctctctc ctgtttcc |
| (SEQ ID NO: 2415) | t ttcctctctc ctgtttccc |
| (SEQ ID NO: 2416) | ttcctctctc ctgtttccct |
| (SEQ ID NO: 2417) | tcctctctc ctgtttccct t |
| (SEQ ID NO: 2418) | cctctctc ctgtttccct tc |
| (SEQ ID NO: 2419) | ctctctc ctgtttccct tcc |
| (SEQ ID NO: 2420) | tctctc ctgtttccct tccc |
| (SEQ ID NO: 2421) | ctctc ctgtttccct tccct |
| (SEQ ID NO: 2422) | tctc ctgtttccct tccctt |
| (SEQ ID NO: 2423) | ctc ctgtttccct tcccttc |
| (SEQ ID NO: 2424) | tc ctgtttccct tcccttcc |
| (SEQ ID NO: 2425) | c ctgtttccct tcccttcca |
| (SEQ ID NO: 2426) | ctgtttccct tcccttccac |
| (SEQ ID NO: 2427) | tgtttccct tcccttccac t |
| (SEQ ID NO: 2428) | gtttccct tcccttccac tg |
| (SEQ ID NO: 2429) | tttccct tcccttccac tgc |
| (SEQ ID NO: 2430) | ttccct tcccttccac tgcc |
| (SEQ ID NO: 2431) | tccct tcccttccac tgcct |
| (SEQ ID NO: 2432) | ccct tcccttccac tgcctc |
| (SEQ ID NO: 2433) | cct tcccttccac tgcctct |
| (SEQ ID NO: 2434) | ct tcccttccac tgcctctg |
| (SEQ ID NO: 2435) | t tcccttccac tgcctctgc |
| (SEQ ID NO: 2436) | tcccttccac tgcctctgcc |
| (SEQ ID NO: 2437) | cccttccac tgcctctgcc t |
| (SEQ ID NO: 2438) | ccttccac tgcctctgcc tt |
| (SEQ ID NO: 2439) | cttccac tgcctctgcc tta |
| (SEQ ID NO: 2440) | ttccac tgcctctgcc ttag |
| (SEQ ID NO: 2441) | tccac tgcctctgcc ttaga |
| (SEQ ID NO: 2442) | ccac tgcctctgcc ttagag |
| (SEQ ID NO: 2443) | cac tgcctctgcc ttagagg |
| (SEQ ID NO: 2444) | ac tgcctctgcc ttagagga |
| (SEQ ID NO: 2445) | c tgcctctgcc ttagaggag |
| (SEQ ID NO: 2446) | tgcctctgcc ttagaggagc |
| (SEQ ID NO: 2447) | gcctctgcc ttagaggagc c |
| (SEQ ID NO: 2448) | cctctgcc ttagaggagc cc |
| (SEQ ID NO: 2449) | ctctgcc ttagaggagc cca |
| (SEQ ID NO: 2450) | tctgcc ttagaggagc ccac |
| (SEQ ID NO: 2451) | ctgcc ttagaggagc ccacg |
| (SEQ ID NO: 2452) | tgcc ttagaggagc ccacgg |
| (SEQ ID NO: 2453) | gcc ttagaggagc ccacggc |
| (SEQ ID NO: 2454) | cc ttagaggagc ccacggct |
| (SEQ ID NO: 2455) | c ttagaggagc ccacggcta |
| (SEQ ID NO: 2456) | ttagaggagc ccacggctaa |
| (SEQ ID NO: 2457) | tagaggagc ccacggctaa g |
| (SEQ ID NO: 2458) | agaggagc ccacggctaa ga |
| (SEQ ID NO: 2459) | gaggagc ccacggctaa gag |
| (SEQ ID NO: 2460) | aggagc ccacggctaa gagg |
| (SEQ ID NO: 2461) | ggagc ccacggctaa gaggc |
| (SEQ ID NO: 2462) | gagc ccacggctaa gaggct |
| (SEQ ID NO: 2463) | agc ccacggctaa gaggctg |
| (SEQ ID NO: 2464) | gc ccacggctaa gaggctgc |
| (SEQ ID NO: 2465) | c ccacggctaa gaggctgct |
| (SEQ ID NO: 2466) | ccacggctaa gaggctgctg |
| (SEQ ID NO: 2467) | cacggctaa gaggctgctg a |
| (SEQ ID NO: 2468) | acggctaa gaggctgctg aa |
| (SEQ ID NO: 2469) | cggctaa gaggctgctg aaa |
| (SEQ ID NO: 2470) | ggctaa gaggctgctg aaaa |
| (SEQ ID NO: 2471) | gctaa gaggctgctg aaaac |
| (SEQ ID NO: 2472) | ctaa gaggctgctg aaaacc |
| (SEQ ID NO: 2473) | taa gaggctgctg aaaacca |
| (SEQ ID NO: 2474) | aa gaggctgctg aaaaccat |
| (SEQ ID NO: 2475) | a gaggctgctg aaaaccatc |
| (SEQ ID NO: 2476) | gaggctgctg aaaaccatct |
| (SEQ ID NO: 2477) | aggctgctg aaaaccatct g |
| (SEQ ID NO: 2478) | ggctgctg aaaaccatct gg |
| (SEQ ID NO: 2479) | gctgctg aaaaccatct ggc |
| (SEQ ID NO: 2480) | ctgctg aaaaccatct ggcc |
| (SEQ ID NO: 2481) | tgctg aaaaccatct ggcct |
| (SEQ ID NO: 2482) | gctg aaaaccatct ggcctg |
| (SEQ ID NO: 2483) | ctg aaaaccatct ggcctgg |
| (SEQ ID NO: 2484) | tg aaaaccatct ggcctggc |
| (SEQ ID NO: 2485) | g aaaaccatct ggcctggcc |
| (SEQ ID NO: 2486) | aaaaccatct ggcctggcct |
| (SEQ ID NO: 2487) | aaaccatct ggcctggcct g |
| (SEQ ID NO: 2488) | aaccatct ggcctggcct gg |
| (SEQ ID NO: 2489) | accatct ggcctggcct ggc |
| (SEQ ID NO: 2490) | ccatct ggcctggcct ggcc |
| (SEQ ID NO: 2491) | catct ggcctggcct ggccc |
| (SEQ ID NO: 2492) | atct ggcctggcct ggccct |
| (SEQ ID NO: 2493) | tct ggcctggcct ggccctg |
| (SEQ ID NO: 2494) | ct ggcctggcct ggccctgc |
| (SEQ ID NO: 2495) | t ggcctggcct ggccctgcc |
| (SEQ ID NO: 2496) | ggcctggcct ggccctgccc |
| (SEQ ID NO: 2497) | gcctggcct ggccctgccc t |
| (SEQ ID NO: 2498) | cctggcct ggccctgccc tg |
| (SEQ ID NO: 2499) | ctggcct ggccctgccc tga |
| (SEQ ID NO: 2500) | tggcct ggccctgccc tgag |
| (SEQ ID NO: 2501) | ggcct ggccctgccc tgagg |
| (SEQ ID NO: 2502) | gcct ggccctgccc tgagga |
| (SEQ ID NO: 2503) | cct ggccctgccc tgaggaa |
| (SEQ ID NO: 2504) | ct ggccctgccc tgaggaag |
| (SEQ ID NO: 2505) | t ggccctgccc tgaggaagg |
| (SEQ ID NO: 2506) | ggccctgccc tgaggaagga |
| (SEQ ID NO: 2507) | gccctgccc tgaggaagga g |
| (SEQ ID NO: 2508) | ccctgccc tgaggaagga gg |
| (SEQ ID NO: 2509) | cctgccc tgaggaagga ggg |
| (SEQ ID NO: 2510) | ctgccc tgaggaagga gggg |
| (SEQ ID NO: 2511) | tgccc tgaggaagga gggga |
| (SEQ ID NO: 2512) | gccc tgaggaagga ggggaa |
| (SEQ ID NO: 2513) | ccc tgaggaagga ggggaag |
| (SEQ ID NO: 2514) | cc tgaggaagga ggggaagc |
| (SEQ ID NO: 2515) | c tgaggaagga ggggaagct |
| (SEQ ID NO: 2516) | tgaggaagga ggggaagctg |
| (SEQ ID NO: 2517) | gaggaagga ggggaagctg c |
| (SEQ ID NO: 2518) | aggaagga ggggaagctg ca |
| (SEQ ID NO: 2519) | ggaagga ggggaagctg cag |
| (SEQ ID NO: 2520) | gaagga ggggaagctg cagc |
| (SEQ ID NO: 2521) | aagga ggggaagctg cagct |
| (SEQ ID NO: 2522) | agga ggggaagctg cagctt |
| (SEQ ID NO: 2523) | gga ggggaagctg cagcttg |
| (SEQ ID NO: 2524) | ga ggggaagctg cagcttgg |
| (SEQ ID NO: 2525) | a ggggaagctg cagcttggg |
| (SEQ ID NO: 2526) | ggggaagctg cagcttggga |
| (SEQ ID NO: 2527) | gggaagctg cagcttggga g |
| (SEQ ID NO: 2528) | ggaagctg cagcttggga ga |
| (SEQ ID NO: 2529) | gaagctg cagcttggga gag |
| (SEQ ID NO: 2530) | aagctg cagcttggga gagc |
| (SEQ ID NO: 2531) | agctg cagcttggga gagcc |
| (SEQ ID NO: 2532) | gctg cagcttggga gagccc |
| (SEQ ID NO: 2533) | ctg cagcttggga gagcccc |
| (SEQ ID NO: 2534) | tg cagcttggga gagcccct |
| (SEQ ID NO: 2535) | g cagcttggga gagcccctg |
| (SEQ ID NO: 2536) | cagcttggga gagcccctgg |
| (SEQ ID NO: 2537) | agcttggga gagcccctgg g |
| (SEQ ID NO: 2538) | gcttggga gagcccctgg gg |
| (SEQ ID NO: 2539) | cttggga gagcccctgg ggc |
| (SEQ ID NO: 2540) | ttggga gagcccctgg ggcc |
| (SEQ ID NO: 2541) | tggga gagcccctgg ggcct |
| (SEQ ID NO: 2542) | ggga gagcccctgg ggccta |
| (SEQ ID NO: 2543) | gga gagcccctgg ggcctag |
| (SEQ ID NO: 2544) | ga gagcccctgg ggcctaga |
| (SEQ ID NO: 2545) | a gagcccctgg ggcctagac |
| (SEQ ID NO: 2546) | gagcccctgg ggcctagact |
| (SEQ ID NO: 2547) | agcccctgg ggcctagact c |
| (SEQ ID NO: 2548) | gcccctgg ggcctagact ct |
| (SEQ ID NO: 2549) | cccctgg ggcctagact ctg |
| (SEQ ID NO: 2550) | ccctgg ggcctagact ctgt |
| (SEQ ID NO: 2551) | cctgg ggcctagact ctgta |
| (SEQ ID NO: 2552) | ctgg ggcctagact ctgtaa |
| (SEQ ID NO: 2553) | tgg ggcctagact ctgtaac |
| (SEQ ID NO: 2554) | gg ggcctagact ctgtaaca |
| (SEQ ID NO: 2555) | g ggcctagact ctgtaacat |
| (SEQ ID NO: 2556) | ggcctagact ctgtaacatc |
| (SEQ ID NO: 2557) | gcctagact ctgtaacatc a |
| (SEQ ID NO: 2558) | cctagact ctgtaacatc ac |
| (SEQ ID NO: 2559) | ctagact ctgtaacatc act |
| (SEQ ID NO: 2560) | tagact ctgtaacatc acta |
| (SEQ ID NO: 2561) | agact ctgtaacatc actat |
| (SEQ ID NO: 2562) | gact ctgtaacatc actatc |
| (SEQ ID NO: 2563) | act ctgtaacatc actatcc |
| (SEQ ID NO: 2564) | ct ctgtaacatc actatcca |
| (SEQ ID NO: 2565) | t ctgtaacatc actatccat |
| (SEQ ID NO: 2566) | ctgtaacatc actatccatg |
| (SEQ ID NO: 2567) | tgtaacatc actatccatg c |
| (SEQ ID NO: 2568) | gtaacatc actatccatg ca |
| (SEQ ID NO: 2569) | taacatc actatccatg cac |
| (SEQ ID NO: 2570) | aacatc actatccatg cacc |
| (SEQ ID NO: 2571) | acatc actatccatg cacca |
| (SEQ ID NO: 2572) | catc actatccatg caccaa |
| (SEQ ID NO: 2573) | atc actatccatg caccaaa |
| (SEQ ID NO: 2574) | tc actatccatg caccaaac |
| (SEQ ID NO: 2575) | c actatccatg caccaaact |
| (SEQ ID NO: 2576) | actatccatg caccaaacta |
| (SEQ ID NO: 2577) | ctatccatg caccaaacta a |
| (SEQ ID NO: 2578) | tatccatg caccaaacta at |
| (SEQ ID NO: 2579) | atccatg caccaaacta ata |
| (SEQ ID NO: 2580) | tccatg caccaaacta ataa |
| (SEQ ID NO: 2581) | ccatg caccaaacta ataaa |
| (SEQ ID NO: 2582) | catg caccaaacta ataaaa |
| (SEQ ID NO: 2583) | atg caccaaacta ataaaac |
| (SEQ ID NO: 2584) | tg caccaaacta ataaaact |
| (SEQ ID NO: 2585) | g caccaaacta ataaaactt |
| (SEQ ID NO: 2586) | caccaaacta ataaaacttt |
| (SEQ ID NO: 2587) | accaaacta ataaaacttt g |
| (SEQ ID NO: 2588) | ccaaacta ataaaacttt ga |
| (SEQ ID NO: 2589) | caaacta ataaaacttt gac |
| (SEQ ID NO: 2590) | aaacta ataaaacttt gacg |
| (SEQ ID NO: 2591) | aacta ataaaacttt gacga |
| (SEQ ID NO: 2592) | acta ataaaacttt gacgag |
| (SEQ ID NO: 2593) | cta ataaaacttt gacgagt |
| (SEQ ID NO: 2594) | ta ataaaacttt gacgagtc |
| (SEQ ID NO: 2595) | a ataaaacttt gacgagtca |
| (SEQ ID NO: 2596) | ataaaacttt gacgagtcac |
| (SEQ ID NO: 2597) | taaaacttt gacgagtcac c |
| (SEQ ID NO: 2598) | aaaacttt gacgagtcac ct |
| (SEQ ID NO: 2599) | aaacttt gacgagtcac ctt |
| (SEQ ID NO: 2600) | aacttt gacgagtcac cttc |
| (SEQ ID NO: 2601) | acttt gacgagtcac cttcc |
| (SEQ ID NO: 2602) | cttt gacgagtcac cttcca |
| (SEQ ID NO: 2603) | ttt gacgagtcac cttccag |
| (SEQ ID NO: 2604) | tt gacgagtcac cttccagg |
| (SEQ ID NO: 2605) | t gacgagtcac cttccagga |
| (SEQ ID NO: 2606) | gacgagtcac cttccaggac |
| (SEQ ID NO: 2607) | acgagtcac cttccaggac c |
| (SEQ ID NO: 2608) | cgagtcac cttccaggac cc |
| (SEQ ID NO: 2609) | gagtcac cttccaggac ccc |
| (SEQ ID NO: 2610) | agtcac cttccaggac ccct |
| (SEQ ID NO: 2611) | gtcac cttccaggac ccctg |
| (SEQ ID NO: 2612) | tcac cttccaggac ccctgg |
| (SEQ ID NO: 2613) | cac cttccaggac ccctggg |
| (SEQ ID NO: 2614) | ac cttccaggac ccctgggt |
| (SEQ ID NO: 2615) | c cttccaggac ccctgggta |
| (SEQ ID NO: 2616) | cttccaggac ccctgggtaa |
| (SEQ ID NO: 2617) | ttccaggac ccctgggtaa a |
| (SEQ ID NO: 2618) | tccaggac ccctgggtaa aa |
| (SEQ ID NO: 2619) | ccaggac ccctgggtaa aaa |
| (SEQ ID NO: 2620) | caggac ccctgggtaa aaaa |
| (SEQ ID NO: 2621) | aggac ccctgggtaa aaaaa |
| (SEQ ID NO: 2622) | ggac ccctgggtaa aaaaaa |
| (SEQ ID NO: 2623) | gac ccctgggtaa aaaaaaa |
| (SEQ ID NO: 2624) | ac ccctgggtaa aaaaaaaa |
| (SEQ ID NO: 2625) | c ccctgggtaa aaaaaaaaa |
| (SEQ ID NO: 2626) | ccctgggtaa aaaaaaaaaa |
| (SEQ ID NO: 2627) | cctgggtaa aaaaaaaaaa a |
| (SEQ ID NO: 2628) | ctgggtaa aaaaaaaaaa aa |
| (SEQ ID NO: 2629) | tgggtaa aaaaaaaaaa aaa |

Accordingly, the present invention extends to an isolated oligonucleotide from DRD2 cDNA when encompassing a polymorphism or mutation associated with a neurological, psychiatric or psychological conditions, phenotype or state selected from SEQ ID NO: 8 through SEQ ID NO:2616.

There are 20 mer oligonucleotides and as indicated above, the present invention extends to oligonucleotides from 3 to 100 nucleotides in length. In fact, the full length cDNA may also be employed. For example, SEQ ID NO:2 provides the full cDNA sequence of DRD2 cDNA comprising a C at position 957.

In a preferred embodiment, one of the at least two primers is involved in an amplification reaction to amplify a target sequence. If this primer is also labeled with a reporter molecule, the amplification reaction will result in the incorporation of any of the label into the amplified product. The terms “amplification product” and “amplicon” may be used interchangeably.

The primers and the amplicons of the present invention may also be modified in a manner which provides either a detectable signal or aids in the purification of the amplified product.

A range of labels providing a detectable signal may be employed. The label may be associated with a primer or amplicon or it may be attached to an intermediate which subsequently binds to the primer or amplicon. The label may be selected from a group including a chromogen, a catalyst, an enzyme, a fluorophore, a luminescent molecule, a chemiluminescent molecule, a lanthanide ion such as Europium (Eu³⁴), a radioisotope and a direct visual label. In the case of a direct visual label, use may be made of a colloidal metallic or non-metallic particular, a dye particle, an enzyme or a substrate, an organic polymer, a latex particle, a liposome, or other vesicle containing a signal producing substance and the like. A large number of enzymes suitable for use as labels is disclosed in U.S. Pat. Nos. 4,366,241, 4,843,000 and 4,849,338. Suitable enzyme labels useful in the present invention include alkaline phosphatase, horseradish peroxidase, luciferase, p-galactosidase, glucose oxidase, lysozyme, malate dehydrogenase and the like. The enzyme label may be used alone or in combination with a second enzyme which is in solution. Alternatively, a fluorophore which may be used as a suitable label in accordance with the present invention includes, but is not limited to, fluorescein-isothiocyanate (FITC), and the fluorochrome is selected from FITC, cyanine-2, Cyanine-3, Cyanine-3.5, Cyanine-5, Cyanine-7, fluorescein, Texas red, rhodamine, lissamine and phycoerythrin.

Examples of fluorophores are provided in Table 3.

TABLE 3

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3, 5 | 581 | 596; (640) |
| Cy5 | (625); 650 | 670 |
| Cy5, 5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bromide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

In order to aid in the purification of an amplicon, the primers or amplicons may additionally incorporate a bead. The beads used in the methods of the present invention may either be magnetic beads or beads coated with streptavidin.

The extension of the hybridized primer to produce an extension product is included herein by the term amplification. Amplification generally occurs in cycles of denaturation followed by primer hybridization and extension. The present invention encompasses form about 1 cycle to about 120 cycles, preferably from about 2 to about 70 cycles, more preferably from about 5 to about 40 cycles, including 10, 15, 20, 25 and 30 cycles, and even more preferably, 35 cycles such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 cycles.

In order for the primers used in the methods of the present invention to anneal to a nucleic acid molecule containing the gene of interest, a suitable annealing temperature must be determined. Determination of an annealing temperatures is based primarily on the genetic make-up of the primer, i.e. the number of A, T, C and Gs, and the length of the primer. Annealing temperatures contemplated by the methods of the present invention are from about 40° C. to about 80° C., preferably from about 50° C. to about 70° C., and more preferably about 65° C. such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80° C.

The PCR amplifications performed in the methods of the present invention include the use of MgCl₂ in the optimization of the PCR amplification conditions. The present invention encompasses MgCl₂ concentrations for about 0.1 to about 10 mM, preferably from 0.5 to about 5 mM, and even more preferably 2.5 mM such as 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 mM.

Polymorphisms of the present invention may be detected due to the presence of a base mis-match in the heteroduplexes formed following PCR amplification. A base mis-match occurs when two nucleotide sequences are aligned with substantial complementarity but at least one base aligns to a base which would result in an “abnormal” binding pair. An abnormal binding pair occurs when thymine (T) were to bind to a base other than adenine (A), if A were to bind to a base other than T, if guanine (G) were to bind to a base other than cytosine (C) or if C was to bind to a base other than G.

In order to detect the presence of a DRD2 allele predisposing an individual to an inability to overcome a neurological, psychiatric or psychological condition, phenotype or state or sub-threshold form thereof or a risk of developing same, a biological sample such as blood is obtained and analyzed for the presence or absence of one or more susceptibility alleles of the DRD2 genetic locus identified as being statistically significantly associated with the neurological, psychiatric or psychological condition, phenotype or state of interest of DRD2. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis. Suitable diagnostic techniques include those described herein as well as those described in U.S. Pat. Nos. 5,837,492; 5,800,998 and 5,891,628.

According to the present invention, a method is also provided for supplying wild-type DRD2 function to a cell which carries a DRD2 allele mutant or polymorphism. Supplying such a function should allow normal functioning of the recipient cells. The wild-type gene or a part of the gene may be introduced into the cell in a vector such that the gene remains extrachromosomal. In such a situation, the gene will be expressed by the cell from the extrachromosomal location. More preferred is the situation where the wild-type gene or a part thereof is introduced into the mutant cell in such a way that it recombines with the endogenous mutant gene present in the cell. Such recombination requires a double recombination event which results in the correction of the gene mutation. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner. Conventional methods are employed, including those described in U.S. Pat. Nos. 5,837,492; 5,800,998 and 5,891,628.

The identification of the association between the DRD2 gene polymorphism/mutations and a psychological phenotype or sub-threshold psychological phenotype permits the early presymptomatic screening of individuals to identify those at risk for developing a neurological, psychiatric or psychological condition, phenotype or state or sub-threshold neurological, psychiatric or psychological condition, phenotype or state such as schizophrenia or to identify the cause of such disorders or the risk that any individual will develop same. To identify such individuals, the alleles are screened as described herein or using conventional techniques, including but not limited to, one of the following methods: fluorescent in situ hybridization (FISH), direct DNA sequencing, PFGE analysis, Southern blot analysis, single stranded conformation analysis (SSCP), linkage analysis, RNase protection assay, allele-specific oligonucleotide (ASO), dot blot analysis and PCR-SSCP analysis. Also useful is the recently developed technique of DNA microchip technology. Such techniques are described in U.S. Pat. Nos. 5,837,492; 5,800,998 and 5,891,628, each incorporated herein by reference.

Genetic testing enables practitioners to identify individuals at risk for certain behavioral states including substance addition or an inability to overcome a neurological, psychiatric or psychological condition, phenotype or state or a sub-threshold form thereof after initial treatment. For particular at risk couples, embryos or fetuses may be tested after conception to determine the genetic likelihood of the offspring being pre-disposed to the neurological, psychiatric or psychological condition, phenotype or state. Certain behavioral or therapeutic protocols may then be introduced from birth or early childhood to reduce the risk of the neurological, psychiatric or psychological condition, phenotype or state developing. Presymptomatic diagnosis will enable better treatment of these disorders, including the use of existing medical therapies. Genetic testing will also enable practitioners to identify individuals having diagnosed disorders (or in an at risk group) which have polymorphism identified in the DRD2 genetic locus. Genotyping of such individuals will be useful for (a) identifying neurological, psychiatric or psychological condition, phenotype or state or a sub-threshold form thereof that will respond to drugs affecting DRD2 activity, (b) identifying a neurological, psychiatric or psychological condition, phenotype or state or sub-threshold neurological, psychiatric or psychological condition, phenotype or state that in an individual which respond well to placebos versus those that respond better to active drugs and (c) guide new drug discovery and testing. Further, the present invention provides a method for screening drug candidates to identify molecules useful for treating neurological, psychiatric or psychological conditions, phenotypes or states involving the DRD2 gene or its expressive product. Drug screening is performed by comparing the activity of native genes and those described herein in the presence and absence of potential drugs. In particular, these drugs may have the affect of masking a polymorphism or mutation or may bind to a particular polymorphism or mutation enabling it to be used as a diagnostic agent. The terms “drug”, “agent”, “therapeutic molecule”, “prophylactic molecule”, “medicament”, “candidate molecule” or “active ingredient” may be used interchangeable in describing this aspect of the present invention.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo or which are specific for a targetable (e.g. a polymorphism) and hence is a useful diagnostic. Several approaches for use in rational drug design include analysis of three-dimensional structure, alanine scans, molecular modeling and use of anti-id antibodies. These techniques are well known to those skilled in the art, including those described in U.S. Pat. Nos. 5,837,492; 5,800,998 and 5,891,628.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing are generally used to avoid randomly screening large numbers of molecules for a target property.

Once the pharmacophore has been found, its structure is modelled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process. A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Briefly, a method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Following identification of a substance which modulates or affects DRD2 activity, the substance may be further investigated. Furthermore, it may be manufactured and/or used in preparation, i.e., a manufacture or formulation, or a composition such as a medicament, pharmaceutical composition or drug. These may be administered to individuals directly or via gene therapy.

The DRD2 polypeptides, antibodies, peptides and nucleic acids of the present invention can be formulated in pharmaceutical compositions, which are prepared according to conventional pharmaceutical compounding techniques. See, for example, Remington's Pharmaceutical Sciences, 18th Ed. 1990, Mack Publishing Co., Easton, Pa. The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g., intravenous, oral, intrathecal, epineural or parenteral.

The present invention provides information necessary for physicians to select drugs for use in the treatment of a neurological, psychiatric or psychological condition, phenotype or state or a sub-threshold form thereof. With the identification that polymorphisms within the DRD2 gene are associated with a neurological, psychiatric or psychological condition, phenotype or state including a sub-threshold form thereof, such as schizophrenia, antipsychotic medications, such as partial agonists or antagonists of DRD2 can be selected for the treatment of such conditions.

The present invention further contemplates a method of treating a neurological, psychiatric or psychological condition, phenotype or state in an individual the method comprising identifying a polymorphism in the DRD2 genetic locus with the neurological, psychiatric or psychological condition, phenotype or state and subjecting the individual to gene therapy to alter the gene or genetic sequence having a different polymorphism or to treat the defect caused by the polymorphism or to subject the individual to behavioral modification protocols to help ameliorate the symptoms.

Using the compositions of the present invention, gene therapy may be recommended when a particular polymorphism conferring, for example, a disease condition or a propensity for development of neurological, psychiatric or psychological condition, phenotype or state is identified in an embryo. Genetically modified stem cells may then be used to alter the genotype of the developing cells. Where an embryo has developed into a fetus or for post-natal subjects, localized gene therapy may still be accomplished. Alternatively, a compound may be identified which effectively masks a particular undesired polymorphic variant or which influences the expression of a more desired phenotype. For example, one polymorphic variant of a receptor may result in an instability of the mRNA transition product.

Accordingly, the present invention also provides genetic test kits which allow the rapid screening of a DRD2 polymorphism or polymorphisms within a test sample or multiple test samples. The kits of the present invention comprise one or more sets of primers, as described herein, which are specific for the amplification of a genetic region of interest. In addition, the genetic testing kits of the present invention provide a PCR mix, comprising MgCl₂. In a preferred aspect, the MgCl₂ is provided at a concentration of 2.5 mM. Additionally, the genetic test kits of the present invention provide instructions for using the primers of the present invention to obtain the desired duplexes, as well as instructions as to the analysis of the duplexes using d-HPLC.

The present invention is further described with reference to the following non-limiting Examples.

### Example 1

### Genotyping Study

### Subject Enrolment

One hundred and fifty three unrelated Caucasian patients (133 males, 20 females) attending various psychiatric units for the treatment of their schizophrenia were recruited for the study. Patients were being treated at the Fortitude Valley Community Mental Health Centre, the Royal Brisbane Mental Health Unit and the Park Psychiatric Hospital. Inclusion criteria were being between 18 and 65 years of age and having a DSM IV diagnosis of schizophrenia. In this particular study potential participants were excluded if they had Schizoaffective Disorder, Bipolar Disorder, Dementia, Organic Brain Syndrome or Major Depressive Disorder with Delusions. The study further included One hundred and forty eight controls (41 females, 107 males). A 10 mL blood sample was drawn from each subject for DNA extraction. All participants provided informed consent and were able to terminate participation at any time without prejudice. Institutional ethics approval was obtained from the university, clinics and hospitals involved.

### Genotyping

DNA was extracted from leucocytes using standard techniques and subsequently used as a template for determination of 957C>T genotypes. Genotyping was performed by real-time PCR using the Applied Biosystems 7000 sequence detection system (Applied Biosystems, Foster City, Calif., USA). Sequence specific primers were designed for the C allele (5′-ATGGTCTCCACAGCACTCTC-3′ SEQ ID NO:4), the T allele (5′-ATGGTCTCCACAGCACTCTT-3′ SEQ ID NO:5) and a common reverse primer (5′-CATTGGGCATGGTCTGGATC-3′ SEQ ID NO:6). A total of 5-10 ng of genomic DNA was amplified in 1×SYBR green PCR master mix (Applied Biosystems) containing 0.4 μM of allele specific forward primer and 0.4 μM of common reverse primer in a 25 μl volume. Amplification conditions were: 50° C. for 2 min, 95° C. for 10 min, followed by 40 cycles of 95° C. for 15 s and 60° C. for 1 min. A cycle time (Cₜ) value was obtained by setting the threshold during geometric phase of amplification and scored relative to the ΔCₜ generated between the matched and mismatched primer pairs.

### Data Analysis

Information coded from interview proformas was entered into a computer data base. Chi-square test were employed to compare differences in non continuous variables between 957C>T genotype groups. A p-value of ≦0.05 was considered to be statistically significant.

### Example 2

### Identification of a Schizophrenia Marker

To evaluate the frequency of 957C and 957T in patients with schizophrenia and alcoholism and in controls, 153 patients meeting DSM-1V criteria for schizophrenia, 132 severely alcoholic subjects and 148 general population controls were genotyped for the 957C>T polymorphism.

The observed allele frequency and genotype frequencies of the 957C>T polymorphism in control, schizophrenic and alcoholic individuals revealed a significant increase in the frequency of the 957C allele in both schizophrenia (Table 4) and alcoholism (Table 5) compared to the controls. The genotype frequencies in the schizophrenic and alcoholic groups also differed significantly from expected values compared to controls although it is interesting to note that the heterozygote frequency is approximately the same in the two groups. The schizophrenic, alcoholic and control groups appeared to be in Hardy-Weinberg equilibrium based on the respective allele frequency of each group.

Using standard population genetics calculations and assuming a prevalence of schizophrenia of 1% in the general population CC alleles account for 25% of the heritability of schizophrenia.

TABLE 4
Allele and genotype frequencies of the 957C > T polymorphism
in schizophrenia.
Allele Frequency

| allele | C | T |
|---|---|---|
| Controls | 124 (41.9%) | 172 (58.1%) |
| Schizophrenia* | 171 (55.5%) | 137 (44.5%) |
| Expected | 129.0 | 179.00 |

Genotype frequency

| Genotype | CC | CT | TT |
|---|---|---|---|
| Controls | 27 (18.2%) | 70 (47.3%) | 51 (34.5%) |
| Expected | 26.0 | 72.1 | 50.0 |
| Schizophrenia** | 48 (31.2%) | 75 (48.7%) | 31 (20.1%) |
| Expected | 27.0 | 75.0 | 52.0 |

*Fisher's exact P P = 0.00085 (2 × 2 contingency test)
*χ² = 11.219 P = 0.00081 (2 × 2 contingency test)
*χ² (goodness of fit) = 23.40; P = 1.25 × 10⁻⁶
**Fisher's exact P P = 0.0043 (2 × 3 contingency test)
**χ² (Pearson) = 10.816 P = 0.0045 (2 × 3 contingency test)
**χ² (goodness of fit) = 24.79; P = 4.2 × 10⁻⁶

TABLE 5
Allele and genotype frequencies of the 957C > T polymorphism
in alcoholism.
Allele Frequency

| allele | C | T |
|---|---|---|
| Controls | 124 (41.9%) | 172 (58.1%) |
| Alcohol dependence* | 133 (50.4%) | 131 (49.6%) |
| Expected | 110.6 | 153.4 |

Genotype frequency

| Genotype | CC | CT | TT |
|---|---|---|---|
| Controls | 27 (18.2%) | 70 (47.3%) | 51 (34.5%) |
| Expected | 26.0 | 72.1 | 50.0 |
| Alcohol dependence** | 37 (28.0%) | 59 (44.7%) | 36 (27.3%) |
| Expected | 23.2 | 64.2 | 44.6 |

*Fisher's exact P P = 0.051 (2 × 2 contingency test)
*χ² = 4.05 P = 0.044 (2 × 2 contingency test)
*χ² (goodness of fit) = 7.81; P = 0.0052
**Fisher's exact P P = 0.1289 (2 × 3 contingency test)
**χ² (Pearson) = 4.192 P = 0.123 (2 × 3 contingency test)
**χ² (goodness of fit) = 10.34; P = 0.0057

### Example 3

### Genotyping Study

### Schizophrenia Subject Enrolment

One hundred and sixty unrelated Caucasian patients (137 males, 23 females) attending various psychiatric units for the treatment of their schizophrenia were recruited for the study. Patients were being treated at the Fortitude Valley Community Mental Health Centre, the Royal Brisbane Mental Health Unit and the Park Psychiatric Hospital, Brisbane, Australia. Inclusion criteria were being between 18 and 65 years of age and having a DSM IV diagnosis of schizophrenia. In this particular study potential participants were excluded if they had Schizoaffective Disorder, Bipolar Disorder, Dementia, Organic Brain Syndrome or Major Depressive Disorder with Delusions. The study further included two hundred and twenty nine controls (134 males, 95 females). A 10 mL blood sample was drawn from each subject for DNA extraction.

### Post Traumatic Stress Disorder Subject Enrolment

One hundred and ten unrelated Caucasian patients (110 males, 0 females) attending Greenslopes Private Hospital, Brisbane, Australia, were collected. Inclusion criteria were being between 18 and 65 years of age and having a DSM IV diagnosis of Post Traumatic Stress Disorder. The study further included two hundred and twenty nine controls (134 males, 95 females). A 10 mL blood sample was drawn from each subject for DNA extraction.

### Alcohol Dependent Subject Enrolment

Two hundred and thirty two unrelated Caucasian patients (151 Males, 81 females) attending treatment at The Royal Brisbane Hospital, Brisbane, Australia, were collected for analysis. Inclusion criteria were being between 18 and 65 years of age and having a DSM IV diagnosis of alcohol dependence. The study further included two hundred and twenty nine controls (134 males, 95 females). A 10 mL blood sample was drawn from each subject for DNA extraction.

### Nicotine Dependent Subject Enrolment

One hundred and fifty unrelated Caucasian patients (76 Males, 74 females) attending The Royal Brisbane Hospital were collected. Inclusion criteria were being between 18 and 65 years of age and having a DSM IV diagnosis of nicotine dependence. The study further included two hundred and twenty nine controls (134 males, 95 females). A 10 mL blood sample was drawn from each subject for DNA extraction.

### Opioid Dependent Subject Enrolment

One hundred and eighteen unrelated Caucasian patients (69 Males, 49 females) attending treatment at The Royal Brisbane Hospital were collected for analysis. Inclusion criteria were being between 18 and 65 years of age and having a DSM IV diagnosis of opioid dependence. The study further included two hundred and twenty nine controls (134 males, 95 females). A 10 mL blood sample was drawn from each subject for DNA extraction.

### Genotyping

### 957C>T

DNA was extracted from leucocytes using standard techniques and subsequently used as a template for determination of 957C>T genotypes. Genotyping was performed by real-time PCR using the Applied Biosystems 7000 sequence detection system (Applied Biosystems, Foster City, Calif., USA). Sequence specific primers were designed for the C allele (5′-ATGGTCTCCACAGCACTCTC-3′ SEQ ID NO:4), the T allele (5′-ATGGTCTCCACAGCACTCTT-3′ SEQ ID NO:5) and a common reverse primer (5′-CATTGGGCATGGTCTGGATC-3′ SEQ ID NO:6). A total of 5-10 ng of genomic DNA was amplified in 1×SYBR green PCR master mix (Applied Biosystems) containing 0.4 μM of allele specific forward primer and 0.4 μM of common reverse primer in a 25 μl volume. Amplification conditions were: 50° C. for 2 min, 95° C. for 10 min, followed by 40 cycles of 95° C. for 15 s and 60° C. for 1 min. A cycle time (Cₜ) value was obtained by setting the threshold during geometric phase of amplification and scored relative to the ΔCₜ generated between the matched and mismatched primer pairs.

### Taq1A

DNA was extracted from leucocytes using standard techniques and subsequently used as a template for determination of Taq1 A (C<T) genotypes. Genotyping was performed by PCR-restriction fragment length polymorphism (RFLP) of amplified PCR fragments. The genomic sequence of 501 bp of the 3′-flanking region of DRD2 was amplified by PCR with the primer pair, forward primer (5′-GCACGTGCCACCATACCC-3′ SEQ ID NO:2630) and a reverse primer (5′-TGCAGAGCAGTCAGGCTG-3′ SEQ ID NO:2631). A total of 5-10 ng of genomic DNA was amplified in a PCR master mix containing 0.2 μM of forward primer and 0.2 μM of reverse primer, 1×PCR buffer, 1.5 mM MgCl₂, 200 μM dNTPs, H₂0 and 1 unit of Platinum Taq DNA Polymerase (Invitrogen) in a 25 μl volume. Amplification conditions were: Step 1: 94° C. for 4 min, Step 2: 94° C. for 30 s, Step 3: 68° C. for 30 s, Step 4: 72° C. for 30 s, Steps 2-4 were repeated by 40 cycles followed by 72° C. for 3 min. Amplified PCR fragments were digested with Taq^{a} I (5′ . . . T^{▾}CGA . . . 3′) restriction enzyme (New England Biolabs) and digested fragments were visualised via 2% agarose gel electrophoresis and ethidium bromide staining.

-141delC

DNA was extracted from leucocytes using standard techniques and subsequently used as a template for determination of -141C Ins/Del genotypes. Genotyping was performed by RFLP of amplified PCR fragments. The genomic sequence of 284 bp of the 5′-flanking region and 274 bp of exon 1 of DRD2 was amplified by PCR with the primer pair, forward primer (5′-ACTGGCGAGCAGACGGTGAGGACCC-3′ SEQ ID NO:2632) and a reverse primer (5′-TGCGCGCGTGAGGCTGCCGGTTCGG-3′ SEQ ID NO:2633). A total of 5-10 ng of genomic DNA was amplified in a PCR master mix containing 0.2 μM of forward primer and 0.2 μM of reverse primer, 1×PCR buffer, 1.5 mM MgCl₂, 200 μM dNTPs, H₂0, 2× enhancer solution (Invitrogen) and 1 unit of Platinum Taq DNA Polymerase (Invitrogen) in a 25 μl volume. Amplification conditions were: Step 1: 95° C. for 3 min, Step 2: 95° C. for 30 s, Step 3: 68° C. for 30 s, Step 4: 72° C. for 30 s, Steps 2-4 were repeated by 40 cycles followed by 72° C. for 2 min. Amplified PCR fragments were digested with BstN1 (5′ . . . CC^{▾}(A/T)GG . . . 3′) restriction enzyme (New England Biolabs) and digested fragments were visualised via 2% w/v agarose gel electrophoresis and ethidium bromide staining.

### Data Analysis

Information coded from interview proformas was entered into a computer data base. Chi-square test were employed to compare differences in non continuous variables between 957C>T genotype groups. A p-value of ≦0.05 was considered to be statistically significant. Two stringent statistical tests were applied to test the null hypothesis that there was no difference between the case and the control groups: Compare 2 Version 1.25 was used for the comparison of two independent groups or samples. Fisher's exact P and Chi square for a 2 by 2 contingency table (allele frequencies) and a 2 by 3 table (for genotype frequencies). A further test (chi-squared for goodness of fit) was applied to determine whether the case population mimicked the control population based on the control allele frequencies. Raw genotype data for each individual for the control and test population were entered into the PHASE v2.1.1 program to generate predicted haplotype numbers and haplotype frequencies. Haplotype numbers generated were analyzed using the Compare 2 Version 1.25 using a 2 by k table to generate a likelihood-ratio Chi square and odds ratios.

### Example 4

### Identification of DRD2 Markers for Schizophrenia, Alcohol Dependence, Nicotine Dependence, Post Traumatic Stress Disorder (PTSD) and No Association of DRD2 Markers with Opiate Dependence

### Genotype Analysis

The analysis on the 957CT polymorphism was extended to schizophrenia by expanding the number of schizophrenic patients and the number of control subjects. This result confirms and strengthens the conclusions from the study in Example 2, i.e. there is a strong association between the DRD2 957C allele and schizophrenia (see Tables 7 to 10).

This study was further extended by including the analysis of 2 further polymorphisms to include a total of 3 polymorphisms (957C>T, Taq 1A and -141delC). These 3 polymorphisms were analyzed in the control and schizophrenia patients and these were also analyzed in a several other patient groups including, post traumatic stress disorder (PTSD), alcohol dependence, nicotine dependence and opiate dependence. First, the patient groups and the control group were tested to see if they were in Hardy-Weinberg equilibrium (Table 6). All groups were in equilibrium for each of the 3 SNPs except the -141delC polymorphism in the alcohol dependence group. The genetic association of each of the 3 polymorphisms was tested in each of the patient groups by analysis of the genotype (Table 7) and allele (Table 9) frequencies. The 957C allele of the 957C>T polymorphism was significantly associated with schizophrenia, PTSD, alcohol dependence and nicotine dependence but there was no association with opiate dependence (when analyzed by goodness-of-fit to the control population and a 2×2 contingency Table 8).

TABLE 6
Hardy-Weinberg equilibrium analysis for control and patient groups.

| | 957C > T | | Taq1A | | −141delC | |
|---|---|---|---|---|---|---|
| | | Chi | | Chi | | Chi |
| | P | squared | P | squared | P | squared |
| Control | 0.98 | 0.04 | 0.82 | 0.40 | 0.68 | 0.77 |
| Schizophrenia | 0.89 | 0.23 | 1.00 | 0.00 | 0.31 | 2.31 |
| PTSD | 0.98 | 0.04 | 0.85 | 0.34 | 0.98 | 0.05 |
| Alcohol | 0.46 | 1.56 | 0.87 | 0.28 | 0.01* | 10.34 |
| dependence | | | | | | |
| Nicotine | 0.70 | 0.70 | 0.83 | 0.37 | 0.94 | 0.12 |
| dependence | | | | | | |
| Opiate | 0.97 | 0.06 | 0.93 | 0.14 | 0.95 | 0.09 |
| dependence | | | | | | |

TABLE 7
Observed and expected allele numbers

| | 957C > T | | Taq1A | | −141delC | |
|---|---|---|---|---|---|---|
| Alleles | C | T | A1 | A2 | C | CC |
| Control | 192 | 260 | 77 | 369 | 48 | 376 |
| Schizophrenia | 178 | 142 | 76 | 242 | 34 | 280 |
| Expected | 135.9 | 184.1 | 54.9 | 263.1 | 35.5 | 278.5 |
| PTSD | 112 | 108 | 47 | 173 | 18 | 180 |
| Expected | 93.5 | 126.5 | 38.0 | 182.0 | 22.4 | 175.6 |
| Alcohol | 230 | 232 | 93 | 353 | 62 | 388 |
| dependence | | | | | | |
| Expected | 196.2 | 265.8 | 77.0 | 369.0 | 50.9 | 399.1 |
| Nicotine | 142 | 142 | 70 | 224 | 27 | 239 |
| dependence | | | | | | |
| Expected | 120.6 | 163.4 | 50.8 | 243.2 | 30.1 | 235.9 |
| Opiate | 105 | 131 | 46 | 182 | 27 | 193 |
| dependence | | | | | | |
| Expected | 100.2 | 135.8 | 39.4 | 188.6 | 24.9 | 195.1 |

TABLE 8
Allele data analysis (Goodness of fit Chi-squared and Fisher's P exact test)

| | 957C > T | | | Taq 1A | | | −141delC | | |
|---|---|---|---|---|---|---|---|---|---|
| | chi | | | chi | | | chi | | |
| | squared | P value | Fisher's P | squared | P value | Fisher's P | squared | P value | Fisher's P |
| Schizophrenia | 22.64 | 0.000002 | 3.4E−4 | 9.80 | 0.001745 | 0.028 | 0.08 | 0.782882 | 0.906 |
| PTSD | 6.40 | 0.011410 | 0.047 | 2.59 | 0.107684 | 0.206 | 0.98 | 0.322036 | 0.485 |
| Alcohol | 10.09 | 0.001489 | 0.029 | 4.02 | 0.045005 | 0.201 | 2.71 | 0.099969 | 0.308 |
| Dependence | | | | | | | | | |
| Nicotine | 6.58 | 0.010333 | 0.048 | 8.82 | 0.002985 | 0.031 | 0.36 | 0.546877 | 0.707 |
| Dependence | | | | | | | | | |
| Opiate | 0.39 | 0.531441 | 0.627 | 1.35 | 0.244845 | 0.399 | 0.20 | 0.655855 | 0.796 |
| Dependence | | | | | | | | | |

The genotype frequencies in the schizophrenia, PTSD, alcohol dependence and nicotine dependence groups also differed significantly from expected values compared to controls although it is interesting to note that the heterozygote frequencies are approximately the same in the patient groups compared to the control group (Table 9).

TABLE 9
Observed and expected genotype numbers

| | 957C > T | | | Taq1A | | | −141delC | | |
|---|---|---|---|---|---|---|---|---|---|
| Alleles | C, C | C, T | T, T | A1, A1 | A1, A2 | A2, A2 | C, C | C, CC | CC, CC |
| control | 40 | 112 | 74 | 8 | 61 | 154 | 4 | 40 | 168 |
| Schizophrenia | 51 | 76 | 33 | 9 | 58 | 92 | 0 | 34 | 123 |
| Expected | 28.9 | 78.2 | 52.9 | 4.7 | 45.4 | 108.8 | 2.0 | 31.5 | 123.5 |
| PTSD | 28 | 56 | 26 | 4 | 39 | 67 | 1 | 16 | 82 |
| Expected | 19.8 | 53.8 | 36.4 | 3.3 | 31.4 | 75.3 | 1.3 | 19.9 | 77.9 |
| Alcohol | 62 | 106 | 63 | 11 | 71 | 141 | 10 | 42 | 173 |
| dependence | | | | | | | | | |
| Expected | 41.7 | 112.9 | 76.4 | 6.6 | 63.7 | 152.6 | 2.9 | 45.2 | 176.9 |
| Nicotine | 38 | 66 | 38 | 7 | 56 | 84 | 1 | 25 | 107 |
| dependence | | | | | | | | | |
| Expected | 25.6 | 69.4 | 47.0 | 4.4 | 42.0 | 100.6 | 1.7 | 26.7 | 104.6 |
| Opiate | 24 | 57 | 37 | 4 | 38 | 72 | 2 | 23 | 85 |
| dependence | | | | | | | | | |
| Expected | 21.3 | 57.7 | 39.0 | 3.4 | 32.6 | 78.0 | 1.4 | 22.1 | 86.5 |

TABLE 10
Genotype data analysis (Goodness of fit Chi-squared)

| | 957C > T | | Taq1A | | −141delC | |
|---|---|---|---|---|---|---|
| | chi squared | P value | chi squared | P value | chi squared | P value |
| Schizophrenia | 24.54 | 0.000005 | 9.92 | 0.007020 | 2.21 | 0.331460 |
| PTSD | 6.41 | 0.040524 | 2.90 | 0.234686 | 1.03 | 0.596260 |
| Alcohol | 12.69 | 0.001759 | 4.57 | 0.101538 | 17.87 | 0.000131 |
| Dependence | | | | | | |
| Nicotine | 7.86 | 0.019606 | 8.98 | 0.011210 | 0.46 | 0.796355 |
| Dependence | | | | | | |
| Opiate | 0.46 | 0.794861 | 1.48 | 0.477209 | 0.31 | 0.855947 |
| Dependence | | | | | | |

### Haplotype Analysis

In order to fully explore the presence of alleles that are associated with disease the data for the individual SNP genotypes was analyzed to generate haplotypes for the 3 SNPs in the control group and each of the patient groups using the PHASE program. The haplotypes were generated for all 3 SNPs at once, to give a total of 8 possible haplotypes or 2 at a time in 3 possible combinations to give a total of 4 possible haplotypes (Table 11).

TABLE 11
Summary of haplotype data generated using PHASE v2.1.1

| (SNPS −141delC, 957C > T, Taq1A) | | | | | −141delC (CC = 1, C = 2), 957C > T (T = 1, C = 2), TaqA1 (A2 = 1, A1 = 2) | | | | | | | | | |
|---|---|---|---|---|---|---|---|---|---|---|---|---|---|---|
| Control 3 SNPs | | | | | Schizophrenin 3 SNPs | | | | | PTSD 3 SNPs | | | | |
| n = 229 | Haplotype | Number | E (freq) | SE | n = 160 | Haplotype | Number | E (freq) | SE | n = 110 | Haplotype | Number | E (freq) | SE |
| 1 | 122 | 51 | 0.0871 | 0.0116 | 1 | 122 | 69 | 0.204 | 0.008 | 1 | 122 | 41 | 0.171 | 0.0104 |
| 2 | 221 | 43 | 0.0912 | 0.0064 | 2 | 221 | 29 | 0.088 | 0.006 | 2 | 221 | 15 | 0.068 | 0.0073 |
| 3 | 211 | 1 | 0.0083 | 0.0043 | 3 | 211 | 5 | 0.019 | 0.006 | 3 | 211 | 3 | 0.018 | 0.0061 |
| 4 | 212 | 2 | 0.0037 | 0.0035 | 4 | 212 | 0 | 4E−04 | 0.001 | 4 | 212 | 0 | 8E−04 | 0.0017 |
| 5 | 121 | 96 | 0.2345 | 0.0139 | 5 | 121 | 80 | 0.264 | 0.009 | 5 | 121 | 56 | 0.269 | 0.0133 |
| 6 | 222 | 2 | 0.0073 | 0.0039 | 6 | 222 | 0 | 5E−04 | 0.001 | 6 | 222 | 0 | 7E−04 | 0.0018 |
| 7 | 111 | 239 | 0.4933 | 0.0114 | 7 | 111 | 130 | 0.392 | 0.008 | 7 | 111 | 99 | 0.431 | 0.0117 |
| 8 | 112 | 22 | 0.0747 | 0.0111 | 8 | 112 | 7 | 0.033 | 0.007 | 8 | 112 | 6 | 0.041 | 0.0099 |
| Alcohol Dependence 3 SNPs | | | | | Nicotine Dependence 3 SNPs | | | | | Opiate Dependence 3 SNPs | | | | |
| n = 229 | Haplotype | Number | E (freq) | SE | n = 149 | Haplotype | Number | E (freq) | SE | n = 118 | Haplotype | Number | E (freq) | SE |
| 1 | 122 | 86 | 0.1786 | 0.0068 | 1 | 122 | 57 | 0.178 | 0.008 | 1 | 122 | 36 | 0.141 | 0.0115 |
| 2 | 221 | 43 | 0.0897 | 0.0054 | 2 | 221 | 22 | 0.08 | 0.008 | 2 | 221 | 23 | 0.095 | 0.0078 |
| 3 | 211 | 17 | 0.0378 | 0.0062 | 3 | 211 | 2 | 0.012 | 0.006 | 3 | 211 | 1 | 0.007 | 0.0067 |
| 4 | 212 | 0 | 0.0018 | 0.0022 | 4 | 212 | 0 | 4E−04 | 0.001 | 4 | 212 | 1 | 0.005 | 0.0044 |
| 5 | 121 | 97 | 0.221 | 0.0063 | 5 | 121 | 68 | 0.237 | 0.011 | 5 | 121 | 42 | 0.19 | 0.0109 |
| 6 | 222 | 1 | 0.0063 | 0.0048 | 6 | 222 | 3 | 0.01 | 0.003 | 6 | 222 | 3 | 0.014 | 0.0076 |
| 7 | 111 | 206 | 0.4402 | 0.0072 | 7 | 111 | 138 | 0.442 | 0.01 | 7 | 111 | 124 | 0.505 | 0.0108 |
| 8 | 112 | 8 | 0.0245 | 0.0054 | 8 | 112 | 8 | 0.042 | 0.008 | 8 | 112 | 6 | 0.043 | 0.0099 |
| (SNPS −141delC, 957C > T) | | | | | −141delC (CC = 1, C = 2), 957C > T (T = 1, C = 2) | | | | | | | | | |
| Control 2 SNPs | | | | | Schizophrenin 2 SNPs | | | | | PTSD 2 SNPs | | | | |
| | Haplotype | Number | E (freq) | SE | | Haplotype | Number | E (freq) | SE | | Haplotype | Number | E (freq) | SE |
| 1 | 11 | 261 | 0.5688 | 0.0038 | 1 | 11 | 139 | 0.413 | 0.009 | 1 | 11 | 105 | 0.465 | 0.0061 |
| 2 | 12 | 147 | 0.3166 | 0.0049 | 2 | 12 | 147 | 0.48 | 0.009 | 2 | 12 | 97 | 0.451 | 0.0069 |
| 3 | 21 | 3 | 0.0104 | 0.0049 | 3 | 21 | 3 | 0.031 | 0.009 | 3 | 21 | 3 | 0.026 | 0.0061 |
| 4 | 22 | 45 | 0.1042 | 0.0038 | 4 | 22 | 31 | 0.076 | 0.009 | 4 | 22 | 15 | 0.058 | 0.0069 |
| Alcohol Dependence 2 SNPs | | | | | Nicotine Dependence 2 SNPs | | | | | Opiate Dependence 2 SNPs | | | | |
| | Haplotype | Number | E (freq) | SE | | Haplotype | Number | E (freq) | SE | | Haplotype | Number | E (freq) | SE |
| 1 | 11 | 220 | 0.4619 | 0.0076 | 1 | 11 | 146 | 0.482 | 0.01 | 1 | 11 | 130 | 0.546 | 0.0048 |
| 2 | 12 | 176 | 0.4001 | 0.0082 | 2 | 12 | 12 | 0.419 | 0.012 | 2 | 12 | 78 | 0.331 | 0.0052 |
| 3 | 21 | 11 | 0.0424 | 0.0076 | 3 | 21 | 2 | 0.015 | 0.007 | 3 | 21 | 2 | 0.013 | 0.0048 |
| 4 | 22 | 51 | 0.0955 | 0.0082 | 4 | 22 | 25 | 0.084 | 0.011 | 4 | 22 | 26 | 0.109 | 0.0052 |
| SNPS −957C > T, Taq1A | | | | | 957C > T (T = 1, C = 2), Taq1A (A2 = 1, A1 = 2) | | | | | | | | | |
| Control 2 SNPs | | | | | Schizophrenin 2 SNPs | | | | | PTSD 2 SNPs | | | | |
| | Haplotype | Number | E (freq) | SE | | Haplotype | Number | E (freq) | SE | | Haplotype | Number | E (freq) | SE |
| 1 | 11 | 243 | 0.4992 | 0.0084 | 1 | 11 | 135 | 0.41 | 0.007 | 1 | 11 | 102 | 0.449 | 0.0099 |
| 2 | 12 | 21 | 0.0813 | 0.0082 | 2 | 12 | 7 | 0.034 | 0.007 | 2 | 12 | 6 | 0.042 | 0.0099 |
| 3 | 21 | 136 | 0.3279 | 0.0089 | 3 | 21 | 109 | 0.353 | 0.008 | 3 | 21 | 71 | 0.337 | 0.0099 |
| 4 | 22 | 56 | 0.0916 | 0.0086 | 4 | 22 | 69 | 0.204 | 0.008 | 4 | 22 | 41 | 0.172 | 0.0099 |
| Alcohol Dependence 2 SNPs | | | | | Nicotine Dependence 2 SNPs | | | | | Opiate Dependence 2 SNPs | | | | |
| | Haplotype | Number | E (freq) | SE | | Haplotype | Number | E (freq) | SE | | Haplotype | Number | E (freq) | SE |
| 1 | 11 | 225 | 0.4833 | 0.0046 | 1 | 11 | 140 | 0.452 | 0.008 | 1 | 11 | 125 | 0.516 | 0.0094 |
| 2 | 12 | 8 | 0.0254 | 0.0046 | 2 | 12 | 8 | 0.042 | 0.007 | 2 | 12 | 7 | 0.043 | 0.0094 |
| 3 | 21 | 138 | 0.3054 | 0.0048 | 3 | 21 | 90 | 0.318 | 0.009 | 3 | 21 | 65 | 0.281 | 0.0096 |
| 4 | 22 | 87 | 0.1859 | 0.0048 | 4 | 22 | 60 | 0.188 | 0.007 | 4 | 22 | 39 | 0.159 | 0.0096 |
| SNPS −141delC, Taq1A | | | | | −14delC (CC = 1, C = 2), Taq1A (A2 = 1, A1 = 2) | | | | | | | | | |
| Control 2 SNPs | | | | | Schizophrenin 2 SNPs | | | | | PTSD 2 SNPs | | | | |
| | Haplotype | Number | E (freq) | SE | | Haplotype | Number | E (freq) | SE | | Haplotype | Number | E (freq) | SE |
| 1 | 11 | 333 | 0.7327 | 0.0047 | 1 | 11 | 212 | 0.658 | 0.005 | 1 | 11 | 155 | 0.697 | 0.0093 |
| 2 | 12 | 75 | 0.1604 | 0.0043 | 2 | 12 | 74 | 0.234 | 0.004 | 2 | 12 | 47 | 0.212 | 0.0039 |
| 3 | 21 | 46 | 0.0946 | 0.004 | 3 | 21 | 34 | 0.106 | 0.004 | 3 | 21 | 18 | 0.09 | 0.0093 |
| 4 | 22 | 2 | 0.0123 | 0.0033 | 4 | 22 | 0 | 0.002 | 0.004 | 4 | 22 | 0 | 0.002 | 0.0039 |
| Alcohol Dependence 2 SNPs | | | | | Nicotine Dependence 2 SNPs | | | | | Opiate Dependence 2 SNPs | | | | |
| | Haplotype | Number | E (freq) | SE | | Haplotype | Number | E (freq) | SE | | Haplotype | Number | E (freq) | SE |
| 1 | 11 | 304 | 0.6644 | 0.0086 | 1 | 11 | 206 | 0.691 | 0.006 | 1 | 11 | 163 | 0.704 | 0.0084 |
| 2 | 12 | 93 | 0.1995 | 0.0071 | 2 | 12 | 65 | 0.216 | 0.005 | 2 | 12 | 45 | 0.177 | 0.0081 |
| 3 | 21 | 60 | 0.1237 | 0.0088 | 3 | 21 | 25 | 0.08 | 0.006 | 3 | 21 | 27 | 0.095 | 0.008 |
| 4 | 22 | 1 | 0.0124 | 0.0071 | 4 | 22 | 2 | 0.013 | 0.004 | 4 | 22 | 1 | 0.025 | 0.0077 |

The association of the haplotypes with disease was tested by performing a 2×8 comparison table, for the 3 SNP haplotypes or a 2×4 table for the 2 SNP haplotypes. For the 3 SNP haplotypes the overall likelihood ratio chi-square gave a significant P value for schizophrenia, PTSD, alcohol dependence and nicotine dependence but there appeared to be no association with opiate dependence. For those diseases that showed a haplotype association, in each case the 122 haplotype was about 2 to 4.5 times more likely to be found with disease than the 111 or 112 haplotypes, respectively. In addition, the 211 haplotype showed a tendency to be found with disease although this was only significant for Alcohol dependence where it was approximately 10 times more likely to be found with disease than the 122 “disease haplotype” and about 40 times more likely to be found with disease than the 112 “healthy haplotype”.

TABLE 12
Analysis of Haplotype Data using a 2 by 8
Contingency Table (Compare2 Version 1.25)
Haplotype Data for 3 SNPs (−141delC, 957C > T, Taq1A)

| | | Position 1 | Position 2 | Position 3 |
|---|---|---|---|---|
| Id | Haplotype | −141delC | 957C > T | Taq1A |
| 1 | 122 | (CC = 1, C = 2) | (T = 1, C = 2) | (A2 = 1, A1 = 2) |
| 2 | 221 | | | |
| 3 | 211 | | | |
| 4 | 212 | | | |
| 5 | 121 | | | |
| 6 | 222 | | | |
| 7 | 111 | | | |
| 8 | 112 | | | |

Legend for the Data Presented Below

Haplotype Data for 3 SNPs (−141delC,
957C > T, Taq1A) - Control vs. Schizophrenia
Comparisons with category 1 (likelihood-ratio tests):

| Categ. | Odds ratio | Chi-sq. | P (multiple comparisons) |
|---|---|---|---|
| 2 | 2.006 | 5.367 | P = 0.144 |
| 3 | 0.271 | 1.760 | P = 1.000 ** |
| 4 | infinity | 3.378 | P = 0.462 ** |
| 5 | 1.624 | 4.153 | P = 0.291 |
| 6 | infinity | 3.378 | P = 0.462 ** |
| 7 | 2.487 | 18.360 | P = 0.000 [1.3E−4] |
| 8 | 4.252 | 10.798 | P = 0.007 [7.1E−3] |

Chi-square test (DF = 7): Likelihood-ratio chi-sq. * = 32.235 P = 0.000 [3.7E−5]
* 0.0000001 added to zero cells to permit computation.
* Zero frequencies changed to 0.0000001
** At least one cell has an expected frequency < 5

Haplotype Data for 3 SNPs (−141delC,
957C > T, Taq1A) - Control vs. PTSD
Comparisons with category 1 (likelihood-ratio tests):

| Categ. | Odds ratio | Chi-sq. | P (multiple comparisons) |
|---|---|---|---|
| 2 | 2.305 | 5.455 | P = 0.137 |
| 3 | 0.268 | 1.468 | P = 1.000 ** |
| 4 | infinity | 2.326 | P = 0.891 ** |
| 5 | 1.378 | 1.422 | P = 1.000 |
| 6 | infinity | 2.326 | P = 0.891 ** |
| 7 | 1.941 | 7.419 | P = 0.045 |
| 8 | 2.948 | 5.131 | P = 0.165 |

Chi-square tests (DF = 7): Likelihood-ratio chi-sq. * = 17.771 P = 0.013
* 0.0000001 added to zero cells to permit computation.

Haplotype Data for 3 SNPs (−141delC, 957C > T,
Taq1A) - Control vs. Alcohol Dependence
Comparisons with category 1 (likelihood-ratio tests):

| Categ. | Odds ratio | Chi-sq. | P (multiple comparisons) |
|---|---|---|---|
| 2 | 1.686 | 3.525 | P = 0.423 |
| 3 | 0.099 | 9.173 | P = 0.017 |
| 4 | infinity | 3.904 | P = 0.337 ** |
| 5 | 1.669 | 5.112 | P = 0.166 |
| 6 | 3.373 | 1.041 | P = 1.000 ** |
| 7 | 1.956 | 11.484 | P = 0.005 [4.9E−3] |
| 8 | 4.637 | 13.188 | P = 0.002 [2.0E−3] |

Chi-square tests (DF = 7): Likelihood-ratio chi-sq. * = 38.627 P = 0.000 [2.3E−6]
* 0.0000001 added to zero cell to permit computation.
* Zero frequencies changed to 0.0000001
** At least one cell has an expected frequency < 5

Haplotype Data for 3 SNPs (−141delC, 957C > T,
Taq1A) - Control vs. Nicotine Dependence
Comparisons with category 1 (likelihood-ratio tests):

| Categ. | Odds ratio | Chi-sq. | P (multiple comparisons) |
|---|---|---|---|
| 2 | 2.184 | 5.939 | P = 0.104 |
| 3 | 0.559 | 0.232 | P = 1.000 ** |
| 4 | infinity | 2.961 | P = 0.597 ** |
| 5 | 1.578 | 3.356 | P = 0.469 |
| 6 | 0.745 | 0.101 | P = 1.000 ** |
| 7 | 1.936 | 8.996 | P = 0.019 |
| 8 | 3.074 | 6.663 | P = 0.069 |

Chi-square tests (DF = 7): Likelihood-ratio chi-sq.* = 16.189 P = 0.023
* 0.0000001 added to zero cell to permit computation.
* Zero frequencies changed to 0.0000001
** At least one cell has an expected frequency < 5

Haplotype Data for 3 SNPs (−141delC, 957C > T,
Taq1A) - Control vs. Opiate Dependence
Comparisons with category 1 (likelihood-ratio tests):

| Categ. | Odds ratio | Chi-sq. | P (multiple comparisons) |
|---|---|---|---|
| 2 | 1.320 | 0.678 | P = 1.000 |
| 3 | 0.706 | 0.059 | P = 1.000 ** |
| 4 | 1.412 | 0.079 | P = 1.000 ** |
| 5 | 1.613 | 2.800 | P = 0.660 |
| 6 | 0.471 | 0.662 | P = 1.000 ** |
| 7 | 1.361 | 1.571 | P = 1.000 |
| 8 | 2.588 | 3.858 | P = 0.347 |

Chi-square tests (DF = 7): Likelihood-ratio chi-sq. = 6.620 P = 0.470
** At least one cell has an expected frequency < 5

For the -141delC/957C>T 2 SNP haplotypes the overall likelihood ratio chi-square gave a significant or nearly significant P value for schizophrenia, PTSD, alcohol dependence and nicotine dependence but there appeared to be no association with opiate dependence (Table 13). However, none of the individual haplotypes showed a significant association with disease relative to the -141delC, C/957C>T, C haplotype.

TABLE 13
Analysis of Haplotype Data using a 2 by 4
Contingency Table (Compare2 Version 1.25)
Haplotype Data for 2 SNPs at a time
(SNPs in the haplotype are indicated above the analysis)

| | | Position 1 | Position 1/2 | Position 2 |
|---|---|---|---|---|
| Id | Haplotype | −141delC | 957C > T | Taq1A |
| 1 | 22 | (CC = 1, C = 2) | (T = 1, C = 2) | (A2 = 1, A1 = 2) |
| 2 | 21 | | | |
| 3 | 12 | | | |
| 4 | 11 | | | |

* The risk group haplotype (22) is used as the reference group in this analysis

The following data are a combination of 2 SNPs of the choices shown above

Haplotype Data for 2 SNPs (−141delC,
957C > T) - Control vs. Schizophrenia
Comparisons with category 1 (likelihood-ratio tests):

| Categ. | Odds ratio | Chi-sq. | P (multiple comparisons) |
|---|---|---|---|
| 2 | 0.689 | 0.192 | P = 1.000 ** |
| 3 | 0.689 | 2.064 | P = 0.452 |
| 4 | 1.294 | 0.999 | P = 0.952 |

Chi-square tests (DF = 3) Likelihood-ratio chi-sq. = 16.445 P = 0.001 [9.2E−4]
** At least one cell has an expected frequency < 5

Haplotype Data for 2 SNPs (−141delC,
957C > T) - Control vs. PTSD
Comparisons with category 1 (likelihood-ratio tests):

| Categ. | Odds ratio | Chi-sq. | P (multiple comparisons) |
|---|---|---|---|
| 2 | 0.333 | 1.548 | P = 0.640 ** |
| 3 | 0.505 | 4.714 | P = 0.090 |
| 4 | 0.829 | 0.354 | P = 1.000 |

Chi-square tests (DF = 3): Likelihood-ratio chi-sq. = 10.532 P = 0.015
** At least one cell has an expected frequency < 5

Haplotype Data for 2 SNPs (−141delC,
957C > T) - Control vs. Alcohol Dependence
Comparisons with category 1 (likelihood-ratio tests):

| Categ. | Odds ratio | Chi-sq. | P (multiple comparisons) |
|---|---|---|---|
| 2 | 0.309 | 3.448 | P = 0.190 |
| 3 | 0.947 | 0.055 | P = 1.000 |
| 4 | 1.345 | 1.750 | P = 0.558 |

Chi-square tests (DF = 3): Likelihood-ratio chi-sq. = 11.337 P = 0.010

Haplotype Data for 2 SNPs (−141delC,
957C > T) - Control vs. Nicotine Dependence
Comparisons with category 1 (likelihood-ratio tests):

| Categ. | Odds ratio | Chi-sq. | P (multiple comparisons) |
|---|---|---|---|
| 2 | 0.833 | 0.037 | P = 1.000 ** |
| 3 | 0.653 | 2.405 | P = 0.363 |
| 4 | 0.993 | 0.001 | P = 1.000 |

Chi-square tests (DF = 3): Likelihood-ratio chi-sq. = 7.363 P = 0.061
** At least one cell has an expected frequency < 5

Haplotype Data for 2 SNPs (−141delC,
957C > T) - Control vs. Opiate Dependence
Comparisons with category 1 (likelihood-ratio tests):

| Categ. | Odds ratio | Chi-sq. | P (multiple comparisons) |
|---|---|---|---|
| 2 | 0.867 | 0.023 | P = 1.000 ** |
| 3 | 1.089 | 0.090 | P = 1.000 |
| 4 | 1.160 | 0.302 | P = 1.000 |

Chi-square tests (DF = 3): Likelihood-ratio chi-sq. = 0.433 P = 0.933
** At least one cell has an expected frequency < 5

Haplotype Data for 2 SNPs (957C > T,
Taq1A) - Control vs. Schizophrenia
Comparisons with category 1 (likelihood-ratio tests):

| Categ. | Odds ratio | Chi-sq. | P (multiple comparisons) |
|---|---|---|---|
| 2 | 1.537 | 3.806 | P = 0.153 |
| 3 | 3.696 | 8.673 | P = 0.010 [9.7E−3] |
| 4 | 2.218 | 14.595 | P = 0.000 [4.0E−4] |

Chi-square tests (DF = 3): Likelihood-ratio chi-sq. = 18.994 P = 0.000 [2.7E−4]

Haplotype Data for 2 SNPs (957C > T,
Taq1A) - Control vs. PTSD
Comparisons with category 1 (likelihood-ratio tests):

| Categ. | Odds ratio | Chi-sq. | P (multiple comparisons) |
|---|---|---|---|
| 2 | 1.402 | 1.786 | P = 0.544 |
| 3 | 2.563 | 3.824 | P = 0.152 |
| 4 | 1.744 | 5.414 | P = 0.060 |

Chi-square tests (DF = 3): Likelihood-ratio chi-sq. = 7.111 P = 0.068

Haplotype Data for 2 SNPs (957C > T,
Taq1A) - Control vs. Alcohol Dependence
Comparisons with category 1 (likelihood-ratio tests):

| Categ. | Odds ratio | Chi-sq. | P (multiple comparisons) |
|---|---|---|---|
| 2 | 1.531 | 4.174 | P = 0.123 |
| 3 | 4.078 | 10.927 | P = 0.003 [2.8E−3] |
| 4 | 1.678 | 7.190 | P = 0.022 |

Chi-square tests (DF = 3): Likelihood-ratio chi-sq. = 13.517 P = 0.004 [3.6E−3]

Haplotype Data for 2 SNPs (957C > T,
Taq1A) - Control vs. Nicotine Dependence
Comparisons with category 1 (likelihood-ratio tests):

| Categ. | Odds ratio | Chi-sq. | P (multiple comparisons) |
|---|---|---|---|
| 2 | 1.619 | 4.396 | P = 0.108 |
| 3 | 2.813 | 5.619 | P = 0.053 |
| 4 | 1.860 | 8.409 | P = 0.011 |

Chi-square tests (DF = 3): Likelihood-ratio chi-sq. = 10.295 P = 0.016

Haplotype Data for 2 SNPs (957C > T,
Taq1A) - Control vs. Opiate Dependence
Comparisons with category 1 (likelihood-ratio tests):

| Categ. | Odds ratio | Chi-sq. | P (multiple comparisons) |
|---|---|---|---|
| 2 | 1.457 | 2.126 | P = 0.435 |
| 3 | 2.089 | 2.486 | P = 0.345 |
| 4 | 1.354 | 1.632 | P = 0.604 |

Chi-square tests (DF = 3): Likelihood-ratio chi-sq. = 3.356 P = 0.340

Haplotype Data for 2 SNPs (−141delC,
Taq1A) - Control vs. Schizophrenia
Comparisons with category 1 (likelihood-ratio tests):

| Categ. | Odds ratio | Chi-sq. | P (multiple comparisons) |
|---|---|---|---|
| 2 | 0.000 | 2.177 | P = 0.420 ** |
| 3 | 0.000 | 2.720 | P = 0.297 ** |
| 4 | 0.000 | 1.966 | P = 0.483 ** |

Chi-square tests (DF = 3): Likelihood-ratio chi-sq.* = 7.717 P = 0.052
* 0.0000001 added to zero cell to permit computation.
* Zero frequencies changed to 0.0000001
** At least one cell has an expected frequency < 5

Haplotype Data for 2 SNPs (−141delC,
Taq1A) - Control vs. PTSD
Comparisons with category 1 (likelihood-ratio tests):

| Categ. | Odds ratio | Chi-sq. | P (multiple comparisons) |
|---|---|---|---|
| 2 | 0.000 | 1.297 | P = 0.764 ** |
| 3 | 0.000 | 1.926 | P = 0.496 ** |
| 4 | 0.000 | 1.685 | P = 0.583 ** |

Chi-square tests (DF = 3): Likelihood-ratio chi-sq.* = 3.743 P = 0.291
* 0.0000001 added to zero cell to permit computation.
* Zero frequencies changed to 0.0000001
** At least one cell has an expected frequency < 5

Haplotype Data for 2 SNPs (−141delC,
Taq1A) - Control vs. Alcohol Dependence
Comparisons with category 1 (likelihood-ratio tests):

| Categ. | Odds ratio | Chi-sq. | P (multiple comparisons) |
|---|---|---|---|
| 2 | 0.383 | 0.640 | P = 1.000 * |
| 3 | 0.403 | 0.579 | P = 1.000 ** |
| 4 | 0.548 | 0.254 | P = 1.000 ** |

Chi-square tests (DF = 3): Likelihood-ratio chi-sq. = 5.443 P = 0.142
** At least one cell has an expected frequency < 5

Haplotype Data for 2 SNPs (−141delC,
Taq1A) - Control vs. Nicotine Dependence
Comparisons with category 1 (likelihood-ratio tests):

| Categ. | Odds ratio | Chi-sq. | P (multiple comparisons) |
|---|---|---|---|
| 2 | 1.840 | 0.346 | P = 1.000 ** |
| 3 | 1.154 | 0.020 | P = 1.000 ** |
| 4 | 1.617 | 0.227 | P = 1.000 ** |

Chi-square tests (DF = 3): Likelihood-ratio chi-sq. = 3.872 P = 0.276
** At least one cell has an expected frequency < 5

Haplotype Data for 2 SNPs (−141delC,
Taq1A) - Control vs. Opiate Dependence
Comparisons with category 1 (likelihood-ratio tests):

| Categ. | Odds ratio | Chi-sq. | P (multiple comparisons) |
|---|---|---|---|
| 2 | 0.852 | 0.017 | P = 1.000 ** |
| 3 | 0.833 | 0.022 | P = 1.000 ** |
| 4 | 1.021 | 0.000 | P = 1.000 ** |

Chi-square tests (DF = 3): Likelihood-ratio chi-sq. = 1.21 P = 0.749
** At least one cell bas an expected frequency < 5

For the -141 delC/Taq1A 2 SNP haplotypes the overall likelihood ratio chi-square was not significantly more likely to be found with disease for any of the diseases studied. The 957C>T/Taq1A 2 SNP haplotypes showed the strongest association with disease. The overall likelihood ratio chi-square values gave strongly significant P values for schizophrenia, alcohol dependence and nicotine dependence, and nearly significant P values for PTSD but there appeared to be no association with opiate dependence. Most of the 957C>T/Taq1A individual haplotypes also showed very strong and significant association relative to the 22 (957C>T, C/Taq1A, A1) “disease haplotype”. In particular, the 22 haplotype was 2.5 to 4 times more likely to be found with disease than the 12 (957C>T, T/Taq1A, A1) haplotype for all disease groups except opiate dependence.

In conclusion, it is clear that the 957C>T polymorphism is very strongly associated with 4 disease groups, schizophrenia, PTSD, alcohol dependence and nicotine dependence. The Taq1A polymorphism is showing the same pattern of association although the strength of the association is weaker, especially for alcohol dependence and PTSD but the -141delC polymorphism is not showing any association with any of the disease groups, possibly because the polymorphism has a low allele frequency. The use of haplotypes containing 2 or 3 SNPs greatly increases the power to detect alleles that are associated with disease. For example the 122 and the 221 haplotypes (-141delC, CC/957C>T, C/Taq1A, A1 and -141delC, C/957C>T, C/Taq1A, A2; respectively) appear to be strongly associated with disease relative to the 111 and 112 haplotypes (-141delC, CC/957C>T, T/Taq1A, A2 and -141delC, CC/957C>T, T/Taq1A, A1; respectively).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Abi-Dargham et al., Proc. Natl. Acad. Sci. 97:8104-8109, 2000
Brennan M D. AM J Pharmacogenomics. 1(4):295-302, 2001
Callicott et. al., Am. J. Psychiatry. 160:709-719, 2003
Cariello Am. J. Human Genetics 42:726-734, 1988
Conner et al., Proc. Natl. Acad. Sci. USA 80:278-282, 1983
Cotton et al. Proc. Natl. Acad. Sci. USA 87:4033-40371988
Edwards et al., Methods Mol. Biol. 226:287-94, 2003
Elghanian et al. Science 277:1078-1081, 1997
Finkelstein et al. Genomics 7:167-172, 1990
Grompe et al. Proc. Natl. Acad. Sci. USA 86:5855-5892, 1989
Kelly et al., Ir. J. Med. Sci. 172:37-40, 2003
Kinszler et al. Science 251:1366-1370, 1991
Kwok et al., Curr Issues Mol Biol. 5(2):43-60, 2003
MacDonald et al., Arch. Gen. Psychiatry. 60:57-65, 2003
Miyamoto et al., Mol. Intervent. 3:27-39, 2004
Modrich Ann. Rev. Genet. 25:229-253, 1991
Mowry B J et. al., Mol. Psychiatry. 2004
Niendam et. al., Am. J. Psychiatry. 160:2060-2062, 2003
Novack et al. Proc. Natl. Acad. Sci. USA 83:586-590, 1986
Orita, et al., Proc. Natl. Acad. Sci. USA. 86:2766-2770, 1989
Owen et al., Mol. Psychiatry. 9:14-27, 2004
Ruano and Kidd Nucl. Acids Res. 17:8392, 1989
Seeman and Kapur Proc. Natl. Acad. Sci. USA 97:7673-7675, 2000
Sheffield et al. Proc. Natl. Acad. Sci. USA 86:232-236, 1989
Shenk et al. Proc. Natl. Acad. Sci. USA 72:989-993, 1975
Taylor et al. Biotechniques 30:661-669, 2001
Tepest et al., Biol, Psychiatry. 54:1234-1240, 2003
Twyman et al., Pharmacogenomics. 4(1):67-79, 2003
Wartell et al. Nucl. Acids Res. 18:2699-2705, 1990
White et al. Genomics 12:301-306, 1992
Winterer et. al., Arch. Gen. Psychiatry. 60:1158-1167, 2003

1. A method for identifying a genetic profile associated with a neurological, psychiatric or psychological condition, phenotype or state including a sub-threshold neurological, psychiatric or psychological condition, phenotype or state in an individual or within a group of individuals, said method comprising screening individuals for a polymorphism in a genetic locus comprising the DRD2 gene including its 5′ and 3′ terminal regions, promoter, introns and exons whilst has a statistically significant linkage or association to symptoms or behaviour characterizing the neurological, psychiatric or psychological condition, phenotype or state or sub-threshold forms thereof.

2. The method of claim 1 wherein the neurological, psychiatric or psychological condition, phenotype or state is selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder, Smoking Dependence and Social Phobia.

3. The method of claim 2 wherein the neurological, psychiatric or psychological condition, phenotype or state is schizophrenia or a related condition or a condition with similar symptoms.

4. The method of claim 3 wherein the neurological, psychiatric or psychological condition, phenotype or state is schizophrenia.

5. The method of claim 1 or 2 or 3 or 4 wherein the polymorphism is at a nucleotide position listed in Table 2.

6. The method of claim 5 wherein the polymorphism is at nucleotide position 957 of the cDNA sequence encoding DRD2.

7. The method of claim 6 wherein the nucleotide at position 957 is a C.

8. The method of claim 6 wherein the nucleotide at position 957 is a T.

9. The method of claim 6 wherein the nucleotide at position 957 is a G.

10. The method of claim 6 wherein the nucleotide at position 957 is an A.

11. The method of claim 6 or 7 or 8 or 9 or 10 wherein the polymorphism is at two genetic loci.

12. The method of claim 11 wherein the second genetic locus is Taq A1.

13. A genetic mutation providing a genetic marker for a neurological, psychiatric or psychological condition, state or phenotype in an individual said genetic marker comprises a C at nucleotide position 957 wherein the presence of a 957C polymorphism is indicative of or a predisposition to developing a neurological, psychiatric or psychological condition, phenotype or state selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder, Smoking Dependence and Social Phobia.

14. The genetic mutation of claim 13 wherein the neurological, psychiatric or psychological condition, phenotype or state in schizophrenia or a selected condition or a condition with similar symptoms.

15. The genetic mutation of claim 14 wherein the neurological, psychiatric or psychological condition, phenotype or state is schizophrenia.

16. A The method for identifying a genetic profile consistent with a neurological, psychiatric or psychological condition, phenotype or state in a individual, said method comprising obtaining or extracting a DNA sample from cells of said individual and screening for or otherwise detecting the presence of a genetic profile in the DRD2 genetic locus including its 5′ or 3′ terminal region, promoter, introns or exons having a statistical significant association with a particular neurological, psychiatric or psychological condition, phenotype or state wherein the presence of that genetic profile is indicative of the neurological, psychiatric or psychological condition, phenotype or state or a sub-threshold form thereof or that the individual is at risk of developing same.

17. The method of claim 16 wherein the neurological, psychiatric or psychological condition, phenotype or state is selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder, Smoking Dependence and Social Phobia.

18. The method of claim 17 wherein the neurological, psychiatric or psychological condition, phenotype or state is schizophrenia or a related condition or a condition with similar symptoms.

19. The method of claim 18 wherein the neurological, psychiatric or psychological condition, phenotype or state is schizophrenia.

20. The method of claim 16 or 17 or 18 or 19 wherein the polymorphism is at a nucleotide position listed in Table 2.

21. The method of claim 20 wherein the polymorphism is at nucleotide position 957 of the cDNA sequence encoding DRD2.

22. The method of claim 21 wherein the polymorphism is a C>T substitution at one or both alleles of the DRD2 genetic locus.

23. A method for identifying a genetic basis behind diagnosing a neurological, psychiatric or psychological condition, phenotype or state in an individual, said method comprising obtaining or extracting a DNA sample from cells of said individual and screening for or otherwise detecting the presence of a genetic profile in the DRD2 genetic locus including its 5′ or 3′ terminal region, promoter, intron or exons which with a statistical significant association with a particular neurological, psychiatric or psychological condition, phenotype or state wherein the presence of that genetic profile is indicative of the neurological, psychiatric or psychological condition, phenotype or state or a sub-threshold form thereof or that the individual is at risk of developing same.

24. The method of claim 23 wherein the neurological, psychiatric or psychological condition, phenotype or state is selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder, Smoking Dependence and Social Phobia.

25. The method of claim 24 wherein the neurological, psychiatric or psychological condition, phenotype or state is schizophrenia or a related condition or a condition with similar symptoms.

26. The method of claim 25 wherein the neurological, psychiatric or psychological condition, phenotype or state is schizophrenia.

27. The method of claim 23 or 24 or 25 or 26 wherein the polymorphism is at a nucleotide position listed in Table 2.

28. The method of claim 27 wherein the polymorphism is at nucleotide position 957 of the cDNA sequence encoding DRD2.

29. The method of claim 28 wherein the polymorphism is a C>TC substitution at one or both alleles of the DRD2 genetic locus.

30. An isolated oligonucleotide which comprises from about 3 to about 100 consecutive nucleotides from the DRD2 genetic locus and which encompass at least one polymorphism or mutation associated with or otherwise likely to be found in individuals with a particular neurological, psychiatric or psychological condition, phenotype or state such as those selected from Addiction, Alzheimer's Disease, Anxiety Disorders, Attention Deficit Hyperactivity Disorder (ADHD), Eating Disorders, Manic-Depressive Illness, Autism, Schizophrenia, Tourette's Syndrome, Obsessive Compulsive Disorder (OCD), Panic Disorder, Post Traumatic Stress Disorder (PTSD), Phobias, borderline personality disorder, bi-polar disorder, sleep disorders, Acute Stress Disorder, Adjustment Disorder, Agoraphobia Without History of Panic Disorder, Alcohol Dependence (Alcoholism), Amphetamine Dependence, Anorexia Nervosa, Antisocial Personality Disorder, Asperger's Disorder, Avoidant Personality Disorder, Brief Psychotic Disorder, Bulimia Nervosa, Cannabis Dependence, Cocaine Dependence, Conduct Disorder, Cyclothymic Disorder, Delirium, Delusional Disorder, Dementia Associated With Alcoholism, Dementia of the Alzheimer Type, Dependent Personality Disorder, Dysthymic Disorder, Generalized Anxiety Disorder, Hallucinogen Dependence, Histrionic Personality Disorder, Inhalant Dependence, Major Depressive Disorder, Manic Depression, Multi-Infarct Dementia, Narcissistic Personality Disorder, Nicotine Dependence, Opioid Dependence, Oppositional Defiant Disorder, Panic Disorder, Paranoid Personality Disorder, Parkinson's Disease, Phencyclidine Dependence, Schizoaffective Disorder, Schizoid Personality Disorder, Schizophreniform Disorder, Schizotypal Personality Disorder, Sedative Dependence, Separation Anxiety Disorder, Shared Psychotic Disorder, Smoking Dependence and Social Phobia.

31. The isolated oligonucleotide of claim 30 wherein the oligonucleotide is selected from SEQ ID NO:8 through to SEQ ID NO:2616.

32. The isolated oligonucleotide of claim 31 wherein the oligonucleotide is SEQ ID NO: 5 or 6.
